SEQUENCE LISTING (11 sequences)

SEQ ID NO: 1 - DNA, 21 bp, synthetic construct
  source 1..21: mol_type = other DNA
  misc_feature 1..21: note = reverse primer (5'-3') (miR-17-5p; miR-19b-3p; miR-21-5p; miR-15b-5p; miR-19a-3p; miR-664a-3p; miR-92a-3p; miR-421)
gtcgtatccagtgcagggtcc

SEQ ID NO: 2 - DNA, 19 bp, synthetic construct
  source 1..19: mol_type = other DNA
  misc_feature 1..19: note = miR-652-3p reverse primer (5'-3')
gtcgtatccagtgcagggt

SEQ ID NO: 3 - DNA, 22 bp, synthetic construct
  source 1..22: mol_type = other DNA
  misc_feature 1..22: note = miR-17-5p forward primer (5'-3')
cgacaggccaaagtgcttacag

SEQ ID NO: 4 - DNA, 22 bp, synthetic construct
  source 1..22: mol_type = other DNA
  misc_feature 1..22: note = miR-19b-3p forward primer (5'-3')
accgaggttgtgcaaatccatg

SEQ ID NO: 5 - DNA, 23 bp, synthetic construct
  source 1..23: mol_type = other DNA
  misc_feature 1..23: note = miR-21-5p forward primer (5'-3')
accaccgtagcttatcagactga

SEQ ID NO: 6 - DNA, 20 bp, synthetic construct
  source 1..20: mol_type = other DNA
  misc_feature 1..20: note = miR-15b-5p forward primer (5'-3')
accaccgtagcagcacatca

SEQ ID NO: 7 - DNA, 22 bp, synthetic construct
  source 1..22: mol_type = other DNA
  misc_feature 1..22: note = miR-19a-3p forward primer (5'-3')
gcggcctgtgtgcaaatctatg

SEQ ID NO: 8 - DNA, 22 bp, synthetic construct
  source 1..22: mol_type = other DNA
  misc_feature 1..22: note = miR-664a-3p forward primer (5'-3')
aggcgtgctattcatttatccc

SEQ ID NO: 9 - DNA, 21 bp, synthetic construct
  source 1..21: mol_type = other DNA
  misc_feature 1..21: note = miR-92a-3p forward primer (5'-3')
ccaggccatattgcacttgtc

SEQ ID NO: 10 - DNA, 21 bp, synthetic construct
  source 1..21: mol_type = other DNA
  misc_feature 1..21: note = miR-652-3p forward primer (5'-3')
aagtactcaatggcgccacta

SEQ ID NO: 11 - DNA, 23 bp, synthetic construct
  source 1..23: mol_type = other DNA
  misc_feature 1..23: note = miR-421 forward primer (5'-3')
agccagcgatcaacagacattaa

(19) United States
(12) Patent Application Publication
(10) Pub. No.: US 2025/0263793 A1
(43) Pub. Date: Aug. 21, 2025

(54) METHODS OF DETECTING AND TREATING GYNECOLOGICAL DISEASES

(71) Applicant: Heranova Lifesciences, Inc., Jacksonville, FL (US)

(72) Inventors: Farideh Bischoff, Houston, TX (US); Wing Hing Wong, Menlo Park, CA (US)

(21) Appl. No.: 19/010,140

(22) Filed: Jan. 5, 2025

Related U.S. Application Data
(60) Provisional application No. 63/617,758, filed on Jan. 4, 2024.
(60) Provisional application No. 63/654,016, filed on May 30, 2024.

(51) Int. Cl. C12Q 1/6883; G01N 33/68

(52) U.S. Cl. CPC C12Q 1/6883; G01N 33/6893; C12Q 2600/158; C12Q 2600/166; C12Q 2600/178; G01N 2333/4725; G01N 2800/364

(57) ABSTRACT

Provided herein are methods of assessing whether a subject has a gynecological disease (e.g., endometriosis) or is predisposed to a gynecological disease (e.g., endometriosis), particularly at early stages, as well as methods of assessing the prognosis of a subject having endometriosis, based on levels of certain miRNAs in a sample of the subject. Also provided herein are methods of distinguishing between subjects having endometriosis and subjects having other gynecological diseases based on levels of certain miRNAs. Kits useful for performing such assessments are also provided. Methods of treating or preventing endometriosis guided by information obtained from methods disclosed herein are also provided.

### 1. CROSS-REFERENCE TO RELATED APPLICATIONS

This application claims the benefit of priority to the following applications, the entire contents of each of which are incorporated by reference herein: U.S. Provisional Application No. 63/617,758, filed Jan. 4, 2024, and U.S. Provisional Application No. 63/654,016, filed May 30, 2024.

### 2. REFERENCE TO SEQUENCE LISTING SUBMITTED ELECTRONICALLY

This application incorporates by reference a Sequence Listing entitled “522A002WO03_SL.XML” created on Dec. 31, 2024 and having a size of 15,340 bytes.

### 3. FIELD

The present invention relates to the field of molecular biology, physiology and pathology.

### 4. BACKGROUND

Endometriosis is a common gynecological disease that affects 176 million girls and women globally. Endometriosis affects 5-10% of women and adolescents within the reproductive age range of 15-49 years, and for those facing infertility, this figure can be up to 50%. Endometriosis can start at the first menstrual period and last until menopause. Between 50% and 80% of women grappling with pelvic pain are found to have endometriosis. The formation of scar tissue (such as adhesions and fibrosis) within the pelvis and other parts of the body can cause severe pain and lead to infertility. Early detection and timely intervention are the best prevention. Although most women with endometriosis report the onset of symptoms during adolescence, diagnosis is often delayed for 6 to 11 years given that the cause of endometriosis remains unknown and that current diagnostic methods are invasive with risks of infection, bleeding, and/or damage to surrounding tissue, which can result in unnecessary suffering and reduced quality of life. Symptoms are nonspecific, do not correlate with clinical stage, and often vary between patients. With a delayed diagnosis, the disease can progress, and other coexisting gynecologic conditions can develop, making diagnosis and treatment even more challenging. The lack of reliable, noninvasive methods to identify and select endometriosis subjects further hinders endometriosis-targeted therapies and clinical trials in drug development. The current standard of diagnosis is laparoscopic visualization and subsequent histological confirmation, which carries risks and is reliant on the operator's surgical experience in locating and identifying endometriosis lesions. The surgical nature of laparoscopy usually results in diagnosis delay. The condition often goes undiagnosed or misdiagnosed for years, with women often accepting years of life-altering pelvic and emotional pain. As early diagnosis and treatment can mitigate pain and prevent disease progression, non-invasive means to detect endometriosis at early onset represents unmet needs. A non-invasive mean will avoid or assist to confirm whether or not surgical intervention is necessary, sparing many women from a highly invasive and often exploratory procedure.

Additionally, gynecologic (adenomyosis, uterine fibroids, polycystic ovarian syndrome-PCOS) and systemic (autoimmune, inflammatory, psychiatric and neurological disorders) comorbidities are commonly described in patients with endometriosis. Endometriosis is sometimes mistaken for other conditions that may cause pelvic pain, such as pelvic inflammatory disease (PID) or ovarian cysts. Endometriosis is often confused with irritable bowel syndrome (IBS), which causes diarrhea, constipation, and abdominal cramping. IBS may coexist with endometriosis, which can complicate diagnosis. Unless the patient is taking estrogen, the signs and symptoms of endometriosis may temporarily improve during pregnancy and may disappear completely during menopause. Methods for early and non-invasive detection of gynecological disease, and to distinguish between endometriosis and other gynecological diseases are currently unavailable.

Methods and systems provided herein address these needs and provide related advantages.

### 5. SUMMARY

Provided herein are methods for detecting gynecological diseases, such as endometriosis, through the measurement of microRNA (miRNA) levels in biological samples. The methods involve measuring at least one miRNA from a specified panel, comparing the detected levels to reference values, and determining altered expression patterns indicative of disease. The miRNAs include but are not limited to miR-17, miR-19b, miR-21, miR-15b, miR-19a, miR-664a, miR-381, miR-23a, miR-654, miR-24, Let-7b, miR-20a, miR-22, miR-34c, miR-1287, miR-625, miR-1294, miR-7704, miR-221, miR-340, miR-450b, miR-548e, miR-502, miR-574, miR-9, miR-299, miR-6789, miR-593, miR-346, miR-34a, miR-449a, miR-7109, miR-3907, miR-557, miR-6801, miR-4420, miR-570, miR-155, miR-199, miR-520d, miR-424, and miR-200, measured in various forms (e.g., -5p, -3p). Measurement techniques include quantitative PCR (qPCR), droplet digital PCR (ddPCR), next-generation sequencing (NGS), fluorescence in situ hybridization (FISH), and other molecular assays.

The methods extend to multiplex analysis of multiple miRNAs and normalization against endogenous controls. Novel endogenous controls such as miR-92a-3p are identified and described. Samples can be derived from peripheral blood, menstrual blood, biopsy, or serum. In some embodiments, samples are collected during the secretory phase of the menstrual cycle. In some embodiments, samples are collected during the proliferative phase of the menstrual cycle. The methods can further involve assessing CA-125 levels as an additional biomarker.

Additionally, kits are described for conducting these diagnostic assays, comprising means for miRNA detection, and ancillary buffers. Kits can also include components for sample collection, processing, and detailed instructions for performing the assay.

### 6. BRIEF DESCRIPTION OF DRAWINGS

FIGS. 1A-1B provide combined expression profiles of miRNA biomarkers Let-7b-5p, miR-155-5p, miR-23a-3p, miR-17-5p, miR-20a-5p, and miR-22-5p in serum sample from normal health controls, patient controls, and endometriosis patients, as measured by qPCR. FIG. 1A provides data from batch 1 samples comparing normal healthy controls to endometriosis patients. FIG. 1B provides data from batch 2 samples comparing normal healthy controls, endometriosis patients, and patient controls.

FIGS. 2A-2D provide expression profiles of individual markers in both healthy population and disease population. FIG. 2A: Let-7b-5p; FIG. 2B: miR-155-5p; FIG. 2C: miR-23a-3p; FIG. 2D: miR-17-5p; FIG. 2E: miR-20a-5p; and FIG. 2F: miR-22-5p.

FIGS. 3A-3C provide expression profiles of four individual biomarkers from samples from patients having endometriosis and patients having other gynecological diseases. FIG. 3A: miR-155-5p; FIG. 3B: miR-23a-3p; FIG. 3C: miR-17-5p; and FIG. 3D: miR-20a-5p.

FIG. 4 provides statistical analysis for each individual marker's prediction power, including CA-125 (protein), miR-34-3P, miR-20a-5p, miR-17-5p, miR-22-3p, miR-199-5p, let-7b-5p, miR-23a-3p, miR-155-5p.

FIGS. 5A-5B provide the area under curve (AUC) value of CA-125 (Protein) and 7 models tested for endometriosis diagnosis, wherein the models included different combination of miRNAs. FIG. 5A: Model 1(34c-3p), Model 2(20a-5p+17-5p), Model 3(20a-5p+17-5p+22-3p+34c-3p), Protein (CA-125); FIG. 5B: Model 1(34c-3p), Model 5(34c-3p+20a-5p), Model 6(34c-3p+17-5p), Model 7(34c-3p+22-3p), Protein (CA-125).

FIGS. 6A-6D provide results of model testing with combination of miRNAs and protein CA-125. FIG. 6A provides AUC value of combination of miR-17-5p, miR-199-5p, miR-34c-3p and protein CA-125 as biomarkers for model testing in proliferative phase. FIG. 6B provides sensitivity and specificity of combination of miR-17-5p, miR-199a-5p, miR-34c-3p and protein CA-125 as biomarkers for model testing in proliferative phase. FIG. 6C provides AUC value of combination of miR-20a-5p, miR-22-3p, miR-34c-3p and protein CA-125 as biomarkers for model testing in secretory phase. FIG. 6D provides sensitivity and specificity of combination of miR-20a-5p, miR-22-3p, miR-34c-3p and protein CA-125 as biomarkers for model testing in secretory phase.

FIG. 7 provides data showing that the 10 miRNA biomarkers identified by ddPCR effectively distinguished between subjects with and without endometriosis. These biomarkers demonstrated consistency in both the direction of effect and data distribution across the discovery batch (1st control and 1st disease) and validation batch (2nd control and 2nd disease). The y-axis denotes the normalized values of the biomarkers against endogenous control (miR-652-3p) while the x-axis denotes the batch information and disease status. Note that miR-106a-5p and 17-5p are equivalent (i.e., indistinguishable in the reference database that was used), so are miR-23a-3p and 23b-3p.

FIG. 8 provides data showing consistency across the discovery batch (1st control and 1st disease) and validation batch (2nd control and 2nd disease) for the newly identified miRNA endogenous controls. The y-axis denotes the normalized counts while the x-axis denotes the batch information and disease status.

FIG. 9 provides data showing that two representative miRNA biomarkers identified by NGS and subsequently validated in ddPCR effectively distinguished between subjects with and without endometriosis when samples were taken at proliferative phase. The y-axis denotes the normalized values of the biomarkers against endogenous control while the x-axis denotes the batch information and disease status (P: patient; C: healthy control).

FIG. 10 provides data for the newly identified miRNA endogenous controls. The y-axis denotes the normalized counts while the x-axis denotes disease status (P: patient; C: healthy control).

### 7. DETAILED DESCRIPTION

Provided herein are non-invasive methods of assessing whether a subject has endometriosis or is at risk of developing endometriosis, and methods of predicting the prognosis in a subject having endometriosis. Methods provided herein include measuring the levels of certain miRNAs in a sample of the subject and comparing them to reference levels. Methods of treating a subject with endometriosis based on the miRNA levels in a sample from the subject, as well as systems and kits for carrying out the methods described herein are also disclosed.

Before the present disclosure is further described, it is to be understood that the disclosure is not limited to the particular embodiments set forth herein, and it is also to be understood that the terminology used herein is for the purpose of describing particular embodiments, and is not intended to be limiting.

### 7.1 Definitions

Unless otherwise defined herein, scientific and technical terms used in the present disclosures shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art.

As used herein in the specification, “a” or “an” may mean one or more. As used herein in the claim(s), when used in conjunction with the word “comprising,” the words “a” or “an” may mean one or more than one.

As used herein, the term “or” in the claims is used to mean “and/or” unless explicitly indicated to refer to alternatives only or the alternatives are mutually exclusive, although the disclosure supports a definition that refers to only alternatives and “and/or.” As used herein “another” or “additional” may mean at least a second or more.

As used herein, the term “about” is used to indicate that a value includes the inherent variation of error for the device, the method being employed to determine the value, or the variation that exists among the study subjects. The term “about” encompasses the exact number recited. In some embodiments, “about” means within plus or minus 10% of a given value or range. In certain embodiments, “about” means that the variation is ±5%, ±4%, ±3%, ±2%, ±1%, ±0.5%, ±0.2%, or ±0.1% of the value to which “about” refers. In some embodiments, “about” means that the variation is ±1%, ±0.5%, ±0.2%, or ±0.1% of the value to which “about” refers.

The terms “nucleic acid,” “polynucleotide,” and their grammatical equivalents, are used interchangeably herein and refer to a polymer or oligomer of nucleotides of any length. The nucleotides can be deoxyribonucleotides, ribonucleotides, modified nucleotides or bases (such as methylated, hydroxymethylated, or glycosylated), non-natural nucleotides, non-nucleotide building blocks that exhibit similar structure and/or function as natural nucleotides (i.e., “nucleotide analogs”), and/or any substrate that can be incorporated into a polymer by DNA or RNA polymerase. The nucleic acids or polynucleotides can be heterogenous or homogenous in composition, can be isolated from naturally occurring sources, or can be artificially or synthetically produced. In addition, the nucleic acids or polynucleotides can be DNA (e.g., cDNA or genomic DNA) or RNA (e.g., mRNA, anti-sense RNA, siRNA, and miRNA), or a mixture thereof, and can exist permanently or transitionally in single-stranded or double-stranded form, including homoduplex, heteroduplex, and hybrid states.

Conventional notation is used herein to describe nucleotide sequences: the left-hand end of a single-stranded nucleotide acid is the 5′-end; the left-hand direction of a double-stranded nucleic acid is referred to as the 5′-direction; the right-hand end of a single-stranded nucleotide acid is the 3′-end; the right-hand direction of a double-stranded nucleic acid is referred to as the 3′-direction. The direction of 5′ to 3′ addition of nucleotides to nascent RNA transcripts is referred to as the transcription direction. The DNA strand having the same sequence as an mRNA is referred to as the “coding strand.” Sequences on the DNA strand which are located 5′ to a reference point on the DNA are referred to as “upstream sequences.” Sequences on the DNA strand which are 3′ to a reference point on the DNA are referred to as “downstream sequences.”

When referring to a nucleotide sequence or protein sequence, the term “identity” is used to denote similarity between two sequences. Sequence similarity or identity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J Mol. Biol. 48,443 (1970), by the search for similarity method of Pearson & Lipman, Proc. Natl. Acad. Sci. USA 85, 2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12, 387-395 (1984), or by inspection. Another algorithm is the BLAST algorithm, described in Altschul et al., J Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460-480 (1996); blast.wustl/edu/blast/README.html. WU-BLAST-2 uses several search parameters, which are optionally set to the default values. The parameters are dynamic values and are established by the program itself depending upon the composition of the particular sequence and composition of the particular database against which the sequence of interest is being searched; however, the values may be adjusted to increase sensitivity. Further, an additional useful algorithm is gapped BLAST as reported by Altschul et al., (1997) Nucleic Acids Res. 25, 3389-3402. Unless otherwise indicated, percent identity is determined herein using the algorithm available at the internet address: blast.ncbi.nlm.nih.gov/Blast.cgi.

As used herein, terms “complementary” and “complementarity” refers to the relationship between two nucleic acid molecules having the capacity to form hydrogen bond(s) with one another by either traditional Watson-Crick base-paring or other non-traditional types of pairing. The two DNA/RNA strands with complementary sequences bind to form a duplex that follows the Watson-Crick base-pairing rules: A binds to T (U) with two hydrogen bonds; G binds to C with three hydrogen bonds. The degree of complementarity between two nucleotide sequences can be indicated by the percentage of nucleotides in a nucleotide sequence which can form hydrogen bonds (e.g., Watson-Crick base pairing) with a second nucleotide sequence (e.g., about 50%, about 60%, about 70%, about 80%, about 90%, and 100% complementary). Two nucleotide sequences are “perfectly complementary” or “100% complementary” if all the contiguous nucleotides of a nucleotide sequence will hydrogen bond with the same number of contiguous nucleotides in a second nucleotide sequence. Two nucleotide sequences are “substantially complementary” if the degree of complementarity between the two nucleotide sequences is at least 60% (e.g., at least 65%, at least 70%, at least 75%, at least 80%, at least 85%, at least 90%, at least 95%, at least 97%, at least 98%, at least 99%, or 100%) over a region of at least 8 nucleotides (e.g., at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, or more nucleotides), or if the two nucleotide sequences hybridize under at least moderate, or, in some embodiments high, stringency conditions. Exemplary stringency conditions are descried in, e.g., Sambrook, J., Molecular Cloning: A Laboratory Manual, Cold Spring Harbor Laboratory Press; 4th edition (Jun. 15, 2012), and Ausubel et al., eds., SHORT PROTOCOLS IN MOLECULAR BIOLOGY, 5th ed., John Wiley & Sons, Inc., Hoboken, N.J. (2002).

The term “hybridization” or “hybridized” when referring to nucleotide sequences is the association formed between and/or among sequences having complementarity.

As used herein, the term “biomarker” or its grammatical equivalent refers to a gene (e.g., a gene encoding a miRNA) that can be either present or absent in individual subjects, or can be present but differentially expressed in individual subjects. The presence of a biomarker, including the expression level of the biomarker, in a sample from a subject can indicate the likelihood of the subject to have or develop a particular condition, or progress into an advanced stage of the condition, such as endometriosis.

As used herein and consistent with its understanding in the art, “microRNA” or “miRNA” refers to small noncoding RNA molecules, generally about 15 to about 50 nucleotides, more often about 17 to about 23 nucleotides in length, which can play a role in regulating gene expression. For example, through a process termed RNA interference (RNAi), the presence of a miRNA that is complementary or antisense to a sequence in a mRNA of a target gene results in inhibition of expression of the target gene. miRNAs are processed from hairpin precursors of about 70 or more nucleotides (pre-miRNA) which are derived from primary transcripts (pri-miRNA) through sequential cleavage by RNAse III enzymes. miRBase is a comprehensive microRNA database located at mirbase.org, hereby incorporated by reference in its entirety.

As used herein, the term “level” of a biomarker refers to the amount of the expression product of a biomarker (e.g., miRNA). If the biomarker is a gene with more than one allele or isoform, the expression level of a biomarker refers to the total amount of accumulation of the expression product of all existing alleles or isoforms for this gene, unless otherwise specified.

As used herein, the term “measure” or its grammatical equivalent, refers to the process of conducting a qualitative, a semi-quantitative or a quantitative means for, e.g., detecting and determining the expression level of a biomarker, using technology available to the skilled artisan. Measurement can be relative or absolute. Measuring the expression of a biomarker can include, e.g., determining whether the expression product of the biomarker is present or absent, or the amount of the expression product of the biomarker.

As used herein, the term “reference level” of expression refers to a predetermined expression level of a biomarker that can be used to determine the significance of the expression level of the biomarker in a sample from a subject. A reference expression level of a biomarker can be the expression level of the biomarker in a sample from a healthy individual. A reference expression level of a biomarker can also be a cut-off value determined by a person of ordinary skill in the art through statistical analysis of the expression levels of the biomarker in a sample population and the of the clinical outcome of the individuals in the sample population. For example, by analyzing the expression levels of certain miRNA in individuals of a sample population and the clinical outcome of these individuals with respect to endometriosis, a person of ordinary skill in the art can determine a cut-off value as the reference expression level of the miRNA, wherein a subject is likely to have, develop, or progress into an advanced stage of endometriosis if the expression level of miRNA of the subject is different from the reference expression level.

Comparing the expression level of a biomarker (e.g., a miRNA) usually means comparison of corresponding parameters or values, e.g., an absolute amount is compared to an absolute reference amount, or an intensity signal obtained from the biomarker in a sample is compared to the same type of intensity signal obtained from a reference sample. The comparison can be carried out manually or assisted by computer. In some embodiments, the comparison is carried out by a computing device. The value of the measured or detected expression level of the biomarker in a sample from a subject and the reference level can be, e.g., compared to each other and the said comparison can be automatically carried out by a computer program executing an algorithm for the comparison. The computer program carrying out the said evaluation can provide the desired assessment in a suitable output format. For a computer-assisted comparison, the value of the measured amount can be compared to values corresponding to suitable references which are stored in a database by a computer program. The computer program can further evaluate the result of the comparison, i.e., automatically provide the desired assessment in a suitable output format.

As used herein and understood in the art, the terms “lowered,” “decreased,” “down-regulated,” or “underexpressed” when used in connection to the expression level of a biomarker (e.g., a miRNA) means that such expression level in the sample is less than the reference level. For example, a decreased expression level of a miRNA disclosed herein detected in a sample of a subject means that the expression level of the miRNA in the sample is lower compared to a reference level. In some embodiments, the expression level of the biomarker can be at least 10%, at least 20%, at least 30%, at least 40%, at least 50%, at least 60%, at least 70%, at least 80%, at least 90% less than the reference level.

As used herein and understood in the art, the terms “elevated,” “increased,” “up-regulated,” or “overexpressed” when used in connection to the expression level of a biomarker (e.g., a miRNA) means that such expression level in the sample is higher than the reference level. For example, an increased expression level of a miRNA disclosed herein detected in a sample of a subject means that the expression level of the miRNA in the sample is higher compared to a reference level. In some embodiments, the expression level of the biomarker can be at least 10%, at least 20%, at least 30%, at least 40%, at least 50%, at least 60%, at least 70%, at least 80%, at least 90%, at least 2.0 fold, at least 3.0 fold, at least 4.0 fold, at least 5.0 fold, at least 6.0 fold, at least 7.0 fold, at least 8.0 fold, at least 9.0 fold, or at least 10.0 fold than the reference level.

As used herein, the term “assess” or its grammatical equivalent, refers to the process of determining the current status of an event or a condition, or the future development of an event or a condition. For example, assessing whether a subject has or is predisposed to endometriosis means determining the likelihood that the subject suffers from endometriosis or is at risk of developing endometriosis. For another example, assessing the prognosis of endometriosis in a subject means determining the likelihood that the subject will progress into an advanced stage of endometriosis. Accordingly, assessing as used herein includes diagnosing endometriosis, predicting the risk for developing endometriosis, and predicting the future development of endometriosis, which can provide guidance for selecting for therapy, monitoring a patient suffering from endometriosis or being treated for endometriosis. In some embodiments, the risk of developing endometriosis or progressing into an advanced stage of endometriosis is predicted within a window between 6 months and two years. In some embodiments, the predictive window is between 6 months to 12 months.

As used herein, the term “endogenous control” refers to the molecules that are used as internal references to ensure reliability and reproducibility of quantitative analyses in expression studies (e.g., miRNA expression studies). These controls are selected based on the assumption that their expression levels remain stable under various experimental conditions, across different biological samples, and between different treatment groups. In the context of miRNA quantification, endogenous controls minimize the effects of technical variation in the experimental process. Since miRNA expression levels can be influenced by multiple factors such as RNA quality, cDNA synthesis, or PCR efficiency, endogenous controls provide a means to correct for such variability.

As used herein, the term “normalize” and its grammatical equivalents refer to the process of adjusting experimental data to account for various sources of technical variability, ensuring that the reported expression levels (e.g., miRNA expression levels) accurately reflect the biological differences between samples. Normalization typically involves comparing the expression level of a target of interest to the expression level of an endogenous control. In the context of miRNA expression studies, normalization reduces differences in RNA input across samples due to variations in sample collection, RNA extraction, or degradation, variability in the amplification efficiencies between different miRNAs and samples during quantitative PCR, and sample-to-sample variability resulted from differences in sample handling, such as differences in cDNA synthesis efficiency or reagent performance.

As used herein, the term “subject” as used herein refers to any animal (e.g., a mammal), including, but not limited to, humans, non-human primates, canines, felines, rodents, and the like. The subject can be a human. The subject can be a human female. The subject can be a healthy subject. A subject can have a particular disease or condition. The subject can have at least one symptom associated with endometriosis, such as pelvic pain, dysmenorrhea, or infertility. In some embodiments, the subject is a young or adolescent human female. In some embodiments, the subject is a human female aged between 12-60 years. In some embodiments, the subject is a human female aged about 20 years old, 30 years old, 40 years old, 50 years old or 60 years old.

As used herein, the term “sample,” refers to a part or piece of a tissue, organ or individual, typically being smaller than such tissue, organ or individual, intended to represent the whole of the tissue, organ or individual. Upon analysis a sample provides information about the tissue status or the health or diseased status of an organ or individual. Examples of samples include but are not limited to fluid samples such as blood, serum, plasma, synovial fluid, urine, saliva, and lymphatic fluid, or solid samples such as tissue extracts, cartilage, bone, synovium, and connective tissue. Chemical analysis includes but is not limited to the detection of the presence or absence of specific indicators or alterations in their amount, concentration or level. A sample can be obtained, reached, or collected in vivo or in situ. The sample is an in vitro sample means that it is to be analyzed in vitro and not transferred back into the body. Analysis of a sample can be accomplished on a visual or chemical basis. Visual analysis includes but is not limited to microscopic imaging or radiographic scanning of a tissue, organ or individual allowing for morphological evaluation of a sample.

As used herein, the term “treat” or its grammatical equivalent refers to executing a protocol or plan, which can include administering one or more drugs or active agents to a patient to alleviate signs or symptoms of the disease or the recurrence of the disease. Treatment can also include medical procedures such as surgeries. Desirable effects of treatment include decreasing the rate of disease progression, ameliorating or palliating the disease state, and remission, increased survival, improved quality of life or improved prognosis. Alleviation or prevention can occur prior to signs or symptoms of the disease or condition appearing, as well as after their appearance. As used herein, a “treatment” does not require complete alleviation of signs or symptoms and does not require a cure. As used herein, the term “therapeutic beneficial” or “therapeutically effective” when used in connection with a treatment refers to the property of the treatment that promotes or enhances the well-being of the subject. This includes, but is not limited to, a reduction in the frequency, severity, or rate of progression of the signs or symptoms of a disease. For example, treatment of endometriosis may result in, for example, a reduction in pain, or pregnancy.

As used herein, the term “administer” or its grammatical equivalent refers to the act of delivering, or causing to be delivered, a compound or a pharmaceutical composition to the body of a subject by a method described herein or otherwise known in the art, and the act of providing a medical procedure on the subject for the purpose of treating the subject. Administering a compound or a pharmaceutical composition includes prescribing a compound or a pharmaceutical composition to be delivered into the body of a patient. Exemplary forms of administration include oral dosage forms, such as tablets, capsules, syrups, suspensions; injectable dosage forms, such as intravenous (IV), intramuscular (IM), or intraperitoneal (IP); transdermal dosage forms, including creams, jellies, powders, or patches; buccal dosage forms; inhalation powders, sprays, suspensions, and rectal suppositories.

Nomenclature for nucleotides, nucleic acids, nucleosides, and amino acids used herein is consistent with International Union of Pure and Applied Chemistry (IUPAC) standards (see, e.g., bioinformatics.org/smsylupac.html). Exemplary genes and polypeptides are described herein with reference to GenBank numbers, GI numbers and/or SEQ ID NOS. It is understood that one skilled in the art can readily identify homologous sequences by reference to sequence sources, including but not limited to Uniprot (https://www.uniprot.org/), GenBank (ncbi.nlm.nih.gov/genbank/) and EMBL (embl.org/).

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range or the characteristics being described.

### 7.2 Gynecological Diseases

Gynecological diseases include a wide range of conditions such as ovarian cysts, endometriosis, uterine fibroids, pelvic inflammatory disease (PID), cervical and ovarian cancer, and many others. Early detection of gynecological diseases is crucial. First, detecting these diseases at an early stage often increases the chances of successful treatment and better outcomes. Timely intervention can help manage symptoms, prevent complications, and potentially save lives. Second, gynecological diseases can cause significant discomfort and impact a woman's quality of life. Early detection allows for prompt management, reducing the duration and intensity of symptoms and promoting overall well-being. Third, some gynecological conditions, if left untreated, can lead to infertility. Early detection enables early intervention, preserving fertility options and increasing the chances of successful reproductive outcomes. Currently, there is a lack of early, noninvasive procedures for the early detection of gynecological diseases. Many diagnostic methods involve invasive procedures such as biopsies, laparoscopy, or imaging techniques like ultrasound or MRI scans. While these procedures are valuable and necessary in certain situations, they can be uncomfortable, expensive, and may carry some risks. Also, their accuracy can be further improved. Noninvasive procedures, such as blood tests or biomarker screenings, which aim to detect gynecological diseases at early stages using simple and less invasive techniques, are urgently needed.

Endometriosis is a chronic inflammatory gynecological disease characterized by the non-tumorigenic spread/metastasis of endometrial tissue outside of the uterus. The endometrium is regularly lost, then proliferates again, throughout a woman's reproductive age. In about 10-15% of reproductive age and in 20-50% infertile women, however, endometrium-like tissues (known as lesions) also grow outside of the uterus, leading to endometriosis. Clinical presentation of endometriosis varies significantly from patient to patient. Endometriosis patients often present with symptoms such as intermenstrual bleeding, painful periods (dysmenorrhea), painful intercourse (dyspareunia), painful defecation (dyschezia) and painful urination (dysuria). The main symptom of endometriosis is pelvic pain, which is often associated with the menstrual period. Although women experience cramping pain during their menstrual period, those with endometriosis often experience more severe menstrual pain. The pain may also get worse over time. Pelvic pain due to endometriosis is usually chronic (lasting >6 months) and is associated with dysmenorrhea (in 50 to 90% of cases), dyspareunia, deep pelvic pain, and lower abdominal pain with or without back and loin pain. The pain can occur unpredictably and intermittently throughout the menstrual cycle or it can be continuous, and it can be dull, throbbing, or sharp, and exacerbated by physical activity. Bladder—and bowel—associated symptoms (nausea, distention, and early satiety) are typically cyclic. Pain often worsens over time and may change in character; infrequently, women report burning or hypersensitivity, symptoms that are suggestive of a neuropathic component. The severity of pain is not necessarily a reliable indicator of the extent of endometriosis. A woman can have mild endometriosis with severe pain or advanced endometriosis with little or no pain. Often, endometriosis can be asymptomatic, only coming to a clinician's attention during evaluation for infertility (Sinaii et al., Fertil Steril. 2008; 89(3): 538-545). Sometimes, endometriosis is the first condition diagnosed in patients seeking infertility treatment. In women with endometriosis, there is a reduced monthly fecundity rate (2-10%) compared with fertile couples (15-20%). Although endometriosis impairs fertility, it does not usually completely prevent conception (Fadhlaoui et al., Front Surg. 2014; 1: 24). Endometriosis affected women may experience fatigue, diarrhea, constipation, bloating, or nausea, especially during menstruation.

The most commonly affected sites of endometriosis are the pelvic organs and peritoneum, although other parts of the body such as the lungs are occasionally affected. The extent of the disease varies from a few, small lesions on otherwise normal pelvic organs to large, ovarian endometriotic cysts (endometriomas) and/or extensive fibrosis and adhesion formation causing marked distortion of pelvic anatomy. Based on the location, endometriotic lesions can be classified into peritoneal endometriosis, ovarian endometriotic cysts (endometrioma) and deep nodules (deep infiltrating endometriosis) (Kennedy et al., Hum Reprod. 2005; 20(10): 2698-2704).

The revised classification system established by the American Society for Reproductive Medicine (ASRM) describes the severity of endometriosis based on the findings at surgery (laparoscopy). The classification is based on the morphology of peritoneal and pelvic implants such as red, white and black lesions, percentage of involvement of each lesion should be included. Number, size, and location endometrial implants, plaques, endometriomas and adhesions are noted. Endometriosis in bowel, urinary tract, fallopian tube, vagina, cervix, skin, or other locations is documented per ASRM guidelines. Stages of endometriosis according to ASRM guidelines are stage I, II, III, and IV determined based on the point scores and correspond to minimal, mild, moderate and severe endometriosis. The rASRM (revised ASRM staging) stages I & II endometriosis (minimal to mild endometriosis) are defined by superficial peritoneal endometriosis, possible presence of small deep lesions, absence of endometrioma and/or mild filmy adhesion. The rASRM stages III and IV endometriosis (moderate to severe endometriosis) are defined by the presence of superficial peritoneal endometriosis, deep infiltrating endometriosis with moderate to extensive adhesions between the uterus and bowels and/or endometrioma cysts with moderate to extensive adhesions involving the ovaries and tubes.

The Visual Analog Scale, or VAS, is an instrument to assess the intensity of pain. The VAS consists of a horizontal line with its extremes marked as ‘no pain’ and ‘worst pain imaginable.’ Each patient ticks her pain level on the line and the distance from ‘no pain’ on the extreme left to the tick mark is measured in centimeters, yielding a pain score from 0 to 10. ‘No pain’ corresponds to a pain score of 0, ‘worst pain imaginable’ corresponds to a pain score of 10. In women with endometriosis dysmenorrhea is associated with the highest perception of pain with a mean VAS score of about 6 (Cozzolino et al., Rev Bras Ginecol Obstet 2019; 41(3): 170-175).

The onset of endometriosis for a subject predisposed to endometriosis can be prevented or delayed by early intervention. A subject that is predisposed to endometriosis means that she is at risk of developing endometriosis. That is, albeit the absence of a diagnosis of endometriosis, the subject shows potential of endometriosis emerging in the future. In some embodiments, the risk of developing endometriosis is associated with early or weak signs or symptoms. In some embodiments, a subject is predisposed to endometriosis despite the absence of any signs or symptoms.

At present, the etiology of endometriosis is unknown, there is no cure and the treatment options available are limited. The disease has a high recurrence rate, which adds to its large socio-economic impact. Despite a number of theories, the molecular mechanisms giving rise to the development of endometriosis such as: Sampson's theory of retrograde menstruation, ectopic implantation, epigenetic factors, immune and inflammatory factors, eutopic endometrial determinism, and stem cell factors; disease pathogenesis is still not fully understood.

Growth of cells derived from the endometrium outside the uterus in endometriosis can be regional or distant, such as the ovaries, peritoneum, intestines and vagina. In a small number of cases (0.5-1%) endometriosis can lead to tumor formation. Underlying mechanisms that have been proposed are similar to malignant tumors such as cell proliferation, differentiation, apoptosis, migration, cell adhesion, invasion, and neuro-vascularization.

The diagnosis of endometriosis remains a major challenge. Its clinical presentation varies widely, ranging from asymptomatic to severe, and no diagnostic biomarker has been approved for routine clinical diagnosis of endometriosis to date. Diagnostic imaging studies such as pelvic ultrasound and magnetic resonance imaging are now used to diagnose endometriosis, but the imaging findings are greatly influenced by the examiner's expertise, ultimately making the diagnosis difficult. In the absence of positive imaging findings, the final diagnosis of superficial endometriosis can only be made by histological analysis of the lesion, which is usually obtained by laparoscopic surgery. However, this procedure is invasive and requires general anesthesia. In addition, due to the associated risks, patients are reluctant to undergo surgery in the absence of severe symptoms, so laparoscopy is rarely performed in the early stages of the disease. The complexity of endometriosis, coupled with the lack of accurate and less invasive diagnostic methods, leads to an average delay of 6.6 years in diagnosis, sometimes up to 11 years. Therefore, there is a great need for accurate and minimally invasive diagnostic tests for endometriosis by doctors and patients.

The gold standard for the diagnosis of endometriosis has been laparoscopy for many years, with direct visualization of the endometriosis and histologic evaluation. During a diagnostic laparoscopy, a gynecologist with training and skills in laparoscopic surgery for endometriosis should perform a systematic inspection of the pelvis. Typical peritoneal endometriosis manifests as purple-blue burning lesions on the surface of the ovaries, the rectouterine pouch, and the uterosacral ligaments. The lesions may be black, dark brown, or blue wrinkled lesions, nodules, or small cysts containing old hemorrhages surrounded by varying degrees of fibrosis. Ovarian endometriosis cysts appear as smooth, dark cysts that often adhere to the surrounding tissues and contain chocolate-like fluid. Deep infiltrating endometriosis refers to lesions that infiltrate 0.5 cm below the peritoneum, commonly found in the uterosacral ligaments, vagina, intestines, bladder, and ureters. In addition, biopsy or lesion resection can be performed simultaneously during surgery to obtain a pathological diagnosis. The pathological diagnosis is that endometrial glands and stroma can be seen in the lesions under the microscope, accompanied by inflammatory reactions and fibrosis. Current guidelines, however, have been updated to require only clinical criteria for an endometriosis diagnosis; thus, reducing the time to diagnosis without the need for pathologic confirmation. With these new guidelines, accuracy and misdiagnosis are likely to be reduced and increased respectively. A noninvasive test proven to be equivalent to laparoscopic/pathologic testing would, therefore be highly beneficial. Until now, there are no non-invasive methods for the diagnosis of endometriosis (Hsu et al., Clin Obstet Gynecol 2010: 53: 413-419). The lack of a non-invasive diagnostic test significantly contributes to the long delay between onset of the symptoms and definitive diagnosis of endometriosis (Signorile and Baldi. J Cell Physiol, 2014; 229: 1731-1735).

When new diagnostic tests are developed, their sensitivity and specificity should be assessed in comparison with the current gold standard method. Direct laparoscopic observation and histopathology have traditionally been considered the gold standard for the diagnosis of endometriosis, compared with all other diagnostic tests with an accuracy of 80-90%. According to the 2015 STARD (Standards for Reporting Diagnostic Accuracy Studies) guidelines, the test index and reference standard should be explained in sufficient detail to allow for reproducibility, as differences in test performance may be a factor in the variation in diagnostic accuracy. It is also important to emphasize the skill level of the personnel using the index and reference standard tests (ultrasonography, image interpretation, surgery), as the accuracy of the test depends on their level of expertise. The term “reference standard” means the best available method(s) for establishing the presence or absence of the target condition (such as, endometriosis).

Non-invasive diagnosis of endometriosis would allow earlier diagnosis and treatment, with the potential to improve quality of life and reduce the societal costs related to endometriosis, and has therefore been selected as a research priority by the World Endometriosis Society (WES) and the World Endometriosis Research Foundation (WERF) (Fassbender et al., Springer, Peripheral Blood Biomarkers for Endometriosis. 2017; Parasar et al., Curr Obstet Gynecol Rep., 2017; 6: 34-41). CA-125, or carbohydrate antigen 125, is a commonly used blood biomarker for endometriosis; however, its diagnostic utility is limited to endometriosis rASRM stages III and IV (Nisenblat et al., Cochrane Database of Systematic Reviews, 2016; 5: CD012179). Also, both sensitivity and specificity of the existing diagnostic assay can be improved. Thus, there is an unmet medical need for a non-invasive test for the diagnosis of endometriosis, in particular for the diagnosis of early-stage endometriosis (rASRM stages I-II). miRNA (microRNA) is a small endogenous non-coding RNA (ribonucleic acid) molecule that is involved in post-transcriptional gene expression regulation. Because they can be found outside cells in different body fluids (such as serum and plasma, vaginal fluid, urine, and saliva), they can be detected and can be used as very promising noninvasive candidate biomarkers for disease diagnosis. The use of miRNA in the diagnosis of endometriosis has not yet been translated into the standard for clinical practice. One of the main reasons may be the heterogeneity of miRNA expression levels due to different tissue types, detection time, and storage time. The translation of research results into clinical applications requires appropriate control groups, standardized quantitative miRNA expression levels, and careful validation at the analytical and clinical levels. In addition, choosing the appropriate time or window of the menstrual cycle for patient testing also can play an important role in correctly detecting differential expression of uterine miRNAs between patients and controls.

Adenomyosis is a gynecological condition where the tissue that normally lines the uterus (endometrium) grows into the muscular wall of the uterus (myometrium). This growth can cause the uterine walls to become thickened and enlarged. Adenomyosis is a non-cancerous condition that primarily affects women of childbearing age. The exact cause of adenomyosis is not fully understood. It may result from the invasion of endometrial cells into the myometrium, tissue damage during childbirth or surgery, or hormonal imbalances, particularly high levels of estrogen.

The symptoms of adenomyosis can vary from mild to severe and include menstrual pain, heavy menstrual bleeding, pelvic pain, painful intercourse, and enlarged uterus. Diagnosing adenomyosis usually requires a combination of medical history, physical examination, and imaging tests such as ultrasound, magnetic resonance imaging (MRI), or a transvaginal ultrasound. In some cases, laparoscopy can be performed to confirm the diagnosis.

Treatment options for adenomyosis aim to manage symptoms and can vary depending on the severity and individual preferences. They may include pain medication, hormonal therapies (such as oral contraceptives or hormonal intrauterine devices), or, in severe cases, surgery (such as a hysterectomy, which involves removal of the uterus).

Approximately 50% of endometriosis patients can have adenomyosis which can influence severity of symptoms.

### 7.3 CA-125 as Biomarker for Endometriosis

Provided herein are non-invasive methods of assessing whether a subject has endometriosis by assessing CA-125 (carbohydrate antigen 125 or cancer Antigen 125) level in the subject. CA-125, also known as mucin 16 (MUC16), is a high molecular weight glycoprotein that is encoded by the MUC16 gene. It is primarily found on the surface of ovarian epithelial cells and is released into the bloodstream. Methods provided herein include measuring the expression level of CA-125 in a sample obtained during the secretory phase of the menstrual cycle of the subject, wherein an increased level compared to a reference level indicates that the subject is likely to have endometriosis. Methods of treating endometriosis based on the expression level of CA-125, as well as systems and kits for carrying out the methods described herein are also disclosed.

Provided herein are methods of assessing whether a subject has endometriosis comprising (a) measuring the expression level of CA-125 in a sample of the subject, and (b) comparing the expression level to a reference level; wherein an increased expression level indicates that the subject is likely to have endometriosis.

Provided herein are methods of assessing whether a subject has endometriosis, comprising (a) measuring the expression level of CA-125 in a sample obtained during the secretory phase of the menstrual cycle of the subject, and (b) comparing the expression level to a reference level; wherein an increased expression level indicates that the subject is likely to have endometriosis. In some embodiments, methods provided herein measure the protein level of CA-125. In some embodiments, methods provided herein measure the mRNA level of CA-125.

An increased expression level of CA-125 indicates that the subject is likely to have endometriosis. In some embodiments, an expression level that is increased by at least 20% compared to a reference level indicates that the subject is likely to have endometriosis. In some embodiments, an expression level that is increased by at least 50% compared to a reference level indicates that the subject is likely to have endometriosis. In some embodiments, an expression level that is increased by at least 80% compared to a reference level indicates that the subject is likely to have endometriosis. In some embodiments, an expression level that is increased by at least 90% compared to a reference level indicates that the subject is likely to have endometriosis. In some embodiments, an expression level that is increased by at least 95% compared to a reference level indicates that the subject is likely to have endometriosis. In some embodiments, an expression level that is increased by at least 20%, at least 30%, at least 40%, at least 50%, at least 60%, at least 70%, at least 80%, at least 90%, at least 95%, or at least 99%, compared to a reference level indicates that the subject is likely to have endometriosis.

The reference level that serves as the cutoff value for CA-125 level in the methods disclosed herein can be determined by a person of ordinary skill in the art. In some embodiments, the reference level ranges from 10 to 35 U/mL. The reference level can be 10 U/mL, 12 U/mL, 15 U/mL, 20 U/mL, 25 U/mL, 30 U/mL or 35 U/mL. In some embodiments, the reference level is 11 U/mL. In some embodiments, the reference level is 20 U/mL. In some embodiments, the reference level is 30 U/mL. In some embodiments, the reference level is 35 U/mL. As known in the art, selection of the cutoff value can impact the sensitivity and specificity of the assay. Thus, the optimal cutoff value can be determined based on clinical context and receiver operating characteristics curves (ROC) to maximize the utility of the assay.

Methods provided herein comprise measuring the expression level of CA-125 in a subject. In some embodiments, the methods provided herein comprise measuring the protein level. Many methods of measuring protein expression are known in the art, including qualitative, semi-quantitative and quantitative methods. It is generally known to the skilled artisan which methods are suitable for qualitative and/or for quantitative detection of a biomarker.

In some embodiments, the protein level is measured by immunohistochemistry (IHC), immunocytochemistry (ICC), an enzyme-linked immunosorbent assay (ELISA), immunoblotting assay (e.g., Western blot), flow cytometry (FACS), a fluorescent immunosorbent assay (FIA), a chemiluminescence immunoassay (CIA), an electrochemiluminescence Immunoassay (ECLIA), a radioimmunoassay (RIA), a solid phase radioimmunoassay (SPROA), or a dot/line-immunoblot assay. Further suitable methods to detect biomarkers include measuring a physical or chemical property specific for the peptide or polypeptide such as its precise molecular mass or NMR spectrum. Said methods comprise, e.g., biosensors, optical devices coupled to immunoassays, biochips, analytical devices such as mass-spectrometers, NMR-analyzers, or chromatography devices. Further, methods include microplate ELISA-based methods, fully-automated or robotic immunoassays (available for example on Elecsys™ analyzers), CBA (an enzymatic Cobalt Binding Assay, available for example on Roche-Hitachi™ analyzers), and latex agglutination assays (available for example on Roche-Hitachi™ analyzers).

In some embodiments, the protein level is measured by IHC. IHC staining of tissue sections has been shown to be a reliable method of assessing or detecting presence of proteins in a sample. Immunohistochemistry techniques utilize an antibody to probe and visualize cellular antigens in situ, generally by chromogenic or fluorescent methods. Thus, antibodies or antisera, preferably polyclonal antisera, and most preferably monoclonal antibodies specific for each marker are used to detect expression. As discussed in greater detail below, the antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. IHC protocols and kits are well known in the art and are commercially available. Automated systems for slide preparation and IHC processing are available commercially. The Ventana® BenchMark XT system is an example of such an automated system. Standard immunological and immunoassay procedures can be found in BASIC AND CLINICAL IMMUNOLOGY (Stites & Terr eds., 7th ed. 1991). Moreover, the immunoassays can be performed in any of several configurations, which are reviewed extensively in ENZYME IMMUNOASSAY (Maggio, ed., 1980). For a review of the general immunoassays, see also METHODS IN CELL BIOLOGY: ANTIBODIES IN CELL BIOLOGY, volume 37 (Asai, ed. 1993); BASIC AND CLINICAL IMMUNOLOGY (Stites & Ten, eds., 7th ed. 1991).

Commonly used assays to detect protein level of a biomarker include noncompetitive assays, e.g., sandwich assays, and competitive assays. A wide range of immunoassay techniques using such an assay format are available, see, e.g., U.S. Pat. Nos. 4,016,043, 4,424,279, and 4,018,653, which are hereby incorporated by reference in their entireties. Sandwich immunoassays are broadly used in the detection of an analyte of interest. In such assay the analyte is “sandwiched” in between a first antibody and a second antibody. Typically, a sandwich assay requires that capture and detection antibody bind to different, non-overlapping epitopes on an analyte of interest. By appropriate means such sandwich complex is measured and the analyte thereby quantified. In a typical sandwich-type assay, a first antibody bound to the solid phase or capable of binding thereto and a detectably-labeled second antibody each bind to the analyte at different and non-overlapping epitopes. The first analyte-specific binding agent (e.g., an antibody) is either covalently or passively bound to a solid surface. The solid surface is typically glass or a polymer, the most commonly used polymers being cellulose, polyacrylamide, nylon, polystyrene, polyvinyl chloride, or polypropylene. The solid supports can be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g., 2-40 minutes or overnight if more convenient) and under suitable conditions (e.g., from room temperature to 40° C. such as between 25° C. and 37° C. inclusive) to allow for binding between the first or capture antibody and the corresponding antigen. Following the incubation period, the solid phase, comprising the first or capture antibody and bound thereto the antigen can be washed, and incubated with a secondary or labeled antibody binding to another epitope on the antigen. The second antibody is linked to a reporter molecule which is used to indicate the binding of the second antibody to the complex of first antibody and the antigen of interest.

Variations of the sandwich assay technique exist. For example, in a simultaneous assay, both sample and labeled antibody are added simultaneously to the bound antibody. These techniques are well known to those skilled in the art, including any minor variations as will be readily apparent. A versatile alternative sandwich assay uses a solid phase coated with the first partner of a binding pair, e.g., paramagnetic streptavidin-coated microparticles. Such microparticles are mixed and incubated with an analyte-specific binding agent bound to the second partner of the binding pair (e.g., a biotinylated antibody), a sample suspected of comprising or comprising the analyte, wherein said second partner of the binding pair is bound to said analyte-specific binding agent, and a second analyte-specific binding agent which is detectably labeled. As obvious to the skilled person these components are incubated under appropriate conditions and for a period of time sufficient for binding the labeled antibody via the analyte, the analyte-specific binding agent (bound to) the second partner of the binding pair and the first partner of the binding pair to the solid phase microparticles. As appropriate such assay can include one or more washing step(s).

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase, and alkaline phosphatase, and other are discussed herein. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labeled antibody is added to the first antibody-molecular marker complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of biomarker which was present in the sample. Alternately, fluorescent compounds, such as fluorescein and rhodamine, can be chemically coupled to antibodies without altering their binding capacity. When activated by illumination with light of a particular wavelength, the fluorochrome-labeled antibody adsorbs the light energy, inducing a state to excitability in the molecule, followed by emission of the light at a characteristic color visually detectable with a light microscope. As in the EIA, the fluorescent labeled antibody is allowed to bind to the first antibody-molecular marker complex. After washing off the unbound reagent, the remaining tertiary complex is then exposed to the light of the appropriate wavelength, the fluorescence observed indicates the presence of the molecular marker of interest. Immunofluorescence and EIA techniques are both very well established in the art and are discussed herein. In some embodiments, an assay such as an ELISA assay can be used. ELISA assays are known in the art and commercially available.

In some embodiments, flow cytometry (FACS) can be used to detect the protein level of a biomarker. Surface proteins can be detected using antibodies against specific biomarkers. The flow cytometer detects and reports the intensity of the fluorochrome-tagged antibody, which indicates the expression level of the biomarker. Non-fluorescent cytoplasmic proteins can also be observed by staining permeabilized cells. The stain can either be a fluorescence compound able to bind to certain molecules, or a fluorochrome-tagged antibody to bind the molecule of choice.

Antibodies that specifically bind CA-125 (e.g., human CA-125) are known in the art and commercially available from many sources, for example, Invitrogen, BioLegend, LifeSpan BioSciences, Thermo Fisher Scientific, Merk, etc.

Methods provided herein comprise measuring the expression level of CA-125 in a subject. In some embodiments, the methods provided herein comprise measuring the mRNA level. Any method as described herein or otherwise known in the art to determine the mRNA level of a gene can be used. The mRNA sequence (e.g., the mRNA of CA-125, or a fragment thereof) can be used to prepare a probe that is at least partially complementary. The probe can then be used to detect the mRNA sequence in a sample, using any suitable assay, such as PCR-based methods, northern blotting, a dipstick assay, and the like.

Measuring mRNA levels traditionally involve isolating an intact RNA fraction from samples, immobilizing it, and detecting and quantifying the RNA transcripts of interest. This can be done using a transcript-specific, labeled probe. Exemplary techniques include: northern blotting, dot blotting, ribonuclease protection assays (RPAs), serial analysis of gene expression (SAGE), and differential or subtractive hybridization.

Current approaches provide greater detection efficiency than immobilized RNA techniques, and they are adaptable for increased target and sample numbers. They usually involve adding multiple probes to an RNA fraction or directly to a cell lysate. These techniques include polymerase chain reaction (PCR)-based methods such as quantitative PCR (qPCR), digital PCR (dPCR), or real-time PCR (RT-PCR), next generation sequencing (NGS), microarrays and panels, and in situ hybridization, including fluorescent in situ hybridization (FISH). In some embodiments, the mRNA is measured using in situ RNA hybridization (e.g., FISH), qPCR, RT-PCR, microarray analysis, SAGE, MassARRAY, or NGS.

In some embodiments, the CA-125 expression is measured using fluorescent dyes. Fluorescent dyes are, e.g., described by Briggs et al., J. Chem. Soc., Perkin-Trans. 1 (1997) 1051-1058). Fluorescent labels or fluorophores include rare earth chelates (europium chelates), fluorescein type labels including xanthene dyes, fluorescein isothiocyanate (FITC), 5-carboxyfluorescein, 6-carboxy fluorescein (FAM), 6 carboxy-2′,4′,7′,4,7-hexachlorofluorescein (HEX), 6 carboxy 4′, 5′ dichloro 2′, T dimethoxyfluorescein (JOE or J), fluorescein chlorotriazinyl, and napthofluorescein eosin; rhodamine type labels including N,N,N′,N′ tetramethyl 6 carboxyrhodamine (TAMRA or T), tetramethylrhodamine, 6 carboxy X rhodamine (ROX or R), 5 carboxyrhodamine 6G (R6G5 or G5), 6 carboxyrhodamine 6G (R6G6 or G6), and rhodamine 110 (R100); cyanines including Cy3, Cy5 and Cy7 dyes; Alexa dyes, e.g., Alexa-fluor-555; coumarin, diethylaminocoumarin, umbelliferone; benzimide dyes (e.g., Hoechst 33258); phenanthridine dyes (e.g., Texas Red); ethidium dyes; acridine dyes; carbazole dyes; phenoxazine dyes; porphyrin dyes; polymethine dyes, BODIPY dyes, quinoline dyes, pyrene, phycoerythrins; dansyl; lissamine; and analogs thereof. The fluorescent labels can be conjugated to an aldehyde group comprised in target molecule using the techniques disclosed herein.

Fluorescent dyes and fluorescent label reagents include those which are commercially available from Invitrogen/Molecular Probes (Eugene, Oregon, USA) and Pierce Biotechnology, Inc. (Rockford, III.).

In some embodiments, the CA-125 expression is measured using luminescent dyes: Luminescent dyes or labels can be further subcategorized into chemiluminescent and electrochemiluminescent dyes. The different classes of chemiluminogenic labels include luminol, acridinium compounds, coelenterazine and analogues, dioxetanes, systems based on peroxyoxalic acid and their derivatives. For immunodiagnostic procedures predominantly acridinium based labels are used (a detailed overview is given in Dodeigne C. et al., Talanta 51 (2000) 415-439). The labels of major relevance used as electrochemiluminescent labels are the Ruthenium- and the Iridium-based electrochemiluminescent complexes, respectively.

Electrochemiluminescense (ECL) can be used in analytical applications as a highly sensitive and selective method. It combines analytical advantages of chemiluminescent analysis (absence of background optical signal) with ease of reaction control by applying electrode potential. In general Ruthenium complexes, especially [Ru (Bpy)3]2+(which releases a photon at −620 nm) regenerating with TPA (Tripropylamine) in liquid phase or liquid-solid interface are used as ECL-labels. ECL uses labels or other reactants that can be induced to luminesce when electrochemically oxidized or reduced in an appropriate chemical environment. Such electrochemiluminescense is triggered by a voltage imposed on a working electrode at a particular time and in a particular manner. The light produced by the label is measured and indicates the presence or quantity of the analyte. For a fuller description of such ECL techniques, reference is made to U.S. Pat. Nos. 5,221,605; 5,591,581; 5,597,910; 5,679,519; and to PCT published applications WO90/05296, WO92/14139, WO90/05301, WO96/24690, WO95/08644, WO96/06946, WO96/33411, WO87/06706, WO96/39534, WO96/41175, WO96/40978, and WO2012107419.

Methods provided herein comprise measuring the expression level of CA-125 in a sample of the subject. In some embodiments, the subject is a mammal. In some embodiments, the subject is a human. In some embodiments, the subject is a human female. In some embodiments, the subject is an adolescent human female. In some embodiments, the subject is a human female aged between 12-24 years. In some embodiments, the subject is a human female exhibiting at least one clinical indicator of endometriosis. Clinical indicators of endometriosis include, e.g., intermenstrual bleeding, dysmenorrhea, dyspareunia, dyschezia, dysuria, chronic pelvic pain, lower abdominal pain, or infertility. In some embodiments, the subject exhibits no clinical indicator of endometriosis. In some embodiments, the subject has a family history of endometriosis. In some embodiments of the methods of prognosis disclosed herein, the subject is known to have endometriosis.

In some embodiments, methods provided herein comprise obtaining the sample from the subject. In some embodiments, the sample is obtained from a subject during a specific time window. The menstrual cycle can be divided into several phases, including (1) menstrual phase, in which the endometrium sheds if no pregnancy occurred during the previous cycle, resulting in menstruation; (2) proliferative phase (also known as the follicular phase), in which rising levels of estrogen stimulate the growth and development of the ovarian follicles, characterized by an increase in the thickness of the endometrial lining; (3) ovulation, in which one or more eggs are released from one of the mature follicles in the ovary; and (4) luteal phase (also known as the secretory phase), in which the ruptured follicle transforms into the corpus luteum, which produces progesterone. If fertilization and implantation do not occur, the corpus luteum degenerates, leading to a drop in progesterone levels and the start of menstruation. The menstrual cycle, although the duration can vary among individuals, is typically around 28 days long for human females. A typical cycle can include (1) menstrual phase (Days 1-5), (2) proliferative phase or (Days 6-14), (3) ovulation (about Day 14); and (4) luteal phase or secretory phase (Days 15-28).

Unexpectedly, it is discovered by inventors that the CA-125 level of a subject during secretory phase of the menstrual cycle of the subject provides much better indication for the presence of endometriosis that that during other phases of the menstrual cycle. Accordingly, in some embodiments, the sample is obtained from a subject during the secretory phase of a menstrual cycle. In some embodiments, methods provided herein comprise obtaining the sample during the secretory phase of the subject and measuring the CA-125 level in the sample. In some embodiments, methods provided herein comprise obtaining the sample during Days 15-28 of a menstrual cycle of a human female and measuring the CA-125 level in the sample.

Hormone (progesterone and estrogen) levels vary throughout these phases and play crucial roles in regulating the cycle. In brief, during menstrual Phase (Days 1-5), both progesterone and estrogen levels are low; during proliferative Phase (Days 6-14), progesterone level is low and estrogen level is increasing; at ovulation (around Day 14), progesterone level is low and estrone level is at peak; and then during the secretory phase (Day 15-28), progesterone level is high and estrogen level is moderate to high. As such, monitoring the fluctuations of progesterone and estrogen levels helps in identifying the different phases of the menstrual cycle. In some embodiments, methods provided herein comprise obtaining the sample when the progesterone level is high, indicating secretory phase. In some embodiments, methods provided herein comprise obtaining the sample when the progesterone level is low and estrogen level is above a reference level, indicating proliferative phase.

The sample used in methods disclosed herein can be derived from any biological source, such as tissues (e.g., endometrial tissue), extracts, cells, or cell cultures, including nucleated cells, cell lysates, conditioned medium from fetal or maternal cells, and physiological fluids, such as, for example, whole blood, plasma, serum, saliva, ocular lens fluid, cerebral spinal fluid, sweat, urine, milk, ascites fluid, amniotic fluid, vaginal fluid, synovial fluid, peritoneal fluid, and the like. In some embodiments, the sample is a peritoneal fluid sample.

In some embodiments, the sample used in methods disclosed herein is a physiological fluid, such as blood. In some embodiments, samples used in methods disclosed herein comprise endometrial cells. In some embodiments, the sample is a menstrual blood. In some embodiments, the sample is peripheral blood. In some embodiments, the sample is serum sample. In some embodiments, the serum is processed from blood within 2 hours of blood draw.

In some embodiments, the sample is a uterine tissue sample. In some embodiments, the sample can be fluids or washings of the uterine lining, or sample prepared by similar techniques involving cervical lavage or brushings. In some embodiments, samples used in methods disclosed herein comprise endometrial cells. In some embodiments, the tissue sample comprises epithelial cells. In some embodiments, the sample comprises cells from an endometrial cyst.

In some embodiments, the sample is a pap smear sample. Standard procedures for pap smear are well known in the art. The pap smear sample is a collection of cells from the cervix, which is commonly obtained in conjunction with a pelvic exam.

In some embodiments, the sample is preserved after being obtained from the subject to minimize the possible loss of DNA, RNA, and/or protein during handling. In some embodiments, the sample is preserved before being processed or analyzed. The sample can be preserved using any means known in the art. In some embodiments, preserving the sample comprises placing the sample in a fixative. The fixative can be liquid. The fixative can be solid. In some embodiments, the fixative is neutral buffered formalin (NBF). NBF typically includes formaldehyde, methanol, and phosphate buffer. In some embodiments, the fixative is aldehyde-based. In some embodiments, the fixative is ethanol-based. The ethanol-based fixative can have >50% (e.g., 60%, 70%, 80%, 90% or 100%) ethanol. In some embodiments, the ethanol-based fixative can have 70% ethanol. In some embodiments, the fixative can have at least one component selected from glycerol, glacial acetic acid, a chao trope or denaturant (for example, guanidinium thiocyanate, guanidinium HCl, or guanidinium acetate), trehalose, polyethylene glycol (e.g., PEG200), ethylenediaminetetraacetic acid (EDTA), ethylene glycol-bis(-aminoethyl ether) ethylenediaminetetraacetic acid (EGTA), acrylamide, trichloroacetic acid, acetate salt (e.g., zinc acetate, copper acetate, or magnesium acetate), acetonitrile, and ethylene glycol. In some embodiments, the fixative is powder.

In some embodiments, the sample used in methods disclosed herein can be directly obtained from the source or pretreated prior to use. In some embodiments, a sample can be treated prior to use, such as preparing plasma from blood, diluting viscous fluids, and the like. Methods of treatment can involve filtration, distillation, extraction, concentration, inactivation of interfering components, the addition of reagents, and the like. In some embodiments, the cells contained in a sample (e.g., a menstrual blood sample or an endometrial biopsy sample) are washed, mixed, allowed to settle, and embedded according to standard procedures. In some embodiments, the sample is treated by appropriate measures to release the gene products (protein and/or mRNA) from cellular constituents. As a result, the gene product(s) of CA-125 is obtained in soluble and easily accessible form. In some embodiments, the sample is diluted into an appropriate incubation buffer. Such incubation buffers are well-known to the skilled artisan. For illustrative purposes, if immunoassays are used, an appropriate buffer is selected that can serve both the purposes to liberate and/or solubilize proteins and to allow for immunological binding and thus for formation of an immunological complex for detection.

In some embodiments, methods provided herein further comprise obtaining the sample from the subject. As such, provided herein are methods of assessing whether a subject has endometriosis, wherein the methods comprise (1) obtaining a blood sample from the subject during the secretory phase of menstrual cycle; (2) processing the blood sample to obtain a serum sample within 2 hours of blood draw; and (3) measuring the expression level of CA-125 in the serum sample. In some embodiments, the expression level of CA-125 in the sample is compared to a reference level; wherein an increased expression level indicates that the subject is likely to have endometriosis.

### 7.4 miRNA Biomarkers

MicroRNAs, or miRNAs, are a class of highly conserved small endogenous noncoding, functional RNA molecules of 19-24 nucleotides. The primary function of miRNAs involves binding to the 3′ untranslated region (UTR) of target messenger RNAs (mRNAs), leading to the degradation of the mRNA or inhibiting its translation into protein. This mechanism enables miRNAs to fine-tune gene expression, influencing various cellular processes such as cell cycle progression, apoptosis, and differentiation. MiRNAs also play a pivotal role in developmental processes, contributing to tissue morphogenesis and organ development. Furthermore, miRNAs are responsive to environmental stressors and contribute to stress responses in cells. They are involved in immune system regulation, modulating the expression of genes associated with immune cell development, function, and signaling. Additionally, miRNAs act as critical players in maintaining cellular homeostasis by influencing responses to oxidative stress, DNA damage, and other cellular stresses. In summary, miRNAs serve as versatile regulators of gene expression, orchestrating a wide array of biological processes essential for proper development, cellular function, and overall organismal health.

Dysregulation of miRNA expression has been implicated in diseases, including cancer, neurodegenerative disorders, and cardiovascular diseases, making them potential biomarkers and therapeutic targets. Methods and systems provided herein are based in part on the surprising finding that a combination of miRNA markers can be used to detect endometriosis with high sensitivity and specificity. miRNAs are involved in molecular pathways associated with endometriosis and serve as putative biomarkers.

Provided herein are miRNA biomarkers or combinations thereof, wherein the expression level or expression pattern can indicate the presence of gynecological disease. In some embodiments, one or more biomarkers associated with endometriosis are up-regulated, or expressed at a higher than normal level. In some embodiments, one or more biomarkers associated with endometriosis are down-regulated, or expressed at a lower than normal level. In some embodiments, the expression level or expression pattern of the miRNA biomarkers can be used to detect endometriosis. In some embodiments, the expression level or expression pattern of the miRNA biomarkers can be used to distinguish endometriosis and other gynecological diseases, including, for example, physiological cyst, hemorrhagic cyst, and leiomyomas. In some embodiments, the expression level or expression pattern of the miRNA biomarkers can be used to distinguish between minimal and mild (Stage I+II) endometriosis versus moderate and severe (Stage III+IV) endometriosis. In some embodiments, the expression level or expression pattern of the miRNA biomarkers can be used to distinguish between patients who have endometriosis only, and those who have both endometriosis and adenomyosis.

Thus, the disclosure relates to compositions and methods useful for the diagnosis, detection, assessment, and characterization of general gynecological diseases, endometriosis, and/or adenomyosis in a subject in need thereof, based upon the expression level or expression pattern of one or more biomarkers disclosed herein.

In exemplary embodiments, the miRNAs are selected from: miR-17, miR-19b, miR-21, miR-15b, miR-19a, miR-664a, miR-381, miR-23a, miR-654, miR-24, Let-7b, miR-20a, miR-22, miR-34c, miR-1287, miR-625, miR-1294, miR-7704, miR-221, miR-340, miR-450b, miR-548e, miR-502, miR-574, miR-9, miR-299, miR-6789, miR-593, miR-346, miR-34a, miR-449a, miR-7109, miR-3907, miR-557, miR-6801, miR-4420, miR-570, miR-155, miR-199, miR-520d, miR-424, and miR-200. In exemplary embodiments, the miRNAs are selected from: miR-34c, miR-155, miR-22, miR-23a, miR-17, miR-20a, Let-7b, miR-199, miR-574, miR-520d, miR-424, and miR-200. In exemplary embodiments, the miRNAs can be miRNAs targeting Cancer Antigen 125 (CA-125), Sirtuin 1 (SIRT1) or B Cell Lymphoma 6 (BCL6). Sequences of the miRNA family members are publicly available from miRBase at (mirbase.org).

miR-155: miR-155 is highly expressed in regulatory T-cells (Tregs), where it is targeted by transcription factor fork head box P3 (FOXP3). FOXP3 also plays a role in the inflammatory aspect of endometriosis. miR-155-5p and miR-155-3p have been identified as two isotypes. In some embodiments, miR-155 can serve as a biomarker for gynecological disease, such as endometriosis. That is, an altered expression level of miR-155 in a sample from a subject as compared to a reference level can indicate that the subject has a gynecological disease, such as endometriosis. In some embodiments, an increase in expression level of miR-155 in a sample from a subject as compared to a reference level can indicate that the subject has a gynecological disease, such as endometriosis. miR-155 includes two isoforms: miR-155-5p and miR-155-3p. In some embodiments, miR-155-5p can serve as a biomarker for gynecological disease, such as endometriosis. In some embodiments, miR-155-3p can serve as a biomarker for gynecological disease, such as endometriosis.

miR-34: miR-34 is a family of microRNAs involved in the regulation of various cellular processes, including cell cycle control, apoptosis, and senescence. miR-34a, miR-34b, and miR-34c are three isoforms within this family, each playing a role in different tissues. miR-34a is known for its tumor-suppressive functions and is primarily expressed in the brain and other tissues, while miR-34b and miR-34c are more commonly found in the lung and other tissues. In some embodiments, miR-34 can serve as a biomarker for gynecological diseases, such as endometriosis. That is, an altered expression level of miR-34 in a sample from a subject as compared to a reference level can indicate that the subject has a gynecological disease, such as endometriosis. In some embodiments, an increase in expression level of miR-34 in a sample from a subject as compared to a reference level can indicate that the subject has a gynecological disease, such as endometriosis. miR-34 includes three isoforms: miR-34a, miR-34b, and miR-34c. In some embodiments, miR-34a can serve as a biomarker for gynecological diseases, such as endometriosis. In some embodiments, miR-34b and miR-34c can also serve as biomarkers for gynecological diseases, such as endometriosis. In some embodiments, miR-34c-5p can serve as a biomarker for gynecological diseases, such as endometriosis. In some embodiments, miR-34c-3p can serve as a biomarker for gynecological diseases, such as endometriosis.

miR-199: miR-199 is a microRNA that is known to play significant roles in various physiological and pathological processes, including inflammation, fibrosis, and cancer. miR-199a and miR-199b are two isoforms of this microRNA, with distinct but overlapping functions. miR-199a is further divided into miR-199a-3p and miR-199a-5p, while miR-199b is divided into miR-199b-3p and miR-199b-5p. In some embodiments, miR-199 can serve as a biomarker for gynecological diseases, such as endometriosis. That is, an altered expression level of miR-199 in a sample from a subject as compared to a reference level can indicate that the subject has a gynecological disease, such as endometriosis. In some embodiments, an altered expression level of miR-199 in a sample from a subject as compared to a reference level can indicate that the subject has a gynecological disease, such as endometriosis. miR-199 includes two main isoforms: miR-199a and miR-199b. In some embodiments, miR-199a-3p and miR-199a-5p can serve as biomarkers for gynecological diseases, such as endometriosis. In some embodiments, miR-199b-3p and miR-199b-5p can also serve as biomarkers for gynecological diseases, such as endometriosis.

miR-22: miR-22 promotes tumor initiation, progression, and metastasis by maintaining Wnt/β-catenin signaling and cancer stem cell function. miR-22-3p is the identified isotype for miR-22. In some embodiments, miR-22 can serve as a biomarker for gynecological disease. That is, an altered expression level of miR-22 in a sample from a subject as compared to a reference level can indicate that the subject has a gynecological disease, such as endometriosis. In some embodiments, an increase in expression level of miR-22 in a sample from a subject as compared to a reference level can indicate that the subject has a gynecological disease, such as endometriosis. miR-22 includes two isoforms: miR-22-3p and miR-22-5p. In some embodiments, miR-22-3p can serve as a biomarker for gynecological disease. In some embodiments, miR-22-5p can serve as a biomarker for gynecological disease.

miR-23a: miR-23a directly regulates EMT through its targeting of SMAD3 to inhibit TGF-β. miR-23a-5p and miR-23a-3p are the two isotypes linked to miR-23a. In some embodiments, miR-23a can serve as a biomarker for gynecological disease. miR-23a includes two isoforms: miR-23a-5p and miR-23a-3p. In some embodiments, miR-23a-5p can serve as a biomarker for gynecological disease. That is, an altered expression level of miR-23a in a sample from a subject as compared to a reference level can indicate that the subject has a gynecological disease, such as endometriosis. In some embodiments, an increase in expression level of miR-23a in a sample from a subject as compared to a reference level can indicate that the subject has a gynecological disease, such as endometriosis. In some embodiments, miR-23a-3p can serve as a biomarker for gynecological disease.

miR-17: miR-17 is involved in promoting cell proliferation and inhibiting apoptosis. Specifically, miR-17 targets anti-proliferative factors such as PTEN and p21, contributing to increased cell proliferation. Moreover, it plays a role in inhibiting apoptosis by targeting pro-apoptotic proteins like Bim. Dysregulation of miR-17 has been observed in various cancers. Two isotypes, miR-17-5p and miR-17-3p, are associated with miR-17. In some embodiments, miR-17 can serve as a biomarker for gynecological disease. That is, an altered expression level of miR-17 in a sample from a subject as compared to a reference level can indicate that the subject has a gynecological disease, such as endometriosis. In some embodiments, an increase in expression level of miR-17 in a sample from a subject as compared to a reference level can indicate that the subject has a gynecological disease, such as endometriosis. miR-17 includes two isoforms: miR-17-5p and miR-17-3p. In some embodiments, miR-17-5p can serve as a biomarker for gynecological disease. In some embodiments, miR-17-3p can serve as a biomarker for gynecological disease.

miR-20a: Similar to miR-17, miR-20a also play critical roles in the pathogenesis of endometriosis and its progression. The target genes of miR-20a include HIF1A, VEGFA, BCL2, CDKN1A/p21, CCND1(cyclinDl), and E2F3, interleukin (IL)8, and transforming growth factor (TGF)β, involved in hypoxia responses and inflammation, angiogenesis, cell proliferation and survival, lesion progression, and epithelial-mesenchymal transition. miR-20a-5p and miR-20a-3p represent the two isotypes of miR-20a. In some embodiments, miR-20a can serve as a biomarker for gynecological disease. That is, an altered expression level of miR-20a in a sample from a subject as compared to a reference level can indicate that the subject has a gynecological disease, such as endometriosis. In some embodiments, an increase in expression level of miR-20a in a sample from a subject as compared to a reference level can indicate that the subject has a gynecological disease, such as endometriosis. miR-20a includes two isoforms: miR-20a-5p and miR-20a-3p. In some embodiments, miR-20a-5p can serve as a biomarker for gynecological disease. In some embodiments, miR-20a-3p can serve as a biomarker for gynecological disease.

Let-7b: Let-7b expression correlates with expression of multiple genes essential to the pathophysiology of endometriosis. These genes include ER-α, ER-β, Cyp19a, KRAS 4A, KRAS 4B and IL-6. Two isotypes, Let-7b-5p and Let-7b-3p, are associated with Let-7b. In some embodiments, Let-7b can serve as a biomarker for gynecological disease. That is, an altered expression level of Let-7b in a sample from a subject as compared to a reference level can indicate that the subject has a gynecological disease, such as endometriosis. In some embodiments, an increase in expression level of Let-7b in a sample from a subject as compared to a reference level can indicate that the subject has a gynecological disease, such as endometriosis. Let-7b includes two isoforms: Let-7b-5p and Let-7b-3p. In some embodiments, Let-7b-5p can serve as a biomarker for gynecological disease. In some embodiments, Let-7b-3p can serve as a biomarker for gynecological disease.

miR-574: miR-574 is a tumor suppressor miRNA. Bioinformatics analysis in published studies revealed that miR-574 can simultaneously target BCL11A and SOX2. thereby inhibiting the proliferation, migration and EMT of TNBC. Data also support that miR-574-3p expression plays a role in inflammation via NFκB. miR-574-5p and miR-574-3p represent the two isotypes of miR-574. In some embodiments, miR-574 can serve as a biomarker for gynecological disease. That is, an altered expression level of miR-574 in a sample from a subject as compared to a reference level can indicate that the subject has a gynecological disease, such as endometriosis. In some embodiments, an altered expression level of miR-574 in a sample from a subject as compared to a reference level can indicate that the subject has a gynecological disease, such as endometriosis. miR-574 includes two isoforms: miR-574-5p and miR-574-3p. In some embodiments, miR-574-5p can serve as a biomarker for gynecological disease. In some embodiments, miR-574-3p can serve as a biomarker for gynecological disease.

miR-520d: miR-520d-3p has been shown to directly target certain oncogenes to exert its suppressive effects in tumor cells, which include pituitary tumor transforming gene 1 (PTTG1) in glioma, anti-silencing function 1B histone chaperone (ASF1B) in melanoma, spindle and kinetochore associated 2 (SKA2) in breast cancer, MIG-7 in osteosarcoma, and ZFP36L2 in cervical cancer. miR-520d-5p and miR-520d-3p are the two isotypes identified for miR-520d. In some embodiments, miR-520d can serve as a biomarker for gynecological disease. That is, an altered expression level of miR-520d in a sample from a subject as compared to a reference level can indicate that the subject has a gynecological disease, such as endometriosis. In some embodiments, an altered expression level of miR-520d in a sample from a subject as compared to a reference level can indicate that the subject has a gynecological disease, such as endometriosis. miR-520d includes two isoforms: miR-520d-5p and miR-520d-3p. In some embodiments, miR-520d-5p can serve as a biomarker for gynecological disease. In some embodiments, miR-520d-3p can serve as a biomarker for gynecological disease.

miR-424: miR-424 is involved in cell cycle regulation by targeting key molecules such as Cyclin D1 and Cyclin E1, impacting the G1 to S phase transition and influencing cell proliferation. Additionally, it plays a significant role in angiogenesis by modulating factors like Vascular Endothelial Growth Factor (VEGF) and endothelial Nitric Oxide Synthase (eNOS). It also participates in hematopoiesis by regulating erythropoiesis through the targeting of Kit and Erythropoietin receptor (EpoR). miR-424 also appears to be involved in tumorigenesis. miR-424-5p and miR-424-3p are the two isotypes associated with miR-424. In some embodiments, miR-424 can serve as a biomarker for gynecological disease. That is, an altered expression level of miR-424 in a sample from a subject as compared to a reference level can indicate that the subject has a gynecological disease, such as endometriosis. In some embodiments, an altered expression level of miR-424 in a sample from a subject as compared to a reference level can indicate that the subject has a gynecological disease, such as endometriosis. miR-424 includes two isoforms: miR-424-5p and miR-424-3p. In some embodiments, miR-424-5p can serve as a biomarker for gynecological disease. In some embodiments, miR-424-3p can serve as a biomarker for gynecological disease.

miR-200: miR-200 is known to regulate epithelial-mesenchymal transition (EMT), a critical process in embryonic development and cancer progression. The miR-200 family microRNAs help maintain epithelial characteristics and inhibit the transition of cells to a mesenchymal state. miR-200 prevents the loss of cell adhesion and polarity associated with the mesenchymal phenotype. Dysregulation of miR-200 has been implicated in various malignancies. The miR-200 family comprises two subfamilies, miR-200a/b/c and miR-141/429, each with distinct isotypes including miR-200a-5p, miR-200a-3p, miR-200b-5p, miR-200b-3p, miR-200c-5p, miR-200c-3p, miR-141-3p, and miR-429-3p, among others. In some embodiments, miR-200 can serve as a biomarker for gynecological disease. That is, an altered expression level of miR-200 family miRNAs in a sample from a subject as compared to a reference level can indicate that the subject has a gynecological disease, such as endometriosis. In some embodiments, an altered expression level of miR-200 family miRNAs in a sample from a subject as compared to a reference level can indicate that the subject has a gynecological disease, such as endometriosis. The miR-200 family consists of two subfamilies, miR-200a/b/c and miR-141/429. Isoforms within each subfamily include miR-200a-5p, miR-200a-3p, miR-200b-5p, miR-200b-3p, miR-200c-5p, miR-200c-3p, miR-141-3p, miR-429-3p, among others. In some embodiments, miR-200a-5p can serve as a biomarker for gynecological disease. In some embodiments, miR-200a-3p can serve as a biomarker for gynecological disease. In some embodiments, miR-200b-5p can serve as a biomarker for gynecological disease. In some embodiments, miR-200b-3p can serve as a biomarker for gynecological disease. In some embodiments, miR-200c-5p can serve as a biomarker for gynecological disease. In some embodiments, miR-200c-3p can serve as a biomarker for gynecological disease. In some embodiments, miR-141-3p can serve as a biomarker for gynecological disease. In some embodiments, miR-429-3p can serve as a biomarker for gynecological disease.

miR-9: miR-9 plays an important role in regulating key physiological pathways in endometrial tissue. Kluz et al., Cancers 16.13 (2024): 2416. Also, miR-9 plays a key role in inhibiting p53-suppressor protein-dependent proliferation. One of the regulatory targets of miR-9 is FOXP1, a gene that contains information about an anti-apoptotic protein known to be overexpressed in the endometrium of patients with diseases such as endometriosis. miR-9-5p and miR-9-3p represent the two isotypes of miR-9. In some embodiments, miR-9 can serve as a biomarker for gynecological disease. That is, an altered expression level of miR-9 in a sample from a subject as compared to a reference level can indicate that the subject has a gynecological disease, such as endometriosis. miR-9 includes two isoforms: miR-9-5p and miR-9-3p. In some embodiments, miR-9-5p can serve as a biomarker for gynecological disease. In some embodiments, miR-9-3p can serve as a biomarker for gynecological disease.

miR-299: miR-299 has been found to play a role in cell proliferation, motility, invasion and angiogenesis via VEGFA. It has been revealed that miR-299 participates in the regulation of hematopoietic progenitor fate, modulating megakaryocytic-granulocytic versus erythroid-monocytic differentiation. Wang et al., The Journal of Gene Medicine 26.1 (2024): e3616; Tenedini et al., Cell death & disease 1.2 (2010): e28-e28. miR-299-5p and miR-299-3p represent the two isotypes of miR-299. In some embodiments, miR-299 can serve as a biomarker for gynecological disease. That is, an altered expression level of miR-299 in a sample from a subject as compared to a reference level can indicate that the subject has a gynecological disease, such as endometriosis. miR-299 includes two isoforms: miR-299-5p and miR-299-3p. In some embodiments, miR-299-5p can serve as a biomarker for gynecological disease. In some embodiments, miR-299-3p can serve as a biomarker for gynecological disease.

miR-6789: miR-6789 has been found to participate in the pathology of venous thromboembolism (VTE). Gabler et al., The American Journal of the Medical Sciences 361.4 (2021): 509-516. miR-6789-5p and miR-6789-3p represent the two isotypes of miR-6789. In some embodiments, miR-6789 can serve as a biomarker for gynecological disease. That is, an altered expression level of miR-6789 in a sample from a subject as compared to a reference level can indicate that the subject has a gynecological disease, such as endometriosis. miR-6789 includes two isoforms: miR-6789-5p and miR-6789-3p. In some embodiments, miR-6789-5p can serve as a biomarker for gynecological disease. In some embodiments, miR-6789-3p can serve as a biomarker for gynecological disease.

miR-593: miR-593 plays an important role in cell proliferation, cell invasion and apoptosis, such as in non-small cell lung cancer (NSCLC) cells by targeting SLUG-associated signaling pathways. miR-593 suppresses the proliferation of lung cancer cells, induces apoptosis in NSCLC cells, and suppresses cellular migration and invasion by impairing epithelial-mesenchymal transition (EMT). Wei et al., Molecular Medicine Reports 20.6 (2019): 5172-5182. miR-593-5p and miR-593-3p represent the two isotypes of miR-593. In some embodiments, miR-593 can serve as a biomarker for gynecological disease. That is, an altered expression level of miR-593 in a sample from a subject as compared to a reference level can indicate that the subject has a gynecological disease, such as endometriosis. miR-593 includes two isoforms: miR-593-5p and miR-593-3p. In some embodiments, miR-593-5p can serve as a biomarker for gynecological disease. In some embodiments, miR-593-3p can serve as a biomarker for gynecological disease.

miR-346: miR-346 plays a key role in the occurrence and development of many diseases. miR-346 can inhibit the proliferation of neural stem cells and promote their apoptosis and can promote the progression of enteritis by down-regulating intestinal mucosal vitamin D receptors. miR-346 has been revealed to play an important role in myocardial inflammation and apoptosis after myocardial infarction via targeting nuclear factor I/B (NFIB). Yang et al., European Review for Medical & Pharmacological Sciences 24.22 (2020). In some embodiments, miR-346 can serve as a biomarker for gynecological disease. That is, an altered expression level of miR-346 in a sample from a subject as compared to a reference level can indicate that the subject has a gynecological disease, such as endometriosis. In some embodiments, miR-346 can serve as a biomarker for gynecological disease.

miR-34a: It has been found that SIRT1 expression and activity are regulated by several miRNAs, and miR-34a is one of them. Over-expressing miR-34a decreases SIRT1 protein level and knocking down miR-34a enhances SIRT1 expression. Studies implicate that miR-34a might regulate p53 through SIRT1 and subsequently FoxO-1 expression in endometriotic tissue. miR-34a is also one of the most recognized tumor suppressor miRNAs in several tumors which has been reported to be a direct transcriptional target of p53. Rezk et al., Non-coding RNA Research 6.1 (2021): 35-41. miR-34a-5p and miR-34a-3p represent the two isotypes of miR-34a. In some embodiments, miR-34a can serve as a biomarker for gynecological disease. That is, an altered expression level of miR-34a in a sample from a subject as compared to a reference level can indicate that the subject has a gynecological disease, such as endometriosis. miR-34a includes two isoforms: miR-34a-5p and miR-34a-3p. In some embodiments, miR-34a-5p can serve as a biomarker for gynecological disease. In some embodiments, miR-34a-3p can serve as a biomarker for gynecological disease.

miR-449a: miR-449a has been revealed to be linked to human endometrial receptivity and have an important impact on caprine endometrial-stromal-cell apoptosis and mice endometrial receptivity. The dynamics of miR-449a expression during uterine cycles are shown to be associated with endometrial development. Naydenov et al., Biology 12.1 (2022): 55. In some embodiments, miR-449a can serve as a biomarker for gynecological disease. That is, an altered expression level of miR-449a in a sample from a subject as compared to a reference level can indicate that the subject has a gynecological disease, such as endometriosis. In some embodiments, miR-449a can serve as a biomarker for gynecological disease.

miR-7109: It has been revealed that miR-7109 critically mediates the regulatory action of long non-coding RNA (IncRNA) LINC00973 on Siglec-15 and Siglec-15 is a direct target of miR-7109. Studies also suggest that miR-7109 might play an important role in the metastasis of oral squamous cell carcinoma (OSCC) by regulating matrix metalloproteinase 7 (MMP7) expression. Liu et al., Cancer Science 111.10 (2020): 3693-3704. miR-7109-5p and miR-7109-3p represent the two isotypes of miR-7109. In some embodiments, miR-7109 can serve as a biomarker for gynecological disease. That is, an altered expression level of miR-7109 in a sample from a subject as compared to a reference level can indicate that the subject has a gynecological disease, such as endometriosis. miR-7109 includes two isoforms: miR-7109-5p and miR-7109-3p. In some embodiments, miR-7109-5p can serve as a biomarker for gynecological disease. In some embodiments, miR-7109-3p can serve as a biomarker for gynecological disease.

miR-3907: miR-3907 has been found to be highly expressed in lung cancer. Studies implicate that miR-3907 promotes the proliferation and migration of sebaceous gland carcinoma (SGC) by downregulating THBS1. miR-3907 is also found to be associated with the pathophysiology of early-onset preeclampsia (EOPE). Zhang et al., Oncology Letters 22.6 (2021): 1-10. In some embodiments, miR-3907 can serve as a biomarker for gynecological disease. That is, an altered expression level of miR-3907 in a sample from a subject as compared to a reference level can indicate that the subject has a gynecological disease, such as endometriosis. In some embodiments, miR-3907 can serve as a biomarker for gynecological disease.

miR-557: miR-557 plays an important role in cell proliferation and invasion probably by negatively regulating the lymphocyte enhancement factor 1 (LEF1) factor, such as in lung cancer cells. Wang et al. RNA biology 18.11 (2021): 1953-1968. Qiu et al., Tumor Biology 39.6 (2017): 1010428317709467. Also, miR-557 involves in angiogenesis. miR-557 has anti-angiogenic function, and reducing miR-557 expression is essential for angiogenesis induced by angiopoietin-1. In some embodiments, miR-557 can serve as a biomarker for gynecological disease. That is, an altered expression level of miR-557 in a sample from a subject as compared to a reference level can indicate that the subject has a gynecological disease, such as endometriosis. In some embodiments, miR-557 can serve as a biomarker for gynecological disease.

miR-6801: miR-6801 is predicted to be involved in various cellular processes and is predicted to be involved in regulating gene expression, potentially impacting cell growth, development and other cellular processes. miR-6801-5p and miR-6801-3p represent the two isotypes of miR-6801. In some embodiments, miR-6801 can serve as a biomarker for gynecological disease. That is, an altered expression level of miR-6801 in a sample from a subject as compared to a reference level can indicate that the subject has a gynecological disease, such as endometriosis. miR-6801 includes two isoforms: miR-6801-5p and miR-6801-3p. In some embodiments, miR-6801-5p can serve as a biomarker for gynecological disease. In some embodiments, miR-6801-3p can serve as a biomarker for gynecological disease.

miR-4420: NF-κB plays an important role in angiogenesis signaling pathway. It has been shown that the target genes of regulated miRNAs by NF-κB such as miR-4420 take part in angiogenesis signaling pathway. Salehi et al, Journal of Cancer Research and Therapeutics 16.Suppl 1 (2020): S90-S94. In some embodiments, miR-4420 can serve as a biomarker for gynecological disease. That is, an altered expression level of miR-4420 in a sample from a subject as compared to a reference level can indicate that the subject has a gynecological disease, such as endometriosis. In some embodiments, miR-4420 can serve as a biomarker for gynecological disease.

miR-570: miR-570 has been shown to involve in cell proliferation, angiogenesis, inflammation and immune response. miR-570 has both direct and indirect effects on many gene products related to the inflammatory response. miR-570 is also found to play an important role in proliferation, angiogenesis and immune escape in cancers such as hepatocellular carcinoma (HCC). miR-570 also involves in cell proliferation, apoptosis and glucose metabolism in chronic myelogenous leukemia (CML) cells through direct targets of insulin receptor substrates (IRS) 1 and IRS2. Roff et al. American Journal of Clinical and Experimental Immunology 3.2 (2014): 68; Lin et al., Cancer Biotherapy and Radiopharmaceuticals 33.6 (2018): 252-257; Zhao et al., Iranian journal of basic medical sciences 20.5 (2017): 481. miR-570-5p and miR-570-3p represent the two isotypes of miR-570. In some embodiments, miR-570 can serve as a biomarker for gynecological disease. That is, an altered expression level of miR-570 in a sample from a subject as compared to a reference level can indicate that the subject has a gynecological disease, such as endometriosis. miR-570 includes two isoforms: miR-570-5p and miR-570-3p. In some embodiments, miR-570-5p can serve as a biomarker for gynecological disease. In some embodiments, miR-570-3p can serve as a biomarker for gynecological disease.

miR-19a/miR-19b: miR-19a and miR-19b (miR-19a/b) are members of the miR-17-92 cluster, which have been implicated in cardiac regeneration, cancer progression, and viral infections. In cardiac regeneration, miR-19a/b have shown protective effects against myocardial infarction (MI) injury. miR-19a/b have also been associated with cancer progression, by promoting cell proliferation, invasion, and epithelial-mesenchymal transition. Wang et al, Mol Ther Oncolytics 2021, 20:290-305; Gao et al., Nat Commun 10, 1802 (2019); Wu et al., Cell Death Dis 5, e1144 (2014). miR-19a-5p and miR-19a-3p are the two isotypes associated with miR-19a. miR-19b-5p and miR-19b-3p are the two isotypes associated with miR-19b. In some embodiments, miR-19a and miR-19b can serve as a biomarker for gynecological disease. That is, an altered expression level of miR-19a and/or miR-19b in a sample from a subject as compared to a reference level can indicate that the subject has a gynecological disease, such as endometriosis. In some embodiments, an increase in expression level of miR-19a, miR-19b, or both in a sample from a subject as compared to a reference level can indicate that the subject has a gynecological disease, such as endometriosis. In some embodiments, miR-19a-5p can serve as a biomarker for gynecological disease. In some embodiments, miR-19a-3p can serve as a biomarker for gynecological disease. In some embodiments, miR-19b-5p can serve as a biomarker for gynecological disease. In some embodiments, miR-19b-3p can serve as a biomarker for gynecological disease.

miR-21: miR-21 is encoded within the intronic region of the TMEM49 gene but is independently transcribed from its own promoter. miR-21 is often classified as an oncomiR due to its upregulation in almost all types of cancers. It contributes to tumor progression through: promoting cell proliferation and invasion, suppressing apoptosis, enhancing metastasis, modulating the tumor microenvironment. Chi et al., Oncogenesis 11, 38 (2022); Kumarswamy, RNA Biol. 2011; 8(5):706-713; Feng & Tsao (2016), Biomedical Reports, 5, 395-402. miR-21 also been implicated in cardiovascular diseases, immune system regulation, central nervous system disorders, embryonic stem cell self-renewal. miR-21-5p and miR-21-3p are the two isotypes associated with miR-21. In some embodiments, miR-21 can serve as a biomarker for gynecological disease. That is, an altered expression level of miR-21 in a sample from a subject as compared to a reference level can indicate that the subject has a gynecological disease, such as endometriosis. In some embodiments, an increase in expression level of miR-21 in a sample from a subject as compared to a reference level can indicate that the subject has a gynecological disease, such as endometriosis. In some embodiments, miR-21-5p can serve as a biomarker for gynecological disease. In some embodiments, miR-21-3p can serve as a biomarker for gynecological disease.

miR-15b: miR-15b belongs to the miR-15/16 cluster. miR-15b exhibits both tumor-suppressive and oncogenic properties in different cancers. It was also implicated in osteoblast differentiation and development of cardiovascular diseases and neurological disorders. Wang et al. (2017). Oncology Reports, 37, 3305-3312; Vimalraj et al., J Cell Physiol. 2014; 229(9):1236-44. Ghafouri-Fard et al., (2022), Front. Oncol. 12:870996. miR-15b-5p and miR-15b-3p are the two isotypes associated with miR-15b. In some embodiments, miR-15b can serve as a biomarker for gynecological disease. That is, an altered expression level of miR-15b in a sample from a subject as compared to a reference level can indicate that the subject has a gynecological disease, such as endometriosis. In some embodiments, an increase in expression level of miR-15b in a sample from a subject as compared to a reference level can indicate that the subject has a gynecological disease, such as endometriosis. In some embodiments, miR-15b-5p can serve as a biomarker for gynecological disease. In some embodiments, miR-15b-3p can serve as a biomarker for gynecological disease.

miR-664a: miR-664A is encoded by MIR664A gene in humans, located on chromosome 1. miR-664a has been found to have both oncogenic and tumor-suppressive properties, depending on the cellular context. The ability of pre-miR-664a to induce apoptosis has led researchers to consider it as a potential nucleic acid drug candidate for cancer therapy. Watanabe et al., Sci Rep 11, 14936 (2021). miR-664a-5p and miR-664a-3p are the two isotypes associated with miR-664a. In some embodiments, miR-664a can serve as a biomarker for gynecological disease. That is, an altered expression level of miR-664a in a sample from a subject as compared to a reference level can indicate that the subject has a gynecological disease, such as endometriosis. In some embodiments, an increased in expression level of miR-664a in a sample from a subject as compared to a reference level can indicate that the subject has a gynecological disease, such as endometriosis. miR-664a includes two isoforms: miR-664a-5p and miR-664a-3p. In some embodiments, miR-664a-5p can serve as a biomarker for gynecological disease. In some embodiments, miR-664a-3p can serve as a biomarker for gynecological disease.

miR-381: miR-381 is encoded by the MIR381 gene, located on chromosome 14q32.31 in humans. miR-381 exhibits diverse functions in different cancer types and exerts its effects through various molecular mechanisms. miR-381 is also implicated in arthritis and intestinal ischemia/reperfusion (I/R) Injury. Liu et al. Cell Death Dis 9, 411 (2018); Tanaka et al., Annals of the Rheumatic Diseases 2016; 75:187; Zhang et al., Oncol Rep 35: 1831-1840, 2016. miR-381-5p and miR-381-3p are the two isotypes associated with miR-381. In some embodiments, miR-381 can serve as a biomarker for gynecological disease. That is, an altered expression level of miR-381 in a sample from a subject as compared to a reference level can indicate that the subject has a gynecological disease, such as endometriosis. In some embodiments, a decrease in expression level of miR-381 in a sample from a subject as compared to a reference level can indicate that the subject has a gynecological disease, such as endometriosis. In some embodiments, miR-381-5p can serve as a biomarker for gynecological disease. In some embodiments, miR-381-3p can serve as a biomarker for gynecological disease.

miR-654: miR-654-3p functions primarily as a tumor suppressor. Its downregulation has been associated with poor prognosis in several cancers. Recent research also highlights its role in cardiac protection. miR-654-5p, derived from the same precursor as miR-654-3p, exhibits distinct functions across different cancers. Zhang et al., Frontiers in genetics 11 (2020): 577948; Liu et al., Int J Mol Sci. 2022; 23(12):6411. miR-654-5p and miR-654-3p are the two isotypes associated with miR-654. In some embodiments, miR-654 can serve as a biomarker for gynecological disease. That is, an altered expression level of miR-654 in a sample from a subject as compared to a reference level can indicate that the subject has a gynecological disease, such as endometriosis. In some embodiments, a decrease in expression level of miR-654 in a sample from a subject as compared to a reference level can indicate that the subject has a gynecological disease, such as endometriosis. In some embodiments, miR-654-5p can serve as a biomarker for gynecological disease. In some embodiments, miR-654-3p can serve as a biomarker for gynecological disease.

miR-24: miR-24 is part of the miR-23-27-24 cluster and plays crucial roles in regulating gene expression, particularly in the context of cancer and cellular differentiation. It exists mainly in two forms: miR-24-3p and miR-24-5p, both of which are involved in various biological processes. miR-24 has a dual role in cancer, acting as both an oncogene and a tumor suppressor depending on the cancer type. miR-24 is involved in several key cellular processes: cell cycle regulation, differentiation, and response to cellular stress. Du et al., J Cell Sci (2013) 126 (6): 1440-1453; Wang et al., Front Oncol. 2020; 10:553714. In some embodiments, miR-24 can serve as a biomarker for gynecological disease. That is, an altered expression level of miR-24 in a sample from a subject as compared to a reference level can indicate that the subject has a gynecological disease, such as endometriosis. In some embodiments, an increase in expression level of miR-24 in a sample from a subject as compared to a reference level can indicate that the subject has a gynecological disease, such as endometriosis. In some embodiments, miR-24-5p can serve as a biomarker for gynecological disease. In some embodiments, miR-24-3p can serve as a biomarker for gynecological disease.

miR-1287: miR-1287 is encoded by the MIR1287 gene. miR-1287-5p, the mature form of miR-1287, has been found to be dysregulated in several types of cancer. Schwarzenbacher et al., Breast Cancer Res. 2019; 21(1):20; Fateh et al., J Gastrointest Cancer. 2016; 47(4):399-403. In some embodiments, miR-1287 can serve as a biomarker for gynecological disease. That is, an altered expression level of miR-1287 in a sample from a subject as compared to a reference level can indicate that the subject has a gynecological disease, such as endometriosis. In some embodiments, an increase in expression level of miR-1287 in a sample from a subject as compared to a reference level can indicate that the subject has a gynecological disease, such as endometriosis. In some embodiments, miR-1287-5p can serve as a biomarker for gynecological disease. In some embodiments, miR-1287-3p can serve as a biomarker for gynecological disease.

miR-625: miR-625 is frequently dysregulated in multiple cancer types. It is often downregulated in several cancers. miR-625 primarily acts as a tumor suppressor and its downregulation is associated with poor prognosis, lymph node metastasis, distant metastasis, and advanced TNM stage in some cancers. Tan et al., Int J Mol Med., 44: 346-356, 2019; Zhang et al., Front Med (Lausanne). 2022; 9:845094. In some embodiments, miR-625 can serve as a biomarker for gynecological disease. That is, an altered expression level of miR-625 in a sample from a subject as compared to a reference level can indicate that the subject has a gynecological disease, such as endometriosis. In some embodiments, a decrease in expression level of miR-625 in a sample from a subject as compared to a reference level can indicate that the subject has a gynecological disease, such as endometriosis. In some embodiments, miR-625-5p can serve as a biomarker for gynecological disease. In some embodiments, miR-625-3p can serve as a biomarker for gynecological disease.

miR-1294: miR-1294 is a microRNA that plays significant roles in various cancers, primarily functioning as a tumor suppressor. It is frequently downregulated in multiple cancer types, including esophageal squamous cell carcinoma (ESCC), gastric cancer (GC), epithelial ovarian cancer (EOC), non-small cell lung cancer (NSCLC), hepatocellular carcinoma (HCC), and cervical cancer. This downregulation is often associated with poor prognosis in several of these cancers. miR-1294 exhibits tumor-suppressive properties by inhibiting proliferation, migration, and invasion of cancer cells, promoting apoptosis, and suppressing epithelial-mesenchymal transition (EMT). Mao et al., Oncol Res. 2023; 31(1):1-12; Guo et al., Eur Rev Med Pharmacol Sci., 2018; 22(22):7646-7652. In some embodiments, miR-1294 can serve as a biomarker for gynecological disease. That is, an altered expression level of miR-1294 in a sample from a subject as compared to a reference level can indicate that the subject has a gynecological disease, such as endometriosis. In some embodiments, a decrease in expression level of miR-1294 in a sample from a subject as compared to a reference level can indicate that the subject has a gynecological disease, such as endometriosis. In some embodiments, miR-1294-5p can serve as a biomarker for gynecological disease. In some embodiments, miR-1294-3p can serve as a biomarker for gynecological disease.

miR-7704: miR-7704 is a microRNA that plays significant roles in various cellular processes and disease contexts. It exhibits context-dependent functions, with potential implications in cancer, hepatic disorders, and immune responses. Arafat et al., Non-coding RNA 9.4 (2023): 42; Naqvi et al., Current Pharmaceutical Design 30.9 (2024): 649-665. In some embodiments, miR-7704 can serve as a biomarker for gynecological disease. That is, an altered expression level of miR-7704 in a sample from a subject as compared to a reference level can indicate that the subject has a gynecological disease, such as endometriosis. In some embodiments, a decrease in expression level of miR-7704 in a sample from a subject as compared to a reference level can indicate that the subject has a gynecological disease, such as endometriosis.

miR-221: miR-221 is often co-expressed with its homolog miR-222, and together they are referred to as the miR-221/222 cluster. miR-221 is involved in several key cellular mechanisms, including regulation of cell proliferation, invasion, and metastasis, modulation of apoptosis, influence on angiogenesis and vascular processes, and promotion of epithelial-mesenchymal transition (EMT). miR-221 primarily functions as an oncogene in many cancer types. Song et al., Frontiers in immunology 8 (2017): 56; Liang et al., npj Breast Cancer 4, 20 (2018). In some embodiments, miR-221 can serve as a biomarker for gynecological disease. That is, an altered expression level of miR-221 in a sample from a subject as compared to a reference level can indicate that the subject has a gynecological disease, such as endometriosis. In some embodiments, an increase in expression level of miR-221 in a sample from a subject as compared to a reference level can indicate that the subject has a gynecological disease, such as endometriosis. In some embodiments, miR-221-5p can serve as a biomarker for gynecological disease. In some embodiments, miR-221-3p can serve as a biomarker for gynecological disease.

miR-340: miR-340 plays significant roles in various cancers and cellular processes. It primarily functions as a tumor suppressor, though its effects can be context-dependent. In cancer, miR-340 has been found to be downregulated in multiple types. Chen et al., Oncol Rep 35: 709-716, 2016; Xi et al., Journal for immunotherapy of cancer 8.1 (2020). In some embodiments, miR-340 can serve as a biomarker for gynecological disease. That is, an altered expression level of miR-340 in a sample from a subject as compared to a reference level can indicate that the subject has a gynecological disease, such as endometriosis. In some embodiments, an increase in expression level of miR-340 in a sample from a subject as compared to a reference level can indicate that the subject has a gynecological disease, such as endometriosis. In some embodiments, miR-340-5p can serve as a biomarker for gynecological disease. In some embodiments, miR-340-3p can serve as a biomarker for gynecological disease.

miR-450b: miR-450b plays significant roles in various cancers and cellular processes. Its functions appear to be context-dependent, acting as both an oncogene and a tumor suppressor depending on the specific cancer type. Ye et al., Oncotarget. 2016; 7(38):61312-61324; Liu et al., International Journal of Molecular Medicine 38.1 (2016): 283-290. In some embodiments, miR-450b can serve as a biomarker for gynecological disease. That is, an altered expression level of miR-450b in a sample from a subject as compared to a reference level can indicate that the subject has a gynecological disease, such as endometriosis. In some embodiments, an increase in expression level of miR-450b in a sample from a subject as compared to a reference level can indicate that the subject has a gynecological disease, such as endometriosis. In some embodiments, miR-450b-5p can serve as a biomarker for gynecological disease. In some embodiments, miR-450b-3p can serve as a biomarker for gynecological disease.

miR-548e: miR-548e is a microRNA that plays significant roles in cancer regulation, particularly in lung cancer. It primarily functions as a tumor suppressor and has been found to be significantly downregulated in tissue specimens. Its low expression is associated with increased cell proliferation, migration, and invasion. Kalhori et al., Scientific reports 10.1 (2020): 1558; Liang et al., BioMed Research International 2012.1 (2012): 679563. In some embodiments, miR-548e can serve as a biomarker for gynecological disease. That is, an altered expression level of miR-548e in a sample from a subject as compared to a reference level can indicate that the subject has a gynecological disease, such as endometriosis. In some embodiments, an increase in expression level of miR-548e in a sample from a subject as compared to a reference level can indicate that the subject has a gynecological disease, such as endometriosis. In some embodiments, miR-548e-5p can serve as a biomarker for gynecological disease. In some embodiments, miR-548e-3p can serve as a biomarker for gynecological disease.

miR-502: The miR-502 gene (MIR502) is located on Chromosome X. miR-502 plays significant roles in various cellular processes and diseases, particularly in cancer. miR-502 primarily functions as a tumor suppressor in various cancers. It exists in two main forms: miR-502-3p and miR-502-5p. Devara et al., Pharmaceuticals (Basel). 2023;16(4):532; Sun et al., Oncol Rep 31: 2085-2092, 2014. In some embodiments, miR-502 can serve as a biomarker for gynecological disease. That is, an altered expression level of miR-502 in a sample from a subject as compared to a reference level can indicate that the subject has a gynecological disease, such as endometriosis. In some embodiments, an increase in expression level of miR-502 in a sample from a subject as compared to a reference level can indicate that the subject has a gynecological disease, such as endometriosis. In some embodiments, miR-502-5p can serve as a biomarker for gynecological disease. In some embodiments, miR-502-3p can serve as a biomarker for gynecological disease.

miRNAs targeting CA-125: miRNAs have been shown to interact with the expression of CA-125 and can influence expression level of CA-125 in patients with CA-125 through targeting the MUC16 gene which encodes CA-125. In some embodiments, a miRNA targeting CA-125 can serve as a biomarker for gynecological disease, such as endometriosis. That is, an altered expression level of a miRNA targeting CA-125 in a sample from a subject as compared to a reference level can indicate that the subject has a gynecological disease, such as endometriosis. In some embodiments, the miRNA targeting CA-125 can be miR-299-3p, miR-299-5p, miR-6789-5p, miR-593-5p, miR-7109-5p, or miR-3907. In some embodiments, miR-299-3p can serve as a biomarker for gynecological disease. In some embodiments, miR-299-5p can serve as a biomarker for gynecological disease. In some embodiments, miR-6789-5p can serve as a biomarker for gynecological disease. In some embodiments, miR-593-5p can serve as a biomarker for gynecological disease. In some embodiments, miR-7109-5p can serve as a biomarker for gynecological disease. In some embodiments, miR-3907 can serve as a biomarker for gynecological disease.

miRNAs targeting SIRT1: SIRT 1, which is Sirtuin 1, is a member of sirtuin family of proteins. SIRT1 links transcriptional regulation directly to intracellular energetics and participates in the coordination of several cellular functions such as cell cycle, response to DNA damage, metabolism, inflammation, angiogenesis, apoptosis and autophagy. SIRT1 is an NAD-dependent deacetylase, and its activity has been implicated in the pathogenesis of various diseases, including endometriosis. miRNAs can be regulators of SIRT1 expression and therefore influence key functions of SIRT1. In some embodiments, a miRNA targeting SIRT1 can serve as a biomarker for gynecological disease, such as endometriosis. That is, an altered expression level of a miRNA targeting SIRT1 in a sample from a subject as compared to a reference level can indicate that the subject has a gynecological disease, such as endometriosis. In some embodiments, the miRNA targeting SIRT1 can be miR-9-5p, miR-34a-5p, miR-449a, miR-4420, or miR-570-3p. In some embodiments, miR-9-5p can serve as a biomarker for gynecological disease. In some embodiments, miR-34a-5p can serve as a biomarker for gynecological disease. In some embodiments, miR-449a can serve as a biomarker for gynecological disease. In some embodiments, miR-4420 can serve as a biomarker for gynecological disease. In some embodiments, miR-570-3p can serve as a biomarker for gynecological disease.

miRNAs targeting BCL6: BCL6, which is B-Cell Lymphoma 6. BCL6 is a transcriptional repressor and functions in immune response, differentiation, proliferation, inflammation, apoptosis and cell cycle. Endometriosis has the characteristics of chronic inflammation and immune dysfunction. miRNAs can be regulators of BCL6 expression and therefore influence key functions of BCL6. In some embodiments, a miRNA targeting BCL6 can serve as a biomarker for gynecological disease, such as endometriosis. That is, an altered expression level of a miRNA targeting BCL6 in a sample from a subject as compared to a reference level can indicate that the subject has a gynecological disease, such as endometriosis. In some embodiments, the miRNA targeting BCL6 can be miR-346, miR-557, or miR-6801-3p. In some embodiments, miR-346 can serve as a biomarker for gynecological disease. In some embodiments, miR-557 can serve as a biomarker for gynecological disease. In some embodiments, miR-6801-3p can serve as a biomarker for gynecological disease.

In some embodiments, provided herein are miRNAs that can serve as biomarkers for detecting a gynecological disease, such as endometriosis, wherein the miRNAs can be miR-17, miR-19b, miR-21, miR-15b, miR-19a, miR-664a, miR-381, miR-23a, miR-654, miR-24, Let-7b, miR-20a, miR-22, miR-34c, miR-1287, miR-625, miR-1294, miR-7704, miR-221, miR-340, miR-450b, miR-548e, miR-502, miR-574, miR-9, miR-299, miR-6789, miR-593, miR-346, miR-34a, miR-449a, miR-7109, miR-3907, miR-557, miR-6801, miR-4420, miR-570, miR-155, miR-199, miR-520d, miR-424, or miR-200, or any combination thereof.

In some embodiments, miR-17 comprises miR-17-5p, miR-17-3p, or both; miR-19b comprises miR-19b-5p, miR-19b-3p, or both; miR-21 comprises miR-21-5p, miR-21-3p, or both; miR-15b comprises miR-15b-5p, miR-15b-3p, or both; miR-19a comprises miR-19a-5p, miR-19a-3p, or both; miR-664a comprises miR-664a-5p, miR-664a-3p, or both; miR-381 comprises miR-381-5p, miR-381-3p, or both; miR-23a comprises miR-23a-5p, miR-23a-3p, or both; miR-654 comprises miR-654-5p, miR-654-3p, or both; miR-24 comprises miR-24-5p, miR-24-3p, or both; Let-7b comprises Let-7b-5p, Let-7b-3p, or both; miR-20a comprises miR-20a-5p, miR-20a-3p, or both; miR-22 comprises miR-22-5p, miR-22-3p, or both; miR-34c comprises miR-34c-5p, miR-34c-3p, or both; miR-1287 comprises miR-1287-5p, miR-1287-3p, or both; miR-625 comprises miR-625-5p, miR-625-3p, or both; miR-1294 comprises miR-1294-5p, miR-1294-3p, or both; miR-7704 comprises miR-7704; miR-221 comprises miR-221-5p, miR-221-3p, or both; miR-340 comprises miR-340-5p, miR-340-3p, or both; miR-450b comprises miR-450b-5p, miR-450b-3p, or both; miR-548e comprises miR-548e-5p, miR-548e-3p, or both; miR-502 comprises miR-502-5p, miR-502-3p, or both; miR-574 comprises miR-574-5p, miR-574-3p, or both; miR-9 comprises miR-9-5p, miR-9-3p, or both; miR-299 comprises miR-299-5p, miR-299-3p, or both; miR-6789 comprises miR-6789-5p, miR-6789-3p, or both; miR-593 comprises miR-593-5p, miR-593-3p, or both; miR-346 comprises miR-346-5p, miR-346-3p, or both; miR-34a comprises miR-34a-5p, miR-34a-3p, or both; miR-449a comprises miR-449a-5p, miR-449a-3p, or both; miR-7109 comprises miR-7109-5p, miR-7109-3p, or both; miR-3907 comprises miR-3907; miR-557 comprises miR-557; miR-6801 comprises miR-6801-5p, miR-6801-3p or both; miR-4420 comprises miR-4420; miR-570 comprises miR-570-5p, miR-570-3p, or both; miR-155 comprises miR-155-5p, miR-155-3p, or both; miR-199 comprises miR-199a-5p, miR-199a-3p, miR-199b-5p, or miR-199b-3p, or any combination thereof; miR-520d comprises miR-520d-5p, miR-520d-3p, or both; miR-424 comprises miR-424-5p, miR-424-3p, or both; miR-200 comprises miR-200a-5p, miR-200a-3p, miR-200b-5p, miR-200b-3p, miR-200c-5p, or miR-200c-3p, or any combination thereof.

In some embodiments, miR-17 comprises miR-17-5p; miR-19b comprises miR-19b-3p; miR-21 comprises miR-21-5p, miR-21-3p, or both; miR-15b comprises miR-15b-5p; miR-19a comprises miR-19a-3p; miR-664a comprises miR-664a-3p; miR-381 comprises miR-381-3p; miR-23a comprises miR-23a-3p; miR-654 comprises miR-654-3p; miR-24 comprises miR-24-3p; Let-7b comprises Let-7b-5p, Let-7b-3p, or both; miR-20a comprises miR-20a-5p; miR-22 comprises miR-22-3p, miR-22-5p, or both; miR-34c comprises miR-34c-3p; miR-1287 comprises miR-1287-5p; miR-625 comprises miR-625-5p; miR-1294 comprises miR-1294-5p; miR-7704 comprises miR-7704; miR-221 comprises miR-221-5p; miR-340 comprises miR-340-5p; miR-450b comprises miR-450b-5p; miR-548e comprises miR-548e-3p; miR-502 comprises miR-502-3p; miR-574 comprises miR-574-3p; miR-9 comprises miR-9-5p; miR-299 comprises miR-299-5p, miR-299-3p, or both; miR-6789 comprises miR-6789-5p; miR-593 comprises miR-593-5p; miR-346 comprises miR-346-5p, miR-346-3p, or both; miR-34a comprises miR-34a-5p; miR-449a comprises miR-449a-5p, miR-449a-3p, or both; miR-7109 comprises miR-7109-5p; miR-3907 comprises miR-3907; miR-557 comprises miR-557; miR-6801 miR-6801-3p; miR-4420 comprises miR-4420; miR-570 comprises miR-570-3p; miR-155 comprises miR-155-5p; miR-199 comprises miR-199a-5p; miR-520d comprises miR-520d-5p, miR-520d-3p, or both; miR-424 comprises miR-424-5p, miR-424-3p, or both; miR-200 comprises miR-200a-5p, miR-200a-3p, miR-200b-5p, miR-200b-3p, miR-200c-5p, or miR-200c-3p, or any combination thereof.

In some embodiments, the miRNA biomarker used for detecting a gynecological disease is selected from: Let-7b, miR-20a, miR-22, miR-34c, miR-574, miR-9, miR-299, miR-6789, miR-593, miR-346, miR-34a, miR-449a, miR-7109, miR-3907, miR-557, miR-6801, miR-4420, and miR-570. In some embodiments, the Let-7b is Let-7b-5p or Let-7b-3p; miR-20a is miR-20a-5p; miR-22 is miR-22-3p; miR-34c is miR-34c-3p; miR-574 is miR-574-3p; miR-9 is miR-9-5p; miR-299 is miR-299-5p or miR-299-3p; miR-6789 is miR-6789-5p; miR-593 is miR-593-5p; miR-34a is miR-34a-5p; miR-7109 is miR-7109-5p; miR-6801 is miR-6801-3p; miR-570 is miR-570-3p.

In some embodiments, the miRNA biomarker used for detecting a gynecological disease is selected from: miR-34c, miR-23a, miR-17, miR-155, Let-7b, miR-199, miR-22, and miR-20a. In some embodiments, the miRNA is selected from: miR-155, miR-22, miR-23a, miR-17, miR-20a, Let-7b, miR-574, and miR-520d. In some embodiments, the miRNA is selected from: miR-155, miR-22, miR-23a, miR-17, miR-20a, and Let-7b. In some embodiments, the miRNA comprises miR-34c. In some embodiments, the miRNA is miR-34c-3p.

In some embodiments, the miRNA biomarker used for detecting a gynecological disease is selected from: miR-17, miR-20a, miR-23a, miR-155 and Let-7b. In some embodiments, the miRNA comprises Let-7b. In some embodiments, the miRNA is Let-7b-5p. In some embodiments, the miRNA is selected from: miR-199, miR-23a, miR-34c and Let-7b. In some embodiments, the miRNA is selected from: miR-155, Let-7b, miR-23a, miR-17, miR-20a, miR-22, miR-199 and miR-34c. In some embodiments, the miRNA comprises miR-17. In some embodiments, the miRNA is miR-17-5p. In some embodiments, the miRNA comprises miR-22.

In some embodiments, the miRNA is miR-22-3p. In some embodiments, the miRNA comprises miR-20a. In some embodiments, the miRNA is miR-20a-5p. In some embodiments, the miRNA comprises miR-34c. In some embodiments, the miRNA is miR-34c-3p. In some embodiments, the miRNA comprises miR-199. In some embodiments, the miRNA is miR-199a-5p.

In some embodiments, the miRNA biomarker used for detecting a gynecological disease is selected from: miR-17, miR-19b, miR-21, miR-15b, miR-19a, miR-664a, miR-381, miR-23a, miR-654, and miR-24. In some embodiments, the miRNA is selected from: miR-17-5p, miR-19b-3p, miR-21-5p, miR-15b-5p, miR-19a-3p, miR-664a-3p, miR-381-3p, miR-23a-3p, miR-654-3p, and miR-24-3p.

In some embodiments, the miRNA biomarker used for detecting a gynecological disease is selected from: miR-21, miR-23a, miR-1287, miR-625, miR-1294, miR-7704, miR-221, miR-340, miR-450b, miR-548e, and miR-502. In some embodiments, the miRNA is selected from: miR-21-3p, miR-22-5p, miR-1287-5p, miR-625-5p, miR-1294-5p, miR-7704, miR-221-5p, miR-340-5p, miR-450b-5p, miR-548e-3p, and miR-502-3p.

In some embodiments, provided herein are methods of detecting a gynecological disease in a subject comprising measuring the level of miR-17 in a sample from the subject and comparing the level to a reference level; wherein an altered level indicates that the subject has a gynecological disease. In some embodiments, miR-17 comprises miR-17-5p, miR-17-3p, or both. In some embodiments, miR-17 comprises miR-17-5p. In some embodiments, the sample is taken at secretory phase of menstrual cycle. In some embodiments, an increase in miR-17 level indicates presence of the gynecological disease. In some embodiments, the gynecological disease is endometriosis.

In some embodiments, provided herein are methods of detecting a gynecological disease in a subject comprising measuring the level of miR-19b in a sample from the subject and comparing the level to a reference level; wherein an altered level indicates that the subject has a gynecological disease. In some embodiments, miR-19b comprises miR-19b-5p, miR-19b-3p, or both. In some embodiments, miR-19b comprises miR-19b-3p. In some embodiments, the sample is taken at secretory phase of menstrual cycle. In some embodiments, an increase in miR-19b level indicates presence of the gynecological disease. In some embodiments, the gynecological disease is endometriosis.

In some embodiments, provided herein are methods of detecting a gynecological disease in a subject comprising measuring the level of miR-21 in a sample from the subject and comparing the level to a reference level; wherein an altered level indicates that the subject has a gynecological disease. In some embodiments, miR-21 comprises miR-21-5p, miR-21-3p, or both. In some embodiments, miR-21 comprises miR-21-5p, and the sample is taken at secretory phase of menstrual cycle. In some embodiments, miR-21 comprises miR-21-3p, and the sample is taken at proliferative phase of menstrual cycle. In some embodiments, an increase in miR-21 level indicates presence of the gynecological disease. In some embodiments, the gynecological disease is endometriosis.

In some embodiments, provided herein are methods of detecting a gynecological disease in a subject comprising measuring the level of miR-15b in a sample from the subject and comparing the level to a reference level; wherein an altered level indicates that the subject has a gynecological disease. In some embodiments, miR-15b comprises miR-15b-5p, miR-15b-3p, or both. In some embodiments, miR-15b comprises miR-15b-5p. In some embodiments, the sample is taken at secretory phase of menstrual cycle. In some embodiments, an increase in miR-15b level indicates presence of the gynecological disease. In some embodiments, the gynecological disease is endometriosis.

In some embodiments, provided herein are methods of detecting a gynecological disease in a subject comprising measuring the level of miR-19a in a sample from the subject and comparing the level to a reference level; wherein an altered level indicates that the subject has a gynecological disease. In some embodiments, miR-19a comprises miR-19a-5p, miR-19a-3p, or both. In some embodiments, miR-19a comprises miR-19a-3p. In some embodiments, the sample is taken at secretory phase of menstrual cycle. In some embodiments, an increase in miR-19a level indicates presence of the gynecological disease. In some embodiments, the gynecological disease is endometriosis.

In some embodiments, provided herein are methods of detecting a gynecological disease in a subject comprising measuring the level of miR-664a in a sample from the subject and comparing the level to a reference level; wherein an altered level indicates that the subject has a gynecological disease. In some embodiments, miR-664a comprises miR-664a-5p, miR-664a-3p, or both. In some embodiments, miR-664a comprises miR-664a-3p. In some embodiments, the sample is taken at secretory phase of menstrual cycle. In some embodiments, an increase in miR-664a level indicates presence of the gynecological disease. In some embodiments, the gynecological disease is endometriosis.

In some embodiments, provided herein are methods of detecting a gynecological disease in a subject comprising measuring the level of miR-381 in a sample from the subject and comparing the level to a reference level; wherein an altered level indicates that the subject has a gynecological disease. In some embodiments, miR-381 comprises miR-381-5p, miR-381-3p, or both. In some embodiments, miR-381 comprises miR-381-3p. In some embodiments, the sample is taken at secretory phase of menstrual cycle. In some embodiments, a decrease in miR-381 level indicates presence of the gynecological disease. In some embodiments, the gynecological disease is endometriosis.

In some embodiments, provided herein are methods of detecting a gynecological disease in a subject comprising measuring the level of miR-23a in a sample from the subject and comparing the level to a reference level; wherein an altered level indicates that the subject has a gynecological disease. In some embodiments, miR-23a comprises miR-23a-5p, miR-23a-3p, or both. In some embodiments, miR-23a comprises miR-23a-3p. In some embodiments, the sample is taken at secretory phase of menstrual cycle. In some embodiments, an increase in miR-23a level indicates presence of the gynecological disease. In some embodiments, the gynecological disease is endometriosis.

In some embodiments, provided herein are methods of detecting a gynecological disease in a subject comprising measuring the level of miR-654 in a sample from the subject and comparing the level to a reference level; wherein an altered level indicates that the subject has a gynecological disease. In some embodiments, miR-654 comprises miR-654-5p, miR-654-3p, or both. In some embodiments, miR-654 comprises miR-654-3p. In some embodiments, the sample is taken at secretory phase of menstrual cycle. In some embodiments, a decrease in miR-654 level indicates presence of the gynecological disease. In some embodiments, the gynecological disease is endometriosis.

In some embodiments, provided herein are methods of detecting a gynecological disease in a subject comprising measuring the level of miR-24 in a sample from the subject and comparing the level to a reference level; wherein an altered level indicates that the subject has a gynecological disease. In some embodiments, miR-24 comprises miR-24-5p, miR-24-3p, or both. In some embodiments, miR-24 comprises miR-24-3p. In some embodiments, the sample is taken at secretory phase of menstrual cycle. In some embodiments, an increase in miR-24 level indicates presence of the gynecological disease. In some embodiments, the gynecological disease is endometriosis.

In some embodiments, provided herein are methods of detecting a gynecological disease in a subject comprising measuring the level of Let-7b in a sample from the subject and comparing the level to a reference level; wherein an altered level indicates that the subject has a gynecological disease. In some embodiments, Let-7b comprises Let-7b-5p, Let-7b-3p, or both. In some embodiments, Let-7b comprises Let-7b-5p. In some embodiments, the sample is taken at secretory phase of menstrual cycle. In some embodiments, the sample is taken at proliferative phase of menstrual cycle. In some embodiments, an increase in Let-7b level indicates presence of the gynecological disease. In some embodiments, the gynecological disease is endometriosis.

In some embodiments, provided herein are methods of detecting a gynecological disease in a subject comprising measuring the level of miR-20a in a sample from the subject and comparing the level to a reference level; wherein an altered level indicates that the subject has a gynecological disease. In some embodiments, miR-20a comprises miR-20a-5p, miR-20a-3p, or both. In some embodiments, miR-20a comprises miR-20a-5p. In some embodiments, the sample is taken at secretory phase of menstrual cycle. In some embodiments, the sample is taken at proliferative phase of menstrual cycle. In some embodiments, an increase in miR-20a level indicates presence of the gynecological disease. In some embodiments, the gynecological disease is endometriosis.

In some embodiments, provided herein are methods of detecting a gynecological disease in a subject comprising measuring the level of miR-22 in a sample from the subject and comparing the level to a reference level; wherein an altered level indicates that the subject has a gynecological disease. In some embodiments, miR-22 comprises miR-22-5p, miR-22-3p, or both. In some embodiments, miR-22 comprises miR-22-3p and the sample is taken at secretory phase of menstrual cycle. In some embodiments, miR-22 comprises miR-22-5p and the sample is taken at proliferative phase of menstrual cycle. In some embodiments, an increase in miR-22 level indicates presence of the gynecological disease. In some embodiments, the gynecological disease is endometriosis.

In some embodiments, provided herein are methods of detecting a gynecological disease in a subject comprising measuring the level of miR-34c in a sample from the subject and comparing the level to a reference level; wherein an altered level indicates that the subject has a gynecological disease. In some embodiments, miR-34c comprises miR-34c-5p, miR-34c-3p, or both. In some embodiments, miR-34c comprises miR-34c-3p. In some embodiments, the sample is taken at secretory phase of menstrual cycle. In some embodiments, the sample is taken at proliferative phase of menstrual cycle. In some embodiments, an increase in miR-34c level indicates presence of the gynecological disease. In some embodiments, the gynecological disease is endometriosis.

In some embodiments, provided herein are methods of detecting a gynecological disease in a subject comprising measuring the level of miR-1287 in a sample from the subject and comparing the level to a reference level; wherein an altered level indicates that the subject has a gynecological disease. In some embodiments, miR-1287 comprises miR-1287-5p, miR-1287-3p, or both. In some embodiments, miR-1287 comprises miR-1287-5p. In some embodiments, the sample is taken at proliferative phase of menstrual cycle. In some embodiments, an increase in miR-1287 level indicates presence of the gynecological disease. In some embodiments, the gynecological disease is endometriosis.

In some embodiments, provided herein are methods of detecting a gynecological disease in a subject comprising measuring the level of miR-625 in a sample from the subject and comparing the level to a reference level; wherein an altered level indicates that the subject has a gynecological disease. In some embodiments, miR-625 comprises miR-625-5p, miR-625-3p, or both. In some embodiments, miR-625 comprises miR-625-5p. In some embodiments, the sample is taken at proliferative phase of menstrual cycle. In some embodiments, a decrease in miR-625 level indicates presence of the gynecological disease. In some embodiments, the gynecological disease is endometriosis.

In some embodiments, provided herein are methods of detecting a gynecological disease in a subject comprising measuring the level of miR-1294 in a sample from the subject and comparing the level to a reference level; wherein an altered level indicates that the subject has a gynecological disease. In some embodiments, miR-1294 comprises miR-1294-5p, miR-1294-3p, or both. In some embodiments, miR-1294 comprises miR-1294-5p. In some embodiments, the sample is taken at proliferative phase of menstrual cycle. In some embodiments, a decrease in miR-1294 level indicates presence of the gynecological disease. In some embodiments, the gynecological disease is endometriosis.

In some embodiments, provided herein are methods of detecting a gynecological disease in a subject comprising measuring the level of miR-7704 in a sample from the subject and comparing the level to a reference level; wherein an altered level indicates that the subject has a gynecological disease. In some embodiments, the sample is taken at proliferative phase of menstrual cycle. In some embodiments, a decrease in miR-7704 level indicates presence of the gynecological disease. In some embodiments, the gynecological disease is endometriosis.

In some embodiments, provided herein are methods of detecting a gynecological disease in a subject comprising measuring the level of miR-221 in a sample from the subject and comparing the level to a reference level; wherein an altered level indicates that the subject has a gynecological disease. In some embodiments, miR-221 comprises miR-221-5p, miR-221-3p, or both. In some embodiments, miR-221 comprises miR-221-5p. In some embodiments, the sample is taken at proliferative phase of menstrual cycle. In some embodiments, an increase in miR-221 level indicates presence of the gynecological disease. In some embodiments, the gynecological disease is endometriosis.

In some embodiments, provided herein are methods of detecting a gynecological disease in a subject comprising measuring the level of miR-340 in a sample from the subject and comparing the level to a reference level; wherein an altered level indicates that the subject has a gynecological disease. In some embodiments, miR-340 comprises miR-340-5p, miR-340-3p, or both. In some embodiments, miR-340 comprises miR-340-5p. In some embodiments, the sample is taken at proliferative phase of menstrual cycle. In some embodiments, an increase in miR-340 level indicates presence of the gynecological disease. In some embodiments, the gynecological disease is endometriosis.

In some embodiments, provided herein are methods of detecting a gynecological disease in a subject comprising measuring the level of miR-450b in a sample from the subject and comparing the level to a reference level; wherein an altered level indicates that the subject has a gynecological disease. In some embodiments, miR-450b comprises miR-450b-5p, miR-450b-3p, or both. In some embodiments, miR-450b comprises miR-450b-5p. In some embodiments, the sample is taken at proliferative phase of menstrual cycle. In some embodiments, an increase in miR-450b level indicates presence of the gynecological disease. In some embodiments, the gynecological disease is endometriosis.

In some embodiments, provided herein are methods of detecting a gynecological disease in a subject comprising measuring the level of miR-548e in a sample from the subject and comparing the level to a reference level; wherein an altered level indicates that the subject has a gynecological disease. In some embodiments, miR-548e comprises miR-548e-5p, miR-548e-3p, or both. In some embodiments, miR-548e comprises miR-548e-3p. In some embodiments, the sample is taken at proliferative phase of menstrual cycle. In some embodiments, an increase in miR-548e level indicates presence of the gynecological disease. In some embodiments, the gynecological disease is endometriosis.

In some embodiments, provided herein are methods of detecting a gynecological disease in a subject comprising measuring the level of miR-502 in a sample from the subject and comparing the level to a reference level; wherein an altered level indicates that the subject has a gynecological disease. In some embodiments, miR-502 comprises miR-502-5p, miR-502-3p, or both. In some embodiments, miR-502 comprises miR-502-3p. In some embodiments, the sample is taken at proliferative phase of menstrual cycle. In some embodiments, an increase in miR-502 level indicates presence of the gynecological disease. In some embodiments, the gynecological disease is endometriosis.

In some embodiments, provided herein are methods of detecting a gynecological disease in a subject comprising measuring the level of miR-155 in a sample from the subject and comparing the level to a reference level; wherein an altered level indicates that the subject has a gynecological disease. In some embodiments, miR-155 comprises miR-155-5p, miR-155-3p, or both. In some embodiments, miR155 comprises miR155-5p. In some embodiments, the sample is taken at secretory phase of menstrual cycle. In some embodiments, the sample is taken at proliferative phase of menstrual cycle. In some embodiments, an increase in miR-155 level indicates presence of the gynecological disease. In some embodiments, the gynecological disease is endometriosis.

In some embodiments, provided herein are methods of detecting a gynecological disease in a subject comprising measuring the level of miR-574 in a sample from the subject and comparing the level to a reference level; wherein an altered level indicates that the subject has a gynecological disease. In some embodiments, the sample is taken at secretory phase of menstrual cycle. In some embodiments, the sample is taken at proliferative phase of menstrual cycle. In some embodiments, miR-574 comprises miR-574-5p, miR-574-3p, or both. In some embodiments, miR-574 comprises miR-574-3p. In some embodiments, the gynecological disease is endometriosis.

In some embodiments, provided herein are methods of detecting a gynecological disease in a subject comprising measuring the level of miR-9 in a sample from the subject and comparing the level to a reference level; wherein an altered level indicates that the subject has a gynecological disease. In some embodiments, the sample is taken at secretory phase of menstrual cycle. In some embodiments, the sample is taken at proliferative phase of menstrual cycle. In some embodiments, miR-9 comprises miR-9-5p, miR-9-3p, or both. In some embodiments, miR-9 comprises miR-9-5p. In some embodiments, the gynecological disease is endometriosis.

In some embodiments, provided herein are methods of detecting a gynecological disease in a subject comprising measuring the level of miR-299 in a sample from the subject and comparing the level to a reference level; wherein an altered level indicates that the subject has a gynecological disease. In some embodiments, the sample is taken at secretory phase of menstrual cycle. In some embodiments, the sample is taken at proliferative phase of menstrual cycle. In some embodiments, miR-299 comprises miR-299-5p, miR-299-3p, or both. In some embodiments, miR-299 comprises miR-299-5p. In some embodiments, miR-299 comprises miR-299-3p. In some embodiments, the gynecological disease is endometriosis.

In some embodiments, provided herein are methods of detecting a gynecological disease in a subject comprising measuring the level of miR-6789 in a sample from the subject and comparing the level to a reference level; wherein an altered level indicates that the subject has a gynecological disease. In some embodiments, the sample is taken at secretory phase of menstrual cycle. In some embodiments, the sample is taken at proliferative phase of menstrual cycle. In some embodiments, miR-6789 comprises miR-6789-5p, miR-6789-3p, or both. In some embodiments, miR-6789 comprises miR-6789-5p. In some embodiments, the gynecological disease is endometriosis.

In some embodiments, provided herein are methods of detecting a gynecological disease in a subject comprising measuring the level of miR-593 in a sample from the subject and comparing the level to a reference level; wherein an altered level indicates that the subject has a gynecological disease. In some embodiments, the sample is taken at secretory phase of menstrual cycle. In some embodiments, the sample is taken at proliferative phase of menstrual cycle. In some embodiments, miR-593 comprises miR-593-5p, miR-593-3p, or both. In some embodiments, miR-593 comprises miR-593-5p. In some embodiments, the gynecological disease is endometriosis.

In some embodiments, provided herein are methods of detecting a gynecological disease in a subject comprising measuring the level of miR-346 in a sample from the subject and comparing the level to a reference level; wherein an altered level indicates that the subject has a gynecological disease. In some embodiments, the sample is taken at secretory phase of menstrual cycle. In some embodiments, the sample is taken at proliferative phase of menstrual cycle. In some embodiments, miR-346 comprises miR-346-5p, miR-346-3p, or both. In some embodiments, the gynecological disease is endometriosis.

In some embodiments, provided herein are methods of detecting a gynecological disease in a subject comprising measuring the level of miR-34a in a sample from the subject and comparing the level to a reference level; wherein an altered level indicates that the subject has a gynecological disease. In some embodiments, miR-34a comprises miR-34a-5p, miR-34a-3p, or both. In some embodiments, miR-34a comprises miR-34a-5p. In some embodiments, the sample is taken at secretory phase of menstrual cycle. In some embodiments, the sample is taken at proliferative phase of menstrual cycle. In some embodiments, the gynecological disease is endometriosis.

In some embodiments, provided herein are methods of detecting a gynecological disease in a subject comprising measuring the level of miR-449a in a sample from the subject and comparing the level to a reference level; wherein an altered level indicates that the subject has a gynecological disease. In some embodiments, the sample is taken at secretory phase of menstrual cycle. In some embodiments, the sample is taken at proliferative phase of menstrual cycle. In some embodiments, miR-449a comprises miR-449a-5p, miR-449a-3p, or both. In some embodiments, the gynecological disease is endometriosis.

In some embodiments, provided herein are methods of detecting a gynecological disease in a subject comprising measuring the level of miR-7109 in a sample from the subject and comparing the level to a reference level; wherein an altered level indicates that the subject has a gynecological disease. In some embodiments, the sample is taken at secretory phase of menstrual cycle. In some embodiments, the sample is taken at proliferative phase of menstrual cycle. In some embodiments, miR-7109 comprises miR-7109-5p, miR-7109-3p, or both. In some embodiments, miR-7109 comprises miR-7109-5p. In some embodiments, the gynecological disease is endometriosis.

In some embodiments, provided herein are methods of detecting a gynecological disease in a subject comprising measuring the level of miR-3907 in a sample from the subject and comparing the level to a reference level; wherein an altered level indicates that the subject has a gynecological disease. In some embodiments, the sample is taken at secretory phase of menstrual cycle. In some embodiments, the sample is taken at proliferative phase of menstrual cycle. In some embodiments, the gynecological disease is endometriosis.

In some embodiments, provided herein are methods of detecting a gynecological disease in a subject comprising measuring the level of miR-557 in a sample from the subject and comparing the level to a reference level; wherein an altered level indicates that the subject has a gynecological disease. In some embodiments, the sample is taken at secretory phase of menstrual cycle. In some embodiments, the sample is taken at proliferative phase of menstrual cycle. In some embodiments, the gynecological disease is endometriosis.

In some embodiments, provided herein are methods of detecting a gynecological disease in a subject comprising measuring the level of miR-6801 in a sample from the subject and comparing the level to a reference level; wherein an altered level indicates that the subject has a gynecological disease. In some embodiments, the sample is taken at secretory phase of menstrual cycle. In some embodiments, the sample is taken at proliferative phase of menstrual cycle. In some embodiments, miR-6801 comprises miR-6801-5p, miR-6801-3p, or both. In some embodiments, miR-6801 comprises miR-6801-3p. In some embodiments, the gynecological disease is endometriosis.

In some embodiments, provided herein are methods of detecting a gynecological disease in a subject comprising measuring the level of miR-4420 in a sample from the subject and comparing the level to a reference level; wherein an altered level indicates that the subject has a gynecological disease. In some embodiments, the sample is taken at secretory phase of menstrual cycle. In some embodiments, the sample is taken at proliferative phase of menstrual cycle. In some embodiments, the gynecological disease is endometriosis.

In some embodiments, provided herein are methods of detecting a gynecological disease in a subject comprising measuring the level of miR-570 in a sample from the subject and comparing the level to a reference level; wherein an altered level indicates that the subject has a gynecological disease. In some embodiments, miR-570 comprises miR-570-5p, miR-570-3p, or both. In some embodiments, miR-570 comprises miR-570-3p. In some embodiments, the sample is taken at secretory phase of menstrual cycle. In some embodiments, the sample is taken at proliferative phase of menstrual cycle. In some embodiments, the gynecological disease is endometriosis.

In some embodiments, provided herein are methods of detecting a gynecological disease in a subject comprising measuring the level of miR-199 in a sample from the subject and comparing the level to a reference level; wherein an altered level indicates that the subject has a gynecological disease. In some embodiments, miR-199 comprises miR-199-5p, miR-199-3p, or both. In some embodiments, miR-199 comprises miR-199-5p. In some embodiments, miR-199-5p comprises miR-199a-5p, miR-199b-5p, or both. In some embodiments, miR-199-3p comprises miR-199a-3p, miR-199b-3p, or both. In some embodiments, the sample is taken at secretory phase of menstrual cycle. In some embodiments, the sample is taken at proliferative phase of menstrual cycle. In some embodiments, the gynecological disease is endometriosis.

In some embodiments, provided herein are methods of detecting a gynecological disease in a subject comprising measuring the level of miR-520d in a sample from the subject and comparing the level to a reference level; wherein an altered level indicates that the subject has a gynecological disease. In some embodiments, miR-520d comprises miR-520d-5p, miR-520d-3p, or both. In some embodiments, miR-520d comprises miR-520d-5p. In some embodiments, the sample is taken at secretory phase of menstrual cycle. In some embodiments, the sample is taken at proliferative phase of menstrual cycle. In some embodiments, the gynecological disease is endometriosis.

In some embodiments, provided herein are methods of detecting a gynecological disease in a subject comprising measuring the level of miR-424 in a sample from the subject and comparing the level to a reference level; wherein an altered level indicates that the subject has a gynecological disease. In some embodiments, miR-424 comprises miR-424-5p, miR-424-3p, or both. In some embodiments, miR-424 comprises miR-424-5p. In some embodiments, the sample is taken at secretory phase of menstrual cycle. In some embodiments, the sample is taken at proliferative phase of menstrual cycle. In some embodiments, the gynecological disease is endometriosis.

In some embodiments, provided herein are methods of detecting a gynecological disease in a subject comprising measuring the level of miR-200 in a sample from the subject and comparing the level to a reference level; wherein an altered level indicates that the subject has a gynecological disease. In some embodiments, miR-200 comprises miR-200-5p, miR-200-3p, or both. In some embodiments, miR-200-5p comprises miR-200a-5p, miR-200b-5p, or miR-200c-5p, or any combination thereof. In some embodiments, miR-200-3p comprises miR-200a-3p, miR-200b-3p, or miR-200c-3p, or any combination thereof. In some embodiments, the sample is taken at secretory phase of menstrual cycle. In some embodiments, the sample is taken at proliferative phase of menstrual cycle. In some embodiments, the gynecological disease is endometriosis.

Panels of the combinations of miRNA biomarkers provided herein can be used to detect gynecological disease, such as endometriosis with increased sensitivity and specificity. In some embodiments, a panel of miRNA biomarkers is provided for the early detection of gynecological disease. In some embodiments, a panel of miRNA biomarkers is provided for the early diagnosis of endometriosis. In some embodiments, the miRNA biomarker panel can include at least two, at least three, at least four, at least five, at least six, at least seven, at least eight, at least nine, or at least ten miRNAs. The miRNA biomarker panel can include two miRNAs. The miRNA biomarker panel can include three miRNAs. The miRNA biomarker panel can include four miRNAs. The miRNA biomarker panel can include five miRNAs. The miRNA biomarker panel can include six miRNAs. The miRNA biomarker panel can include seven miRNAs. The miRNA biomarker panel can include eight miRNAs. The miRNA biomarker panel can include nine miRNAs. The miRNA biomarker panel can include ten miRNAs.

In some embodiments, the panel of miRNA biomarkers includes miR-17, miR-199 and miR-34c. In some embodiments, the panel of miRNA biomarkers includes miR-17-5p, miR-199a-5p and miR-34c-3p. In some embodiments, the panel of miRNA biomarkers includes miR-20a, miR-22 and miR-34c. In some embodiments, the panel of miRNA biomarkers includes miR-20a-5p, miR-22-3p and miR-34c-3p. In some embodiments, the panel of miRNA biomarkers includes miR-17, miR-22, miR-20a, miR-34c and miR-155. In some embodiments, the panel of miRNA biomarkers includes miR-17-5p, miR-22-3p, miR-20a-5p, miR-34c-3p and miR-155-5p. In some embodiments, the panel of miRNA biomarkers includes miR-34c, miR-23a, miR-17, miR-155, let-7b, miR-199, miR-22, and miR-20a. In some embodiments, the panel of miRNA biomarkers includes miR-34c-3p, miR-23a-3p, miR-17-5p, miR-155-5p, let-7b-5p, miR-199-5p, miR-22-3p, and miR-20a-5p. In some embodiments, the panel of miRNA biomarkers includes miR-34c and miR-20a. In some embodiments, the panel of miRNA biomarkers includes miR-34c-3p and miR-20a-5p. In some embodiments, the panel of miRNA biomarkers includes miR-34c and miR-17. In some embodiments, the panel of miRNA biomarkers includes miR-34c-3p and miR-17-5p. In some embodiments, the panel of miRNA biomarkers includes miR-34c and miR-22. In some embodiments, the panel of miRNA biomarkers includes miR-34c-3p and miR-22-5p. In some embodiments, the panel of miRNA biomarkers includes miR-155, miR-22 and miR-23a. In some embodiments, the panel of miRNA biomarkers include miR155-5p, miR-22-5p and miR-23a-3p. In some embodiments, the panel of miRNA biomarkers include miR-155-5p, miR-22-3p and miR-23a-3p. In some embodiments, the panel of miRNA biomarkers further includes Let-7b, miR-17, and miR-20a. In some embodiments, the panel of miRNA biomarkers further includes Let-7b-5p, miR-17-5p, and miR-20a-5p. In some embodiments, the panel of miRNA biomarkers further includes Let-7b-3p, miR-17-5p, and miR-20a-5p. In some embodiments, a panel of miRNA biomarkers is provided for the early diagnosis of endometriosis, including Let-7b-5p, miR-155-5p, miR-23a-3p, miR-17-5p, miR-20a-5p, and miR-22-5p. In some embodiments, a panel of miRNA biomarkers is provided for the early diagnosis of endometriosis, including Let-7b-5p, miR-155-5p, miR-23a-3p, miR-17-5p, miR-20a-5p, and miR-22-3p. In some embodiments, the panel of miRNA biomarkers further includes miR-574, miR-520d, or both. In some embodiments, the panel of miRNA biomarkers further includes miR-574-3p, miR-520d-5p, or both.

In some embodiments, the panel of miRNA biomarkers comprise miR-155-5p, miR-22-3p, miR-23a-3p, miR-17-5p, miR-20a-5p, Let-7b-5p, and Let-7b-3p. In some embodiments, the panel of miRNA biomarkers comprise miR-155-5p, miR-22-5p, miR-23a-3p, miR-17-5p, miR-20a-5p, and Let-7b-5p. In some embodiments, the panel of miRNA biomarkers comprise miR-155-5p, miR-22-3p, miR-23a-3p, miR-17-5p, miR-20a-5p, Let-7b-5p, Let-7b-3p, miR-574-3p, and miR-520d-5p. In some embodiments, the panel of miRNA biomarkers comprise miR-155-5p, miR-22-5p, miR-23a-3p, miR-17-5p, miR-20a-5p, Let-7b-5p, miR-574-3p, and miR-520d-5p.

In some embodiments, provided herein are miRNAs that can serve as biomarkers for detecting a gynecological disease, such as endometriosis, wherein the miRNAs can be miRNAs targeting CA-125, SIRT1 or BCL6. In some embodiments, the miRNAs can be miRNAs targeting CA-125. In some embodiments, the miRNA targeting CA-125 is miR-299-3p, miR-299-5p, miR-6789-5p, miR-593-5p, miR-7109-5p, and miR-3907, or any combination thereof. In some embodiments, the miRNAs can be miRNAs targeting SIRT1. In some embodiments, the miRNA targeting SIRT1 is miR-9-5p, miR-34a-5p, miR-449a, miR-4420, and miR-570-3p, or any combination thereof. In some embodiments, the miRNA targeting BCL6 is miR-346, miR-557, and miR-6801-3p, or any combination thereof. In some embodiments, the miRNA is miR-299-3p, miR-299-5p, miR-6789-5p, miR-593-5p, miR-7109-5p, miR-3907, miR-9-5p, miR-34a-5p, miR-449a, miR-4420, miR-570-3p, miR-346, miR-557, and miR-6801-3p, or any combination thereof.

In some embodiments, the panel of miRNA biomarkers comprise at least two, at least three, at least four, at least five, at least six, at least seven, at least eight, at least nine, or ten miRNAs selected from: miR-17, miR-19b, miR-21, miR-15b, miR-19a, miR-664a, miR-381, miR-23a, miR-654, and miR-24. In some embodiments, the panel of miRNA biomarkers comprise at least two, at least three, at least four, at least five, at least six, at least seven, at least eight, at least nine, or ten miRNAs selected from: miR-17-5p, miR-19b-3p, miR-21-5p, miR-15b-5p, miR-19a-3p, miR-664a-3p, miR-381-3p, miR-23a-3p, miR-654-3p, and miR-24-3p.

In some embodiments, the panel of miRNA biomarkers comprise at least two, at least three, at least four, at least five, at least six, at least seven, at least eight, at least nine, at least or eleven miRNAs selected from: miR-21, miR-23a, miR-1287, miR-625, miR-1294, miR-7704, miR-221, miR-340, miR-450b, miR-548e, and miR-502. In some embodiments, the panel of miRNA biomarkers comprise at least two, at least three, at least four, at least five, at least six, at least seven, at least eight, at least nine, at least ten, or eleven miRNAs selected from: miR-21-3p, miR-22-5p, miR-1287-5p, miR-625-5p, miR-1294-5p, miR-7704, miR-221-5p, miR-340-5p, miR-450b-5p, miR-548e-3p, and miR-502-3p.

In some embodiments, the expression pattern of the panel of miRNA biomarkers can be further used to distinguish endometriosis alone from other gynecological disease, including, for example, physiological cyst, hemorrhagic cyst, and leiomyomas. In some embodiments, the expression pattern of miRNA biomarkers Let-7b, miR-17, miR-20a, or any combination thereof, can be used to distinguish endometriosis alone from other gynecological disease. In some embodiments, the expression pattern of miRNA biomarkers Let-7b-5p, miR-17-5p, miR-20a-5p, or any combination thereof, can be used to distinguish endometriosis alone from other gynecological diseases. In some embodiments, the panel for distinguishing endometriosis alone from other gynecological disease comprises Let-7b-5p, miR-17-5p, and miR-20a-5p. In some embodiments, the expression pattern of miRNA biomarkers miR-155, Let-7b, miR-23a, miR-17, or miR-20a, or any combination thereof, can be used to distinguish endometriosis alone from other gynecological diseases. In some embodiments, the expression pattern of miRNA biomarkers miR-155-5p, Let-7b-5p, miR-23a-3p, miR-17-5p, or miR-20a-5p, or any combination thereof, can be used to distinguish endometriosis alone from other gynecological diseases. In some embodiments, the panel for distinguishing endometriosis alone from other gynecological disease comprises miR-155-5p, miR-23a-3p, miR-17-5p, and miR-20a-5p. In some embodiments, the panel can further include miR-424, miR-200 family miRNA, or both for distinguishing endometriosis and gynecological disease. In some embodiments, the panel further includes miR-424-5p. In some embodiments, the panel further includes miR-200a, miR-200b, miR-200c, or any combination thereof.

In some embodiments, a panel of miRNA biomarkers is provided for endometriosis staging, namely, to distinguish mild (Stage I or II) and moderate/severe (Stage III or IV) endometriosis. In some embodiments, the panel for detecting late-stage endometriosis comprises miR-17, miR-20a, miR-22, or any combination thereof. In some embodiments, the panel for detecting late-stage endometriosis comprises miR-17-5p, miR-20a-5p, miR-22-5p, or any combination thereof. In some embodiments, the panel for detecting late-stage endometriosis comprises miR-17-5p, miR-20a-5p and miR-22-5p. In some embodiments, the panel can further include miR-424, miR-200 family miRNA, or both for distinguishing endometriosis and gynecological disease. In some embodiments, the panel further includes miR-424-5p. In some embodiments, the panel further includes miR-200a, miR-200b, miR-200c, or any combination thereof.

In some embodiments, a panel of miRNA biomarkers is provided for detecting adenomyosis. In some embodiments, a panel of miRNA biomarkers is provided for distinguishing patients having endometriosis only and patients having both endometriosis and adenomyosis. In some embodiments, the difference between the expression level of a miRNA biomarker and the expression level can be used. In some embodiments, the difference is compared to a cutoff value, wherein a difference that is higher than cutoff indicates that the subject has both endometriosis and adenomyosis, and a difference that is lower than cutoff indicates that the subject has endometriosis without adenomyosis.

Provided herein are methods useful for assessing the risk for gynecological disease, such as endometriosis. The miRNA biomarkers provided here can, for example, distinguish gynecological disease from normal healthy control. In some embodiments, provided herein are methods for detecting gynecological disease. The miRNA biomarkers provided here can, for example, distinguish endometriosis from normal healthy control. In some embodiments, provided herein are methods for detecting endometriosis. In some embodiments, provided herein are methods for diagnosing endometriosis in a subject, for determining whether a subject is at risk of developing endometriosis, for determining the prognosis in a subject having endometriosis, and for monitoring efficacy of a treatment for endometriosis in a subject, based upon the expression level or expression pattern of one or more miRNA biomarkers associated with endometriosis. Accordingly, the present disclosure provides methods of assessing the level of at least one miRNA associated with endometriosis.

In some embodiments, the expression level or expression pattern of miRNA biomarkers disclosed herein can be used to classify mild (Stage I or II) and moderate/severe (Stage III or IV) endometriosis. In some embodiments, the expression level or expression pattern of miRNA biomarkers disclosed herein can be used to determine whether an endometriosis patient also has adenomyosis. Accordingly, further provided herein are methods of determining the stage of endometriosis in a patient by measuring the expression of the miRNA biomarkers disclosed herein. In some embodiments, further provided herein are methods of determining whether a patient has endometriosis also or endometriosis and adenomyosis by measuring the expression of the miRNA biomarkers disclosed herein.

In some embodiments, provided herein are genetic screening assays of a subject to determine the level of expression of at least one miRNA associated with endometriosis in the subject. In some embodiments, the assays described herein are in vitro assays.

The methods disclosed herein relate to the discovery that the expression levels of certain miRNA biomarkers individually or the expression pattern of certain miRNA biomarkers in combination are associated with endometriosis and can be used to detect endometriosis and to predict development or prognosis of endometriosis with high sensitivity and specificity. As used herein and consistent with its understanding in the art, “sensitivity” refers to the ability of a biomarker-based assay to correctly identify individuals who have the condition being tested for. It measures the proportion of true positive results among all individuals who actually have the condition. A highly sensitive assay has a low rate of false negative results. Also, as used herein and consistent with its understanding in the art, “specificity” refers to the ability of a biomarker-based assay to correctly identify individuals who do not have the condition being tested for. A highly specific assay has a low rate of false positive results.

In some embodiments, the present disclosure relates to a screening assay of a subject to determine whether the subject has an altered expressed level or pattern of one or more of miR-17, miR-19b, miR-21, miR-15b, miR-19a, miR-664a, miR-381, miR-23a, miR-654, miR-24, Let-7b, miR-20a, miR-22, miR-34c, miR-1287, miR-625, miR-1294, miR-7704, miR-221, miR-340, miR-450b, miR-548e, miR-502, miR-574, miR-9, miR-299, miR-6789, miR-593, miR-346, miR-34a, miR-449a, miR-7109, miR-3907, miR-557, miR-6801, miR-4420, miR-570, miR-155, miR-199, miR-520d, miR-424, and miR-200. In some embodiments, the present disclosure relates to a screening assay of one or more of miR-17-5p, miR-17-3p, miR-19b-5p, miR-19b-3p, miR-21-5p, miR-21-3p, miR-15b-5p, miR-15b-3p, miR-19a-5p, miR-19a-3p, miR-664a-5p, miR-664a-3p, miR-381-5p, miR-381-3p, miR-23a-5p, miR-23a-3p, miR-654-5p, miR-654-3p, miR-24-5p, miR-24-3p, Let-7b-5p, Let-7b-3p, miR-20a-5p, miR-20a-3p, miR-22-5p, miR-22-3p, miR-34c-5p, miR-34c-3p, miR-1287-5p, miR-1287-3p, miR-625-5p, miR-625-3p, miR-1294-5p, miR-1294-3p, miR-7704, miR-221-5p, miR-221-3p, miR-340-5p, miR-340-3p, miR-450b-5p, miR-450b-3p, miR-548e-5p, miR-548e-3p, miR-502-5p, miR-502-3p, miR-574-5p, miR-574-3p, miR-9-5p, miR-9-3p, miR-299-5p, miR-299-3p, miR-6789-5p, miR-6789-3p, miR-593-5p, miR-593-3p, miR-346-5p, miR-346-3p, miR-34a-5p, miR-34a-3p, miR-449a-5p, miR-449a-3p, miR-7109-5p, miR-7109-3p, miR-3907, miR-557, miR-6801-5p, miR-6801-3p, miR-4420, miR-570-5p, miR-570-3p, miR-155-5p, miR-155-3p, miR-199a-5p, miR-199a-3p, miR-199b-5p, miR-199b-3p, miR-520d-5p, miR-520d-3p, miR-424-5p, miR-424-3p, miR-200a-5p, miR-200a-3p, miR-200b-5p, miR-200b-3p, miR-200c-5p, and miR-200c-3p. In some embodiments, the present disclosure relates to a screening assay of one or more of miR-17-5p, miR-19b-3p, miR-21-5p, miR-21-3p, miR-15b-5p, miR-19a-3p, miR-664a-3p, miR-381-3p, miR-23a-3p, miR-654-3p, miR-24-3p, Let-7b-5p, miR-20a-5p, miR-22-5p, miR-22-3p, miR-34c-3p, miR-1287-5p, miR-625-5p, miR-1294-5p, miR-7704, miR-221-5p, miR-340-5p, miR-450b-5p, miR-548e-3p, miR-502-3p, miR-574-3p, miR-9-5p, miR-299-5p, miR-299-3p, miR-6789-5p, miR-593-5p, miR-346, miR-34a-5p, miR-449a, miR-7109-5p, miR-3907, miR-557, miR-6801-3p, miR-4420, miR-570-3p, miR-155-5p, miR-199-5p, miR-520d-5p, miR-424-5p, and miR-200.

In some embodiments, the present disclosure relates to a screening assay of a subject to determine whether the subject has an altered expressed level or pattern of one or more of Let-7b, miR-20a, miR-22, miR-34c, miR-574, miR-9, miR-299, miR-6789, miR-593, miR-346, miR-34a, miR-449a, miR-7109, miR-3907, miR-557, miR-6801, miR-4420, and miR-570. In some embodiments, the present disclosure relates to a screening assay of one or more of Let-7b-5p, Let-7b-3p, miR-20a-5p, miR-22-3p, miR-34c-3p, miR-574-3p, miR-9-5p, miR-299-5p, miR-299-3p, miR-6789-5p, miR-593-5p, miR-346, miR-34a-5p, miR-449a, miR-7109-5p, miR-3907, miR-557, miR-6801-3p, miR-4420, and miR-570-3p. In some embodiments, the present disclosure relates to a screening assay of a subject to determine whether the subject has an altered expressed level or pattern of one or more of miR-34c, miR-155, miR-22, miR-23a, miR-17, miR-20a, Let-7b, miR-199, miR-574, miR-520d, miR-424, and miR-200. In some embodiments, the present disclosure relates to a screening assay of one or more of miR-34c-5p, miR-34c-3p, miR-155-5p, miR-155-3p, miR-22-5p, miR-22-3p, miR-23a-5p, miR-23a-3p, miR-17-5p, miR-17-3p, miR-20a-5p, miR-20a-3p, Let-7b-5p, Let-7b-3p, miR-199a-5p, miR-199a-3p, miR-199b-5p, miR-199b-3p, miR-574-5p, miR-574-3p, miR-520d-5p, miR-520d-3p, miR-424-5p, miR-424-3p, miR-200a-5p, miR-200a-3p, miR-200b-5p, miR-200b-3p, miR-200c-5p, and miR-200c-3p. In some embodiments, the present disclosure relates to a screening assay of a subject to determine whether the subject has an altered expressed level or pattern of one or more of miR-155, miR-22, miR-23a, miR-17, miR-20a, Let-7b, miR-574, miR-520d, miR-424, and miR-200. In some embodiments, the present disclosure relates to a screening assay of one or more of miR-155-5p, miR-155-3p, miR-22-3p, miR-23a-5p, miR-23a-3p, miR-17-5p, miR-17-3p, miR-20a-5p, miR-20a-3p, Let-7b-5p, miR-574-5p, miR-574-3p, miR-520d-5p, miR-520d-3p, miR-424-5p, miR-424-3p, miR-200a-5p, miR-200a-3p, miR-200b-5p, miR-200b-3p, miR-200c-5p, and miR-200c-3p.

In some embodiments, the present disclosure relates to a screening assay of a subject to determine whether the subject has an altered expressed level or pattern of one or more of miR-17, miR-19b, miR-21, miR-15b, miR-19a, miR-664a, miR-381, miR-23a, miR-654, and miR-24. In some embodiments, the present disclosure relates to a screening assay of one or more of miR-17-5p, miR-19b-3p, miR-21-5p, miR-15b-5p, miR-19a-3p, miR-664a-3p, miR-381-3p, miR-23a-3p, miR-654-3p, and miR-24-3p. In some embodiments, the sample is taken at secretory phase of menstrual cycle.

In some embodiments, the present disclosure relates to a screening assay of a subject to determine whether the subject has an altered expressed level or pattern of one or more of miR-21, miR-23a, miR-1287, miR-625, miR-1294, miR-7704, miR-221, miR-340, miR-450b, miR-548e, and miR-502. In some embodiments, the present disclosure relates to a screening assay of one or more of miR-21-3p, miR-22-5p, miR-1287-5p, miR-625-5p, miR-1294-5p, miR-7704, miR-221-5p, miR-340-5p, miR-450b-5p, miR-548e-3p, and miR-502-3p. In some embodiments, the sample is taken at proliferative phase of menstrual cycle.

In some embodiments, the disclosure relates to a screening assay of a subject to determine whether the subject has an elevated level of expression of one or more miRNA biomarkers. In some embodiments, the disclosure relates to a screening assay of a subject to determine whether the subject has a reduced level of expression of one or more miRNA biomarkers. The present disclosure provides methods of assessing the level of at least one miRNA in a subject or a sample from a subject.

The present disclosure provides improved methods for assessing the risk for endometriosis in a subject, including the risk for having endometriosis, the risk for developing endometriosis, the risk for advancing into a later stage of endometriosis, and the risk of having reoccurrence of endometriosis. The risk for endometriosis can be assessed by measuring one or more of the miRNA biomarkers described herein, and comparing the measured values to reference values. Such a comparison can be undertaken with mathematical algorithms or formulae to combine information from results of multiple individual biomarkers and other parameters into a single measurement or index. For example, in some embodiments, the comparison is undertaken with a quantitative algorithm, as described in further detail below.

Subjects identified as having an enhanced risk for endometriosis can optionally be selected to receive treatment regimens. As such, in some embodiments, methods provided herein further comprise administering a treatment for endometriosis to the subject that has an altered level of the miRNA. In some embodiments, provided herein are methods of treating a subject for endometriosis, comprising (a) measuring a level of a miRNA selected from: miR-17, miR-19b, miR-21, miR-15b, miR-19a, miR-664a, miR-381, miR-23a, miR-654, miR-24, Let-7b, miR-20a, miR-22, miR-34c, miR-1287, miR-625, miR-1294, miR-7704, miR-221, miR-340, miR-450b, miR-548e, miR-502, miR-574, miR-9, miR-299, miR-6789, miR-593, miR-346, miR-34a, miR-449a, miR-7109, miR-3907, miR-557, miR-6801, miR-4420, miR-570, miR-155, miR-199, miR-520d, miR-424, and miR-200 in a sample of the subject, and (b) administering a treatment for endometriosis to the subject if the level of the miRNA is altered compared to a reference level.

Treatment options include such as laparoscopic surgery or administration of therapeutic compounds to prevent, treat or delay the occurrence, reoccurrence or progression of endometriosis. Identifying a subject as having an enhanced risk for endometriosis recurrence after laparoscopy allows for the selection and initiation of various therapeutic interventions or treatment regimens in order to delay, reduce or prevent recurrence in those at risk. Further, identifying a subject with a low risk, or those who do not have an enhanced risk, for endometriosis recurrence allows for the sparing of unnecessary additional therapy administered to the subject. Data concerning the biomarkers of the present disclosure can also be combined or correlated with other data or test results, such as, without limitation, measurements of clinical parameters or other algorithms for endometriosis. Other data includes age, ethnicity, and other genomic data or protein expression data, specifically expression values of other gene signatures relevant to endometriosis outcomes, and the like. The can also comprise subject information such as medical history and any relevant family history.

In some embodiments, one or more biomarkers associated with endometriosis are up-regulated, or expressed at a level higher than a reference level. In some embodiments, one or more biomarkers associated with endometriosis are down-regulated, or expressed at a level lower than a reference level. In some embodiments of methods disclosed herein, the level of expression of the biomarker is determined to be elevated or increased when the level of expression of the biomarker is increased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1,000%, by at least 1,500%, by at least 2,000%, by at least 2,500%, by at least 3,000%, by at least 4,000%, or by at least 5,000%, when compared with a reference level.

In some embodiments of methods disclosed herein, the level of expression of the biomarker is determined to be elevated or increased when the level of expression of the biomarker in the biological sample is increased by at least 1-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, at least 3.5-fold, at least 4-fold, at least 4.5-fold, at least 5-fold, at least 5.5-fold, at least 6-fold, at least 6.5-fold, at least 7-fold, at least 7.5-fold, at least 8-fold, at least 8.5-fold, at least 9-fold, at least 9.5-fold, at least 10-fold, at least 11-fold, at least 12-fold, at least 13-fold, at least 14-fold, at least 15-fold, at least 20-fold, at least 25-fold, at least 30-fold, at least 40-fold, at least 50-fold, at least 75-fold, at least 100-fold, at least 200-fold, at least 250-fold, at least 500-fold, or at least 1000-fold, when compared with a reference level.

In some embodiments of methods disclosed herein, the level of expression of the biomarker is determined to be reduced or decreased when the level of expression of the biomarker is reduced or decreased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1,000%, by at least 1,500%, by at least 2,000%, by at least 2,500%, by at least 3,000%, by at least 4,000%, or by at least 5,000%, when compared with a reference level.

In some embodiments of methods disclosed herein, the level of expression of the biomarker is determined to be reduced or decreased when the level of expression of the biomarker is reduced or decreased by at least 1-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, at least 3.5-fold, at least 4-fold, at least 4.5-fold, at least 5-fold, at least 5.5-fold, at least 6-fold, at least 6.5-fold, at least 7-fold, at least 7.5-fold, at least 8-fold, at least 8.5-fold, at least 9-fold, at least 9.5-fold, at least 10-fold, at least 11-fold, at least 12-fold, at least 13-fold, at least 14-fold, at least 15-fold, at least 20-fold, at least 25-fold, at least 30-fold, at least 40-fold, at least 50-fold, at least 75-fold, at least 100-fold, at least 200-fold, at least 250-fold, at least 500-fold, or at least 1,000-fold, when compared with a reference level.

Provided herein are methods of diagnosing a gynecological disease (e.g., endometriosis and/or adenomyosis) in a subject, comprising (a) measuring a level of a miRNA selected from: miR-155, miR-22, miR-23a, miR-17, miR-20a, Let-7b, miR-574, miR-520d, miR-424, and miR-200, in a sample of the subject, and (b) comparing the level to a reference level; wherein an altered level indicates that the subject has a gynecological disease (e.g., endometriosis and/or adenomyosis). In some embodiments, a decreased level indicates that the subject has a gynecological disease (e.g., endometriosis and/or adenomyosis).

The miRNA biomarkers disclosed herein can also be used to assess the risk for a subject to develop endometriosis. Accordingly, provided herein are methods of determining whether a subject is at risk of developing a gynecological disease (e.g., endometriosis and/or adenomyosis), comprising (a) measuring a level of a miRNA selected from: miR-34c, miR-155, miR-22, miR-23a, miR-17, miR-20a, Let-7b, miR-199, miR-574, miR-520d, miR-424, and miR-200 in a sample of the subject, and (b) comparing the level to a reference level; wherein an altered level indicates that the subject is at risk of developing a gynecological disease (e.g., endometriosis and/or adenomyosis). In some embodiments, a decreased level indicates that the subject is at risk of developing a gynecological disease (e.g., endometriosis and/or adenomyosis). The methods provided herein can determine whether a subject is at risk of developing endometriosis at high sensitivity and specificity.

Provided herein are methods of determining whether a subject is at risk of developing a gynecological disease (e.g., endometriosis and/or adenomyosis), comprising (a) measuring a level of a miRNA selected from: miR-17, miR-19b, miR-21, miR-15b, miR-19a, miR-664a, miR-381, miR-23a, miR-654, miR-24, Let-7b, miR-20a, miR-22, miR-34c, miR-1287, miR-625, miR-1294, miR-7704, miR-221, miR-340, miR-450b, miR-548e, miR-502, miR-574, miR-9, miR-299, miR-6789, miR-593, miR-346, miR-34a, miR-449a, miR-7109, miR-3907, miR-557, miR-6801, miR-4420, miR-570, miR-155, miR-199, miR-520d, miR-424, and miR-200 in a sample of the subject, and (b) comparing the level to a reference level; wherein an altered level indicates that the subject is at risk of developing a gynecological disease (e.g., endometriosis and/or adenomyosis). The methods provided herein can determine whether a subject is at risk of developing endometriosis at high sensitivity and specificity.

Provided herein are methods of determining whether a subject is at risk of developing a gynecological disease (e.g., endometriosis and/or adenomyosis), comprising (a) measuring a level of a miRNA selected from: Let-7b, miR-20a, miR-22, miR-34c, miR-574, miR-9, miR-299, miR-6789, miR-593, miR-346, miR-34a, miR-449a, miR-7109, miR-3907, miR-557, miR-6801, miR-4420, and miR-570, in a sample of the subject, and (b) comparing the level to a reference level; wherein an altered level indicates that the subject is at risk of developing a gynecological disease (e.g., endometriosis and/or adenomyosis). The methods provided herein can determine whether a subject is at risk of developing endometriosis at high sensitivity and specificity.

Provided herein are methods of determining whether a subject is at risk of developing a gynecological disease (e.g., endometriosis and/or adenomyosis), comprising (a) measuring a level of a miRNA selected from: Let-7b, miR-20a, miR-22, miR-34c, miR-574, miR-9, miR-299, miR-6789, miR-593, miR-346, miR-34a, miR-449a, miR-7109, miR-3907, miR-557, miR-6801, miR-4420, and miR-570 in a sample of the subject, and (b) comparing the level to a reference level; wherein an altered level indicates that the subject is at risk of developing a gynecological disease (e.g., endometriosis and/or adenomyosis). The methods provided herein can determine whether a subject is at risk of developing endometriosis at high sensitivity and specificity.

Provided herein are methods of determining whether a subject is at risk of developing a gynecological disease (e.g., endometriosis and/or adenomyosis), comprising (a) measuring a level of a miRNA selected from: miR-17, miR-19b, miR-21, miR-15b, miR-19a, miR-664a, miR-381, miR-23a, miR-654, and miR-24 in a sample of the subject, and (b) comparing the level to a reference level; wherein an altered level indicates that the subject is at risk of developing a gynecological disease (e.g., endometriosis and/or adenomyosis). The methods provided herein can determine whether a subject is at risk of developing endometriosis at high sensitivity and specificity. In some embodiments, methods provided herein comprise measuring a level of a miRNA selected from: miR-17-5p, miR-19b-3p, miR-21-5p, miR-15b-5p, miR-19a-3p, miR-664a-3p, miR-381-3p, miR-23a-3p, miR-654-3p, and miR-24-3p. In some embodiments, the sample is taken at secretory phase of menstrual cycle.

Provided herein are methods of determining whether a subject is at risk of developing a gynecological disease (e.g., endometriosis and/or adenomyosis), comprising (a) measuring a level of a miRNA selected from: miR-21, miR-23a, miR-1287, miR-625, miR-1294, miR-7704, miR-221, miR-340, miR-450b, miR-548e, miR-502 in a sample of the subject, and (b) comparing the level to a reference level; wherein an altered level indicates that the subject is at risk of developing a gynecological disease (e.g., endometriosis and/or adenomyosis). The methods provided herein can determine whether a subject is at risk of developing endometriosis at high sensitivity and specificity. In some embodiments, methods provided herein comprise measuring a level of a miRNA selected from: miR-21-3p, miR-22-5p, miR-1287-5p, miR-625-5p, miR-1294-5p, miR-7704, miR-221-5p, miR-340-5p, miR-450b-5p, miR-548e-3p, miR-502-3p. In some embodiments, the sample is taken at proliferative phase of menstrual cycle.

In some embodiments, the miR-17 comprises miR-17-5p, miR-17-3p, or both; miR-19b comprises miR-19b-5p, miR-19b-3p, or both; miR-21 comprises miR-21-5p, miR-21-3p, or both; miR-15b comprises miR-15b-5p, miR-15b-3p, or both; miR-19a comprises miR-19a-5p, miR-19a-3p, or both; miR-664a comprises miR-664a-5p, miR-664a-3p, or both; miR-381 comprises miR-381-5p, miR-381-3p, or both; miR-23a comprises miR-23a-5p, miR-23a-3p, or both; miR-654 comprises miR-654-5p, miR-654-3p, or both; miR-24 comprises miR-24-5p, miR-24-3p, or both; Let-7b comprises Let-7b-5p, Let-7b-3p, or both; miR-20a comprises miR-20a-5p, miR-20a-3p, or both; miR-22 comprises miR-22-5p, miR-22-3p, or both; miR-34c comprises miR-34c-5p, miR-34c-3p, or both; miR-1287 comprises miR-1287-5p, miR-1287-3p, or both; miR-625 comprises miR-625-5p, miR-625-3p, or both; miR-1294 comprises miR-1294-5p, miR-1294-3p, or both; miR-7704 comprises miR-7704; miR-221 comprises miR-221-5p, miR-221-3p, or both; miR-340 comprises miR-340-5p, miR-340-3p, or both; miR-450b comprises miR-450b-5p, miR-450b-3p, or both; miR-548e comprises miR-548e-5p, miR-548e-3p, or both; miR-502 comprises miR-502-5p, miR-502-3p, or both; miR-574 comprises miR-574-5p, miR-574-3p, or both; miR-9 comprises miR-9-5p, miR-9-3p, or both; miR-299 comprises miR-299-5p, miR-299-3p, or both; miR-6789 comprises miR-6789-5p, miR-6789-3p, or both; miR-593 comprises miR-593-5p, miR-593-3p, or both; miR-346 comprises miR-346-5p, miR-346-3p, or both; miR-34a comprises miR-34a-5p, miR-34a-3p, or both; miR-449a comprises miR-449a-5p, miR-449a-3p, or both; miR-7109 comprises miR-7109-5p, miR-7109-3p, or both; miR-3907 comprises miR-3907; miR-557 comprises miR-557; miR-6801 comprises miR-6801-5p, miR-6801-3p or both; miR-4420 comprises miR-4420; miR-570 comprises miR-570-5p, miR-570-3p, or both; miR-155 comprises miR-155-5p, miR-155-3p, or both; miR-199 comprises miR-199a-5p, miR-199a-3p, miR-199b-5p, or miR-199b-3p, or any combination thereof; miR-520d comprises miR-520d-5p, miR-520d-3p, or both; miR-424 comprises miR-424-5p, miR-424-3p, or both; miR-200 comprises miR-200a-5p, miR-200a-3p, miR-200b-5p, miR-200b-3p, miR-200c-5p, or miR-200c-3p, or any combination thereof.

In some embodiments, miR-17 comprises miR-17-5p; miR-19b comprises miR-19b-3p; miR-21 comprises miR-21-5p, miR-21-3p, or both; miR-15b comprises miR-15b-5p; miR-19a comprises miR-19a-3p; miR-664a comprises miR-664a-3p; miR-381 comprises miR-381-3p; miR-23a comprises miR-22-5p, miR-23a-3p, or both; miR-654 comprises miR-654-3p; miR-24 comprises miR-24-3p; Let-7b comprises Let-7b-5p, Let-7b-3p, or both; miR-20a comprises miR-20a-5p; miR-22 comprises miR-22-3p; miR-34c comprises miR-34c-3p; miR-1287 comprises miR-1287-5p; miR-625 comprises miR-625-5p; miR-1294 comprises miR-1294-5p; miR-7704 comprises miR-7704; miR-221 comprises miR-221-5p; miR-340 comprises miR-340-5p; miR-450b comprises miR-450b-5p; miR-548e comprises miR-548e-3p; miR-502 comprises miR-502-3p; miR-574 comprises miR-574-3p; miR-9 comprises miR-9-5p; miR-299 comprises miR-299-5p, miR-299-3p, or both; miR-6789 comprises miR-6789-5p; miR-593 comprises miR-593-5p; miR-346 comprises miR-346-5p, miR-346-3p, or both; miR-34a comprises miR-34a-5p; miR-449a comprises miR-449a-5p, miR-449a-3p, or both; miR-7109 comprises miR-7109-5p; miR-3907 comprises miR-3907; miR-557 comprises miR-557; miR-6801 miR-6801-3p; miR-4420 comprises miR-4420; miR-570 comprises miR-570-3p; miR-155 comprises miR-155-5p; miR-199 comprises miR-199a-5p; miR-520d comprises miR-520d-5p, miR-520d-3p, or both; miR-424 comprises miR-424-5p, miR-424-3p, or both; miR-200 comprises miR-200a-5p, miR-200a-3p, miR-200b-5p, miR-200b-3p, miR-200c-5p, or miR-200c-3p, or any combination thereof.

In some embodiments, methods provided herein comprise detecting miR-34c. In some embodiments, methods provided herein comprise detecting miR-34c-3p. In some embodiments, methods provided herein comprise detecting Let-7b. In some embodiments, methods provided herein comprise detecting Let-7b-5p. In some embodiments, methods provided herein comprise detecting miR-17. In some embodiments, methods provided herein comprise detecting miR-17-5p. In some embodiments, methods provided herein comprise detecting miR-19b. In some embodiments, methods provided herein comprise detecting miR-19b-3p. In some embodiments, methods provided herein comprise detecting miR-21. In some embodiments, methods provided herein comprise detecting miR-21-5p. In some embodiments, methods provided herein comprise detecting miR-21-3p. In some embodiments, methods provided herein comprise detecting miR-15b. In some embodiments, methods provided herein comprise detecting miR-15b-5p. In some embodiments, methods provided herein comprise detecting miR-19a. In some embodiments, methods provided herein comprise detecting miR-19-3p. In some embodiments, methods provided herein comprise detecting miR-664a. In some embodiments, methods provided herein comprise detecting miR-664a-3p. In some embodiments, methods provided herein comprise detecting miR-381. In some embodiments, methods provided herein comprise detecting miR-381-3p. In some embodiments, methods provided herein comprise detecting miR-23a. In some embodiments, methods provided herein comprise detecting miR-23a-3p. In some embodiments, methods provided herein comprise detecting miR-654. In some embodiments, methods provided herein comprise detecting miR-654-3p. In some embodiments, methods provided herein comprise detecting miR-24. In some embodiments, methods provided herein comprise detecting miR-24-3p. In some embodiments, methods provided herein comprise detecting miR-22. In some embodiments, methods provided herein comprise detecting miR-22-5p. In some embodiments, methods provided herein comprise detecting miR-22-3p. In some embodiments, methods provided herein comprise detecting miR-20a. In some embodiments, methods provided herein comprise detecting miR-20a-5p. In some embodiments, methods provided herein comprise detecting miR-199. In some embodiments, methods provided herein comprise detecting miR-199-5p. In some embodiments, methods provided herein comprise detecting miR-155. In some embodiments, methods provided herein comprise detecting miR-155-5p. In some embodiments, methods provided herein comprise detecting miR-1287. In some embodiments, methods provided herein comprise detecting miR-1287-5p. In some embodiments, methods provided herein comprise detecting miR-625. In some embodiments, methods provided herein comprise detecting miR-625-5p. In some embodiments, methods provided herein comprise detecting miR-1294. In some embodiments, methods provided herein comprise detecting miR-1294-5p. In some embodiments, methods provided herein comprise detecting miR-7704. In some embodiments, methods provided herein comprise detecting miR-7704. In some embodiments, methods provided herein comprise detecting miR-221. In some embodiments, methods provided herein comprise detecting miR-221-5p. In some embodiments, methods provided herein comprise detecting miR-340. In some embodiments, methods provided herein comprise detecting miR-340-5p. In some embodiments, methods provided herein comprise detecting miR-450b. In some embodiments, methods provided herein comprise detecting miR-450b-5p. In some embodiments, methods provided herein comprise detecting miR-548e. In some embodiments, methods provided herein comprise detecting miR-548e-3p. In some embodiments, methods provided herein comprise detecting miR-502. In some embodiments, methods provided herein comprise detecting miR-502-3p.

In some embodiments, methods provided herein comprise detecting a miRNA panel including miR-17, miR-199 and miR-34c. In some embodiments, methods provided herein comprise detecting a miRNA panel including miR-17-5p, miR-199a-5p and miR-34c-3p. In some embodiments, methods provided herein comprise detecting a miRNA panel including miR-20a, miR-22 and miR-34c. In some embodiments, methods provided herein comprise detecting a miRNA panel including miR-20a-5p, miR-22-3p and miR-34c-3p. In some embodiments, methods provided herein comprise detecting a miRNA panel including miR-17, miR-22, miR-20a, miR-34c and miR-155. In some embodiments, methods provided herein comprise detecting a miRNA panel including miR-17-5p, miR-22-3p, miR-20a-5p, miR-34c-3p and miR-155-5p. In some embodiments, methods provided herein comprise detecting a miRNA panel including miR-34c, miR-23a, miR-17, miR-155, Let-7b, miR-199, miR-22, and miR-20a. In some embodiments, methods provided herein comprise detecting a miRNA panel including miR-34c-3p, miR-23a-3p, miR-17-5p, miR-155-5p, Let-7b-5p, miR-199-5p, miR-22-3p, and miR-20a-5p.

In some embodiments, methods provided herein comprise detecting a miRNA panel including miR-34c and miR-20a. In some embodiments, methods provided herein comprise detecting a miRNA panel including miR-34c-3p and miR-20a-5p. In some embodiments, methods provided herein comprise detecting a miRNA panel including miR-34c and miR-17. In some embodiments, methods provided herein comprise detecting a miRNA panel including miR-34c-3p and miR-17-5p. In some embodiments, methods provided herein comprise detecting a miRNA panel including miR-34c and miR-22. In some embodiments, methods provided herein comprise detecting a miRNA panel including miR-34c-3p and miR-22-5p.

In some embodiments, methods provided herein comprise detecting a miRNA panel including miR-155 (e.g., miR-155-5p), miR-22 (e.g., miR-22-3p or miR-22-5p), and miR-23a (miR-23a-3p or miR-23a-5p). In some embodiments, the biomarker further includes Let-7b (e.g., Let-7b-5p or Let-7b-3p), miR-17 (e.g., miR-17-5p), and miR-20a (e.g., miR-20a-5p). In some embodiments, the biomarker further includes miR-574 (e.g., miR-574-3p), miR-520d (e.g., miR-520d-5p), or both. In some embodiments, provided herein are methods of diagnosing a gynecological disease (e.g., endometriosis and/or adenomyosis) in a subject, comprising (a) measuring a level of a miRNA selected from: Let-7b-5p, miR-155-5p, miR-23a-3p, miR-17-5p, miR-20a-5p, and miR-22-5p in a sample of the subject, and (b) comparing the level to a reference level; wherein an altered level indicates that the subject has a gynecological disease (e.g., endometriosis and/or adenomyosis). In some embodiments, provided herein are methods of diagnosing a gynecological disease (e.g., endometriosis and/or adenomyosis) in a subject, comprising (a) measuring a level of a miRNA selected from: Let-7b-5p, miR-155-5p, miR-23a-3p, miR-17-5p, miR-20a-5p, and miR-22-3p in a sample of the subject, and (b) comparing the level to a reference level; wherein an altered level indicates that the subject has a gynecological disease (e.g., endometriosis and/or adenomyosis). In some embodiments, a lowered total expression indicates that the subject has a gynecological disease (e.g., endometriosis and/or adenomyosis). In some embodiments, weighted sum of the expression of the biomarker panel is used to compare with a reference level, wherein an altered expression indicates the presence of a gynecological disease (e.g., endometriosis and/or adenomyosis).

Provided herein are methods of determining whether a subject is at risk of developing a gynecological disease (e.g., endometriosis and/or adenomyosis), comprising (a) measuring a level of a miRNA targeting CA-125, SIRT1 or BCL6 in a sample of the subject, and (b) comparing the level to a reference level; wherein an altered level indicates that the subject is at risk of developing a gynecological disease (e.g., endometriosis and/or adenomyosis). The methods provided herein can determine whether a subject is at risk of developing endometriosis at high sensitivity and specificity. In some embodiments, a miRNA targeting CA-125 can be miR-299-3p, miR-299-5p, miR-6789-5p, miR-593-5p, miR-7109-5p, or miR-3907. In some embodiments, a miRNA targeting SIRT1 can be miR-9-5p, miR-34a-5p, miR-449a, miR-4420, or miR-570-3p. In some embodiments, a miRNA targeting BCL6 can be miR-346, miR-557, or miR-6801-3p.

Provided herein are methods of determining whether a subject is at risk of developing a gynecological disease (e.g., endometriosis and/or adenomyosis), comprising (a) measuring a level of a miRNA selected from: miR-299-3p, miR-299-5p, miR-6789-5p, miR-593-5p, miR-7109-5p, miR-3907, miR-9-5p, miR-34a-5p, miR-449a, miR-4420, miR-570-3p, miR-346, miR-557, and miR-6801-3p in a sample of the subject, and (b) comparing the level to a reference level; wherein an altered level indicates that the subject is at risk of developing a gynecological disease (e.g., endometriosis and/or adenomyosis). The methods provided herein can determine whether a subject is at risk of developing endometriosis at high sensitivity and specificity.

As used herein and consistent with its understanding in the art, “diagnosis” refers to detecting a disease or disorder (e.g., endometriosis). Usually, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition. The diagnostic methods can be used independently, or in combination with other suitable diagnosing and/or staging methods.

In some embodiments, the expression pattern of the panel of miRNA biomarkers can be further used to distinguish endometriosis alone from other gynecological disease, including, for example, physiological cyst, hemorrhagic cyst, and leiomyomas. In some embodiments, provided herein are methods of distinguishing subject having endometriosis from those with other gynecological disease, comprising measuring a level of a miRNA selected from: Let-7b (e.g., Let-7b-5p), miR-17 (e.g., miR-17-5p), miR-20a (miR-20a-5p), in a sample of the subject, wherein the expression level of the miRNA indicates whether the subject has endometriosis or a different gynecological disease. In some embodiments, provided herein are methods of distinguishing subject having endometriosis from those with other gynecological disease, comprising measuring a level of a miRNA selected from: miR-155 (e.g., miR-155-5p), Let-7b (e.g., Let-7b-5p), miR-23a (e.g., miR-23a-3p), miR-17 (e.g., miR-17-5p), miR-20a (miR-20a-5p) in a sample of the subject, wherein the expression level of the miRNA indicates whether the subject has endometriosis or a different gynecological disease. In some embodiments, provided herein are methods of distinguishing subject having endometriosis from those with other gynecological disease, comprising measuring a level of a miRNA selected from: miR-155 (e.g., miR-155-5p), miR-23a (e.g., miR-23a-3p), miR-17 (e.g., miR-17-5p), and miR-20a (miR-20a-5p) in a sample of the subject, wherein the expression level of the miRNA indicates whether the subject has endometriosis or a different gynecological disease. In some embodiments, the panel can further include miR-424, miR-200 family miRNA, or both for distinguishing endometriosis and gynecological disease. In some embodiments, the panel further includes miR-424-5p. In some embodiments, the panel further includes miR-200a, miR-200b, miR-200c, or any combination thereof.

Provided herein are also methods of assessing the prognosis in a subject having endometriosis, comprising (a) measuring a level of a miRNA selected from: miR-17, miR-19b, miR-21, miR-15b, miR-19a, miR-664a, miR-381, miR-23a, miR-654, miR-24, Let-7b, miR-20a, miR-22, miR-34c, miR-1287, miR-625, miR-1294, miR-7704, miR-221, miR-340, miR-450b, miR-548e, miR-502, miR-574, miR-9, miR-299, miR-6789, miR-593, miR-346, miR-34a, miR-449a, miR-7109, miR-3907, miR-557, miR-6801, miR-4420, miR-570, miR-155, miR-199, miR-520d, miR-424, and miR-200 in a sample of the subject, and (b) comparing the level to a reference level; wherein an altered level indicates that the endometriosis is likely to progress into an advanced stage in the subject, as determined by rASRM staging. Provided herein are also methods of assessing the prognosis in a subject having endometriosis, comprising (a) measuring a level of a miRNA selected from: miR-17-5p, miR-19b-3p, miR-21-5p, miR-15b-5p, miR-19a-3p, miR-664a-3p, miR-381-3p, miR-23a-3p, miR-654-3p, miR-24-3p, Let-7b-5p, miR-20a-5p, miR-22-3p, miR-34c-3p, miR-1287-5p, miR-1287-3p, miR-625-5p, miR-625-3p, miR-1294-5p, miR-1294-3p, miR-7704, miR-221-5p, miR-221-3p, miR-340-5p, miR-340-3p, miR-450b-5p, miR-450b-3p, miR-548e-5p, miR-548e-3p, miR-502-5p, miR-502-3p, miR-574-3p, miR-9-5p, miR-299-5p, miR-299-3p, miR-6789-5p, miR-593-5p, miR-346, miR-34a-5p, miR-449a, miR-7109-5p, miR-3907, miR-557, miR-6801-3p, miR-4420, miR-570-3p, miR-155-5p, miR-199a-5p, miR-520d-5p, miR-424, miR-200 in a sample of the subject, and (b) comparing the level to a reference level; wherein an altered level indicates that the endometriosis is likely to progress into an advanced stage in the subject, as determined by rASRM staging.

Provided herein are also methods of assessing the prognosis in a subject having endometriosis, comprising (a) measuring a level of a miRNA selected from: miR-17, miR-19b, miR-21, miR-15b, miR-19a, miR-664a, miR-381, miR-23a, miR-654, and miR-24 in a sample of the subject, and (b) comparing the level to a reference level; wherein an altered level indicates that the endometriosis is likely to progress into an advanced stage in the subject, as determined by rASRM staging. Provided herein are also methods of assessing the prognosis in a subject having endometriosis, comprising (a) measuring a level of a miRNA selected from: miR-17-5p, miR-19b-3p, miR-21-5p, miR-15b-5p, miR-19a-3p, miR-664a-3p, miR-381-3p, miR-23a-3p, miR-654-3p, and miR-24-3p in a sample of the subject, and (b) comparing the level to a reference level; wherein an altered level indicates that the endometriosis is likely to progress into an advanced stage in the subject, as determined by rASRM staging. In some embodiments, the sample is taken at secretory phase of menstrual cycle.

Provided herein are also methods of assessing the prognosis in a subject having endometriosis, comprising (a) measuring a level of a miRNA selected from: miR-21, miR-23a, miR-1287, miR-625, miR-1294, miR-7704, miR-221, miR-340, miR-450b, miR-548e, and miR-502 in a sample of the subject, and (b) comparing the level to a reference level; wherein an altered level indicates that the endometriosis is likely to progress into an advanced stage in the subject, as determined by rASRM staging. Provided herein are also methods of assessing the prognosis in a subject having endometriosis, comprising (a) measuring a level of a miRNA selected from: miR-21-3p, miR-22-5p, miR-1287-5p, miR-625-5p, miR-1294-5p, miR-7704, miR-221-5p, miR-340-5p, miR-450b-5p, miR-548e-3p, and miR-502-3p in a sample of the subject, and (b) comparing the level to a reference level; wherein an altered level indicates that the endometriosis is likely to progress into an advanced stage in the subject, as determined by rASRM staging. In some embodiments, the sample is taken at proliferative phase of menstrual cycle.

Provided herein are also methods of assessing the prognosis in a subject having endometriosis, comprising (a) measuring a level of a miRNA selected from: Let-7b, miR-20a, miR-22, miR-34c, miR-1287, miR-625, miR-1294, miR-7704, miR-221, miR-340, miR-450b, miR-548e, miR-502, miR-574, miR-9, miR-299, miR-6789, miR-593, miR-346, miR-34a, miR-449a, miR-7109, miR-3907, miR-557, miR-6801, miR-4420, and miR-570 in a sample of the subject, and (b) comparing the level to a reference level; wherein an altered level indicates that the endometriosis is likely to progress into an advanced stage in the subject, as determined by rASRM staging.

In some embodiments, miR-17 comprises miR-17-5p; miR-19b comprises miR-19b-3p; miR-21 comprises miR-21-5p, miR-21-3p, or both; miR-15b comprises miR-15b-5p; miR-19a comprises miR-19a-3p; miR-664a comprises miR-664a-3p; miR-381 comprises miR-381-3p; miR-23a comprises miR-23a-3p; miR-654 comprises miR-654-3p; miR-24 comprises miR-24-3p; Let-7b comprises Let-7b-5p, Let-7b-3p, or both; miR-20a comprises miR-20a-5p; miR-22 comprises miR-22-5p, miR-22-3p, or both; miR-34c comprises miR-34c-3p; miR-1287 comprises miR-1287-5p; miR-625 comprises miR-625-5p; miR-1294 comprises miR-1294-5p; miR-7704 comprises miR-7704; miR-221 comprises miR-221-5p; miR-340 comprises miR-340-5p; miR-450b comprises miR-450b-5p; miR-548e comprises miR-548e-3p; miR-502 comprises miR-502-3p; miR-574 comprises miR-574-3p; miR-9 comprises miR-9-5p; miR-299 comprises miR-299-5p, miR-299-3p, or both; miR-6789 comprises miR-6789-5p; miR-593 comprises miR-593-5p; miR-346 comprises miR-346-5p, miR-346-3p, or both; miR-34a comprises miR-34a-5p; miR-449a comprises miR-449a-5p, miR-449a-3p, or both; miR-7109 comprises miR-7109-5p; miR-3907 comprises miR-3907; miR-557 comprises miR-557; miR-6801 miR-6801-3p; miR-4420 comprises miR-4420; miR-570 comprises miR-570-3p; miR-155 comprises miR-155-5p; miR-199 comprises miR-199a-5p; miR-520d comprises miR-520d-5p, miR-520d-3p, or both; miR-424 comprises miR-424-5p, miR-424-3p, or both; miR-200 comprises miR-200a-5p, miR-200a-3p, miR-200b-5p, miR-200b-3p, miR-200c-5p, or miR-200c-3p, or any combination thereof. In some embodiments, the Let-7b is Let-7b-5p; miR-20a is miR-20a-5p; miR-22 is miR-22-3p; miR-34c is miR-34c-3p; miR-574 is miR-574-3p; miR-9 is miR-9-5p; miR-299 is miR-299-5p or miR-299-3p; miR-6789 is miR-6789-5p; miR-593 is miR-593-5p; miR-34a is miR-34a-5p; miR-7109 is miR-7109-5p; miR-6801 is miR-6801-3p; miR-570 is miR-570-3p. As used herein and consistent with its understanding in the art, “prognosis” refers to prediction of the probable course and outcome of a disease or a condition (e.g., endometriosis), including prediction of severity, duration, chances of recovery, etc. The methods can also be used to devise a suitable therapeutic plan. In some embodiments, provided herein are methods for detecting late-stage endometriosis comprises measuring the expression of a miRNA panel comprising miR-17 (miR-17-5p), miR-20a (miR-20a-5p), miR-22 (miR-22-5p), or any combination thereof. In some embodiments, the panel can further include miR-424, miR-200 family miRNA, or both for distinguishing endometriosis and gynecological disease. In some embodiments, the panel further includes miR-424-5p. In some embodiments, the panel further includes miR-200a, miR-200b, or miR-200c, or any combination thereof.

Monitoring the levels of at least one biomarker also allows for a course of treatment to be monitored. In some embodiments, biomarkers also are useful for monitoring subjects undergoing treatments and therapies for endometriosis. For example, a sample can be provided from a subject undergoing treatment regimens or therapeutic interventions, and the level of one or more biomarkers can be determined. The level of one or more biomarkers can be compared to a sample derived from the subject before and after treatment or can be compared to samples derived from one or more subjects who have shown improvements in risk factors as a result of such treatment or exposure. The present disclosure also provides methods for identifying agents for treating endometriosis that are appropriate or otherwise customized for a specific subject.

Accordingly, provided herein are methods of monitoring efficacy of a treatment for endometriosis in a subject, comprising (a) measuring a level of a miRNA selected from: miR-17, miR-19b, miR-21, miR-15b, miR-19a, miR-664a, miR-381, miR-23a, miR-654, miR-24, Let-7b, miR-20a, miR-22, miR-34c, miR-1287, miR-625, miR-1294, miR-7704, miR-221, miR-340, miR-450b, miR-548e, miR-502, miR-574, miR-9, miR-299, miR-6789, miR-593, miR-346, miR-34a, miR-449a, miR-7109, miR-3907, miR-557, miR-6801, miR-4420, miR-570, miR-155, miR-199, miR-520d, miR-424, and miR-200 in a sample of the subject, and (b) comparing the level to a reference level; wherein a decrease in the disparity indicates the efficacy of the treatment. Provided herein are methods of monitoring efficacy of a treatment for endometriosis in a subject, comprising (a) measuring a level of a miRNA selected from: miR-17-5p, miR-19b-3p, miR-21-5p, miR-21-3p, miR-15b-5p, miR-19a-3p, miR-664a-3p, miR-381-3p, miR-23a-3p, miR-654-3p, miR-24-3p, Let-7b-5p, miR-20a-5p, miR-22-5p, miR-22-3p, miR-34c-3p, miR-1287-5p, miR-625-5p, miR-1294-5p, miR-7704, miR-221-5p, miR-340-5p, miR-450b-5p, miR-548e-3p, miR-502-3p, miR-574-3p, miR-9-5p, miR-299-5p, miR-299-3p, miR-6789-5p, miR-593-5p, miR-346, miR-34a-5p, miR-449a, miR-7109-5p, miR-3907, miR-557, miR-6801-3p, miR-4420, miR-570-3p, miR-155-5p, miR-199a-5p, miR-520d-5p, miR-424, miR-200 in a sample of the subject, and (b) comparing the level to a reference level; wherein a decrease in the disparity indicates the efficacy of the treatment.

Provided herein are methods of monitoring efficacy of a treatment for endometriosis in a subject, comprising (a) measuring a level of a miRNA selected from: miR-17, miR-19b, miR-21, miR-15b, miR-19a, miR-664a, miR-381, miR-23a, miR-654, and miR-24 in a sample of the subject, and (b) comparing the level to a reference level; wherein a decrease in the disparity indicates the efficacy of the treatment. Provided herein are methods of monitoring efficacy of a treatment for endometriosis in a subject, comprising (a) measuring a level of a miRNA selected from: miR-17-5p, miR-19b-3p, miR-21-5p, miR-15b-5p, miR-19a-3p, miR-664a-3p, miR-381-3p, miR-23a-3p, miR-654-3p, and miR-24-3p in a sample of the subject, and (b) comparing the level to a reference level; wherein a decrease in the disparity indicates the efficacy of the treatment. In some embodiments, the sample is taken at secretory phase of menstrual cycle.

Provided herein are methods of monitoring efficacy of a treatment for endometriosis in a subject, comprising (a) measuring a level of a miRNA selected from: miR-21, miR-23a, miR-1287, miR-625, miR-1294, miR-7704, miR-221, miR-340, miR-450b, miR-548e, and miR-502 in a sample of the subject, and (b) comparing the level to a reference level; wherein a decrease in the disparity indicates the efficacy of the treatment. Provided herein are methods of monitoring efficacy of a treatment for endometriosis in a subject, comprising (a) measuring a level of a miRNA selected from: miR-21-3p, miR-22-5p, miR-1287-5p, miR-625-5p, miR-1294-5p, miR-7704, miR-221-5p, miR-340-5p, miR-450b-5p, miR-548e-3p, and miR-502-3p in a sample of the subject, and (b) comparing the level to a reference level; wherein a decrease in the disparity indicates the efficacy of the treatment. In some embodiments, the sample is taken at proliferative phase of menstrual cycle.

These biomarkers also are useful for selecting or modifying therapies and treatments that would be efficacious in subjects having endometriosis and subjects who have had endometriosis. In some embodiments, provided herein are methods for identifying agents for treating endometriosis that are appropriate or otherwise customized for a specific subject.

### 7.4.1 Methods of Detection

Provided herein are methods for assessing the risk for a gynecological disease (e.g., endometriosis) in a subject, including the risk for having a gynecological disease (e.g., endometriosis), the risk for developing a gynecological disease (e.g., endometriosis), the risk for advancing into a later stage of endometriosis, and the risk of having reoccurrence of endometriosis, comprising (a) measuring a level of a miRNA selected from: miR-17, miR-19b, miR-21, miR-15b, miR-19a, miR-664a, miR-381, miR-23a, miR-654, miR-24, Let-7b, miR-20a, miR-22, miR-34c, miR-1287, miR-625, miR-1294, miR-7704, miR-221, miR-340, miR-450b, miR-548e, miR-502, miR-574, miR-9, miR-299, miR-6789, miR-593, miR-346, miR-34a, miR-449a, miR-7109, miR-3907, miR-557, miR-6801, miR-4420, miR-570, miR-155, miR-199, miR-520d, miR-424, and miR-200 in a sample of the subject, and (b) comparing the level to a reference level; wherein an altered level indicates the risk for a gynecological disease (e.g., endometriosis).

In some embodiments, an “altered” level means the difference between the quantified value of the expression level of a particular miRNA biomarker present in a sample as compared to a reference value is at least about 10%, at least about 15%, at least about 20%, at least about 25%, at least about 30%, at least about 35%, at least about 40%, at least about 50%, at least about 60%, at least about 75%, or at least about 80% or more. In some embodiments, an “altered” level means that there exists a statistically significant difference between the quantified value of the expression level of a particular miRNA biomarker present in a sample as compared to a reference value. For example, in some embodiments, the measured expression level of the biomarker can fall outside of about 1.0 standard deviations, about 1.5 standard deviations, about 2.0 standard deviations, or about 2.5 stand deviations of the mean of a control or reference group.

In some embodiments, the subject is a mammal. In some embodiments, the subject is a human. In some embodiments, the subject is a human female. In some embodiments, the subject is an adolescent human female. In some embodiments, the subject is a postpubescent female subject. In some embodiments, the subject is a human female aged between 12-60 years. In some embodiments, the subject is a human female aged about 20 years old, 30 years old, 40 years old, 50 years old or 60 years old. In some embodiments, the subject has a family history of endometriosis. In some embodiments, the subject is diagnosed with endometriosis. In some embodiments, the subject is suspected of having endometriosis. In some embodiments, the subject is at risk of a recurrence of endometriosis. In some embodiments, the subject is at risk of developing endometriosis. In some embodiments, the subject is a human female exhibiting at least one clinical indicator of endometriosis. Clinical indicators of endometriosis include, e.g., intermenstrual bleeding, dysmenorrhea, dyspareunia, dyschezia, dysuria, chronic pelvic pain, lower abdominal pain, or infertility. In some embodiments, the subject exhibits no clinical indicator of endometriosis.

In some embodiments, the subject is a human female diagnosed rASRM stage I or II endometriosis.

In embodiments, the endometriosis assessed is selected from the group consisting of stage I endometriosis according to rASRM staging, stage II endometriosis according to rASRM staging, stage III endometriosis according to rASRM staging, stage IV endometriosis according to rASRM staging. In some embodiments, the endometriosis assessed is stage I, stage II, stage III, or stage IV endometriosis. In embodiments, endometriosis is early endometriosis, in particular stage I endometriosis according to rASRM staging or stage II endometriosis according to rASRM staging. In particular embodiments, the endometriosis assessed is stage III or stage IV endometriosis.

In some embodiments, the endometriosis assessed is peritoneal endometriosis, endometriomas or deep infiltrating endometriosis (DIE). In some embodiments, the endometriosis assessed is peritoneal endometriosis of stage I or II according to rASRM staging.

In some embodiments, the assessment is performed independent of the rASRM staging. In particular, the assessment is performed without performing laparoscopy. In some embodiments, the assessment is performed without assessing the presence or severity of endometriosis in the patient using laparoscopy and/or the rASRM staging.

In some embodiments, methods of detecting endometriosis as disclosed herein can be combined with one or more additional approaches that aid the diagnosis of endometriosis, such as pelvic exam, ultrasound, magnetic resonance imaging (MRI), or laparoscopy. During a pelvic exam, doctors manually palpat areas in pelvis for abnormalities, such as cysts on reproductive organs or scars behind uterus. Ultrasound, including both abdomen ultrasound and transvaginal ultrasound, can be used. Both use high-frequency sound waves to create images of the uterus and can identify cysts associated with endometriosis (endometriomas). MRI uses a magnetic field and radio waves to create detailed images of the organs and tissues within the body. For some, an MRI helps with surgical planning, giving the surgeon detailed information about the location and size of endometrial implants. Laparoscopy is a surgical procedure usually performed under general anesthesia. The surgeon uses a laparoscope to look for signs of endometrial tissue outside the uterus, which can provide information about the location, extent and size of the endometrial implants.

In some embodiments, the methods provided herein further comprise the assessment of the presence of dysmenorrhea and/or lower abdominal pain in the patient. In some embodiments, the presence of dysmenorrhea and/or lower abdominal pain is assessed according to the VAS scale. In some embodiments, dysmenorrhea VAS score of 4 or higher indicated moderate or severe dysmenorrhea. In some embodiments, scores of 3 or less indicate no or mild dysmenorrhea.

### 7.4.1.1 Combination with CA-125

In some embodiments, provided herein are methods of assessing whether a subject has endometriosis, comprising (a) measuring the expression level of CA-125 in a sample of the subject, wherein the sample is obtained during the secretory phase of a menstrual cycle; and comparing the expression level of CA-125 to a reference level; and (b) measuring a level of a miRNA selected from: miR-17, miR-19b, miR-21, miR-15b, miR-19a, miR-664a, miR-381, miR-23a, miR-654, miR-24, Let-7b, miR-20a, miR-22, miR-34c, miR-574, miR-9, miR-299, miR-6789, miR-593, miR-346, miR-34a, miR-449a, miR-7109, miR-3907, miR-557, miR-6801, miR-4420, miR-570, miR-155, miR-199, miR-520d, miR-424, and miR-200 in a second sample of the subject, and comparing the level of the miRNA to a reference level; wherein an increased expression level of CA-125 indicates that the subject is likely to have endometriosis and an altered level of the miRNA corroborates that the subject likely has endometriosis. In some embodiments, the altered level of miRNA indicates the staging of endometriosis. The details of using the CA-125 level, especially the CA-125 serum level during the secretory phase of menstrual cycle as a biomarker for endometriosis diagnosis, as well as details of using the miRNA panels as biomarkers to further corroborate the diagnosis or to provide information to aid staging, are provided in sections above.

### 7.4.1.2 Sample Preparation

Methods provided herein comprise measuring the expression level of miRNA biomarkers in a sample of the subject. In some embodiments, the test sample is prepared from a biological sample obtained from the subject. The biological sample can be a sample from any source which contains a nucleic acid comprising endometriosis associated miRNA, such as a body fluid (e.g., blood, plasma, serum, saliva, urine, etc.), or a tissue, or an exosome, or a cell, or a combination thereof. A biological sample can be obtained by appropriate methods, such as, by way of examples, biopsy or fluid draw. The biological sample can be used as the test sample; alternatively, the biological sample can be processed to enhance access to polypeptides, nucleic acids, or copies of nucleic acids (e.g., copies of nucleic acids comprising a miRNA associated with endometriosis), and the processed biological sample can then be used as the test sample. For example, in various embodiments, nucleic acid is prepared from a biological sample, for use in the methods. Alternatively, or in addition, an amplification method can be used to amplify nucleic acids comprising all or a fragment of a nucleic acid in a biological sample, for use as the test sample in the assessment of the expression level of a miRNA associated with endometriosis.

In some embodiments, the sample is derived from tissues (e.g., endometrial tissue), extracts, cells, or cell cultures, including nucleated cells, cell lysates, conditioned medium from fetal or maternal cells, and physiological fluids.

In some embodiments, the sample can be physiological fluid such as, for example, whole blood, plasma, serum, saliva, mucous, phlegm, sputum, tears, ocular lens fluid, lymphatic fluid, cerebral spinal fluid, sweat, urine, milk, ascites fluid, amniotic fluid, vaginal fluid, pleural fluid, synovial fluid, peritoneal fluid, and the like. Methods of obtaining fluid samples include but are not limited to aspirations or drawing of blood or other fluids.

In some embodiments, a physiological fluid sample can be acellular fluids. Acellular fluids include body fluid samples in which cells are absent or are present in such low amounts that the miRNA level determined reflects its level in the liquid portion of the sample, rather than in the cellular portion. Such acellular body fluids are generally produced by processing a cell-containing body fluid by, for example, centrifugation or filtration, to remove the cells. Typically, an acellular body fluid contains no intact cells or very minimal amounts of cells; however, some acellular body fluids may contain cell fragments or cellular debris. Examples of acellular fluids include plasma or serum, or body fluids from which cells have been removed.

In some embodiments, the sample used in methods disclosed herein is a physiological fluid, such as blood. In some embodiments, the sample is a serum sample. In some embodiments, the sample is a plasma sample.

In some embodiments, samples used in methods disclosed herein comprise endometrial cells. In some embodiments, the sample is a menstrual blood. In some embodiments, the sample is peripheral blood. Menstrual blood refers to the red blood cells containing fluids which during menses can be collected by various means from vaginal bleeding. In some embodiments, menstrual blood is directly collected from the posterior vaginal fornix. Other methods of collecting menstrual blood are the use of absorptives like pads and tampons and their appropriate extraction. In some embodiments, timed samples of menstrual blood are collected and used in methods disclosed herein. For example, the sample can be collected at the second day of a menstrual cycle.

In some embodiments, the sample is a uterine tissue sample. In some embodiments, the sample can be fluids or washings of the uterine lining, or sample prepared by similar techniques involving cervical lavage or brushings. In some embodiments, samples used in methods disclosed herein comprise endometrial cells. In some embodiments, the tissue sample comprises epithelial cells. In some embodiments, the sample comprises cells from an endometrial cyst.

In some embodiments, the sample is a pap smear sample. Standard procedures for pap smear are well known in the art. The pap smear sample is a collection of cells from the cervix, which is commonly obtained in conjunction with a pelvic exam.

In some embodiments, the sample is a biopsy sample. In some embodiments, the sample is a biopsy of the endometrium. In some embodiments, the sample is a formalin fixed, paraffin embedded biopsy section thereof. In some embodiments, the biopsy is obtained prior to laparoscopy. Harvested tissue can be maintained on ice and used for the test within four hours. Tests can be performed on either unfractioned biopsies or epithelial cells isolated from the biopsy. In some embodiments, methods provided herein further comprise isolating endometrial cells from the sample.

In some embodiments, the sample is preserved after being obtained from the subject to minimize the possible loss of DNA, RNA, and/or protein during handling. In some embodiments, the sample is preserved before being processed or analyzed. The sample can be preserved using any means known in the art. In some embodiments, preserving the sample comprises freezing it at about −20° C. to about −70° C. In some embodiments, preserving the sample comprises placing the sample in a fixative. The fixative can be liquid. The fixative can be solid. In some embodiments, the fixative is neutral buffered formalin (NBF). NBF typically includes formaldehyde, methanol, and phosphate buffer. In some embodiments, the fixative is aldehyde-based. In some embodiments, the fixative is ethanol-based. The ethanol-based fixative can have >50% (e.g., 60%, 70%, 80%, 90% or 100%) ethanol. In some embodiments, the ethanol-based fixative can have 70% ethanol. In some embodiments, the fixative can have at least one component selected from glycerol, glacial acetic acid, a chao trope or denaturant (for example, guanidinium thiocyanate, guanidinium HCl, or guanidinium acetate), trehalose, polyethylene glycol (e.g., PEG200), ethylenediaminetetraacetic acid (EDTA), ethylene glycol-bis(-aminoethyl ether) ethylenediaminetetraacetic acid (EGTA), acrylamide, trichloroacetic acid, acetate salt (e.g., zinc acetate, copper acetate, or magnesium acetate), acetonitrile, and ethylene glycol. In some embodiments, the fixative is powder.

In some embodiments, the sample used in methods disclosed herein can be directly obtained from the source or pretreated prior to use. In some embodiments, a sample can be treated prior to use, such as preparing plasma from blood, diluting viscous fluids, and the like. Methods of treatment can involve filtration, distillation, extraction, concentration, inactivation of interfering components, the addition of reagents, and the like. In some embodiments, the cells contained in a sample (e.g., a menstrual blood sample or an endometrial biopsy sample) are washed, mixed, allowed to settle, and embedded according to standard procedures. In some embodiments, methods provided herein further comprise enriching endometrial cells in the sample. In some embodiments, the sample is treated with appropriate measures to release the expression products of the biomarkers (e.g., miRNA) from cellular constituents. As a result, the gene product(s) of biomarkers can be obtained in soluble and easily accessible form. In some embodiments, the sample is diluted into an appropriate incubation buffer. Such incubation buffers are well-known to the skilled artisan. In some embodiments, a sample can be processed to enhance access to the nucleic acids, or copies of the nucleic acids, and the processed biological sample can then be used as the test sample. In some embodiments, nucleic acids are prepared from a sample. Nucleic acids can be obtained from the biological sample using suitable techniques. The nucleic acid content can be obtained from an extraction performed on a fresh or fixed biological sample.

In some embodiments, the nucleic acids are amplified in sample processing. Amplification can be performed with any method for increasing the number of copies of a nucleic acid sequence. For example, amplification can be performed with a polymerase, e.g., in one or more polymerase chain reactions (PCRs), including AFLP (amplified fragment length polymorphism) PCR, allele-specific PCR, Alu PCR, assembly, asymmetric PCR, colony PCR, helicase dependent PCR, hot start PCR, inverse PCR, in situ PCR, intersequence-specific PCR or IS SR PCR, digital PCR, droplet digital PCR, linear-after-the-exponential-PCR or Late PCR, long PCR, nested PCR, real-time PCR, duplex PCR, multiplex PCR, quantitative PCR, or single cell PCR. Other amplification methods may also be used, including ligase chain reaction (LCR), nucleic acid sequence-based amplification (NASBA), linear amplification, isothermal linear amplification, Q-beta-replicase method, 3 SR, Transcription Mediated Amplification (TMA), Strand Displacement Amplification (SDA), or Rolling Circle Amplification (RCA).

For illustrative purposes, in some embodiments, methods provided herein further comprise obtaining the sample from the subject. As such, provided herein are methods of assessing whether a subject has endometriosis or is predisposed to endometriosis, or the prognosis of a subject having endometriosis, wherein the methods comprise (1) obtaining a peripheral blood sample the subject; (2) within 2 hours, processing the blood to separate serum or plasma; (3a) optionally, if the sample is not immediately analyzed, freezing the serum or plasma sample for storage or shipping; (3b) if frozen, thawing the sample; (4) optionally, treating the sample with DNase; (5) extracting RNA from the sample; and (6) measuring the miRNA biomarkers in the sample using, e.g., qPCR. Levels of endogenous miRNA (e.g., miR-16-5p) or Spike-in mixture (e.g., UniSp2/4/5) can be used for normalization. In some embodiments, an altered expression pattern (e.g., weighted sum of a selected panel) of the miRNA biomarkers indicates that the subject has a gynecological disease (e.g., endometriosis). In some embodiments, the expression pattern of the miRNA biomarkers can distinguish endometriosis from other gynecological diseases. In some embodiments, the expression pattern of the miRNA biomarkers can distinguish endometriosis alone versus endometriosis in combination with adenomyosis. In some embodiments, the expression pattern of the miRNA biomarkers can distinguish minimal and mild (Stage I+II) endometriosis versus moderate and severe (Stage III+IV) endometriosis.

In some embodiments, methods provided herein comprise obtaining the sample during a specific window of the subject for miRNA level detection. In some embodiments, methods provided herein comprise obtaining the sample during the proliferative phase of the subject for miRNA level detection. In some embodiments, methods provided herein comprise obtaining the sample during the secretory phase of the subject for miRNA level detection. In some embodiments, methods provided herein comprise obtaining the sample when the progesterone level is high, indicating secretory phase. In some embodiments, methods provided herein comprise obtaining the sample during Days 6-14 of a menstrual cycle of a human female. In some embodiments, the miRNA panels are used in combination with CA-125 for endometriosis diagnosis. Accordingly, methods provided herein comprise obtaining a sample for measuring the CA-125 level and obtaining a second sample for measuring the miRNA level(s). In some embodiments, the second sample used for measuring the miRNA level(s) is different from the sample used for measuring CA-125 level. In some embodiments, the second sample used for measuring miRNA level(s) is obtained during proliferative phase of menstrual cycle and the sample used for measuring CA-125 level is obtained during secretory phase menstrual cycle. In some embodiments, the second sample is the same as the sample used for measuring CA-125 level obtained from secretory phase.

Sample size determination is based on a 50% prevalence of disease among the population of symptomatic women undergoing surgery for determination of disease. The term “prevalence” means the frequency of a condition of interest at a given point in time expressed as a fraction of the number of individuals in a specified group with the condition of interest compared to the total number of individuals. The estimated number of patient samples needed to achieve 95% confidence with 5% marginal error for a test that is 90% sensitive and specific. Calculation is based on “User's guide to sample size estimation in diagnostic accuracy studies” by Haldun Akoglu, 2022.

miRNA biomarkers for endometriosis are selected based on the following criteria: 1) significant differences in tissue expression levels between patients and controls; 2) detected in serum; and 3) reproducible and consistent differential expression in patient serum samples. The expression profile of the miRNA biomarkers are highly correlated with endometriosis pathophysiology associated with inflammation and cellular transformation and/or proliferation during the menstrual cycle.

### 7.4.1.3 Endogenous Controls

The development of diagnostic laboratory-developed tests (LDTs) and in vitro diagnostics (IVDs) relies extensively on technologies like quantitative PCR (qPCR) and droplet digital PCR (ddPCR). These platforms enable robust, high-sensitivity, and high-specificity point-of-care diagnostics, making them indispensable in both commercial and hospital laboratories. The effectiveness of diagnostic tests is fundamentally contingent on the biomarkers selected, which must reliably distinguish between disease and control states. The identification and selection of such biomarkers depend heavily on prior data and domain-specific knowledge of underlying biological pathways.

Traditionally, this prior data comes from previous qPCR/ddPCR studies or exploratory next-generation sequencing (NGS) data. NGS has been particularly influential in mutation profiling for applications such as early detection and minimal residual disease (MRD) monitoring. Mutational biomarkers are considered deterministic since their presence or absence is generally absolute, though their utility still depends on the assay's sensitivity and specificity. Advanced techniques, such as the incorporation of unique molecular identifiers (UMIs), enhance the precision of mutation detection by mitigating sequencing errors and enabling accurate quantification at low abundance levels.

The translation of transcriptomic next-generation sequencing (NGS) data into laboratory-developed tests (LDTs) and in vitro diagnostics (IVDs) has faced notable challenges, especially when compared to the relative success of mutation profiling. Unlike mutation data, which are deterministic and rely on clear presence/absence signals, transcriptomic data are inherently more variable and context-dependent. This variability necessitates substantial downstream processing, including batch correction, normalization, and careful statistical modeling, which complicates their integration into clinical workflows. Despite their promise for applications like gene expression profiling and biomarker discovery, the immaturity of analytical pipelines and the lack of standardization have limited the clinical utility of transcriptomic NGS data in diagnostic test development.

Normalization stands out as one of the primary barriers to the adoption of transcriptomic NGS data for LDTs and IVDs. In qPCR and ddPCR workflows, normalization typically relies on housekeeping genes, which are assumed to have stable expression across conditions. However, this assumption often fails in practice. For instance, U6 and miR-16 are commonly used as reference genes in microRNA (miRNA) studies, yet accumulating evidence indicates that their expression can vary significantly depending on experimental conditions, disease states, or sample types (e.g., Davoren et al., 2008; Blondal et al., 2013). Such variability undermines the reliability of these housekeeping genes for normalization, leading to skewed results that fail to recapitulate findings from NGS-based exploratory analyses. This disconnect between NGS-derived insights and qPCR/ddPCR-based validation creates a bottleneck in the translation of transcriptomic biomarkers into meaningful diagnostic assays.

Normalization in NGS transcriptomic workflows takes a fundamentally different approach. Instead of relying on housekeeping genes, NGS data are normalized based on factors like sequencing depth, gene length, and the total number of mapped reads (e.g., Robinson and Oshlack, 2010). While these methods are effective for exploratory analyses, they do not align well with the requirements for clinical diagnostics, where data must be reproducible, interpretable, and robust across varying conditions. This divergence in normalization strategies adds another layer of complexity, as improperly normalized data can obscure biological signals, reduce diagnostic accuracy, and impair reproducibility.

Several studies illustrate these challenges. Pantel and Speicher (2016) highlight the increasing utility of NGS in liquid biopsies, particularly for cancer diagnostics, but note that the variability and noise inherent in transcriptomic data pose significant obstacles to clinical implementation. Similarly, Risso et al. (2014) detail how batch effects in transcriptomic datasets can confound downstream analyses, necessitating careful preprocessing to preserve biological relevance. Bustin et al. (2009) emphasize the reliability of qPCR in diagnostics but caution against the indiscriminate use of housekeeping genes without rigorous validation.

To address these challenges, the selection of housekeeping markers for qPCR and ddPCR normalization must be carefully optimized to align with observations from NGS-based analyses. For instance, studies have begun identifying alternative reference genes or developing normalization methods tailored to specific experimental contexts (Peltier and Latham, 2008). Additionally, integrating NGS-derived normalization techniques, such as using spike-in controls or external standards, into qPCR/ddPCR workflows may help bridge the gap between exploratory research and clinical diagnostics.

In some embodiments, methods provided herein comprise normalizing the miRNA level using an endogenous control. In some embodiments, the endogenous control is miR-16-5p. In some embodiments, the sample is taken at secretory phase and the endogenous control is selected from: miR-92a-3p, miR-652-3p, miR-92b-3p, miR-532-5p, miR-338-5p, miR-374a-5p, and miR-421. In some embodiments, the endogenous control is miR-16-5p. In some embodiments, the endogenous control is miR-92a-3p. In some embodiments, the endogenous control is miR-652-3p. In some embodiments, the endogenous control is miR-92b-3p. In some embodiments, the endogenous control is miR-532-5p. In some embodiments, the endogenous control is miR-338-5p. In some embodiments, the endogenous control is miR-374a-5p. In some embodiments, the endogenous control is miR-421.

In some embodiments, the sample is taken at proliferative phase and the endogenous control is selected from: miR-181a-2-3p, miR-181d-5p, Let-7g-5p, miR-181a-3p, miR-24-2-5p, miR-130b-3p, miR-628-3p, miR-19a-3p, miR-181a-5p and miR-424-3p. In some embodiments, the endogenous control is miR-181a-2-3p. In some embodiments, the endogenous control is miR-181d-5p. In some embodiments, the endogenous control is Let-7g-5p. In some embodiments, the endogenous control is miR-181a-3p. In some embodiments, the endogenous control is miR-24-2-5p. In some embodiments, the endogenous control is miR-130b-3p. In some embodiments, the endogenous control is miR-628-3p. In some embodiments, the endogenous control is miR-19a-3p. In some embodiments, the endogenous control is miR-181a-5p. In some embodiments, the endogenous control is miR-424-3p.

### 7.4.1.4 Measurement of Expression Levels

Methods provided herein comprise measuring the expression levels of the miRNAs disclosed herein as biomarkers for endometriosis in a sample from a subject. Any methods available in the art for detecting and measuring miRNA can be adopted. In some embodiments, the miRNAs are detected using in situ RNA hybridization (e.g., fluorescence in situ hybridization, or FISH), quantitative polymerase chain reaction (qPCR), real-time polymerase chain reaction (RT-PCR), digital PCR (dPCR), such as droplet dPCR (ddPCR), microarray analysis, next generation sequencing (NGS), Northern blotting, or a luminex-based assay.

In some embodiments, methods provided herein use a nucleic acid that hybridizes with a miRNA disclosed herein. Such nucleic acids can be probes or primers. The probes or primers are of adequate length so as to minimize the amount of non-specific binding. The oligonucleotide probes or primers herein described can be prepared by any suitable methods such as chemical synthesis methods. In some embodiments, methods provided herein use a primer that is complementary to a nucleotide sequence of the miRNA of interest. In some embodiments, the primer includes 12 or more contiguous nucleotides substantially complementary to the sequence of the miRNA of interest. In some embodiments, a primer includes a nucleotide sequence sufficiently complementary to hybridize to a nucleic acid sequence of about 12 to 25 nucleotides. The primer differs by no more than 1, 2, or 3 nucleotides from the target nucleotide sequence. In another aspect, the length of the primer can vary in length, preferably about 15 to 28 nucleotides in length (e.g., 15, 16, 17, 18, 19, 20, 21, 22, 23, 24, 25, 26, 27 or 28 nucleotides in length).

The probes and primers according to the disclosure can be labeled directly or indirectly with a detectable agent. The detectable agent can be a radioactive isotope, for example, ³²P, ³³P, ³⁵S or ³H. The detectable agent also includes ligands such as biotin, avidin, streptavidin or digoxigenin, haptenes, dyes, and luminescent agents such as radioluminescent, chemoluminescent, bioluminescent, fluorescent or phosphorescent agents.

Alternatively, a peptide nucleic acid (PNA) probe can be used instead of a nucleic acid probe in the hybridization methods described herein. PNA is a DNA mimic having a peptide-like, inorganic backbone, such as N-(2-aminoethyl) glycine units, with an organic base (A, G, C, T or U) attached to the glycine nitrogen via a methylene carbonyl linker (see, for example, 1994, Nielsen et al., Bioconjugate Chemistry 5:1). The PNA probe can be designed to specifically hybridize to a nucleic acid sequence comprising at least one miRNA of interest. Hybridization of the PNA probe to a nucleic acid sequence is indicative of the presence of a miRNA of interest.

In some embodiments, methods provided herein include obtaining the nucleic acids from a sample. Nucleic acids can be obtained from the biological sample using suitable techniques. The nucleic acid content can be obtained from an extraction performed on a fresh or fixed biological sample. Nucleic acids include DNA and RNA (mRNA, miRNA, etc.). The nucleic acids can be double-stranded or single-stranded (i.e., a sense or an antisense single strand).

In some embodiments, methods provided herein comprise measuring miRNAs using quantitative PCR (qPCR). In a typical qPCR reaction, miRNAs in the sample are reverse transcribed into complementary DNA (cDNA), and then amplified by real-time PCR. Fluorescent dyes or probes can be used to monitor the amplification in real-time. See e.g., Mestdagh et al. (2009), Genome Biology, 10(6), R64. Typical qPCRs used in the art include SYBR Green-based qPCR, wherein SYBR Green dye binds to double-stranded DNA, and fluorescence increases proportionally to the amount of DNA synthesized during each PCR cycle, and TaqMan assays, in which TaqMan probes are designed to hybridize specifically to the target miRNA during PCR. The probe contains a fluorescent reporter dye and a quencher, allowing for specific detection.

For illustrative purposes, a typical qPCR Protocol for miRNA measurement can include the following steps: (1) RNA Extraction: isolate total RNA, including small RNAs, from cells or tissues using a suitable method (e.g., TRIzol reagent, miRNeasy Mini Kit). (2) Reverse Transcription (cDNA Synthesis): perform reverse transcription to convert miRNAs into complementary DNA (cDNA). This step often involves the use of miRNA-specific stem-loop primers. Commonly used enzymes include reverse transcriptase (e.g., SuperScript III). (3) qPCR Setup: Prepare the qPCR reaction mix containing cDNA, primers, and master mix. Appropriate negative controls and a standard curve for quantification are included. (4) PCR Amplification: Use a real-time PCR instrument to amplify the cDNA. Cycling conditions depend on the specific primers and master mix used. (5) Data Analysis: Monitor amplification curves in real-time and calculate cycle threshold (Ct) values. Normalize Ct values to an internal reference (endogenous control) or use a spike-in control for absolute quantification. (6) Expression Analysis: Analyze relative miRNA expression using the AACt method or absolute quantification based on the standard curve.

For illustrative purposes, a typical dPCR Protocol for miRNA measurement can include the following steps: (1) RNA Extraction: isolate total RNA, including small RNAs, from cells or tissues using a suitable method (e.g., TRIzol reagent, miRNeasy Mini Kit). (2) Reverse Transcription (cDNA Synthesis): perform reverse transcription to convert miRNAs into complementary DNA (cDNA). This step often involves the use of miRNA-specific stem-loop primers or poly (T) universal primers. Commonly used enzymes include reverse transcriptase (e.g., SuperScript III). (3) dPCR Setup: Prepare the dPCR reaction mix containing cDNA, primers, and master mix. Appropriate negative controls are included. (4) PCR Amplification: Use a digital PCR instrument to amplify the cDNA. Cycling conditions depend on the specific primers and master mix used. (5) Data Analysis: Monitor amplification curves in real-time and calculate cycle threshold (Ct) values. Positive and negative wells/droplest's fluorescence signal are identified based on fluorescence intensity. Using Poisson distribution algorithms to calculate the absolute concentration of the target molecules. Normalize copy numbers to an internal reference (endogenous control) or use a spike-in control. (6) Expression Analysis: Analyze relative miRNA expression using the ratio or absolute quantification. ddPCR is a type of dPCR where the sample is partitioned into thousands to millions of water-in-oil droplets. Each droplet undergoes PCR amplification individually. Examples include Bio-Rad's QX200™ or QX ONE™ droplet digital PCR systems.

Poly(T) adaptor RT-PCR is a PCR method that is useful in the methods of the disclosure to amplify and quantify miRNAs of interest (See Rui et al., 2012, Methods Mol Biol. 822:53-66; Jae et al., 2018, Mol Bio Rep. 45(4): 611-619; Varkonyi-Gasic et al., 2011, Methods Mol Biol.744:145-57). Briefly, the method includes two steps: RT and real-time PCR. First, target miRNAs are poly(A) tailed by poly(A) polymerase followed by cDNA synthesis using poly(T) primer with a reverse transcriptase. Then, the RT products are quantified using conventional real-time PCR.

In some embodiments, the RNA extraction step further includes treatment with DNase to remove or eliminate gDNA background. In some embodiments, the sample is subject to DNase treatment if it is processed at least 2 hours after it is obtained from patient. For example, in some embodiments, the sample is a blood sample, and is subject to DNase treatment if it is processed at least 2 hours after the blood draw. In some embodiment, the blood sample is subject to DNase treatment if it is processed at least 3 hours after the blood draw.

For normalization in a qPCR assay, suitable reference genes or internal controls can be used normalization. Common choices include small nuclear RNAs (snRNAs) or small nucleolar RNAs (snoRNAs). In some embodiments, controls with no-reverse transcription and non-template controls are included to assess background signal and contamination. For assay validation, synthetic miRNA standards can be used.

In some embodiments of the methods disclosed herein, endogenous miRNAs that can serve as internal control include, for example, miR-103a (e.g., miR-103a-3p), miR-191 (e.g., miR-191-5p), miR-16 (e.g., miR-16-5p), and Let-7a (e.g., Let-7a-5p).

In some embodiments, the PCR reaction is characterized in that the amplifications are real-time amplifications performed using a labeled probe, preferably a labeled hydrolysis-probe, capable of specifically hybridizing in stringent conditions with a segment of a nucleic acid sequence, or polymorphic nucleic acid sequence. The labeled probe is capable of emitting a detectable signal every time each amplification cycle occurs. Real-time amplification, such as real-time PCR, can be performed in various different iterations; it can be performed, e.g., using various categories of probes, such as hydrolysis probes, hybridization adjacent probes, or molecular beacons. The techniques employing hydrolysis probes or molecular beacons are based on the use of a fluorescence quencher/reporter system, and the hybridization adjacent probes are based on the use of fluorescence acceptor/donor molecules. Hydrolysis probes with a fluorescence quencher/reporter system are available in the market and are for example commercialized by the Applied Biosystems group (USA). Many fluorescent dyes may be employed, such as FAM dyes (6-carboxy-fluorescein), or any other dye phosphoramidite reagents.

Stem-loop RT-PCR is a PCR method that is useful in the methods of the disclosure to amplify and quantify miRNAs of interest (See Caifu et al., 2005, Nucleic Acids Research 33:el79; Mestdagh et al., 2008, Nucleic Acids Research 36:el43; Varkonyi-Gasic et al., 2011, Methods Mol Biol.744:145-57). Briefly, the method includes two steps: RT and real-time PCR. First, a stem-loop RT primer is hybridized to a miRNA molecule and then reverse transcribed with a reverse transcriptase. Then, the RT products are quantified using conventional real-time PCR.

In some embodiments, methods provided herein comprise measuring miRNAs using microarray analysis. Arrays of oligonucleotide probes that are complementary to target nucleic acid sequences from a subject can be used to detect, identify and quantify miRNAs associated with a gynecological disease (e.g., endometriosis). For example, in some embodiments, an oligonucleotide array is used, which comprises a plurality of different oligonucleotide probes that are coupled to a surface of a substrate in different pre-defined or addressed locations. These arrays can generally be produced using mechanical synthesis methods or light directed synthesis methods which incorporate a combination of photolithographic methods and solid phase oligonucleotide synthesis methods. After an oligonucleotide array is prepared, a sample containing miRNA is hybridized with the array and scanned for miRNAs. The level of detectable signals, such as fluorescence signals, from labeled miRNAs provide information on miRNA expression levels. Microarray platforms can be used for simultaneous analysis of multiple miRNAs. See e.g., Liu et al. (2004). PNAS, 101(26), 9740-44.

Direct sequence analysis can also be used to detect miRNAs of interest. A sample comprising nucleic acid can be used, and PCR or other appropriate methods can be used to amplify all or a fragment of the nucleic acid, and/or its flanking sequences. In some embodiments, methods provided herein comprise measuring miRNAs using Next-Generation Sequencing (NGS): NGS can provide a comprehensive analysis of miRNA profiles by sequencing small RNA libraries. The term “next generation” is well-understood in the art and generally refers to any high-throughput sequencing approach including, but not limited to one or more of the following: massively-parallel signature sequencing, pyrosequencing (e.g., using a Roche 454 sequencing device), Illumina (Solexa) sequencing, sequencing by synthesis (Illumina), Ion torrent sequencing, sequencing by ligation (e.g., SOLiD sequencing), single molecule real-time (SMRT) sequencing (e.g., Pacific Bioscience), Polony sequencing, DNA nanoball sequencing, Heliscope single molecule sequencing (Helicos Biosciences), and nanopore sequencing (e.g., Oxford Nanopore). In some embodiments, the sequencing assay uses nanopore sequencing. In some cases, the sequencing assay includes some form of Sanger sequencing. In some cases, the sequencing involves shotgun sequencing; in some cases, the sequencing includes bridge PCR. In some cases, the sequencing is broad spectrum. In some cases, the sequencing is targeted. See e.g., Morin et al. (2008) Genome Research, 18(4), 610-621; Voelkerding et al., 2009, Clinical Chemistry 55:641-658; Su et al., 2011, Expert Rev Mol Diagn. 11:333-343; Ji and Myllykangas, 2011, Biotechnol Genet Eng Rev 27: 135-158.

In some embodiments, methods provided herein comprise measuring miRNAs using Northern blotting. Northern blotting generally involves the separation of miRNAs by gel electrophoresis, transfer to a membrane, and hybridization with labeled probes. The probes can be labeled with a radioactive isotope, a fluorescent dye or any other conveniently detectable moiety. Northern blotting provides information on miRNA size and allows for the detection of multiple miRNAs simultaneously. See e.g., Lee & Ambros (2001) Science, 294(5543), 862-864.

In some embodiments, methods provided herein comprise measuring miRNAs using fluorescence in situ hybridization (FISH). FISH uses fluorescently labeled probes that specifically bind to miRNA sequences within cells or tissues, allowing for the visualization of miRNA localization. As such, FISH can provide spatial information on miRNA expression within cells. See e.g., Raj et al. (2008) Nature Methods, 5(10), 877-879.

In some embodiments, methods provided herein comprise measuring miRNAs using a luminex-based assay. Luminex technology uses color-coded beads with miRNA-specific capture probes, and the detection is based on fluorescence intensity. Luminex-based assays have high-throughput capability and the ability to measure multiple miRNAs simultaneously. See e.g., Chen et al. (2005) Nucleic Acids Research, 33(20), e179.

Other methods of nucleic acid analysis can be used to detect miRNAs of interest. Representative methods include automated fluorescent sequencing; single-stranded conformation polymorphism assays (SSCP); clamped denaturing gel electrophoresis (CDGE); denaturing gradient gel electrophoresis (DGGE) (Sheffield et al., 1981, Proc. Natl. Acad. Sci. USA 86:232-236), mobility shift analysis (Biophys. Chem. 265: 1275; 1991, Keen et al, Trends Genet. 7:5); RNase protection assays (Myers et al., 1985, Science 230:1242); high-throughput sequencing (HTS) (Gundry and Vijg, 2011, Mutat Res); and/or ion semiconductor sequencing (Rusk, 2011, Nature Methods; Rothberg et al, 2011, Nature 475:348-352). These and other methods, alone or in combination, can be used to detect and quantity of at least one miRNA of interest, in a biological sample obtained from a subject. In some embodiments of the disclosure, the methods of assessing a biological sample to detect and quantify a miRNA of interest, as described herein, are used to diagnose, provide prognosis, monitor disease progression or treatment efficacy, or otherwise assess and characterize endometriosis in a subject in need thereof.

### 7.4.1.5 Determination of Reference Levels

Provided herein are methods for assessing the risk for endometriosis in a subject, including the risk for having endometriosis, the risk for developing endometriosis, the risk for advancing into a later stage of endometriosis, and the risk of having reoccurrence of endometriosis, comprising (a) measuring a level of a miRNA selected from: miR-17, miR-19b, miR-21, miR-15b, miR-19a, miR-664a, miR-381, miR-23a, miR-654, miR-24, Let-7b, miR-20a, miR-22, miR-34c, miR-1287, miR-625, miR-1294, miR-7704, miR-221, miR-340, miR-450b, miR-548e, miR-502, miR-574, miR-9, miR-299, miR-6789, miR-593, miR-346, miR-34a, miR-449a, miR-7109, miR-3907, miR-557, miR-6801, miR-4420, miR-570, miR-155, miR-199, miR-520d, miR-424, and miR-200 in a sample of the subject, and (b) comparing the level to a reference level; wherein an altered level indicates the risk for a gynecological disease (e.g., endometriosis).

A reference level, or alternatively a control level, is the level to which the expression level of the biomarker in a sample of interest is compared. A reference level thereby provides a standard allowing for the evaluation of the information obtained from the sample of interest. The reference level can be a positive control, a negative control, a normal control, a wild-type control, a historical control, a historical norm, or the level of another reference molecule in the biological sample.

In some embodiments, a reference level can be derived from a healthy or normal tissue, organ or individual, thereby providing a standard of a healthy status of a tissue, organ or individual. Differences between the status of the normal reference sample and the status of the sample of interest can be indicative of the risk of disease development or the presence or further progression of such disease or disorder. A reference level can also be derived from an abnormal or diseased tissue, organ or individual thereby providing a standard of a diseased status of a tissue, organ or individual. Differences between the status of the abnormal reference level and the status of the sample of interest can be indicative of a lowered risk of disease development or the absence or bettering of such disease or disorder.

A reference level can be an internal or an external reference. An internal reference sample is used, i.e., the marker level(s) is(are) assessed in the test sample as well as in one or more other sample(s) taken from the same subject to determine if there are any changes in the level(s) of said marker(s) in the test sample. For an external reference, the presence or amount of a marker in a sample derived from the individual is compared to its presence or amount in an individual known to suffer from, or known to be at risk of, a given condition; or an individual known to be free of a given condition, i.e., “normal individual” or “healthy individual.”

As understood by the skilled artisan, external reference levels can be obtained from a single individual or a reference population that is age-matched and free of confounding diseases. Typically, samples from 100 or more well-characterized individuals from the appropriate reference population are used to establish a reference level. However, reference population can also be chosen to consist of about 20, about 30, about 50, about 200, about 500 or about 1000 individuals. Healthy individuals represent a preferred reference population for establishing a reference level.

In some embodiments, the reference expression level of a particular miRNA biomarker can be determined based on statistical analysis of data from previous clinical studies, including clinical presentation of a group of patients. Many statistical methods are well known in the art to determine the reference level (or referred to as the “cut-off value”) of one or more biomarkers when used to assessing the risk of a subject for having, developing, or further progressing into an advanced stage of a condition.

One method includes analyzing gene expression profiles for biomarkers to determine the reference expression level for one or more biomarkers. For example, comparison of the expression levels of a particular miRNA biomarker between subjects having or not having endometriosis can be performed using the Mann-Whitney U-test, Chi-square test, or Fisher's Exact test. Analysis of descriptive statistics and comparisons can be performed using SigmaStat Software (Systat Software, Inc).

In some embodiments, a classification and regression tree (CART) analysis can be adopted to determine the reference level. CART analysis is based on a binary recursive partitioning algorithm and allows for the discovery of complex predictor variable interactions that may not be apparent with more traditional methods, such as multiple linear regression. Binary recursive partitioning refers to the analysis that is: 1) binary, meaning there were two possible outcome variables, namely “healthy” and “diseased,” with the effect of splitting patients into 2 groups; 2) recursive, meaning the analysis can be performed multiple times; and 3) partitioned, meaning the entire data set can be split into sections. This analysis also can eliminate predictor variables with poor performance. The classification tree can be built using Salford Predictive Modeler v6.6 (Salford Systems, San Diego, CA, USA).

Receiver Operator Characteristic (ROC) analysis can be utilized to determine the reference expression level, or test the overall predictive value of individual genes and/or multigene classifiers. A review of the ROC analysis can be found in Soreide, J Clin Pathol 10.1136 (2008), which is hereby incorporated by reference in its entirety. The reference level can be determined from the ROC curve of the training set to ensure both high sensitivity and high specificity. To determine how many biomarkers are needed to be included in the predictor, leave-one-out cross validation (LOOCV) can be used. The response scores for the ‘left-out’ samples based on different numbers of genes are recorded. The performances of the predictors with different numbers of genes can be assessed based on misclassification error rate, sensitivity, specificity, etc.

The Top Scoring Pair (TSP) algorithm first introduced by Geman et al. (2004) can be used. In essence, the algorithm ranks all the gene pairs (genes i and j) based on the absolute difference (Dij) in the frequency of event where gene i has higher expression value than gene j in samples among class CI to C2. In the cases of there are multiple top scoring pairs (all sharing the same Dij), the top pair by a secondary rank score that measures the magnitude to which inversions of gene expression levels occur from one class to the other within a pair of genes is selected. The top pair with highest frequency of absolute Dij>2 fold in all samples will be selected as candidate pair. The candidate pair can then be assessed in an independent testing data set. LOOCV can be carried out in the training data set to evaluate how the algorithm perform. The performances of the predictors can be assessed based on maximum misclassification error rate. All the statistical analyses can be done using R (R Development Core Team, 2006).

A review of the methods and statistical tools useful for determining a reference level can be found in James Westgard, Ph.D., Basic Methods Validation, 3d edition (2008), which is hereby incorporated by reference in its entirety. Specific references are made to Chapter 9 (“How is reportable range of a method determined”) and Chapter 15 (“How is a reference interval verified”).

Clinically reportable range (CRR) is the range of analyte values that a method can measure, allowing for specimen dilution, concentration, or other pretreatment used to extend the direct analytical measurement range. As provided in the Basic Methods Validation by Dr. Westgard, the experiment to be performed is often called a “linearity experiment,” though there technically is no requirement that a method provide a linear response unless two-point calibration is being used. This range can also be referred to as the “linear range,” “analytical range,” or “working range” for a method. The reportable range is assessed by inspection of the linearity graph. That inspection can involve manually drawing the best straight line through the linear portion of the points, drawing a point-to-point line through all the points then comparing with the best straight line, or fitting a regression line through the points in the linear range. There are more complicated statistical calculations that are recommended in some guidelines, such as Clinical Laboratory Standards Institute (CLSI)'s EP-6 protocol for evaluating the linearity of analytical methods. But it is commonly accepted that the reportable range can be adequately determined from a “visual” assessment, i.e., by manually drawing the best straight line that fits the lowest points in the series. The Clinical Laboratory Standards Institute (CLSI) recommends a minimum of at least 4- preferably 5-different levels of concentrations. More than 5 can be used, particularly if the upper limit of reportable range needs to be maximized, but 5 levels are convenient and almost always sufficient.

A reference level is typically established by assaying specimens that are obtained from individuals that meet carefully defined criteria (reference sample group). Protocols such as those of the International Federation of Clinical Chemistry (IFCC) Expert Panel on Theory of Reference Values and the CLSI delineate comprehensive systematic processes that use carefully selected reference sample groups to establish reference intervals. These protocols typically need a minimum of 120 reference individuals for each group (or subgroup) that needs to be characterized.

The CLSI Approved Guideline C28-A2 describes different ways for a laboratory to validate the transference of established reference intervals to the individual laboratory that includes 1. Divine judgment, wherein the laboratory simply reviews the information submitted and subjectively verifies that the reference intervals are applicable to the adopting laboratory's patient population and test methods; 2. Verification with 20 samples, wherein experimental validation is performed by collecting and analyzing specimens from 20 individuals who represent the reference sample population; 3. Estimation with 60 samples, wherein an experimental validation is performed by collecting and analyzing specimens from 60 individuals who represent the reference sample population, and the actual reference interval is estimated and compared to the claimed or reported interval using a statistical formula comparing the means and standard deviations of the two populations; and 4. Calculation from comparative method, wherein one can adjust or correct the claimed or reported reference intervals on the basis of the observed methodological bias and the mathematical relationship demonstrated between the analytical methods being used.

Depending on the intended use, an appropriate control sample is chosen, and a reference value established therein. As also clear to the skilled artisan, the absolute marker values established in a control sample depend on the assay used for measuring the expression level.

In some embodiments, the method comprises using a quantitative algorithm to determine if the expression level of a set of biomarkers in the biological sample is statistically different than the expression level in a control sample. The algorithm can be a trained algorithm. In some embodiments, the algorithm is drawn from the group consisting essentially of: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; a plurality of algorithms arranged in a committee network; and forward floating search or backward floating search algorithms. Such algorithms may be used in supervised or unsupervised learning modes. In various embodiments, quantitative algorithms according to the disclosure can be used to determine the extent, severity, or stage of disease, to determine the right treatment approach (e.g., oral contraceptives, disease-specific therapy, surgical intervention), to select the appropriate dose for a medical treatment, to determine whether a patient is likely to respond to a particular medical or surgical treatment, to monitor response to treatment, or to monitor disease progression.

In some embodiments, the methods according to the disclosure include deriving a numerical value, index or score from the quantitative algorithm or mathematical formula. In some embodiments, the derived numerical value can serve as a cut off value for distinguishing between two or more potential outcomes (e.g., high or low risk of disease presence, progression or recurrence or stage of disease.) In some embodiments, a derived numerical value serves as a cutoff value for an expression level of at least one miRNA. In some embodiments, a derived numerical value serves as a cutoff value for an expression level of at least one miRNA in order determine the extent, severity, or stage of disease. In some embodiments, a derived numerical value serves as a cutoff value for an expression level of at least one miRNA to determine the right treatment approach (e.g., oral contraceptives, disease-specific therapy, surgical intervention). In some embodiments, a derived numerical value serves as a cutoff value for an expression level of at least one miRNA to select the appropriate dose for a medical treatment. In some embodiments, a derived numerical value serves as a cutoff value for an expression level of at least one miRNA to determine whether a patient is likely to respond to a particular medical or surgical treatment. In some embodiments, a derived numerical value serves as a cutoff value for an expression level of at least one miRNA to monitor response to treatment. In some embodiments, a derived numerical value serves as a cutoff value for an expression level of at least one miRNA to monitor disease progression.

The present disclosure also provides for software for guiding the risk analysis for endometriosis. The software combines one or more of the methods described elsewhere herein to diagnose or guide treatment of endometriosis.

In some embodiments, the present disclosure includes software executing instructions and algorithms relating to the methods provided herein. Such software can be stored on a non-transitory computer-readable medium, wherein the software performs some or all of the steps according to the present disclosure when executed on a processor.

Aspects of the disclosure relate to algorithms of the disclosure executed in computer software. Though certain embodiments can be described as written in particular programming languages, or executed on particular operating systems or computing platforms, it is understood that the systems and methods of the present disclosure are not limited to any particular computing language, platform, or combination thereof. Software executing the algorithms described herein may be written in any programming language compiled or interpreted, including but not limited to C, C++, C#, Objective-C, Java, JavaScript, Python, PHP, Perl, Ruby, or Visual Basic. It is further understood that elements of the present disclosure can be executed on any acceptable computing platform, including but not limited to a server, a cloud instance, a workstation, a thin client, a mobile device, an embedded microcontroller, a television, or any other suitable computing device.

Parts of this disclosure provide for software running on a computing device. Though software described herein may be disclosed as operating on one particular computing device (e.g., a dedicated server or a workstation), it is understood in the art that software is intrinsically portable and that most software running on a dedicated server can also be run, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device. Similarly, embodiments according to the present disclosure are described as communicating over a variety of wireless or wired computer networks. As used herein, the words “network,” “networked,” and “networking” are understood to encompass wired Ethernet, fiber optic connections, wireless connections including any of the various 802.11 standards, cellular WAN infrastructures such as 3G or 4G/LTE networks, Bluetooth®, Bluetooth® Low Energy (BLE) or Zigbee® communication links, or any other method by which one electronic device is capable of communicating with another. In some embodiments, elements of the networked portion of embodiments according to the present disclosure may be implemented over a Virtual Private Network (VPN).

### 7.5 Methods of Treatment

In some embodiments, methods provided herein also comprise treating the subject assessed to have endometriosis with a therapy for endometriosis. In some embodiments, methods provided herein comprise providing a prophylactic treatment to the subject who is assessed to be predisposed to endometriosis. In some embodiments, methods provided herein comprise providing an appropriate therapy for the subject having endometriosis that is assessed to be likely to progress into an advanced stage, as determined by rASRM staging.

In some embodiments, provided herein are methods of treating a subject who has endometriosis or is predisposed to endometriosis, comprising (a) measuring a level of a miRNA selected from: miR-17, miR-19b, miR-21, miR-15b, miR-19a, miR-664a, miR-381, miR-23a, miR-654, miR-24, Let-7b, miR-20a, miR-22, miR-34c, miR-1287, miR-625, miR-1294, miR-7704, miR-221, miR-340, miR-450b, miR-548e, miR-502, miR-574, miR-9, miR-299, miR-6789, miR-593, miR-346, miR-34a, miR-449a, miR-7109, miR-3907, miR-557, miR-6801, miR-4420, miR-570, miR-155, miR-199, miR-520d, miR-424, and miR-200 in a sample of the subject, and (b) administering a treatment for endometriosis to the subject if the level of the miRNA is altered compared to a reference level.

Provided herein are also methods of treating a subject exhibiting at least one clinical indicator for endometriosis, comprising (a) measuring a level of a miRNA selected from miR-17, miR-19b, miR-21, miR-15b, miR-19a, miR-664a, miR-381, miR-23a, miR-654, miR-24, Let-7b, miR-20a, miR-22, miR-34c, miR-1287, miR-625, miR-1294, miR-7704, miR-221, miR-340, miR-450b, miR-548e, miR-502, miR-574, miR-9, miR-299, miR-6789, miR-593, miR-346, miR-34a, miR-449a, miR-7109, miR-3907, miR-557, miR-6801, miR-4420, miR-570, miR-155, miR-199, miR-520d, miR-424, and miR-200 in a sample of the subject, and (b) administering a treatment for endometriosis to the subject if the level of the miRNA is altered compared to a reference level. Clinical indicators of endometriosis include, e.g., intermenstrual bleeding, dysmenorrhea, dyspareunia, dyschezia, dysuria, chronic pelvic pain, lower abdominal pain, or infertility. In some embodiments, the subject exhibits no clinical indicator of endometriosis. In some embodiments, the subject has a family history of endometriosis. In some embodiments of the methods of prognosis disclosed herein, the subject is known to have endometriosis.

In some embodiments of the methods provided herein, the subject is a mammal. In some embodiments, the subject is a human. In some embodiments, the subject is a human female. In some embodiments, the subject is a human female aged between 12-60 years. In some embodiments, the subject is a human female aged about 20 years old, 30 years old, 40 years old, 50 years old or 60 years old. In some embodiments, the subject is a human female diagnosed rASRM stage I or II endometriosis.

Treatment for endometriosis can involve medication or surgery, depending on severity of the symptoms and the goal of treatment (e.g., need for pregnancy).

Treatment can also be a hormonal treatment, an oral contraceptive, a progestin, a GnRH agonist, an androgen, an aromatase inhibitor, a non-steroidal anti-inflammatory drug, or any combination thereof. Treatment of endometriosis commonly include pain medication, such as the nonsteroidal anti-inflammatory drugs (NSAIDs) ibuprofen or naproxen sodium. Treatment of endometriosis can also include hormone therapies, which can slow endometrial tissue growth and prevent new implants of endometrial tissue. Supplemental hormones include hormonal contraceptives, including birth control pills, patches and vaginal rings. Supplemental hormones also include gonadotropin-releasing hormone (Gn-RH) agonists and antagonists, which block the production of ovarian-stimulating hormones, lowering estrogen levels and preventing menstruation. Supplemental hormones also include progestin therapy. A variety of progestin therapies, including an intrauterine device with levonorgestrel, contraceptive implant, contraceptive injection or progestin pill, can halt menstrual periods and the growth of endometrial implants. Supplemental hormones also include aromatase inhibitors, which are a class of medicines that reduce the amount of estrogen. An aromatase inhibitor can be used in combination with a progestin or combination hormonal contraceptive to treat endometriosis.

Treatment of endometriosis can include surgeries. In some embodiments, the subject is treated with a conservative surgery to remove the endometriosis implants while preserving uterus and ovaries. The surgeries can be done laparoscopically or, less commonly, through traditional abdominal surgery in more-extensive cases. The surgery can include removing the lesions (excising), destroying the lesions with intense heat (cauterizing or vaporizing), or removing the endometriosis patches (laparotomy). In some embodiments, the treatment is a surgical procedure. In some embodiments, the surgical procedure selected from: laparoscopy, laparotomy, presacral neurectomy, laparoscopic uterine nerve ablation (LUNA), and hysterectomy.

In some embodiments, the treatment includes surgical removal of the uterus (hysterectomy) is needed. If the ovaries have endometriosis on them or if damage is severe, the treatment can also include removal of ovaries and fallopian tubes along with the uterus (total hysterectomy and bilateral salpingo-oophorectomy). In some embodiments, if the pain is in the center of the abdomen, treatment can include surgery to sever pelvic nerves, which can be done during either laparoscopy or laparotomy. Two procedures are used to sever different nerves in the pelvis: presacral neurectomy, which severs the nerves connected to the uterus, and laparoscopic uterine nerve ablation (LUNA), which severs nerves in the ligaments that secure the uterus.

Endometriosis can lead to trouble conceiving. In some embodiments, fertility treatment is administered, which ranges from stimulating ovaries to in vitro fertilization.

### 7.6 Methods of Monitoring or Prognosis

The level of one or more miRNA biomarkers disclosed herein can also be used to monitor the effectiveness of treatment or the prognosis of disease. In some embodiments, the level of one or more miRNA biomarkers in a sample obtained from a treated patient can be compared to the level from a reference sample obtained from that patient before initiation of a treatment. Clinical monitoring of treatment typically entails that each patient serves as his or her own baseline control. In some embodiments, samples are obtained at multiple time points following administration of the treatment. In these embodiments, measurement of level of one or more miRNA biomarkers in the test samples provides an indication of the extent and duration of in vivo effect of the treatment.

In some embodiments, the disclosure provides a method for monitoring the levels of miRNA biomarkers disclosed herein in response to treatment. For example, in some embodiments, the disclosure provides for a method of determining the efficacy of treatment in a subject, by measuring the levels of one or more miRNAs described herein. In some embodiments, the level of the one or more miRNAs can be measured over time, where the level at one timepoint after the initiation of treatment is compared to the level at another timepoint after the initiation of treatment. In some embodiments, the level of the one or more miRNAs can be measured over time, where the level at one timepoint after the initiation of treatment is compared to the level before initiation of treatment. Accordingly, the disclosure provides methods of monitoring efficacy of a treatment for endometriosis in a subject, comprising (a) measuring a level of a miRNA selected from: miR-17, miR-19b, miR-21, miR-15b, miR-19a, miR-664a, miR-381, miR-23a, miR-654, miR-24, Let-7b, miR-20a, miR-22, miR-34c, miR-1287, miR-625, miR-1294, miR-7704, miR-221, miR-340, miR-450b, miR-548e, miR-502, miR-574, miR-9, miR-299, miR-6789, miR-593, miR-346, miR-34a, miR-449a, miR-7109, miR-3907, miR-557, miR-6801, miR-4420, miR-570, miR-155, miR-199, miR-520d, miR-424, and miR-200 in a sample of the subject, and (b) comparing the level to a reference level; wherein a decrease in the disparity indicates the efficacy of the treatment.

To identify therapeutics or drugs that are appropriate for a specific subject, a sample from the subject can also be exposed to a therapeutic agent or a drug, and the level of one or more biomarkers can be determined. Biomarker levels can be compared to a sample derived from the subject before and after treatment or exposure to a therapeutic agent or a drug or can be compared to samples derived from one or more subjects who have shown improvements relative to a disease as a result of such treatment or exposure. Thus, in one aspect, the disclosure provides a method of assessing the efficacy of a therapy with respect to a subject comprising taking a first measurement of a biomarker panel in a first sample from the subject; effecting the therapy with respect to the subject; taking a second measurement of the biomarker panel in a second sample from the subject and comparing the first and second measurements to assess the efficacy of the therapy.

Additionally, therapeutic agents suitable for administration to a particular subject can be identified by detecting one or more biomarkers in an effective amount from a sample obtained from a subject and exposing the subject-derived sample to a test compound that determines the amount of the biomarker(s) in the subject-derived sample. Accordingly, treatments or therapeutic regimens for use in subjects having endometriosis can be selected based on the amounts of biomarkers in samples obtained from the subjects and compared to a reference value. Two or more treatments or therapeutic regimens can be evaluated in parallel to determine which treatment or therapeutic regimen would be the most efficacious for use in a subject to delay onset, or slow progression of a disease. In some embodiments, a recommendation is made on whether to initiate or continue treatment of a disease.

The miRNA biomarkers disclosed herein can also be used for assessing the probable course and outcome of a disease or a condition (e.g., endometriosis), including prediction of severity, duration, chances of recovery, etc., in a subject having endometriosis. Prognosis can be expressed in various ways; for example, prognosis can be expressed as the likelihood that a condition may be manageable, treatable or curable, or the likelihood that a disease will go into remission or progress into an advanced severe stage. In the case of endometriosis, prognosis can include, for example, determining the likelihood that the disease can be cured, that the symptoms can be managed, and/or that the patient is likely to have a successful pregnancy. The prognosis for endometriosis can also include determining the likelihood that the disease is unlikely to be treated, the disease might progress into a more advanced stage, that the patient is likely to have reoccurrence of the symptoms (and optionally, the frequency and intensity of recurrence), and/or that the patient is unlikely to have a successful pregnancy.

In certain embodiments, the levels of one or more miRNA biomarkers disclosed herein are used as indicators of an unfavorable prognosis. Provided herein are methods include determining the prognosis of a subject, including, e.g., whether the subject is likely to progress into an advanced stage as determined by rASRM staging, comprising (a) measuring a level of a miRNA selected from: miR-155, miR-574, miR-23, miR-17, miR-20, miR-22, and Let-7b in a sample of the subject, and (b) comparing the level to a reference level; wherein an altered level indicates an unfavorable prognosis, including, e.g., that the endometriosis is likely to progress into an advanced stage in the subject, as determined by rASRM staging.

According to the current disclosure, the determination of prognosis can be performed by comparing the measured miRNA level to levels corresponding with favorable or unfavorable outcomes. According to the method, values can be collected from a series of patients with endometriosis to determine appropriate reference ranges of the miRNA biomarkers disclosed herein. Various techniques are available for performing a retrospective study that compares the determined levels to the observed outcome of the patients and establishing ranges of levels that can be used to designate the prognosis of the patients with a particular disorder. In some embodiments, the level of miRNA biomarker in a test sample from a patient relates to the prognosis of a patient in a continuous fashion, the determination of prognosis can be performed using statistical analyses to relate the determined miRNA levels to the prognosis of the patient. A skilled artisan can design appropriate statistical methods. For example, the methods may employ the chi-squared test, the Kaplan-Meier method, the log-rank test, multivariate logistic regression analysis, Cox's proportional-hazard model and the like in determining the prognosis. Computers and computer software programs may be used in organizing data and performing statistical analyses. The approach by Giles et al., British Journal of Hematology, 121:578-585, is exemplary. As in Giles et al., associations between categorical variables (e.g., miRNA levels and clinical characteristics) can be assessed via cross-tabulation and Fisher's exact test. Unadjusted survival probabilities can be estimated using the method of Kaplan and Meier. The Cox proportional hazards regression model also can be used to assess the ability of patient characteristics (such as miRNA levels) to predict survival, with ‘goodness of fit’ assessed by the Grambsch-Themeau test, Schoenfeld residual plots, martingale residual plots and likelihood ratio statistics (see Grambsch et al., 1995). In some embodiments, this approach can be adapted as a simple computer program that can be used with personal computers or personal digital assistants (PDA).

In some embodiments, methods provided herein include providing prognosis for a subject having endometriosis, and further using the prognosis to determine treatment for the subject. Conservative treatments, such as pain medications, hormonal therapies, and lifestyle modifications, can provide relief for individuals with favorable prognosis. Conversely, more aggressive approaches, including surgical interventions such as laparoscopy or, in severe cases, hysterectomy, may be necessary for others.

In some embodiments, multiple measurements of the miRNA biomarker levels can be made, and a temporal change in activity can be used to determine a prognosis. For example, comparative measurements are made of the miRNA biomarkers in samples from a patient taken at multiple time points, and a comparison of the expression levels of a miRNA biomarker at two or more time points can be indicative of a particular prognosis.

### 7.7 Kits

Provided herein are also kits useful in the methods of the disclosure. In some embodiments, kits provided herein are for assessing the risk for gynecological disease (e.g., endometriosis). In some embodiments, provided herein are kits for diagnosing gynecological disease (e.g., endometriosis) in a subject, for determining whether a subject is at risk of developing gynecological disease (e.g., endometriosis), for determining the prognosis in a subject having endometriosis, and for monitoring efficacy of a treatment for endometriosis in a subject, based upon the expression level or expression pattern of one or more miRNA biomarkers associated with endometriosis. Kits provided herein comprise a means for measuring the expression level of a particular miRNA biomarker disclosed herein, and an ancillary reagent. In some embodiments, provided herein are kits for assessing whether a subject has a gynecological disease (e.g., endometriosis) comprising a means for measuring a level of a miRNA selected from: miR-17, miR-19b, miR-21, miR-15b, miR-19a, miR-664a, miR-381, miR-23a, miR-654, miR-24, Let-7b, miR-20a, miR-22, miR-34c, miR-1287, miR-625, miR-1294, miR-7704, miR-221, miR-340, miR-450b, miR-548e, miR-502, miR-574, miR-9, miR-299, miR-6789, miR-593, miR-346, miR-34a, miR-449a, miR-7109, miR-3907, miR-557, miR-6801, miR-4420, miR-570, miR-155, miR-199, miR-520d, miR-424, and miR-200 in a sample of the subject, and an ancillary reagent. In some embodiments, the miRNA is selected from: miR-17-5p, miR-17-3p, miR-19b-5p, miR-19b-3p, miR-21-5p, miR-21-3p, miR-15b-5p, miR-15b-3p, miR-19a-5p, miR-19a-3p, miR-664a-5p, miR-664a-3p, miR-381-5p, miR-381-3p, miR-23a-5p, miR-23a-3p, miR-654-5p, miR-654-3p, miR-24-5p, miR-24-3p, Let-7b-5p, Let-7b-3p, miR-20a-5p, miR-20a-3p, miR-22-5p, miR-22-3p, miR-34c-5p, miR-34c-3p, miR-1287-5p, miR-1287-3p, miR-625-5p, miR-625-3p, miR-1294-5p, miR-1294-3p, miR-7704, miR-221-5p, miR-221-3p, miR-340-5p, miR-340-3p, miR-450b-5p, miR-450b-3p, miR-548e-5p, miR-548e-3p, miR-502-5p, miR-502-3p, miR-574-5p, miR-574-3p, miR-9-5p, miR-9-3p, miR-299-5p, miR-299-3p, miR-6789-5p, miR-6789-3p, miR-593-5p, miR-593-3p, miR-346-5p, miR-346-3p, miR-34a-5p, miR-34a-3p, miR-449a-5p, miR-449a-3p, miR-7109-5p, miR-7109-3p, miR-3907, miR-557, miR-6801-5p, miR-6801-3p, miR-4420, miR-570-5p, miR-570-3p, miR-155-5p, miR-155-3p, miR-199a-5p, miR-199a-3p, miR-199b-5p, miR-199b-3p, miR-520d-5p, miR-520d-3p, miR-424-5p, miR-424-3p, miR-200a-5p, miR-200a-3p, miR-200b-5p, miR-200b-3p, miR-200c-5p, and miR-200c-3p. In some embodiments, the he miRNA is selected from: miR-17-5p, miR-19b-3p, miR-21-5p, miR-21-3p, miR-15b-5p, miR-19a-3p, miR-664a-3p, miR-381-3p, miR-23a-3p, miR-654-3p, miR-24-3p, Let-7b-5p, miR-20a-5p, miR-22-5p, miR-22-3p, miR-34c-3p, miR-1287-5p, miR-625-5p, miR-1294-5p, miR-7704, miR-221-5p, miR-340-5p, miR-450b-5p, miR-548e-3p, miR-502-3p, miR-574-3p, miR-9-5p, miR-299-5p, miR-299-3p, miR-6789-5p, miR-593-5p, miR-346, miR-34a-5p, miR-449a, miR-7109-5p, miR-3907, miR-557, miR-6801-3p, miR-4420, miR-570-3p, miR-155-5p, miR-199a-5p, miR-520d-5p, miR-424, and miR-200.

In some embodiments, provided herein are kits for assessing whether a subject has a gynecological disease (e.g., endometriosis) comprising a means for measuring a level of a miRNA selected from: miR-17, miR-19b, miR-21, miR-15b, miR-19a, miR-664a, miR-381, miR-23a, miR-654, and miR-24 in a sample of the subject, and an ancillary reagent. In some embodiments, the miRNA is selected from: miR-17-5p, miR-19b-3p, miR-21-5p, miR-15b-5p, miR-19a-3p, miR-664a-3p, miR-381-3p, miR-23a-3p, miR-654-3p, and miR-24-3p.

In some embodiments, provided herein are kits for assessing whether a subject has a gynecological disease (e.g., endometriosis) comprising a means for measuring a level of a miRNA selected from: miR-21, miR-23a, miR-1287, miR-625, miR-1294, miR-7704, miR-221, miR-340, miR-450b, miR-548e, and miR-502 in a sample of the subject, and an ancillary reagent. In some embodiments, the miRNA is selected from: miR-21-3p, miR-22-5p, miR-1287-5p, miR-625-5p, miR-1294-5p, miR-7704, miR-221-5p, miR-340-5p, miR-450b-5p, miR-548e-3p, and miR-502-3p.

In some embodiments, provided herein are kits for assessing whether a subject has a gynecological disease (e.g., endometriosis) comprising a means for measuring a level of a miRNA selected from: Let-7b, miR-20a, miR-22, miR-34c, miR-574, miR-9, miR-299, miR-6789, miR-593, miR-346, miR-34a, miR-449a, miR-7109, miR-3907, miR-557, miR-6801, miR-4420, and miR-570 in a sample of the subject, and an ancillary reagent. In some embodiments, the miRNA is selected from: Let-7b-5p, Let-7b-3p, miR-20a-5p, miR-22-3p, miR-34c-3p, miR-574-3p, miR-9-5p, miR-299-5p, miR-299-3p, miR-6789-5p, miR-593-5p, miR-346, miR-34a-5p, miR-449a, miR-7109-5p, miR-3907, miR-557, miR-6801-3p, miR-4420, and miR-570-3p.

In some embodiments, kits provided herein comprise means for measuring miRNA biomarkers provided herein for detecting gynecological disease (e.g., endometriosis). In some embodiments, the miRNA biomarker comprises miR-34c. In some embodiments, the miRNA biomarker comprises miR-34c-3p. In some embodiments, the miRNA biomarker comprises Let-7b. In some embodiments, the miRNA biomarker comprises Let-7b-5p. In some embodiments, the miRNA biomarker comprises miR-17. In some embodiments, the miRNA biomarker comprises miR-17-5p. In some embodiments, the miRNA biomarker comprises miR-22. In some embodiments, the miRNA biomarker comprises miR-22-3p. In some embodiments, the miRNA biomarker comprises miR-20a. In some embodiments, the miRNA biomarker comprises miR-20a-5p. In some embodiments, the miRNA biomarker comprises miR-34c. In some embodiments, the miRNA biomarker comprises miR-34c-3p. In some embodiments, the miRNA biomarker comprises miR-199. In some embodiments, the miRNA biomarker comprises miR-199a-5p.

In some embodiments, kits provided herein comprise means for measuring a panel of the miRNA biomarkers provided herein for detecting gynecological disease (e.g., endometriosis). In some embodiments, the panel of miRNA biomarkers includes miR-17, miR-199 and miR-34c. In some embodiments, the panel of miRNA biomarkers includes miR-17-5p, miR-199a-5p and miR-34c-3p. In some embodiments, the panel of miRNA biomarkers includes miR-20a, miR-22 and miR-34c. In some embodiments, the panel of miRNA biomarkers includes miR-20a-5p, miR-22-3p and miR-34c-3p. In some embodiments, the panel of miRNA biomarkers includes miR-17, miR-22, miR-20a, miR-34c and miR-155. In some embodiments, the panel of miRNA biomarkers includes miR-17-5p, miR-22-3p, miR-20a-5p, miR-34c-3p and miR-155-5p. In some embodiments, the panel of miRNA biomarkers includes miR-34c, miR-23a, miR-17, miR-155, Let-7b, miR-199, miR-22, and miR-20a. In some embodiments, the panel of miRNA biomarkers includes miR-34c-3p, miR-23a-3p, miR-17-5p, miR-155-5p, Let-7b-5p, miR-199-5p, miR-22-3p, and miR-20a-5p. In some embodiments, the panel of miRNA biomarkers includes miR-34c and miR-20a. In some embodiments, the panel of miRNA biomarkers includes miR-34c-3p and miR-20a-5p. In some embodiments, the panel of miRNA biomarkers includes miR-34c and miR-17. In some embodiments, the panel of miRNA biomarkers includes miR-34c-3p and miR-17-5p. In some embodiments, the panel of miRNA biomarkers includes miR-34c and miR-22. In some embodiments, the panel of miRNA biomarkers includes miR-34c-3p and miR-22-5p. In some embodiments, the panel of miRNA biomarkers includes miR-155, miR-22 and miR-23a. In some embodiments, the panel of miRNA biomarkers includes miR155-5p, miR-22-5p and miR-23a-3p. In some embodiments, the panel of miRNA biomarkers includes miR-155-5p, miR-22-3p and miR-23a-3p. In some embodiments, the panel of miRNA biomarkers further includes Let-7b, miR-17, and miR-20a. In some embodiments, the panel of miRNA biomarkers further includes Let-7b-5p, miR-17-5p, and miR-20a-5p. In some embodiments, the panel of miRNA biomarkers further includes Let-7b-3p, miR-17-5p, and miR-20a-5p. In some embodiments, a panel of miRNA biomarkers is provided for the early diagnosis of endometriosis, including Let-7b-5p, miR-155-5p, miR-23a-3p, miR-17-5p, miR-20a-5p, and miR-22-5p. In some embodiments, a panel of miRNA biomarkers is provided for the early diagnosis of endometriosis, including Let-7b-5p, miR-155-5p, miR-23a-3p, miR-17-5p, miR-20a-5p, and miR-22-3p. In some embodiments, the panel of miRNA biomarkers further includes miR-574, miR-520d, or both. In some embodiments, the panel of miRNA biomarkers further includes miR-574-3p, miR-520d-5p, or both. In some embodiments, a panel of miRNA biomarkers is provided for the early diagnosis of endometriosis, including miR-155-5p, miR-22-3p, miR-23a-3p, miR-17-5p, miR-20a-5p, Let-7b-5p, Let-7b-3p, miR-574-3p, and miR-520d-5p. In some embodiments, a panel of miRNA biomarkers is provided for the early diagnosis of endometriosis, including miR-155-5p, miR-22-5p, miR-23a-3p, miR-17-5p, miR-20a-5p, Let-7b-5p, miR-574-3p, and miR-520d-5p.

In some embodiments, provided herein are kits for assessing whether a subject has a gynecological disease (e.g., endometriosis) comprising a means for measuring a level of a miRNA targeting CA-125, SIRT1 or BCL6 in a sample of the subject, and an ancillary reagent. In some embodiments, the miRNA targeting CA-125 can be miR-299-3p, miR-299-5p, miR-6789-5p, miR-593-5p, miR-7109-5p, and miR-3907. In some embodiments, the miRNA targeting SIRT1 can be miR-9-5p, miR-34a-5p, miR-449a, miR-4420, and miR-570-3p. In some embodiments, the miRNA targeting BCL16 can be miR-346, miR-557, and miR-6801-3p. In some embodiments, provided herein are kits for assessing whether a subject has a gynecological disease (e.g., endometriosis) comprising a means for measuring a level of a miRNA selected from: miR-299-3p, miR-299-5p, miR-6789-5p, miR-593-5p, miR-7109-5p, miR-3907, miR-9-5p, miR-34a-5p, miR-449a, miR-4420, miR-570-3p, miR-346, miR-557, and miR-6801-3p.

In some embodiments, kits provided herein comprise means for measuring the panel of miRNA biomarkers for distinguishing endometriosis alone from other gynecological disease. In some embodiments, the panel of miRNA biomarkers comprises miR-155, Let-7b, miR-23a, miR-17, or miR-20a, or any combination thereof. In some embodiments, the panel comprises miR-155-5p, Let-7b-5p, miR-23a-3p, miR-17-5p, miR-20a-5p, or any combination thereof. In some embodiments, the panel of miRNA biomarkers comprises Let-7b, miR-17, miR-20a, or any combination thereof. In some embodiments, the panel comprises Let-7b-5p, miR-17-5p, miR-20a-5p, or any combination thereof. In some embodiments, the panel comprises Let-7b-5p, miR-17-5p, and miR-20a-5p. In some embodiments, the panel comprises miR-155, miR-23a, miR-17, or miR-20a, or any combination thereof. In some embodiments, the panel comprises miR-155-5p, miR-23a-3p, miR-17-5p, or miR-20a-5p, or any combination thereof. In some embodiments, the panel comprises miR-155-5p, miR-23a-3p, miR-17-5p, and miR-20a-5p. In some embodiments, the panel can further include miR-424, miR-200 family miRNA, or both. In some embodiments, the panel further includes miR-424-5p. In some embodiments, the panel further includes miR-200a, miR-200b, miR-200c, or any combination thereof.

In some embodiments, kits provided herein comprise means for measuring the panel of miRNA biomarkers for endometriosis staging, namely, to distinguish mild (Stage I or II) and moderate/severe (Stage III or IV) endometriosis. In some embodiments, the panel for detecting late-stage endometriosis comprises miR-17, miR-20a, miR-22, or any combination thereof. In some embodiments, the panel for detecting late-stage endometriosis comprises miR-17-5p, miR-20a-5p, miR-22-5p, or any combination thereof. In some embodiments, the panel for detecting late-stage endometriosis comprises miR-17-5p, miR-20a-5p and miR-22-5p. In some embodiments, the panel can further include miR-424, miR-200 family miRNA, or both for distinguishing endometriosis and gynecological disease. In some embodiments, the panel further includes miR-424-5p. In some embodiments, the panel further includes miR-200a, miR-200b, miR-200c or any combination thereof.

In some embodiments, kits provided herein comprise means for measuring the panel for detecting adenomyosis. In some embodiments, In some embodiments, kits provided herein comprise means for measuring the panel of miRNA biomarkers for distinguishing patients having endometriosis only and patients having both endometriosis and adenomyosis. In some embodiments, the difference between the expression level of a miRNA biomarker and the expression level can be used. In some embodiments, the difference is compared to a cutoff value, wherein a difference that is higher than cutoff indicates that the subject has both endometriosis and adenomyosis, and a difference that is lower than cutoff indicates that the subject has endometriosis without adenomyosis.

In some embodiments, the means included in kits disclosed herein comprises one or more nucleic acid probes or primers for detecting one or more miRNA biomarkers disclosed herein. In some embodiments, the means comprises one or more primer pairs for amplifying one or more miRNA biomarkers disclosed herein. The probes or primers can be labeled. In some embodiments of the disclosure, the kit comprises 2 or more probes or primers. In some embodiments, the kits contain 3, 4, 5, 6, 7, 8, 9, 10, 15, 20, 25, or more probes or primers. In some embodiments, the kit can comprise a microarray.

In some embodiments, provided herein are kits for assessing whether a subject has a gynecological disease (e.g., endometriosis) comprising a means for measuring a level of a miRNA selected from: miR-17-5p, miR-19b-3p, miR-21-5p, miR-15b-5p, miR-19a-3p, and miR-664a-3p. In some embodiments, the means for measuring the miRNA level comprise at least one primer pair provided below:

| biomarker | Forward primer (5′-3′) | Reverse primer (5′-3′) |
|---|---|---|
| miR-17-5p | CGACAGGCCAAAGTGCTTACAG | GTCGTATCCAGTGCAGGGTCC |
| | (SEQ ID NO: 3) | (SEQ ID NO: 1) |
| miR-19b-3p | ACCGAGGTTGTGCAAATCCATG | GTCGTATCCAGTGCAGGGTCC |
| | (SEQ ID NO: 4) | (SEQ ID NO: 1) |
| miR-21-5p | ACCACCGTAGCTTATCAGACTGA | GTCGTATCCAGTGCAGGGTCC |
| | (SEQ ID NO: 5) | (SEQ ID NO: 1) |
| miR-15b-5p | ACCACCGTAGCAGCACATCA | GTCGTATCCAGTGCAGGGTCC |
| | (SEQ ID NO: 6) | (SEQ ID NO: 1) |
| miR-19a-3p | GCGGCCTGTGTGCAAATCTATG | GTCGTATCCAGTGCAGGGTCC |
| | (SEQ ID NO: 7) | (SEQ ID NO: 1) |
| miR-664a-3p | AGGCGTGCTATTCATTTATCCC | GTCGTATCCAGTGCAGGGTCC |
| | (SEQ ID NO: 8) | (SEQ ID NO: 1) |

In some embodiments, kits provided herein comprise the primer pair having nucleotide sequences of SEQ ID NOs:3 and 1. In some embodiments, kits provided herein comprise the primer pair having nucleotide sequences of SEQ ID NOs:4 and 1. In some embodiments, kits provided herein comprise the primer pair having nucleotide sequences of SEQ ID NOs:5 and 1. In some embodiments, kits provided herein comprise the primer pair having nucleotide sequences of SEQ ID NOs:6 and 1. In some embodiments, kits provided herein comprise the primer pair having nucleotide sequences of SEQ ID NOs:7 and 1. In some embodiments, kits provided herein comprise the primer pair having nucleotide sequences of SEQ ID NOs:8 and 1.

In some embodiments, kits provided herein further comprise a means for measuring a level of an endogenous control miRNA selected from: miR-92a-3p, miR-652-3p, miR-92b-3p, miR-532-5p, miR-338-5p, miR-374a-5p, and miR-421. In some embodiments, the means for measuring the endogenous control miRNA level comprise at least one primer pair provided below:

| biomarker | Forward primer (5′-3′) | Reverse primer (5′-3′) |
|---|---|---|
| miR-92a-3p | CCAGGCCATATTGCACTTGTC | GTCGTATCCAGTGCAGGGTCC |
| | (SEQ ID NO: 9) | (SEQ ID NO: 1) |
| miR-652-3p | AAGTACTCAATGGCGCCACTA | GTCGTATCCAGTGCAGGGT |
| | (SEQ ID NO: 10) | (SEQ ID NO: 2) |
| miR-421 | AGCCAGCGATCAACAGACATTAA | GTCGTATCCAGTGCAGGGTCC |
| | (SEQ ID NO: 11) | (SEQ ID NO: 1) |

In some embodiments, kits provided herein comprise the primer pair having nucleotide sequences of SEQ ID NOs:9 and 1. In some embodiments, kits provided herein comprise the primer pair having nucleotide sequences of SEQ ID NOs:10 and 2. In some embodiments, kits provided herein comprise the primer pair having nucleotide sequences of SEQ ID NOs:11 and 1.

In some embodiments, kits disclosed herein further comprise a reverse transcriptase, a DNA polymerase, or a DNA polymerase with reverse transcriptase activity. In some embodiments, the kits disclosed herein further comprise a DNase. In some embodiments, kits disclosed herein include restriction enzymes. In some embodiments, kits provided herein further comprise positive and negative controls. The kits can additionally include materials and reagents for isolating RNA from a biological sample. In some embodiments, the kits can contain a DNase. The kit can be tailored for in-home use, clinical use, or research use.

In some embodiments, provided herein are kits for measuring the expression level of one or more miRNA biomarkers disclosed herein by in situ hybridization. The kits can comprise a probe, The probe can be double-stranded DNA (dsDNA), single-stranded DNA (ssDNA), RNA, or synthetic oligonucleotide (e.g., PNA, LNA). The probe can be detectably labeled. In some embodiments, the kits further comprise a labeling regent. The detectable label can be a radioactive isotope such as 32P, 35S, or 3H. The detectable label can also be biotin, digoxigenin, fluorescent dye (FISH), or any other label described herein or otherwise known in the art. The kits can further include a solid surface, such as a slide, hybridization buffer, wash buffer, mounting buffer, or any combination thereof. The kits can further include reagents for RNA isolation. Further, the kits can include instructions for performing in situ hybridization and methods for interpreting and analyzing the data resulting from the performance of the assay.

In some embodiments, kits for measuring the expression of one or more miRNA biomarkers disclosed herein are provided herein, which comprise materials and reagents that are necessary for PCR assays. For example, an RT-PCR kit can be produced for a specific condition and contain the reagents and materials necessary for measuring the levels of the miRNA disclosed herein. In some embodiments, such kits can further include materials and reagents for synthesizing cDNA from RNA isolated from a sample. The kits generally include probes attached to a solid support surface. In one such embodiment, probes can be either be oligonucleotides or longer length probes including probes ranging from 150 nucleotides in length to 800 nucleotides in length. The probes can be attached to a detectable label. Included within the kits are probes specific for miRNA biomarkers disclosed herein. In some embodiments, such kits can include primers for PCR as well as probes for Quantitative PCR. The kits can include instructions for performing the assay and methods for interpreting and analyzing the data resulting from the performance of the assay. The kits can also include hybridization reagents and/or reagents necessary for detecting a signal produced when a probe hybridizes to a target nucleic acid sequence. Generally, the materials and reagents for the kits are in one or more containers. Each component of the kit is generally in its own a suitable container.

For Quantitative PCR, the kits can include pre-selected primers for specific nucleic acid sequences. The Quantitative PCR kits can also include enzymes suitable for amplifying nucleic acids (e.g., polymerases such as Taq), and deoxynucleotides and buffers needed for the reaction mixture for amplification. The Quantitative PCR kits can also include probes specific for the nucleic acid sequences associated with or indicative of a condition. The probes can be labeled with a fluorophore. The probes can also be labeled with a quencher molecule. In some embodiments the Quantitative PCR kits can also include components suitable for reverse-transcribing RNA including enzymes (e.g., reverse transcriptases such as AMV, MMLV and the like) and primers for reverse transcription along with deoxynucleotides and buffers needed for the reverse transcription reaction. Each component of the quantitative PCR kit is generally in its own suitable container. Thus, these kits generally include distinct containers suitable for each individual reagent, enzyme, primer and probe. Further, the quantitative PCR kits can include instructions for performing the assay and methods for interpreting and analyzing the data resulting from the performance of the assay.

The kits provided herein can further comprise a means for measuring the expression level of CA-125 in a sample of the subject. In some embodiments, the means for measuring the expression level of CA-125 comprises an anti-CA-125 antibody. In some embodiments, the kits provided herein comprise a secondary antibody.

For antibody based kits (e. CA-125., IHC kits, or ELISA kits), the kit can include, for example: (1) a first antibody which binds to NALCN; and, optionally, (2) a second, different antibody which binds to either CA-125, or the first antibody and is conjugated to a detectable label (e.g., a fluorescent label, radioactive isotope or enzyme). The first antibody can be attached to a solid support. In a specific embodiment, the polypeptide or protein of interest is a biomarker provided herein. The antibody-based kits can also include beads for conducting an immunoprecipitation. The kits can further comprise blocking buffers, diluents, mounting medium (e.g., mounting reagents for fluorescent dye, or mounting medium for IHC with chromogen), or any combination thereof. Each component of the antibody-based kits is generally in its own suitable container. Thus, these kits generally include distinct containers suitable for each antibody. Further, the antibody-based kits can include instructions for performing the assay and methods for interpreting and analyzing the data resulting from the performance of the assay.

Kits provided herein also include an ancillary reagent. In some embodiments, the ancillary reagent can be a secondary antibody, a detection agent, a reaction buffer, a stop buffer, a dilution buffer, or a wash buffer, or any combination thereof.

Secondary antibodies can be monoclonal or polyclonal antibodies. Secondary antibodies can be derived from any mammalian organism, including bovine, mice, rats, hamsters, goats, camels, chicken, rabbit, and others. Secondary antibodies can include, for example, an anti-human IgA antibody, an anti-human IgD antibody, an anti-human IgE antibody, an anti-human IgG antibody, or an anti-human IgM antibody. Secondary antibodies can be conjugated to enzymes (e.g., horseradish peroxidase (HRP), alkaline phosphatase (AP), luciferase, and the like) or dyes (e.g., colorimetric dyes, fluorescent dyes, fluorescence resonance energy transfer (FRET)-dyes, time-resolved (TR)-FRET dyes, and the like). In some embodiments, the secondary antibody is a polyclonal rabbit-anti-human IgG antibody, which is HRP-conjugated.

Any detection reagent known in the art can be included in a kit of this disclosure. In some embodiments, the detection reagent is a colorimetric detection reagent, a fluorescent detection reagent, or a chemiluminescent detection reagent. In some embodiments, the colorimetric detection reagent includes PPP (p-nitrophenyl phosphate), ABTS (2,2′-azino-bis(3-ethylbenzothiazoline-6-sulphonic acid)) or OPD (o-phenylenediamine). In some embodiments, the fluorescent detection reagent includes QuantaBlu™ or QuantaRed™ (Thermo Scientific, Waltham, MA). In some embodiments, the luminescent detection reagent includes luminol or luciferin. In some embodiments, the detection reagent includes a trigger (e.g., H202) and a tracer (e.g., isoluminol-conjugate). Any detection buffer known in the art can be included in a kit of this disclosure. In some embodiments the detection buffer is a citrate-phosphate buffer (e.g., about pH 4.2).

Any stop solution known in the art can be included in a kit of this disclosure. The stop solutions of this disclosure terminate or delay the further development of the detection reagent and corresponding assay signals. Stop solutions can include, for example, low-pH buffers (e.g., glycine-buffer, pH 2.0), chaotrophic agents (e.g., guanidinium chloride, sodium-dodecylsulfate (SDS)) or reducing agents (e.g., dithiothreitol, mecaptoethanol), or the like.

A large selection of washing buffers is known in the art, such as tris(hydroxymethyl)aminom ethane (Tris)-based buffers (e.g., Tris-buffered saline, TBS) or phosphate buffers (e.g., phosphate-buffered saline, PBS). Washing buffers can include detergents, such as ionic or non-ionic detergents. In some embodiments, the washing buffer is a PBS buffer (e.g., about pH 7.4) including Tween®20 (e.g., about 0.05% Tween®20).

Any dilution buffer known in the art can be included in a kit of this disclosure. Dilution buffers can include a carrier protein (e.g., bovine serum albumin, BSA) and a detergent (e.g., Tween®20). In some embodiments, the dilution buffer is PBS (e.g., about pH 7.4) including BSA (e.g., about 1% BSA) and Tween®20 (e.g., about 0.05% Tween®20).

In some embodiments, the ancillary reagent is an immobilization reagent, which can be any immobilization reagent known in the art, including covalent and non-covalent immobilization reagents. Covalent immobilization reagents can include any chemical or biological reagent that can be used to covalently immobilize a peptide or a nucleic acid on a surface. Covalent immobilization reagents can include, for example, a carboxyl-to-amine reactive group (e.g., carbodiimides such as EDC or DCC), an amine reactive group (e.g., N-hydroxysuccinimide (NHS) esters, imidoesters), a sulfhydryl-reactive crosslinker (e.g., maleimides, haloacetyls, pyridyl disulfides), a carbonyl-reactive crosslinker groups (e.g., hydrazides, alkoxyamines), a photoreactive crosslinker (e.g., aryl azides, dizirines), or a chemoselective ligation group (e.g., a Staudinger reaction pair). Non-covalent immobiliazation reagents include any chemical or biological reagent that can be used to immobilize a nucleic acid non-covalently on a surface, such as affinity tags (e.g., biotin) or capture ragents (e.g., streptavidin). The kits of this disclosure can include combinations of immobilization reagents. Such combinations include, for example, EDC and NHS, which can be used, for example, to immobilize a protein of this disclosure on a surface, such as a carboxylated dextrane matrix (e.g., on a BIAcore™ CM5 chip or a dextrane-based bead). Combinations of immobilization reagents can be stored as premixed reagent combinations or with one or more immobilization reagents of the combination being stored separately from other immobilization reagents.

In some embodiments, the kit of this disclosure includes a cleaning reagent for an automated assay system. An automated assay system can include systems by any manufacturer. In some embodiments, the automated assay systems include, for example, the BIO-FLASH™, the BEST 2000TM, the DS2TM, the ELx50 WASHER, the ELx800 WASHER, and the ELx800 READER. A cleaning reagent can include any cleaning reagent known in the art.

In some embodiments, kits provided herein further comprise a solid support. Examples of solid support suitable for carrying out the methods disclosed herein include beads, particles, colloids, single surfaces, tubes, multiwell plates, microtiter plates, slides, membranes, gels and electrodes. The solid support of the kit can be, for example, a plastic, silicon, a metal, a resin, glass, a membrane, a particle, a precipitate, a gel, a polymer, a sheet, a sphere, a polysaccharide, a capillary, a film, a plate, or a slide. When the solid phase is a particulate material (e.g., beads), it is, in one embodiment, distributed in the wells of multi-well plates to allow for parallel processing of the solid phase supports. The kits provided herein can employ, for example, a dipstick, a membrane, a chip, a disk, a test strip, a filter, a microsphere, a slide, a multiwell plate, or an optical fiber.

In some embodiments, kits provided herein further comprise a container for sample collection. In some embodiments, the kits provided herein include one or more containers and components for conducting in situ hybridization, RT-PCR, qPCR, deep sequencing, NGS, or a microarray.

In some embodiments, kits provided herein further comprise instructions. Exemplary instructions include a publication, a recording, a diagram, or any other medium of expression which can be used to communicate the usefulness, methods and/or protocols of the components disclosed in the kit.

It is noted that any combination of the above-listed embodiments, for example, with respect to one or more reagents, such as, without limitation, nucleic acid primers, solid support and the like, are also contemplated in relation to any of the various methods and/or kits provided herein.

For illustrative purposes, in some embodiments, provided herein are kits for diagnosing endometriosis in a human female, comprising a means for detecting the expression of miRNA biomarkers Let-7b-5p, miR-155-5p, miR-23a-3p, miR-17-5p, miR-20a-5p, and miR-22-5p. The kits can comprise in situ hybridization reagents or qPCR regents, all of which are well known in the art. The kits can further include a sampler for collects a sample from the human female. The kits can further comprise a control sample which provides a reference level of the biomarker. The kits can further comprise instructions for conducting the assay and/or for interpreting the results.

For digital PCR including nanoplate digital PCR and droplet digital PCR, the kits can include pre-selected primers for specific nucleic acid sequences. The digital PCR kits can also include enzymes suitable for amplifying nucleic acids (e.g., polymerases such as Taq), and deoxynucleotides and buffers needed for the reaction mixture for amplification. The digital PCR kits can also include probes specific for the nucleic acid sequences associated with or indicative of a condition. The probes can be labeled with a fluorophore. The probes can also be labeled with a quencher molecule. In some embodiments the digital PCR kits can also include primers specific for the nucleic acid sequences with or indicative of a condition, and DNA-binding Dye (e.g., Evagreen). In some embodiments the digital PCR kits can also include components suitable for reverse-transcribing RNA including enzymes (e.g., reverse transcriptases such as AMV, MMLV and the like) and primers for reverse transcription along with deoxynucleotides and buffers needed for the reverse transcription reaction. Each component of the digital PCR kit is generally in its own suitable container. Thus, these kits generally include distinct containers suitable for each individual reagent, enzyme, primer and probe. Further, the digital PCR kits can include instructions for performing the assay and methods for interpreting and analyzing the data resulting from the performance of the assay.

The practice of the invention employs, unless otherwise indicated, conventional techniques in molecular biology, cell biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Maniatis et al. (1982) MOLECULAR CLONING: A LABORATORY MANUAL, Cold Spring Harbor Laboratory Press; Sambrook et al. (1989), MOLECULAR CLONING: A LABORATORY MANUAL, Second Edition, Cold Spring Harbor Laboratory Press; Sambrook et al. (2001) MOLECULAR CLONING: A LABORATORY MANUAL, Cold Spring Harbor Laboratory Press, Cold Spring Harbor, NY; Ausubel et al., CURRENT PROTOCOLS IN MOLECULAR BIOLOGY, John Wiley & Sons (1987 and annual updates); CURRENT PROTOCOLS IN IMMUNOLOGY, John Wiley & Sons (1987 and annual updates) Gait (ed.) (1984) OLIGONUCLEOTIDE SYNTHESIS: A PRACTICAL APPROACH, IRL Press; Eckstein (ed.) (1991) OLIGONUCLEOTIDES AND ANALOGUES: A PRACTICAL APPROACH, IRL Press; Birren et al. (eds.) (1999) GENOME ANALYSIS: A LABORATORY MANUAL, Cold Spring Harbor Laboratory Press; Borrebaeck (ed.) (1995); each of which is incorporated herein by reference in its entirety.

### 7.8 Illustrative Embodiments

### 7.8.1 Set 1

Embodiment 1: A method of detecting a gynecological disease in a subject, comprising (a) measuring a level of at least one miRNA selected from: miR-17, miR-19b, miR-21, miR-15b, miR-19a, miR-664a, miR-381, miR-23a, miR-654, miR-24, Let-7b, miR-20a, miR-22, miR-34c, miR-1287, miR-625, miR-1294, miR-7704, miR-221, miR-340, miR-450b, miR-548e, miR-502, miR-574, miR-9, miR-299, miR-6789, miR-593, miR-346, miR-34a, miR-449a, miR-7109, miR-3907, miR-557, miR-6801, miR-4420, miR-570, miR-155, miR-199, miR-520d, miR-424, and miR-200, in a sample of the subject, and (b) comparing the level to a reference level; wherein an altered level indicates that the subject has a gynecological disease.

Embodiment 2: The method of Embodiment 1, wherein: miR-17 comprises miR-17-5p, miR-17-3p, or both; miR-19b comprises miR-19b-5p, miR-19b-3p, or both; miR-21 comprises miR-21-5p, miR-21-3p, or both; miR-15b comprises miR-15b-5p, miR-15b-3p, or both; miR-19a comprises miR-19a-5p, miR-19a-3p, or both; miR-664a comprises miR-664a-5p, miR-664a-3p, or both; miR-381 comprises miR-381-5p, miR-381-3p, or both; miR-23a comprises miR-23a-5p, miR-23a-3p, or both; miR-654 comprises miR-654-5p, miR-654-3p, or both; miR-24 comprises miR-24-5p, miR-24-3p, or both; Let-7b comprises Let-7b-5p, Let-7b-3p, or both; miR-20a comprises miR-20a-5p, miR-20a-3p, or both; miR-22 comprises miR-22-5p, miR-22-3p, or both; miR-34c comprises miR-34c-5p, miR-34c-3p, or both; miR-1287 comprises miR-1287-5p, miR-1287-3p, or both; miR-625 comprises miR-625-5p, miR-625-3p, or both; miR-1294 comprises miR-1294-5p, miR-1294-3p, or both; miR-7704 comprises miR-7704; miR-221 comprises miR-221-5p, miR-221-3p, or both; miR-340 comprises miR-340-5p, miR-340-3p, or both; miR-450b comprises miR-450b-5p, miR-450b-3p, or both; miR-548e comprises miR-548e-5p, miR-548e-3p, or both; miR-502 comprises miR-502-5p, miR-502-3p, or both; miR-574 comprises miR-574-5p, miR-574-3p, or both; miR-9 comprises miR-9-5p, miR-9-3p, or both; miR-299 comprises miR-299-5p, miR-299-3p, or both; miR-6789 comprises miR-6789-5p, miR-6789-3p, or both; miR-593 comprises miR-593-5p, miR-593-3p, or both; miR-346 comprises miR-346-5p, miR-346-3p, or both; miR-34a comprises miR-34a-5p, miR-34a-3p, or both; miR-449a comprises miR-449a-5p, miR-449a-3p, or both; miR-7109 comprises miR-7109-5p, miR-7109-3p, or both; miR-3907 comprises miR-3907; miR-557 comprises miR-557; miR-6801 comprises miR-6801-5p, miR-6801-3p or both; miR-4420 comprises miR-4420; miR-570 comprises miR-570-5p, miR-570-3p, or both; miR-155 comprises miR-155-5p, miR-155-3p, or both; miR-199 comprises miR-199-5p, or miR-199-3p, or both; miR-520d comprises miR-520d-5p, miR-520d-3p, or both; miR-424 comprises miR-424-5p, miR-424-3p, or both; miR-200 comprises miR-200a-5p, miR-200a-3p, miR-200b-5p, miR-200b-3p, miR-200c-5p, or miR-200c-3p, or any combination thereof.

Embodiment 3: The method of Embodiment 1, wherein: miR-17 comprises miR-17-5p; miR-19b comprises miR-19b-3p; miR-21 comprises miR-21-5p, miR-21-3p, or both; miR-15b comprises miR-15b-5p; miR-19a comprises miR-19a-3p; miR-664a comprises miR-664a-3p; miR-381 comprises miR-381-3p; miR-23a comprises miR-23a-3p; miR-654 comprises miR-654-3p; miR-24 comprises miR-24-3p; Let-7b comprises Let-7b-5p; miR-20a comprises miR-20a-5p; miR-22 comprises miR-22-3p; miR-34c comprises miR-34c-3p; miR-1287 comprises miR-1287-5p; miR-625 comprises miR-625-5p; miR-1294 comprises miR-1294-5p; miR-7704 comprises miR-7704; miR-221 comprises miR-221-5p; miR-340 comprises miR-340-5p; miR-450b comprises miR-450b-5p; miR-548e comprises miR-548e-3p; miR-502 comprises miR-502-3p; miR-574 comprises miR-574-3p; miR-9 comprises miR-9-5p; miR-299 comprises miR-299-5p, miR-299-3p, or both; miR-6789 comprises miR-6789-5p; miR-593 comprises miR-593-5p; miR-346 comprises miR-346-5p, miR-346-3p, or both; miR-34a comprises miR-34a-5p; miR-449a comprises miR-449a-5p, miR-449a-3p, or both; miR-7109 comprises miR-7109-5p; miR-3907 comprises miR-3907; miR-557 comprises miR-557; miR-6801 miR-6801-3p; miR-4420 comprises miR-4420; miR-570 comprises miR-570-3p; miR-155 comprises miR-155-5p; miR-199 comprises miR-199-5p; miR-520d comprises miR-520d-5p; miR-424 comprises miR-424-5p; miR-200 comprises miR-200a-5p, miR-200a-3p, miR-200b-5p, miR-200b-3p, miR-200c-5p, or miR-200c-3p, or any combination thereof.

Embodiment 4: The method of Embodiment 1 or 2, comprising measuring the level of miR-17.

Embodiment 5: The method of Embodiment 4, wherein miR-17 comprises miR-17-5p.

Embodiment 6: The method of Embodiment 1 or 2, comprising measuring the level of miR-19b.

Embodiment 7: The method of Embodiment 6, wherein miR-19b comprises miR-19b-3p.

Embodiment 8: The method of Embodiment 1 or 2, comprising measuring the level of miR-21.

Embodiment 9: The method of Embodiment 8, wherein miR-21 comprises miR-21-5p.

Embodiment 10: The method of Embodiment 8, wherein miR-21 comprises miR-21-3p.

Embodiment 11: The method of Embodiment 1 or 2, comprising measuring the level of miR-15b.

Embodiment 12: The method of Embodiment 11, wherein miR-15b comprises miR-15b-5p.

Embodiment 13: The method of Embodiment 1 or 2, comprising measuring the level of miR-19a.

Embodiment 14: The method of Embodiment 13, wherein miR-19a comprises miR-19a-3p.

Embodiment 15: The method of Embodiment 1 or 2, comprising measuring the level of miR-664a.

Embodiment 16: The method of Embodiment 15, wherein miR-664a comprises miR-664a-3p.

Embodiment 17: The method of Embodiment 1 or 2, comprising measuring the level of miR-381.

Embodiment 18: The method of Embodiment 17, wherein miR-381 comprises miR-381-3p.

Embodiment 19: The method of Embodiment 1 or 2, comprising measuring the level of miR-23a.

Embodiment 20: The method of Embodiment 19, wherein miR-23a comprises miR-23a-3p.

Embodiment 21: The method of Embodiment 1 or 2, comprising measuring the level of miR-654.

Embodiment 22: The method of Embodiment 21, wherein miR-654 comprises miR-654-3p.

Embodiment 23: The method of Embodiment 1 or 2, comprising measuring the level of miR-24.

Embodiment 24: The method of Embodiment 23, wherein miR-24 comprises miR-24-3p.

Embodiment 25: The method of Embodiment 1 or 2, comprising measuring the level of Let-7b.

Embodiment 26: The method of Embodiment 25, wherein Let-7b comprises Let-7b-5p.

Embodiment 27: The method of Embodiment 1 or 2, comprising measuring the level of miR-20a.

Embodiment 28: The method of Embodiment 27, wherein miR-20a comprises miR-20a-5p.

Embodiment 29: The method of Embodiment 1 or 2, comprising measuring the level of miR-22.

Embodiment 30: The method of Embodiment 29, wherein miR-22 comprises miR-22-5p.

Embodiment 31: The method of Embodiment 29, wherein miR-22 comprises miR-22-3p.

Embodiment 32: The method of Embodiment 1 or 2, comprising measuring the level of miR-34c.

Embodiment 33: The method of Embodiment 32, wherein miR-34c comprises miR-34c-3p.

Embodiment 34: The method of Embodiment 1 or 2, comprising measuring the level of miR-155.

Embodiment 35: The method of Embodiment 34, wherein miR-155 comprises miR155-5p.

Embodiment 36: The method of Embodiment 1 or 2, comprising measuring the level of miR-1287.

Embodiment 37: The method of Embodiment 36, wherein miR-1287 comprises miR-1287-5p.

Embodiment 38: The method of Embodiment 1 or 2, comprising measuring the level of miR-625.

Embodiment 39: The method of Embodiment 38, wherein miR-625 comprises miR-625-5p.

Embodiment 40: The method of Embodiment 1 or 2, comprising measuring the level of miR-1294.

Embodiment 41: The method of Embodiment 40, wherein miR-1294 comprises miR-1294-5p.

Embodiment 42: The method of Embodiment 1 or 2, comprising measuring the level of miR-7704.

Embodiment 43: The method of Embodiment 1 or 2, comprising measuring the level of miR-221.

Embodiment 44: The method of Embodiment 43, wherein miR-221 comprises miR-221-5p.

Embodiment 45: The method of Embodiment 1 or 2, comprising measuring the level of miR-340.

Embodiment 46: The method of Embodiment 45, wherein miR-340 comprises miR-340-5p.

Embodiment 47: The method of Embodiment 1 or 2, comprising measuring the level of miR-450b.

Embodiment 48: The method of Embodiment 47, wherein miR-450b comprises miR-450b-5p.

Embodiment 49: The method of Embodiment 1 or 2, comprising measuring the level of miR-548e.

Embodiment 50: The method of Embodiment 49, wherein miR-548e comprises miR-548e-3p.

Embodiment 51: The method of Embodiment 1 or 2, comprising measuring the level of miR-502.

Embodiment 52: The method of Embodiment 51, wherein miR-502 comprises miR-502-3p.

Embodiment 53: The method of Embodiment 1 or 2, wherein the miRNA is selected from: miR-17, miR-19b, miR-21, miR-15b, miR-19a, miR-664a, miR-381, miR-23a, miR-654, and miR-24.

Embodiment 54: The method of Embodiment 53, wherein the miRNA is selected from: miR-17-5p, miR-19b-3p, miR-21-5p, miR-15b-5p, miR-19a-3p, miR-664a-3p, miR-381-3p, miR-23a-3p, miR-654-3p, and miR-24-3p.

Embodiment 55: The method of Embodiment 1 or 2, wherein the miRNA is selected from: miR-21, miR-23a, miR-1287, miR-625, miR-1294, miR-7704, miR-221, miR-340, miR-450b, miR-548e, and miR-502.

Embodiment 56: The method of Embodiment 55, wherein the miRNA is selected from: miR-21-3p, miR-22-5p, miR-1287-5p, miR-625-5p, miR-1294-5p, miR-7704, miR-221-5p, miR-340-5p, miR-450b-5p, miR-548e-3p, and miR-502-3p.

Embodiment 57: The method of Embodiment 1 or 2, wherein the miRNA is selected from: miR-17, miR-23a, Let-7b, miR-20a, miR-22, miR-155, miR-34c, and miR-199.

Embodiment 58: The method of Embodiment 57, wherein the miRNA is selected from: miR-17-5p, miR-23a-3p, Let-7b-5p, miR-20a-5p, miR-22-3p, miR-155-5p, miR-34c-3p, and miR-199-5p.

Embodiment 59: The method of Embodiment 1 or 2, wherein the miRNA is selected from: miR-17, miR-23a, Let-7b, miR-20a, miR-22, and miR-155.

Embodiment 60: The method of Embodiment 59, wherein the miRNA is selected from: miR-17-5p, miR-23a-3p, Let-7b-5p, miR-20a-5p, miR-22-3p, and miR-155-5p.

Embodiment 61: The method of Embodiment 1 or 2, wherein the miRNA is selected from: Let-7b, miR-20a, miR-22, miR-34c, miR-574, miR-9, miR-299, miR-6789, miR-593, miR-346, miR-34a, miR-449a, miR-7109, miR-3907, miR-557, miR-6801, miR-4420, and miR-570.

Embodiment 62: The method of Embodiment 61, wherein the miRNA is selected from: Let-7b-5p, miR-20a-5p, miR-22-3p, miR-34c-3p, miR-574-3p, miR-9-5p, miR-299-3p, miR-299-5p, miR-6789-5p, miR-593-5p, miR-346, miR-34a-5p, miR-449a, miR-7109-5p, miR-3907, miR-557, miR-6801-3p, miR-4420, and miR-570-3p.

Embodiment 63: The method of any one of Embodiments 1 to 62, comprising measuring levels of at least two, at least three, at least four, at least five, at least six, at least seven, at least eight, at least nine, or at least ten miRNAs.

Embodiment 64: The method of Embodiment 63, comprising measuring levels of miR-17, miR-19b, miR-21, miR-15b, miR-19a, miR-664a, miR-381, miR-23a, miR-654, and miR-24.

Embodiment 65: The method of Embodiment 64, comprising measuring levels of: miR-17-5p, miR-19b-3p, miR-21-5p, miR-15b-5p, miR-19a-3p, miR-664a-3p, miR-381-3p, miR-23a-3p, miR-654-3p, and miR-24-3p.

Embodiment 66: The method of Embodiment 63, wherein the miRNA is selected from: miR-21, miR-23a, miR-1287, miR-625, miR-1294, miR-7704, miR-221, miR-340, miR-450b, miR-548e, and miR-502.

Embodiment 67: The method of Embodiment 66, wherein the miRNA is selected from: miR-21-3p, miR-22-5p, miR-1287-5p, miR-625-5p, miR-1294-5p, miR-7704, miR-221-5p, miR-340-5p, miR-450b-5p, miR-548e-3p, and miR-502-3p.

Embodiment 68: The method of Embodiment 63, comprising measuring levels of: miR-17, miR-23a, Let-7b, miR-20a, miR-22, miR-155, miR-34c, and miR-199.

Embodiment 69: The method of Embodiment 68, comprising measuring levels of: miR-17-5p, miR-23a-3p, Let-7b-5p, miR-20a-5p, miR-22-3p, miR-155-5p, miR-34c-3p, and miR-199-5p.

Embodiment 70: The method of Embodiment 63, comprising measuring levels of: miR-17, miR-23a, Let-7b, miR-20a, miR-22, and miR-155.

Embodiment 71: The method of Embodiment 70, comprising measuring levels of: miR-17-5p, miR-23a-3p, Let-7b-5p, miR-20a-5p, miR-22-3p, and miR-155-5p.

Embodiment 72: The method of Embodiment 70 or 71, further comprising measuring levels of miR-574 and miR-520d.

Embodiment 73: The method of Embodiment 72, comprising measuring levels of miR-574-3p and miR-520d-5p.

Embodiment 74: The method of Embodiment 63, comprising measuring levels of at least two miRNAs selected from: miR-34c, miR-20a, miR-17, and miR-22.

Embodiment 75: The method of Embodiment 74, comprising measuring levels of at least two miRNAs selected from: miR-34c-3p, miR-20a-5p, miR-17-5p, and miR-22-3p.

Embodiment 76: The method of Embodiment 75, comprising measuring levels of miR-20a-5p and miR-34c-3p.

Embodiment 77: The method of Embodiment 75, comprising measuring levels of miR-17-5p and miR-34c-3p.

Embodiment 78: The method of Embodiment 75, comprising measuring levels of miR-22-3p and miR-34c-3p.

Embodiment 79: The method of Embodiment 75, comprising measuring levels of miR-17-5p, miR-20a-5p, miR-22-3p and miR-34c-3p.

Embodiment 80: The method of any one of Embodiments 1 to 79, wherein the miRNA level is normalized using an endogenous control.

Embodiment 81: The method of Embodiment 80, wherein the endogenous control is selected from: miR-16-5p, miR-92a-3p, miR-652-3p, miR-92b-3p, miR-532-5p, miR-338-5p, miR-374a-5p, and miR-421.

Embodiment 82: The method of Embodiment 80, wherein the endogenous control is selected from: miR-181a-2-3p, miR-181d-5p, Let-7g-5p, miR-181a-3p, miR-24-2-5p, miR-130b-3p, miR-628-3p, miR-19a-3p, miR-181a-5p and miR-424-3p.

Embodiment 83: The method of any one of Embodiments 1 to 82, wherein the gynecological disease is endometriosis.

Embodiment 84: The method of any one of Embodiments 1 to 83, further comprising determining the difference between the measured level and the reference level, and comparing the difference with a cutoff value, wherein a higher difference than the cutoff value indicates that the subject also has adenomyosis.

Embodiment 85: The method of Embodiment 1, wherein the miRNA comprises miR-155, miR-22, Let-7b, miR-23a, miR-17, or miR-20a, or any combination thereof, and wherein the method comprises determining the difference between the measured level and the reference level and comparing the difference with a cutoff value, wherein a higher difference than the cutoff value indicates the subject has endometriosis, and a lower difference than the cutoff value indicates the subject has a different gynecological disease.

Embodiment 86: The method of Embodiment 85, wherein the miRNA comprises miR-155-5p, miR-22-3p, Let-7b-5p, miR-23a-3p, miR-17-5p, miR-20a-5p, or any combination thereof.

Embodiment 87: The method of any one of Embodiments 1 to 86, wherein the level of the miRNA is measured using in situ RNA hybridization (e.g., fluorescence in situ hybridization, or FISH), quantitative polymerase chain reaction (qPCR), real-time polymerase chain reaction (RT-PCR), droplet digital PCR (ddPCR), microarray analysis, next generation sequencing (NGS), northern blotting, or a luminex-based assay.

Embodiment 88: The method of Embodiment 87, wherein the miRNA level is measured using NGS.

Embodiment 89: The method of Embodiment 87, wherein the miRNA level is measured using qPCR.

Embodiment 90: The method of Embodiment 87, wherein the miRNA level is measured using ddPCR.

Embodiment 91: The method of any one of Embodiments 87 to 90, wherein the sample is treated with DNase before the miRNA level is measured.

Embodiment 92: The method of any one of Embodiments 1 to 91, wherein the sample is a peripheral blood sample, a menstrual blood sample, a pap smear sample, a uterus biopsy sample, or an endometrial biopsy sample, a plasma sample or a serum sample.

Embodiment 93: The method of any one of Embodiments 1 to 92, further comprising obtaining the sample from the subject.

Embodiment 94: The method of Embodiment 93, wherein the sample is obtained during the secretory phase of a menstrual cycle.

Embodiment 95: The method of Embodiment 93, wherein the sample is obtained during the proliferative phase of a menstrual cycle.

Embodiment 96: The method of Embodiment 94 or 95, wherein the sample is a serum sample.

Embodiment 97: The method of Embodiment 94 or 95, wherein the serum sample is obtained within 2 hours of blood draw.

Embodiment 98: The method of any one of Embodiments 1 to 97, further comprising measuring the expression level of CA-125 (Cancer Antigen 125) in a sample of the subject, comparing the expression level to a reference level; wherein an increased expression level indicates that the subject is likely to have endometriosis.

Embodiment 99: A method of assessing whether a subject has endometriosis, comprising (a) measuring the expression level of CA-125 (Cancer Antigen 125) in a sample of the subject, wherein the sample is obtained during the secretory phase of a menstrual cycle; and (b) comparing the expression level to a reference level; wherein an increased expression level indicates that the subject is likely to have endometriosis.

Embodiment 100: The method of Embodiment 98 or 99, wherein the expression level is increased by at least 20%, at least 50%, at least 80%, or at least 90% compared to the reference level.

Embodiment 101: The method of any one of Embodiments 98 to 100, comprising measuring the protein level of CA-125.

Embodiment 102: The method of Embodiment 101, wherein the protein level is measured by immunohistochemistry (IHC), immunocytochemistry (ICC), an enzyme-linked immunosorbent assay (ELISA), immunoblotting assay (e.g., Western blot), flow cytometry (FACS), a fluorescent immunosorbent assay (FIA), a chemiluminescence immunoassay (CIA), an electrochemiluminescence immunoassay (ECLIA), a radioimmunoassay (RIA), a solid phase radioimmunoassay (SPROA), or a dot/line-immunoblot assay.

Embodiment 103: The method of any one of Embodiments 98 to 102, wherein the sample for measuring CA-125 is a blood sample or a peritoneal fluid sample.

Embodiment 104: The method of any one of Embodiments 98 to 102, wherein the sample for measuring CA-125 is a serum sample.

Embodiment 105: The method of Embodiment 104, wherein the serum sample is obtained within 2 hours of blood draw.

Embodiment 106: The method of Embodiment 104 or 105, wherein the reference level is 15 U/mL, 20 U/mL, 30 U/mL, or 35 U/mL.

Embodiment 107: The method of any one of Embodiments 1 to 106, wherein the subject is a human female.

Embodiment 108: The method of any one of Embodiments 1 to 107, wherein the subject has a clinical indicator for endometriosis, wherein the indicator is dysmenorrhea, lower abdominal pain, chronic pelvic pain, deep dyspareunia, dysuria, dyschezia, fatigue, abnormal menstrual bleeding, or infertility, or any combination thereof.

Embodiment 109: The method of any one of Embodiments 1 to 108, further comprising administering a treatment for endometriosis to the subject.

Embodiment 110: The method of Embodiment 109, wherein the treatment for endometriosis is pain medication, a hormone therapy, or a surgical procedure.

Embodiment 111: The method of Embodiment 109, wherein the treatment is a surgical procedure selected from: laparoscopy, laparotomy, presacral neurectomy, laparoscopic uterine nerve ablation (LUNA), and hysterectomy.

Embodiment 112: The method of Embodiment 109, wherein the treatment is a hormonal treatment, an oral contraceptive, a progestin, a GnRH agonist, an androgen, an aromatase inhibitor, a non-steroidal anti-inflammatory drug, or any combination thereof.

Embodiment 113: A kit for assessing whether a subject has a gynecological disease comprising a means for measuring a level of a miRNA selected from: miR-17, miR-19b, miR-21, miR-15b, miR-19a, miR-664a, miR-381, miR-23a, miR-654, miR-24, Let-7b, miR-20a, miR-22, miR-34c, miR-1287, miR-625, miR-1294, miR-7704, miR-221, miR-340, miR-450b, miR-548e, miR-502, miR-574, miR-9, miR-299, miR-6789, miR-593, miR-346, miR-34a, miR-449a, miR-7109, miR-3907, miR-557, miR-6801, miR-4420, miR-570, miR-155, miR-199, miR-520d, miR-424, and miR-200 in a sample of the subject, and an ancillary reagent.

Embodiment 114: The kit of Embodiment 113, wherein: miR-17 comprises miR-17-5p, miR-17-3p, or both; miR-19b comprises miR-19b-5p, miR-19b-3p, or both; miR-21 comprises miR-21-5p, miR-21-3p, or both; miR-15b comprises miR-15b-5p, miR-15b-3p, or both; miR-19a comprises miR-19a-5p, miR-19a-3p, or both; miR-664a comprises miR-664a-5p, miR-664a-3p, or both; miR-381 comprises miR-381-5p, miR-381-3p, or both; miR-23a comprises miR-23a-5p, miR-23a-3p, or both; miR-654 comprises miR-654-5p, miR-654-3p, or both; miR-24 comprises miR-24-5p, miR-24-3p, or both; Let-7b comprises Let-7b-5p, Let-7b-3p, or both; miR-20a comprises miR-20a-5p, miR-20a-3p, or both; miR-22 comprises miR-22-5p, miR-22-3p, or both; miR-34c comprises miR-34c-5p, miR-34c-3p, or both; miR-1287 comprises miR-1287-5p, miR-1287-3p, or both; miR-625 comprises miR-625-5p, miR-625-3p, or both; miR-1294 comprises miR-1294-5p, miR-1294-3p, or both; miR-7704 comprises miR-7704; miR-221 comprises miR-221-5p, miR-221-3p, or both; miR-340 comprises miR-340-5p, miR-340-3p, or both; miR-450b comprises miR-450b-5p, miR-450b-3p, or both; miR-548e comprises miR-548e-5p, miR-548e-3p, or both; miR-502 comprises miR-502-5p, miR-502-3p, or both; miR-574 comprises miR-574-5p, miR-574-3p, or both; miR-9 comprises miR-9-5p, miR-9-3p, or both; miR-299 comprises miR-299-5p, miR-299-3p, or both; miR-6789 comprises miR-6789-5p, miR-6789-3p, or both; miR-593 comprises miR-593-5p, miR-593-3p, or both; miR-346 comprises miR-346-5p, miR-346-3p, or both; miR-34a comprises miR-34a-5p, miR-34a-3p, or both; miR-449a comprises miR-449a-5p, miR-449a-3p, or both; miR-7109 comprises miR-7109-5p, miR-7109-3p, or both; miR-3907 comprises miR-3907; miR-557 comprises miR-557; miR-6801 comprises miR-6801-5p, miR-6801-3p or both; miR-4420 comprises miR-4420; miR-570 comprises miR-570-5p, miR-570-3p, or both; miR-155 comprises miR-155-5p, miR-155-3p, or both; miR-199 comprises miR-199-5p, miR-199-3p, or both; miR-520d comprises miR-520d-5p, miR-520d-3p, or both; miR-424 comprises miR-424-5p, miR-424-3p, or both; miR-200 comprises miR-200a-5p, miR-200a-3p, miR-200b-5p, miR-200b-3p, miR-200c-5p, or miR-200c-3p, or any combination thereof.

Embodiment 115: The kit of Embodiment 113, wherein: miR-17-5p; miR-19b comprises miR-19b-3p; miR-21 comprises miR-21-5p, miR-21-3p, or both; miR-15b comprises miR-15b-5p; miR-19a comprises miR-19a-3p; miR-664a comprises miR-664a-3p; miR-381 comprises miR-381-3p; miR-23a comprises miR-23a-3p; miR-654 comprises miR-654-3p; miR-24 comprises miR-24-3p; Let-7b comprises Let-7b-5p; miR-20a comprises miR-20a-5p; miR-22 comprises miR-22-5p, miR-22-3p, or both; miR-34c comprises miR-34c-3p; miR-1287 comprises miR-1287-5p; miR-625 comprises miR-625-5p; miR-1294 comprises miR-1294-5p; miR-7704 comprises miR-7704; miR-221 comprises miR-221-5p; miR-340 comprises miR-340-5p; miR-450b comprises miR-450b-5p; miR-548e comprises miR-548e-3p; miR-502 comprises miR-502-3p; miR-574 comprises miR-574-3p; miR-9 comprises miR-9-5p; miR-299 comprises miR-299-5p, miR-299-3p, or both; miR-6789 comprises miR-6789-5p; miR-593 comprises miR-593-5p; miR-346 comprises miR-346-5p, miR-346-3p, or both; miR-34a comprises miR-34a-5p; miR-449a comprises miR-449a-5p, miR-449a-3p, or both; miR-7109 comprises miR-7109-5p; miR-3907 comprises miR-3907; miR-557 comprises miR-557; miR-6801 miR-6801-3p; miR-4420 comprises miR-4420; miR-570 comprises miR-570-3p; miR-155 comprises miR-155-5p; miR-199 comprises miR-199-5p; miR-520d comprises miR-520d-5p; miR-424 comprises miR-424-5p; miR-200 comprises miR-200a-5p, miR-200a-3p, miR-200b-5p, miR-200b-3p, miR-200c-5p, or miR-200c-3p, or any combination thereof.

Embodiment 116: The kit of Embodiment 113 or 114, comprising a means for measuring the level of miR-17.

Embodiment 117: The kit of Embodiment 116, wherein miR-17 comprises miR-17-5p.

Embodiment 118: The kit of Embodiment 113 or 114, comprising a means for measuring the level of miR-19b.

Embodiment 119: The kit of Embodiment 118, wherein miR-19b comprises miR-19b-3p.

Embodiment 120: The kit of Embodiment 113 or 114, comprising a means for measuring the level of miR-21.

Embodiment 121: The kit of Embodiment 120, wherein miR-21 comprises miR-21-5p.

Embodiment 122: The kit of Embodiment 120, wherein miR-21 comprises miR-21-3p.

Embodiment 123: The kit of Embodiment 113 or 114, comprising a means for measuring the level of miR-15b.

Embodiment 124: The kit of Embodiment 123, wherein miR-15b comprises miR-15b-5p.

Embodiment 125: The kit of Embodiment 113 or 114, comprising a means for measuring the level of miR-19a.

Embodiment 126: The kit of Embodiment 125, wherein miR-19a comprises miR-19a-3p.

Embodiment 127: The kit of Embodiment 113 or 114, comprising a means for measuring the level of miR-664a.

Embodiment 128: The kit of Embodiment 127, wherein miR-664a comprises miR-664a-3p.

Embodiment 129: The kit of Embodiment 113 or 114, comprising a means for measuring the level of miR-381.

Embodiment 130: The kit of Embodiment 129, wherein miR-381 comprises miR-381-3p.

Embodiment 131: The kit of Embodiment 113 or 114, comprising a means for measuring the level of miR-23a.

Embodiment 132: The kit of Embodiment 131, wherein miR-23a comprises miR-23a-3p.

Embodiment 133: The kit of Embodiment 113 or 114, comprising a means for measuring the level of miR-654.

Embodiment 134: The kit of Embodiment 133, wherein miR-654 comprises miR-654-3p.

Embodiment 135: The kit of Embodiment 113 or 114, comprising a means for measuring the level of miR-24.

Embodiment 136: The kit of Embodiment 135, wherein miR-24 comprises miR-24-3p.

Embodiment 137: The kit of Embodiment 113 or 114, comprising a means for measuring the level of Let-7b.

Embodiment 138: The kit of Embodiment 137, wherein Let-7b comprises Let-7b-5p.

Embodiment 139: The kit of Embodiment 113 or 114, comprising a means for measuring the level of miR-20a.

Embodiment 140: The kit of Embodiment 139, wherein miR-20a comprises miR-20a-5p.

Embodiment 141: The kit of Embodiment 113 or 114, comprising a means for measuring the level of miR-22.

Embodiment 142: The kit of Embodiment 141, wherein miR-22 comprises miR-22-5p.

Embodiment 143: The kit of Embodiment 141, wherein miR-22 comprises miR-22-3p.

Embodiment 144: The kit of Embodiment 113 or 114, comprising a means for measuring the level of miR-34c.

Embodiment 145: The kit of Embodiment 144, wherein miR-34c comprises miR-34c-3p.

Embodiment 146: The kit of Embodiment 113 or 114, comprising a means for measuring the level of miR-155.

Embodiment 147: The kit of Embodiment 146, wherein miR-155 comprises miR155-5p.

Embodiment 148: The kit of Embodiment 113 or 114, comprising a means for measuring the level of miR-1287.

Embodiment 149: The kit of Embodiment 148, wherein miR-1287 comprises miR-1287-5p.

Embodiment 150: The kit of Embodiment 113 or 114, comprising a means for measuring the level of miR-625.

Embodiment 151: The kit of Embodiment 150, wherein miR-625 comprises miR-625-5p.

Embodiment 152: The kit of Embodiment 113 or 114, comprising a means for measuring the level of miR-1294.

Embodiment 153: The kit of Embodiment 152, wherein miR-1294 comprises miR-1294-5p.

Embodiment 154: The kit of Embodiment 113 or 114, comprising a means for measuring the level of miR-7704.

Embodiment 155: The kit of Embodiment 113 or 114, comprising a means for measuring the level of miR-221.

Embodiment 156: The kit of Embodiment 155, wherein miR-221 comprises miR-221-5p.

Embodiment 157: The kit of Embodiment 113 or 114, comprising a means for measuring the level of miR-340.

Embodiment 158: The kit of Embodiment 157, wherein miR-340 comprises miR-340-5p.

Embodiment 159: The kit of Embodiment 113 or 114, comprising a means for measuring the level of miR-450b.

Embodiment 160: The kit of Embodiment 159, wherein miR-450b comprises miR-450b-5p.

Embodiment 161: The kit of Embodiment 113 or 114, comprising a means for measuring the level of miR-548e.

Embodiment 162: The kit of Embodiment 161, wherein miR-548e comprises miR-548e-3p.

Embodiment 163: The kit of Embodiment 113 or 114, comprising a means for measuring the level of miR-502.

Embodiment 164: The kit of Embodiment 163, wherein miR-502 comprises miR-502-3p.

Embodiment 165: The kit of Embodiment 113 or 114, wherein the miRNA is selected from: miR-17, miR-19b, miR-21, miR-15b, miR-19a, miR-664a, miR-381, miR-23a, miR-654, and miR-24.

Embodiment 166: The kit of Embodiment 163, wherein the miRNA is selected from: miR-17-5p, miR-19b-3p, miR-21-5p, miR-15b-5p, miR-19a-3p, miR-664a-3p, miR-381-3p, miR-23a-3p, miR-654-3p, and miR-24-3p.

Embodiment 167: The kit of Embodiment 113 or 114, wherein the miRNA is selected from: miR-21, miR-23a, miR-1287, miR-625, miR-1294, miR-7704, miR-221, miR-340, miR-450b, miR-548e, and miR-502.

Embodiment 168: The kit of Embodiment 167, wherein the miRNA is selected from: miR-21-3p, miR-22-5p, miR-1287-5p, miR-625-5p, miR-1294-5p, miR-7704, miR-221-5p, miR-340-5p, miR-450b-5p, miR-548e-3p, and miR-502-3p.

Embodiment 169: The kit of Embodiment 113 or 114, wherein the miRNA is selected from: miR-17, miR-23a, Let-7b, miR-20a, miR-22, miR-155, miR-34c, and miR-199.

Embodiment 170: The kit of Embodiment 169, wherein the miRNA is selected from: miR-17-5p, miR-23a-3p, Let-7b-5p, miR-20a-5p, miR-22-3p, miR-155-5p, miR-34c-3p, and miR-199-5p.

Embodiment 171: The kit of Embodiment 113 or 114, wherein the miRNA is selected from: miR-17, miR-23a, Let-7b, miR-20a, miR-22, and miR-155.

Embodiment 172: The kit of Embodiment 171, wherein the miRNA is selected from: miR-17-5p, miR-23a-3p, Let-7b-5p, miR-20a-5p, miR-22-3p, and miR-155-5p.

Embodiment 173: The kit of Embodiment 113 or 114, wherein the miRNA is selected from: Let-7b, miR-20a, miR-22, miR-34c, miR-574, miR-9, miR-299, miR-6789, miR-593, miR-346, miR-34a, miR-449a, miR-7109, miR-3907, miR-557, miR-6801, miR-4420, and miR-570.

Embodiment 174: The kit of Embodiment 173, wherein the miRNA is selected from: Let-7b-5p, miR-20a-5p, miR-22-3p, miR-34c-3p, miR-574-3p, miR-9-5p, miR-299-3p, miR-299-5p, miR-6789-5p, miR-593-5p, miR-346, miR-34a-5p, miR-449a, miR-7109-5p, miR-3907, miR-557, miR-6801-3p, miR-4420, and miR-570-3p.

Embodiment 175: The kit of any Embodiment 113 or 114, comprising means for measuring levels of at least two, at least three, at least four, at least five, at least six, at least seven, or at least eight miRNA.

Embodiment 176: The kit of Embodiment 175, comprising means for measuring levels of miR-17, miR-19b, miR-21, miR-15b, miR-19a, miR-664a, miR-381, miR-23a, miR-654, and miR-24.

Embodiment 177: The kit of Embodiment 176, comprising means for measuring levels of: miR-17-5p, miR-19b-3p, miR-21-5p, miR-15b-5p, miR-19a-3p, miR-664a-3p, miR-381-3p, miR-23a-3p, miR-654-3p, and miR-24-3p.

Embodiment 178: The kit of Embodiment 175, comprising means for measuring levels of: miR-21, miR-23a, miR-1287, miR-625, miR-1294, miR-7704, miR-221, miR-340, miR-450b, miR-548e, and miR-502.

Embodiment 179: The kit of Embodiment 178, comprising means for measuring levels of: miR-21-3p, miR-22-5p, miR-1287-5p, miR-625-5p, miR-1294-5p, miR-7704, miR-221-5p, miR-340-5p, miR-450b-5p, miR-548e-3p, and miR-502-3p.

Embodiment 180: The kit of Embodiment 175, comprising means for measuring levels of: miR-17, miR-23a, Let-7b, miR-20a, miR-22, miR-155, miR-34c, and miR-199.

Embodiment 181: The kit of Embodiment 180, comprising means for measuring levels of: miR-17-5p, miR-23a-3p, Let-7b-5p, miR-20a-5p, miR-22-3p, miR-155-5p, miR-34c-3p, and miR-199-5p.

Embodiment 182: The kit of Embodiment 175, comprising means for measuring levels of: miR-17, miR-23a, Let-7b, miR-20a, miR-22, and miR-155.

Embodiment 183: The kit of Embodiment 182, comprising means for measuring levels of: miR-17-5p, miR-23a-3p, Let-7b-5p, miR-20a-5p, miR-22-3p, and miR-155-5p.

Embodiment 184: The kit of any Embodiment 182 or 183, further comprising measuring levels of miR-574 and miR-520d.

Embodiment 185: The kit of Embodiment 184, comprising means for measuring levels of miR-574-3p and miR-520d-5p.

Embodiment 186: The kit of Embodiment 175, comprising means for measuring levels of at least two miRNAs selected from: miR-34c, miR-20a, miR-17, and miR-22.

Embodiment 187: The kit of Embodiment 186, comprising means for measuring levels of at least two miRNAs selected from: miR-34c-3p, miR-20a-5p, miR-17-5p, and miR-22-3p.

Embodiment 188: The kit of Embodiment 187, comprising means for measuring levels of miR-20a-5p and miR-34c-3p.

Embodiment 189: The kit of Embodiment 187, comprising means for measuring levels of miR-17-5p and miR-34c-3p.

Embodiment 190: The kit of Embodiment 187, comprising means for measuring levels of miR-22-3p and miR-34c-3p.

Embodiment 191: The kit of Embodiment 187, comprising means for measuring levels of miR-17-5p, miR-22-3p and miR-34c-3p.

Embodiment 192: The kit of any one of Embodiments 113 to 191, further comprising means for measuring an endogenous control.

Embodiment 193: The kit of Embodiment 192, wherein the endogenous control is selected from: miR-16-5p, miR-92a-3p, miR-652-3p, miR-92b-3p, miR-532-5p, miR-338-5p, miR-374a-5p, and miR-421.

Embodiment 194: The kit of Embodiment 192, wherein the endogenous control is selected from: miR-181a-2-3p, miR-181d-5p, Let-7g-5p, miR-181a-3p, miR-24-2-5p, miR-130b-3p, miR-628-3p, miR-19a-3p, miR-181a-5p and miR-424-3p.

Embodiment 195: The kit of any one of Embodiments 113 to 194, wherein the gynecological disease is endometriosis.

Embodiment 196: The kit of Embodiment 195, wherein the gynecological disease is endometriosis in combination with adenomyosis.

Embodiment 197: The kit of any one of Embodiments 113 to 196, wherein the means comprises one or more nucleic acid probes for detecting the miRNA.

Embodiment 198: The kit of any one of Embodiments 113 to 196, wherein the means comprises one or more primer pairs for amplifying the miRNA.

Embodiment 199: The kit of Embodiment 198, wherein the primer pair comprises a forward primer and a reverse primer having nucleotide sequences of (1) SEQ ID NOs: 3 and 1, respectively; (2) SEQ ID NOs: 4 and 1, respectively; (3) SEQ ID NOs: 5 and 1, respectively; (4) SEQ ID NOs: 6 and 1, respectively; (5) SEQ ID NOs: 7 and 1, respectively; or (6) SEQ ID NOs: 8 and 1, respectively.

Embodiment 200: The kit of Embodiment 199, further comprising a primer pair for measuring an endogenous control, comprising a forward primer and a reverse primer having the nucleotide sequences of (1) SEQ ID NOs: 9 and 1, respectively; (2) SEQ ID NOs: 10 and 2, respectively; or (3) SEQ ID NOs: 11 and 1, respectively.

Embodiment 201: The kit of any one of Embodiments 198 to 200, further comprising a reverse transcriptase, a DNA polymerase, or a DNA polymerase with reverse transcriptase activity.

Embodiment 202: The kit of Embodiment 201, further comprising a DNase.

Embodiment 203: The kit of any one of Embodiments 113 to 202, further comprising a means for measuring the expression level of CA-125 in a sample of the subject.

Embodiment 204: The kit of Embodiment 203, wherein the means for measuring the expression level of CA-125 comprises an anti-CA-125 antibody.

Embodiment 205: The kit of Embodiment 204, further comprising a secondary antibody.

Embodiment 206: The kit of any one of Embodiments 113 to 205, wherein the ancillary reagent comprises a reaction buffer, a dilution buffer, or a wash buffer, or any combination thereof.

Embodiment 207: The kit of any one of Embodiments 113 to 206, wherein said kit further comprises a solid support.

Embodiment 208: The kit of any one of Embodiments 113 to 207, wherein said kit further comprises a container for sample collection.

Embodiment 209: The kit of any one of Embodiments 113 to 208, wherein said kit further comprises instructions.

### 7.8.2 Set 2

Embodiment 1: A method of detecting a gynecological disease in a subject, comprising
(a) measuring a level of a miRNA selected from the group consisting of miR-34c, miR-155, miR-22, miR-23a, miR-17, miR-20a, Let-7b, miR-199, miR-574, miR-520d, miR-424, and miR-200 in a sample of the subject, and
(b) comparing the level to a reference level; wherein an altered level indicates that the subject has a gynecological disease.

Embodiment 2: The method of Embodiment 1, wherein the miR-34c is miR-34c-5p or miR-34c-3p; miR-155 is miR-155-5p or miR-155-3p; miR-199 is miR-199-5p or miR-199-3p; miR-22 is miR-22-5p or miR-22-3p; miR-23a is miR-23a-5p or miR-23a-3p; miR-17 is miR-17-5p or miR-17-3p, miR-20a is miR-20a-5p or miR-20a-3p, Let-7b is Let-7b-5p or Let-7b-3p, miR-574 is miR-574-5p or miR-574-3p, miR-520d is miR-520d-5p or miR-520d-3p, miR-424 is miR-424-5p or miR-424-3p, miR-200 is miR-200a-5p, miR-200a-3p, miR-200b-5p, miR-200b-3p, miR-200c-5p, or miR-200c-3p.

Embodiment 3: The method of Embodiment 1 or 2, wherein the miRNA is selected from the group consisting of miR-34c, miR-23a, miR-17, miR-155, let-7b, miR-199, miR-22, and miR-20a.

Embodiment 4: The method of Embodiment 1 or 2, wherein the miRNA comprises miR-34c.

Embodiment 5: The method of Embodiment 4, where the miRNA is miR-34c-5p.

Embodiment 6: The method of Embodiment 1 or 2, wherein the miRNA is selected from the group consisting of miR-155, miR-22, miR-23a, miR-17, miR-20a, Let-7b, miR-574, and miR-520d.

Embodiment 7: The method of Embodiment 1 or 2, wherein the miRNA is selected from the group consisting of miR-155, miR-22, miR-23a, miR-17, miR-20a, and Let-7b.

Embodiment 8: The method of Embodiment 1 or 2, comprising measuring levels of at least two, at least three, at least four, at least five, at least six, at least seven, or at least eight miRNAs.

Embodiment 9: The method of Embodiment 8, wherein the miRNAs comprise miR-34c and miR-20a.

Embodiment 10:The method of Embodiment 9, wherein the miRNAs comprise miR-34c-5p and miR-20a-5p.

Embodiment 11:The method of Embodiment 8, wherein the miRNAs comprise miR-34c and miR-17.

Embodiment 12:The method of Embodiment 11, wherein the miRNAs comprise miR-34c-5p and miR-17-5p.

Embodiment 13: The method of Embodiment 8, wherein the miRNAs comprise miR-34c and miR-22.

Embodiment 14: The method of Embodiment 13, wherein the miRNAs comprise miR-34c-5p and miR-22-5p.

Embodiment 15: The method of Embodiment 8, wherein the miRNAs comprise miR-155, miR-22, and miR-23a.

Embodiment 16: The method of Embodiment 8, wherein the miRNAs comprise miR-155-5p, miR-22-3p, and miR-23a-3p.

Embodiment 17: The method of Embodiment 8, wherein the miRNAs comprise miR-155-5p, miR-22-5p, and miR-23a-3p.

Embodiment 18: The method of any one of Embodiments 15 to 17, wherein the miRNAs further comprise miR-17, miR-20a, and Let-7b.

Embodiment 19: The method of Embodiment 18, wherein the miRNAs comprise miR-155-5p, miR-22-3p, miR-23a-3p, miR-17-5p, miR-20a-5p, Let-7b-5p, and Let-7b-3p.

Embodiment 20: The method of Embodiment 18, wherein the miRNAs comprise miR-155-5p, miR-22-5p, miR-23a-3p, miR-17-5p, miR-20a-5p, and Let-7b-5p.

Embodiment 21: The method of any one of Embodiments 15 to 20, wherein the miRNAs further comprise miR-574 and miR-520.

Embodiment 22: The method of Embodiment 21, wherein the miRNAs comprise miR-155-5p, miR-22-3p, miR-23a-3p, miR-17-5p, miR-20a-5p, Let-7b-5p, Let-7b-3p, miR-574-3p, and miR-520d-5p.

Embodiment 23: The method of Embodiment 22, wherein the miRNAs comprise miR-155-5p, miR-22-5p, miR-23a-3p, miR-17-5p, miR-20a-5p, Let-7b-5p, miR-574-3p, and miR-520d-5p.

Embodiment 24: The method of any one of Embodiments 1 to 23, wherein a decrease in the miRNA level indicates that the subject has a gynecological disease.

Embodiment 25: The method of Embodiment 24, wherein the gynecological disease is endometriosis.

Embodiment 26: The method of Embodiment 24, further comprising determining the difference between the measured level and the reference level, and comparing the difference with a cutoff value, wherein a higher difference than the cutoff value indicates that the subject also has adenomyosis.

Embodiment 27: The method of Embodiment 1, wherein the miRNA comprises miR-155, Let-7b, miR-23a, miR-17, or miR-20a, or any combination thereof, and wherein the method comprises determining the difference between the measured level and the reference level and comparing the difference with a cutoff value, wherein a higher difference than the cutoff value indicates the subject has endometriosis, and a lower difference than the cutoff value indicates the subject has a different gynecological disease.

Embodiment 28: The method of Embodiment 27, wherein the miRNA comprises miR-155-5p, Let-7b-5p, miR-23a-3p, miR-17-5p, miR-20a-5p, or any combination thereof.

Embodiment 29: The method of Embodiment 27, wherein the mRNAs comprise Let-7b, miR-17, and miR-20a.

Embodiment 30:Embodiment 3: The method of Embodiment 29, wherein the miRNAs comprise Let-7b-5p, miR-17-5p, and miR-20a-5p.

Embodiment 31: The method of any one of Embodiments 27 to 30, wherein mRNAs further comprise miR-424, miR-200, or both.

Embodiment 32: The method of any one of Embodiments 27 to 30, wherein mRNAs further comprise miR-424-5p, miR-200a, miR-200b, miR-200c, or any combination thereof.

Embodiment 33: The method of any one of Embodiments 1 to 32, wherein the level of the miRNA is measured using in situ RNA hybridization (e.g., fluorescence in situ hybridization, or FISH), quantitative polymerase chain reaction (qPCR), real-time polymerase chain reaction (RT-PCR), microarray analysis, next generation sequencing (NGS), northern blotting, or a luminex-based assay.

Embodiment 34: The method of Embodiment 33, wherein the miRNA level is measured using NGS.

Embodiment 35: The method of Embodiment 33, wherein the miRNA level is measured using qPCR.

Embodiment 36: The method of any one of Embodiments 33 to 35, wherein the sample is treated with DNase before the miRNA level is measured.

Embodiment 37: The method of any one of Embodiments 1 to 35, wherein the sample is a peripheral blood sample, a menstrual blood sample, a pap smear sample, a uterus biopsy sample, or an endometrial biopsy sample.

Embodiment 38: The method of Embodiment 37, wherein the sample is a plasma sample or a serum sample.

Embodiment 39: The method of any one of Embodiments 1 to 38, further comprising obtaining the sample from the subject.

Embodiment 40: The method of Embodiment 39, wherein the sample is obtained during the secretory phase of a menstrual cycle.

Embodiment 41: The method of any one of Embodiments 1 to 40, wherein the subject is a human female.

Embodiment 42: The method of any one of Embodiments 1 to 41, wherein the subject has a clinical indicator for endometriosis, wherein the indicator is dysmenorrhea, lower abdominal pain, chronic pelvic pain, deep dyspareunia, dysuria, dyschezia, fatigue, or infertility, or any combination thereof.

Embodiment 43: The method of any one of Embodiments 24 to 42, further comprising administering a treatment for endometriosis to the subject.

Embodiment 44: The method of Embodiment 43, wherein the treatment for endometriosis is pain medication, a hormone therapy, or a surgical procedure.

Embodiment 45: A method of assessing whether a subject has endometriosis, comprising
(a) measuring the expression level of CA-125 (Cancer Antigen 125) in a sample of the subject, wherein the sample is obtained during the secretory phase of a menstrual cycle; and
(b) comparing the expression level to a reference level; wherein an increased expression level indicates that the subject is likely to have endometriosis.

Embodiment 46: The method of Embodiment 45, wherein the expression level is increased by at least 20%, at least 50%, at least 80%, or at least 90% compared to the reference level.

Embodiment 47: The method of Embodiment 45 or 46, comprising measuring the protein level of CA-125.

Embodiment 48: The method of Embodiment 47, wherein the protein level is measured by immunohistochemistry (IHC), immunocytochemistry (ICC), an enzyme-linked immunosorbent assay (ELISA), immunoblotting assay (e.g., Western blot), flow cytometry (FACS), a fluorescent immunosorbent assay (FIA), a chemiluminescence immunoassay (CIA), an electrochemiluminescence Immunoassay (ECLIA), a radioimmunoassay (RIA), a solid phase radioimmunoassay (SPROA), or a dot/line-immunoblot assay.

Embodiment 49: The method of any one of Embodiments 45 to 48, wherein the sample is a blood sample or a peritoneal fluid sample.

Embodiment 50: The method of any one of Embodiments 45 to 48, wherein the sample is a serum sample.

Embodiment 51: The method of Embodiment 50, wherein the serum sample is obtained within 2 hours of blood draw.

Embodiment 52: The method of Embodiment 50 or 51, wherein the reference level is 15 U/mL, 20 U/mL, 30 U/mL, or 35 U/mL.

Embodiment 53: The method of any one Embodiment 45 to 52, further comprising (a) measuring a level of a miRNA selected from the group consisting of miR-34c, miR-155, miR-22, miR-23a, miR-17, miR-20a, Let-7b, miR-199, miR-574, miR-520d, miR-424, and miR-200 in a second sample of the subject, and (b) comparing the level to a reference level; wherein an altered level corroborates that the subject likely has endometriosis or indicates the staging the endometriosis.

Embodiment 54: The method of Embodiment 53, wherein the miR-34c is miR-34c-5p or miR-34c-3p; miR-155 is miR-155-5p or miR-155-3p; miR-199 is miR-199-5p or miR-199-3p; miR-22 is miR-22-5p or miR-22-3p; miR-23a is miR-23a-5p or miR-23a-3p; miR-17 is miR-17-5p or miR-17-3p, miR-20a is miR-20a-5p or miR-20a-3p, Let-7b is Let-7b-5p or Let-7b-3p, miR-574 is miR-574-5p or miR-574-3p, miR-520d is miR-520d-5p or miR-520d-3p, miR-424 is miR-424-5p or miR-424-3p, miR-200 is miR-200a-5p, miR-200a-3p, miR-200b-5p, miR-200b-3p, miR-200c-5p, or miR-200c-3p.

Embodiment 55: The method of Embodiment 53 or 54, wherein the miRNA comprises miR-34c.

Embodiment 56: The method of Embodiment 55, where the miRNA is miR-34c-5p.

Embodiment 57: The method of Embodiment 53 or 54, wherein the miRNA is selected from the group consisting of miR-155, miR-22, miR-23a, miR-17, miR-20a, Let-7b, miR-574, and miR-520d.

Embodiment 58: The method of Embodiment 53 or 54, wherein the miRNA is selected from the group consisting of miR-155, miR-22, miR-23a, miR-17, miR-20a, and Let-7b.

Embodiment 59: The method of Embodiment 53 or 54, comprising measuring levels of at least two, at least three, at least four, at least five, at least six, at least seven, or at least eight miRNAs.

Embodiment 60: The method of Embodiment 59, wherein the miRNAs comprise miR-34c and miR-20a.

Embodiment 61: The method of Embodiment 60, wherein the miRNAs comprise miR-34c-5p and miR-20a-5p.

Embodiment 62: The method of Embodiment 59, wherein the miRNAs comprise miR-34c and miR-17.

Embodiment 63: The method of Embodiment 62, wherein the miRNAs comprise miR-34c-5p and miR-17-5p.

Embodiment 64:Embodiment 6: The method of Embodiment 59, wherein the miRNAs comprise miR-34c and miR-22.

Embodiment 65: The method of Embodiment 64, wherein the miRNAs comprise miR-34c-5p and miR-22-5p.

Embodiment 66: The method of Embodiment 59, wherein the miRNAs comprise miR-155, miR-22, and miR-23a.

Embodiment 67: The method of Embodiment 59, wherein the miRNAs comprise miR-155-5p, miR-22-3p, and miR-23a-3p.

Embodiment 68: The method of Embodiment 59, wherein the miRNAs comprise miR-155-5p, miR-22-5p, and miR-23a-3p.

Embodiment 69: The method of any one of Embodiments 66 to 68, wherein the miRNAs further comprise miR-17, miR-20a, and Let-7b.

Embodiment 70: The method of Embodiment 69, wherein the miRNAs comprise miR-155-5p, miR-22-3p, miR-23a-3p, miR-17-5p, miR-20a-5p, Let-7b-5p, and Let-7b-3p.

Embodiment 71: The method of Embodiment 69, wherein the miRNAs comprise miR-155-5p, miR-22-5p, miR-23a-3p, miR-17-5p, miR-20a-5p, and Let-7b-5p.

Embodiment 72: The method of any one of Embodiments 66 to 71, wherein the miRNAs further comprise miR-574 and miR-520.

Embodiment 73: The method of Embodiment 72, wherein the miRNAs comprise miR-155-5p, miR-22-3p, miR-23a-3p, miR-17-5p, miR-20a-5p, Let-7b-5p, Let-7b-3p, miR-574-3p, and miR-520d-5p.

Embodiment 74: The method of Embodiment 73, wherein the miRNAs comprise miR-155-5p, miR-22-5p, miR-23a-3p, miR-17-5p, miR-20a-5p, Let-7b-5p, miR-574-3p, and miR-520d-5p.

Embodiment 75: The method of any one of Embodiments 53 to 74, wherein a decrease in the miRNA level corroborates that the subject has endometriosis.

Embodiment 76: The method of any one of Embodiments 53 to 75, wherein the level of the miRNA is measured using in situ RNA hybridization (e.g., fluorescence in situ hybridization, or FISH), quantitative polymerase chain reaction (qPCR), real-time polymerase chain reaction (RT-PCR), microarray analysis, next generation sequencing (NGS), northern blotting, or a luminex-based assay.

Embodiment 77: The method of Embodiment 76, wherein the miRNA level is measured using NGS.

Embodiment 78: The method of Embodiment 76, wherein the miRNA level is measured using qPCR.

Embodiment 79: The method of any one of Embodiments 53 to 78, wherein the second sample is a peripheral blood sample, a menstrual blood sample, a pap smear sample, a uterus biopsy sample, or an endometrial biopsy sample.

Embodiment 80: The method of Embodiment 79, wherein the second sample is a serum sample or plasma sample.

Embodiment 81: The method of any one of Embodiments 53 to 80, wherein the second sample is treated with DNase before the miRNA level is measured.

Embodiment 82: The method of any one of Embodiments 45 to 81, further comprising administering a therapy for endometriosis to the subject assessed as likely having endometriosis.

Embodiment 83: The method of Embodiment 82, wherein the therapy for endometriosis is pain medication, a hormone therapy, or a surgical procedure.

Embodiment 84: The method of Embodiment 82, wherein the therapy for endometriosis is a surgical procedure selected from the group consisting of laparoscopy, laparotomy, presacral neurectomy, laparoscopic uterine nerve ablation (LUNA), and hysterectomy.

Embodiment 85: A kit for assessing whether a subject has a gynecological disease comprising a means for measuring a level of a miRNA selected from the group consisting of miR-34c, miR-155, miR-22, miR-23a, miR-17, miR-20a, Let-7b, miR-199, miR-574, miR-520d, miR-424, miR-200 in a sample of the subject, and an ancillary reagent.

Embodiment 86: The kit of Embodiment 85, wherein the miR-34c is miR-34c-5p or miR-34c-3p; miR-155 is miR-155-5p or miR-155-3p; miR-199 is miR-199-5p or miR-199-3p; miR-22 is miR-22-5p or miR-22-3p; miR-23a is miR-23a-5p or miR-23a-3p; miR-17 is miR-17-5p or miR-17-3p, miR-20a is miR-20a-5p or miR-20a-3p, Let-7b is Let-7b-5p or Let-7b-3p, miR-574 is miR-574-5p or miR-574-3p, miR-520d is miR-520d-5p or miR-520d-3p, miR-424 is miR-424-5p or miR-424-3p, miR-200 is miR-200a-5p, miR-200a-3p, miR-200b-5p, miR-200b-3p, miR-200c-5p, or miR-200c-3p.

Embodiment 87: The kit of Embodiment 85 or 86, wherein the miRNA is selected from the group consisting of miR-34c, miR-23a, miR-17, miR-155, let-7b, miR-199, miR-22, and miR-20a.

Embodiment 88: The kit of Embodiment 85 or 86, wherein the miRNA comprises miR-34c.

Embodiment 89: The kit of Embodiment 88, where the miRNA is miR-34c-5p.

Embodiment 90: The kit of Embodiment 85 or 86, wherein the miRNA is selected from the group consisting of miR-155, miR-22, miR-23a, miR-17, miR-20a, Let-7b, miR-574, and miR-520d.

Embodiment 91: The kit of Embodiment 85 or 86, wherein the miRNA is selected from the group consisting of miR-155, miR-22, miR-23a, miR-17, miR-20a, and Let-7b.

Embodiment 92: The kit of any Embodiment 85 or 86, comprising means for measuring levels of at least two, at least three, at least four, at least five, at least six, at least seven, or at least eight miRNA.

Embodiment 93: The kit of Embodiment 92, wherein the miRNAs comprise miR-34c and miR-20a.

Embodiment 94: The kit of Embodiment 93, wherein the miRNAs comprise miR-34c-5p and miR-20a-5p.

Embodiment 95: The kit of Embodiment 92, wherein the miRNAs comprise miR-34c and miR-17.

Embodiment 96: The kit of Embodiment 95, wherein the miRNAs comprise miR-34c-5p and miR-17-5p.

Embodiment 97: The kit of Embodiment 92, wherein the miRNAs comprise miR-34c and miR-22.

Embodiment 98: The kit of Embodiment 97, wherein the miRNAs comprise miR-34c-5p and miR-22-5p.

Embodiment 99: The kit of Embodiment 92, wherein the miRNAs comprise miR-155, miR-22, and miR-23a.

Embodiment 100: The kit of Embodiment 92, wherein the miRNAs comprise miR-155-5p, miR-22-3p, and miR-23a-3p.

Embodiment 101: The kit of Embodiment 92, wherein the miRNAs comprise miR-155-5p, miR-22-5p, and miR-23a-3p.

Embodiment 102: The kit of any one of Embodiments 99 to 101, wherein the miRNAs further comprise miR-17, miR-20a, and Let-7b.

Embodiment 103: The kit of Embodiment 102, wherein the miRNAs comprise miR-155-5p, miR-22-3p, miR-23a-3p, miR-17-5p, miR-20a-5p, Let-7b-5p, and Let-7b-3p.

Embodiment 104: The kit of Embodiment 102, wherein the miRNAs comprise miR-155-5p, miR-22-5p, miR-23a-3p, miR-17-5p, miR-20a-5p, and Let-7b-5p.

Embodiment 105: The kit of any one of Embodiments 99 to 104, wherein the miRNAs further comprise miR-574 and miR-520.

Embodiment 106: The kit of Embodiment 105, wherein the miRNAs comprise miR-155-5p, miR-22-3p, miR-23a-3p, miR-17-5p, miR-20a-5p, Let-7b-5p, Let-7b-3p, miR-574-3p, and miR-520d-5p.

Embodiment 107: The kit of Embodiment 105, wherein the miRNAs comprise miR-155-5p, miR-22-5p, miR-23a-3p, miR-17-5p, miR-20a-5p, Let-7b-5p, miR-574-3p, and miR-520d-5p.

Embodiment 108: The kit of any one of Embodiments 85 to 107, wherein the gynecological disease is endometriosis.

Embodiment 109: The kit of Embodiment 108, wherein the gynecological disease is endometriosis in combination with adenomyosis.

Embodiment 110: The kit of Embodiments 85, wherein the kit is for assessing whether the subject has endometriosis or a different gynecological disease, and wherein the miRNA comprises miR-155, Let-7b, miR-23a, miR-17, or miR-20a, or a combination thereof.

Embodiment 111: The kit of Embodiment 110, wherein the miRNA comprises miR-155-5p, Let-7b-5p, miR-23a-3p, miR-17-5p, miR-20a-5p, or any combination thereof.

Embodiment 113: The kit of Embodiment 110, wherein the miRNA comprises Let-7b, miR-17, and miR-20a.

Embodiment 113: The kit of Embodiment 110, wherein the miRNA comprises Let-7b-5p, miR-17-5p, and miR-20a-5p.

Embodiment 114: The kit of any one of Embodiments 110 to 113, wherein the miRNAs further comprise miR-424, miR-200, or both.

Embodiment 115: The kit of any one of Embodiments 110 to 113, wherein the miRNAs further comprise miR-424-5p, miR-200a, miR-200b, miR-200c, or any combination thereof.

Embodiment 116: The kit of any one of Embodiments 85 to 115, wherein the means comprises one or more nucleic acid probes for detecting the miRNA.

Embodiment 117: The kit of any one of Embodiments 85 to 115, wherein the means comprises one or more primer pairs for amplifying the miRNA.

Embodiment 118: The kit of Embodiment 117, further comprising a reverse transcriptase, a DNA polymerase, or a DNA polymerase with reverse transcriptase activity.

Embodiment 119: The kit of Embodiment 118, further comprising a DNase.

Embodiment 120: The kit of any one of Embodiments 85 to 119, further comprising a means for measuring the expression level of CA-125 in a sample of the subject.

Embodiment 121: The kit of Embodiment 120, wherein the means for measuring the expression level of CA-125 comprises an anti-CA-125 antibody.

Embodiment 122: The kit of Embodiment 121, further comprising a secondary antibody.

Embodiment 123: The kit of any one of Embodiments 85 to 122, wherein the ancillary reagent comprises a reaction buffer, a dilution buffer, or a wash buffer, or any combination thereof.

Embodiment 124: The kit of any one of Embodiments 85 to 123, wherein said kit further comprises a solid support.

Embodiment 125: The kit of any one of Embodiments 85 to 124, wherein said kit further comprises a container for sample collection.

Embodiment 126: The kit of any one of Embodiments 85 to 125, wherein said kit further comprises instructions.

### 7.9 Experimental

The examples provided below are for purposes of illustration only, which are not intended to be limiting unless otherwise specified. Thus, the invention should in no way be construed as being limited to the following examples, but rather, should be construed to encompass any and all variations which become evident as a result of the teaching provided herein.

### 7.9.1 Example 1: Detecting miRNA Biomarkers in Serum/Plasma Samples

The following protocol was followed for detecting the expression of miRNA in serum/plasma samples:

### Sample Preparation

Peripheral blood samples were processed within 2 hours after they were obtained from patients to separate serum and plasma. The processed serum/plasma samples were then frozen and stored/shipped for analysis.

### RNA Extraction

The RNA Spike-In Kit (Qiagen), miRNeasy Serum/Plasma Advanced Kit (Qiagen) and RNase-Free DNase Set (Qiagen) were used for RNA extraction.

1. Serum/plasma samples were thawed and centrifuged for 5 min at 3000×g and 4° C.; 200 μl supernatant was transferred into empty tubes.

2 (optional). 2 μl DNase I (Qiagen) was added to the 200 μl plasma/serum sample, incubated at room temperature for 15 minutes, then centrifuged briefly.

3. 1 μl UniSp2-4-5 RNA spike-in mix (e.g., containing UniSp2, UniSp4, and UniSp5) was combined with 60 μl lysis Buffer RPL, and added to the 200 μl plasma/serum sample. The mixture was vortexed for >5 s and incubated at room temperature for 3 min. 20 μl Buffer RPP was added. The mixture was vortexed for >20 s, incubated at room temperature for 3 min, and centrifuged at 12000×g for 3 min at room temperature to pellet the precipitate.

4. The supernatant (˜230 μl) was transferred to a new tube, added with 1 volume isopropanol, and mixed by upside down 10˜20 times. The entire sample was transferred to RNeasy UCP MinElute column (Qiagen), and centrifuged for 15 s at 8000×g. The column was then added with 700 μl Buffer RWT, centrifuged for 15 s at 8000×g, and added with 500 μl Buffer RPE, centrifuged for 15 s at 8000×g, added with 500 μl of 80% ethanol, and then centrifuged for 2 min at 8000×g.

5. The RNeasy UCP MinElute column was placed in a new collection tube, centrifuged at full speed for 5 min to dry the membrane. Then the column was placed in a new collection tube, added with 20 μl RNase-free water, incubated for 2 min, and centrifuged for 1 min at 15,000×g to elute the RNA.

Measurement of miRNA
cDNA Reverse Transcription

The RNA Spike-In Kit (Qiagen), miRCURY LNA RT Kit (Qiagen) were used for cDNA reverse transcription.

Reverse transcription reaction (RT reaction) mixtures were prepared, with each 10 μl RT reaction mixture including: 0.5 μl UniSp6 and cel-miR-39-3p RNA spike-in mix; 5.7 μl RNase-free water; 2 μl 5× miRCURY RT Reaction Buffer; 1 μl 10× miRCURY RT Enzyme Mix; and 0.8 μl Template RNA. The PCR tubes were then placed into the thermocycler with program: 42° C. 60 min, following 95° C. 5 min to synthesis the cDNA.

qPCR

The miRCURY LNA SYBR Green Kits (Qiagen) and target miRCURY LNA miRNA PCR assays (Qiagen) were used for qPCR. Primers used included hsa-miR-155-5p (Qiagen, 339306-YP02119311); hsa-miR-22-3p (Qiagen, 339306-YP00204606), hsa-miR-23a-3p (Qiagen, 339306-YP00204772), hsa-miR-17-5p (Qiagen, 339306-YP02119304), hsa-miR-20a-5p (Qiagen, 339306-YP00204292), hsa-let-7b-5p (Qiagen, 339306-YP00204750).

5 μl cDNA was diluted into 145 μl of RNase-free water. The qPCR reaction mixtures were prepared, which included 5 μl 2× miRCURY SYBR Green Master Mix, 1 μl miRCURY LNA miRNA assay, 0.05 μl ROX, 3 μl diluted cDNA, and 0.95 μl RNase-free water. The plates with qPCR reaction mixtures were then placed into real-time PCR system with program: 95° C. 2 min; 40 cycles of 95° C. 10 s and 56° C. 60 s; and 60-90° C. for melting curve analysis.

### 7.9.2 Example 2: Detecting and Classifying Endometriosis and Other Gynecological Diseases Using miRNA Biomarkers

Serum samples were obtained from normal healthy controls (no pathology), patient controls (women with pathology and symptoms) and confirmed endometriosis patients. Specifically, samples subjected to this analysis were obtained from patients having the following gynecological diseases. Rdx1, Rdx2, and Rdx3 were samples from healthy controls.

| Sample | 13-160 | 13-183 | 13-513 |
|---|---|---|---|
| Diseases | physiological | hemorrhagic cyst | leiomyomas |
| | cysts | | |

| Sample | 13-464 | 14-097 | 14-747 | 16-164 |
|---|---|---|---|---|
| Diseases | endometriosis | endometriosis | endometriosis | endometriosis |

| Sample | 16-769 | 18-1097 | 23-0249 |
|---|---|---|---|
| Diseases | endometriosis and | endometriosis | endometriosis and |
| | adenomyosis | | adenomyosis |

All samples were processed as described in Example 1 and subjected to qPCR for measuring the expression of miRNA biomarkers Let-7b-5p, miR-155-5p, miR-23a-3p, miR-17-5p, and miR-20a-5p, and miR-22-5p, the combined expression of 6 miRNA biomarkers were calculated and normalized by UniSp5 level.

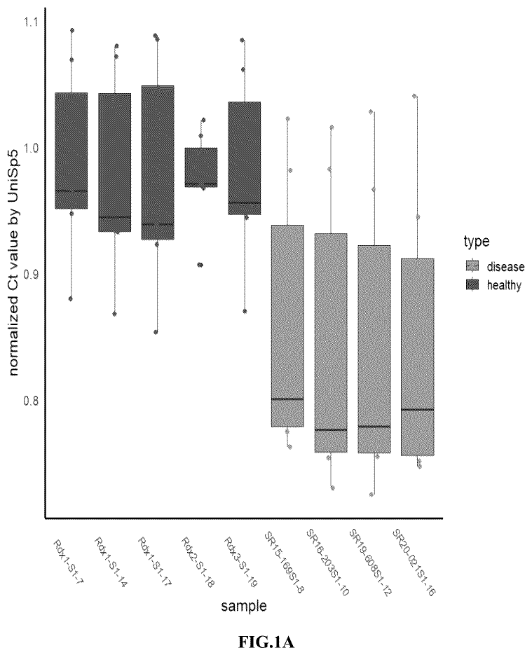

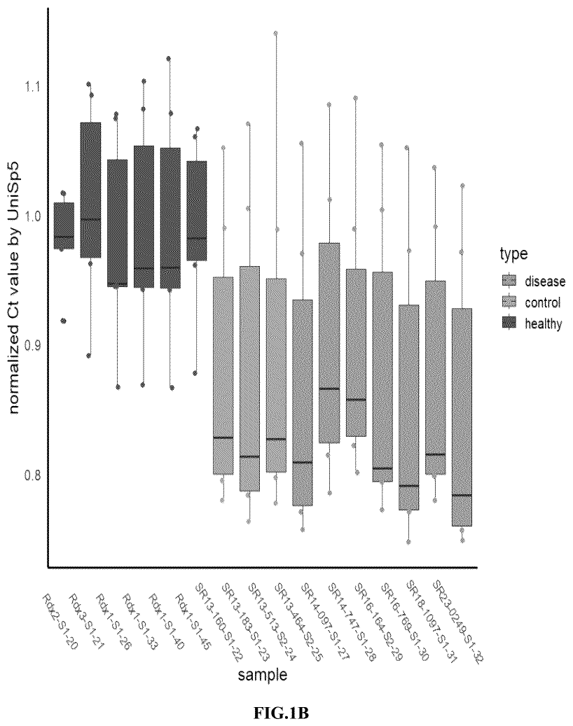

As shown in FIGS. 1A (batch 1) and 1B (batch 2), the expression profile of this biomarker panel was clearly distinct between healthy controls and diseased population. The box plot data as depicted in FIGS. 1A and 1B represent the weighted sum of the mean Ct value for the combined markers.

Additionally, as shown in FIG. 1B, samples from patients with both endometriosis and adenomyosis (16-769 and 23-0249) and those from patients with endometriosis only had different Ct values, demonstrating that Ct cut-off values could distinguish between patients with both endometriosis and adenomyosis and those with endometriosis only.

Furthermore, the clear distinction between healthy control and patient controls (13-160; 13-183; and 13-513) indicated that the marker panels could also be used to detect gynecological disease in general.

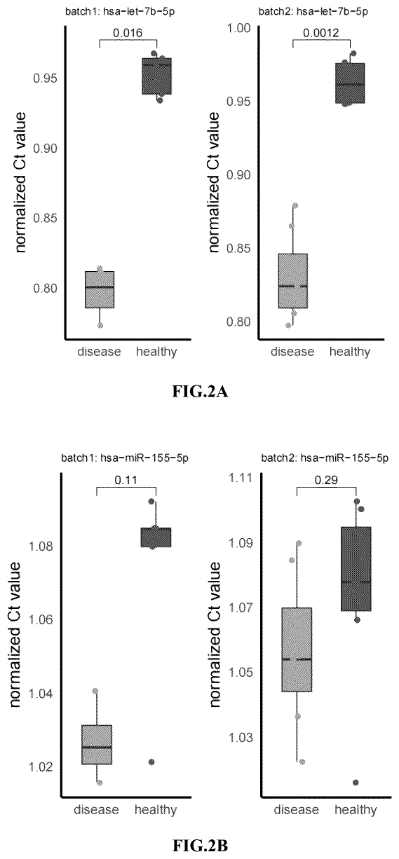

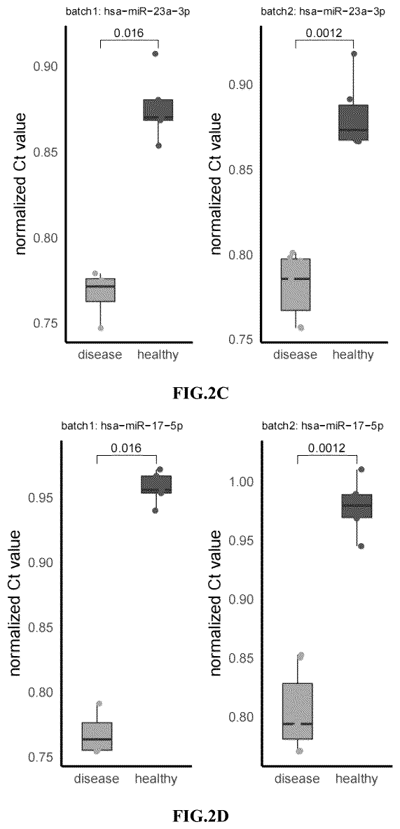

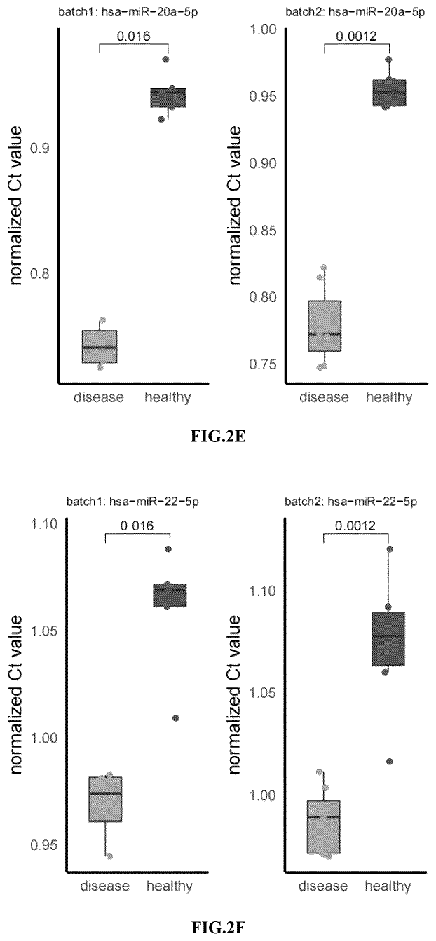

The expression profiles of the individual miRNA biomarkers (i.e., biomarkers Let-7b-5p, miR-155-5p, miR-23a-3p, miR-17-5p, and miR-20a-5p, and miR-22-5p) were also analyzed for their correlation with endometriosis and gynecological disease in general. As shown in FIGS. 2A-2F, all six markers showed very distinct expression levels between patients and normal healthy controls, supporting the use of these markers to detect presence of gynecological conditions.

### 7.9.3 Example 3: Distinguishing Endometriosis from Other Gynecological Diseases Using miRNA Biomarkers

A four-biomarker panel was used to analyze samples from endometriosis patients and patients with a variety of other gynecological diseases. The expression levels of these miRNAs were measured and analyzed as described in Example 1.

Samples subjected to this analysis were obtained from patients having the following gynecological diseases.

| Sample | 19-608 | 18-1097 | 16-203 |
|---|---|---|---|
| Diseases | Endometriosis | Endometriosis | Endometriosis |

| Sample | 15-125 | 13-076 | 13-183 |
|---|---|---|---|
| Diseases | Surrounding uninvolved | Squamous | Hemorrhagic |
| | splenic parenchyma with | metaplasia | cyst |
| | congestion | | |

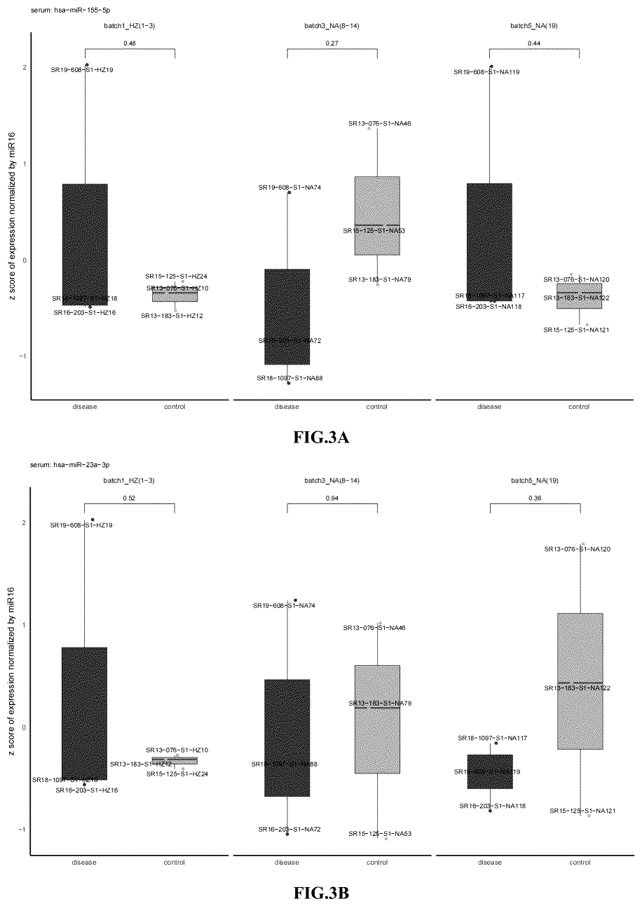

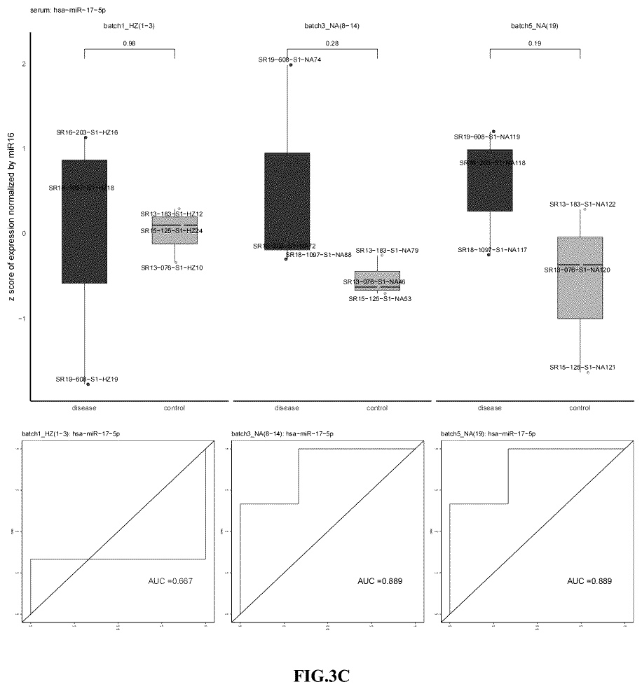

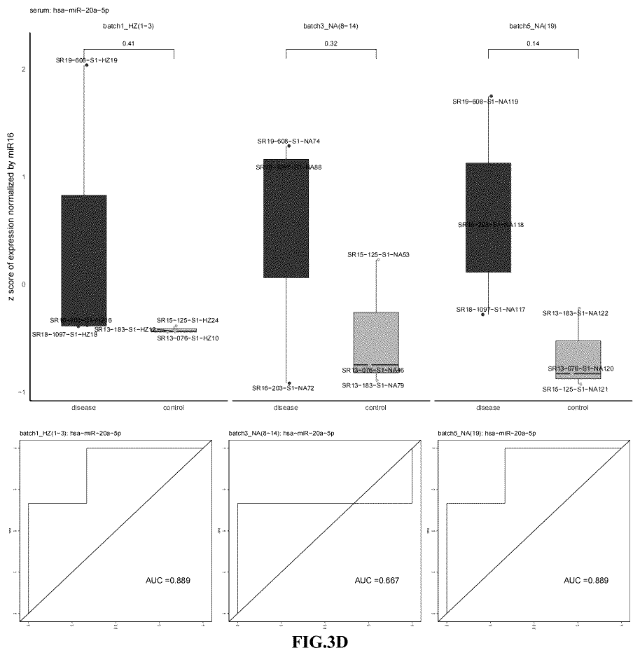

As shown in FIGS. 3A-3D, the expression of miR-155 (FIG. 3A), miR-20a (FIG. 31B), miR-17-5p (FIG. 3C) and miR-23a (FIG. 31D) could distinguish patients with endometriosis from those with other gynecological diseases.

### 7.9.4 Example 4: CA-125 Level at Secretory Phase for Endometriosis Diagnosis

140 Serum samples from surgically confirmed endometriosis patients (“Patient”) and control patients (“Patient Control”) who had other gynecological conditions were analyzed for CA-125 level. The samples included 62 prospective patients/controls and 82 retrospective patient/controls based on medical records. The samples were taken either at the secretory phase of menstrual cycle or the proliferative phase of menstrual cycle. CA-125 protein levels were detected based on standardized Cobus assay (cut-off >30 ng/mL). Menstrual phase was distinguished based on path report and/or dates of last menstrual period. As shown in the table below, both sensitive and specificity of serum CA-125 level as the marker for endometriosis were increased when samples were obtained from secretory phase as compared to proliferative phase.

| | Secretory (n = 51) | | Proliferative (n = 89) | |
|---|---|---|---|---|
| | Patient | Patient Control | Patient | Patient Control |
| CA-125 | (n = 30) | (n = 21) | (n = 33) | (n = 56) |
| Positive | 29 (96.7%) | 2 | 26 (78.8%) | 12 |
| Negative | 1 | 19 (90.5%) | 7 | 44 (78.6%) |

| | | |
|---|---|---|
| Sensitivity | 93.5% | 68.4% |
| Specificity | 95.0% | 86.3% |
| Accuracy | 94.1% | 78.7% |

Combined (n = 140)

| | Patient | Patient Control |
|---|---|---|
| CA-125 | (n = 63) | (n = 77) |
| Positive | 55 (87.3%) | 14 |
| Negative | 8 | 63 (81.8%) |

| | |
|---|---|
| Sensitivity | 79.7% |
| Specificity | 88.7% |
| Accuracy | 84.3% |

### 7.9.5 Example 5: Individual miRNA Markers at Secretory Phase for Endometriosis Diagnosis

Serum samples were obtained during secretory phase of the menstrual cycle of surgically confirmed endometriosis patients and control patients, and were analyzed for CA-125 level and miRNA levels.

FIG. 4 provides the results of statistical analysis for each individual marker, including CA-125 (protein), miR-34c-3p, miR-20a-5p, miR-17-5p, miR-22-3p, miR-199-5p, let-7b-5p, miR-23a-3p, miR-155-5p. As shown, CA-125 (protein), miR-34c-3p, miR-20a-5p, miR-17-5p each demonstrated excellent predictive performance even as individual markers (AUC close to 1), when samples were taken at secretory phase.

### 7.9.6 Example 6: Combinations of miRNAs Markers at Secretory Phase for Endometriosis Diagnosis

Models with different marker or combination of markers were analyzed for their prediction power for endometriosis diagnosis. Sample were taken during secretory phase of menstrual cycle from surgically confirmed endometriosis patients and control patients.

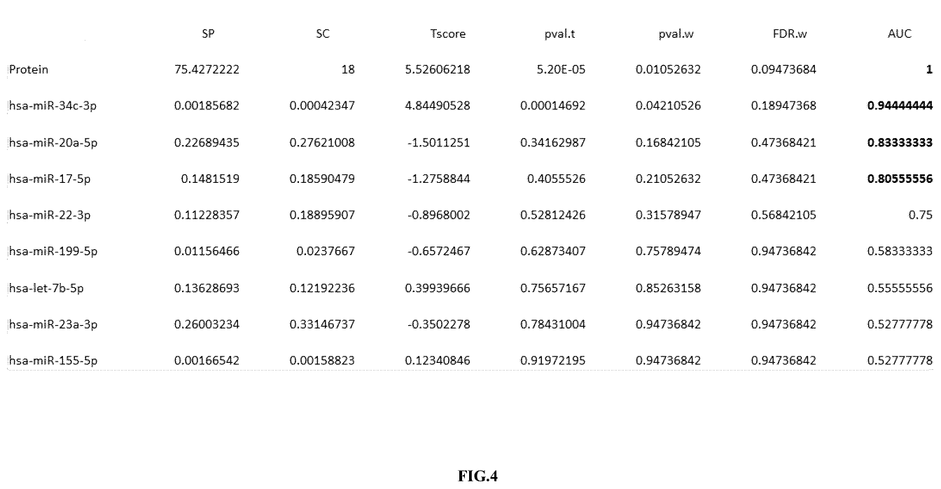

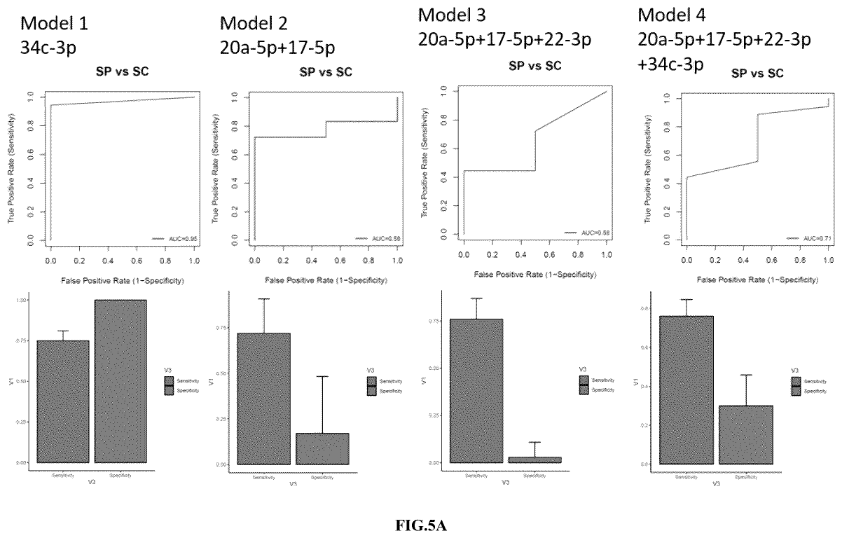

FIGS. 5A-5B provide the area under curves (AUC) of CA-125 (Protein) and 7 study models. As shown, the 7 models demonstrated varied prediction power, of which Model 1(34c-3p), Model 5(34c-3p+20a-5p), Model 6(34c-3p+17-5p) and Model 7(34c-3p+22-3p) performed better than the rest (AUC>0.8).

### 7.9.7 Example 7: Workflow for Endometriosis Diagnosis Using Combination of Protein Marker and miRNA Markers

Provided herein is a workflow for endometriosis using a combination of the protein marker (CA-125) and the miRNA markers.

| | Protein+ | Protein− |
|---|---|---|
| miRNA+ | Positive for Endometriosis | Indeterminant - retest |
| miRNA− | Indeterminant - protein false | Negative for Endometriosis |
| | positive* | |

In general, the candidate patient can present with symptoms such as abnormal abdominal/pelvic pain, abnormal bleeding/heavy or irregular, and/or infertility. Clinical practice tools include clinical exam, the transvaginal ultrasound (US) and magnetic resonance imaging (MRI), which can rule out ovarian and severe endometriosis (EMS) (two or more types of endometrioses), adenomyosis and leiomyomas. However, mild and/or peritoneal forms of EMS are difficult to detect and account for >70% of EMS cases left undiagnosed. Here, to avoid misdiagnosis or missed diagnosis, patients with negative imaging/clinical findings can be subjected to the assay disclosed herein. To do so, serum samples can be taken from patients suspected of having endometriosis at the secretory phase of their menstrual cycle. Both miRNA and CA-125 levels can be measured and compared with a predetermined cutoff. The miRNA markers can be any combination of miR-34c-3p, miR-23a-3p, miR-17-5p, miR-155-5p, let-7b-5p, miR-199-5p, miR-22-3p, miR-20a-5p. A patient who is positive for both the protein marker and the miRNA marker(s) can be diagnosed with endometriosis. Conversely, a patient who is negative for both the protein marker and the miRNA marker(s) can be excluded from an endometriosis diagnosis. A patient who is positive for the protein marker and negative for the miRNA marker(s), or positive for the miRNA marker(s) and negative for the protein marker is undetermined.

### 7.9.8 Example 8: Clinical Study Workflow for Detecting the Presence or Absence of Endometriosis Among Symptomatic Women of Ages 18-50 Years

Provided herein is a clinical study workflow for detecting the presence or absence of endometriosis among symptomatic women of ages 18-50 years using a combination of the protein marker (CA-125) and the miRNA markers. The primary objective is to determine whether a miRNA panel can confirm accurate disease association, such as greater than 90% concordance with laparoscopy/histopathology.

This study includes 350 reproductive women of ages 18-50 years, including 300 symptomatic women scheduled for surgery for the presence and absence of endometriosis (with symptoms of pelvic pain, abnormal menstrual bleeding, and/or infertility) and 50 normal healthy women ((not surgically confirmed)). All subjects provide prior written informed consent. 150 women confirmed by biopsy/histology to have endometriosis are classified as “Patients/Cohort 1”. Cohort 1 includes subjects with endometriosis of any stage and any type, and with or without adenomyosis. 150 women found not to have endometriosis surgically with or without other gynecologic conditions (any gynecological conditions, such as uterine fibroids, cysts, pelvic inflammation, and leiomyomas) are classified as “Patient Controls/Cohort 2”. Cohort 2 does not include subjects with adenomyosis. 50 normal healthy women (with no history of disease or infertility) serving as disease negative controls are classified as “Healthy Controls/Cohort 3”. Each subject completes a symptom and quality-of-life questionnaire, assessing level of pain and bleeding symptoms that leads to surgery. Medical information pertaining to infertility, abdominal pain/severity, and abnormal bleeding symptoms is required. If subjects do not have cycles, either due to endometrial ablation or continuous hormonal use, phase can be determined by measurement of blood progesterone levels.

Blood is drawn from all subjects prior to surgery during the proliferative or secretory phase of the menstrual cycle. Serum samples are isolated from whole blood within 2 hours of blood draw to avoid influence of background DNA/RNA levels using the standard operating procedure, aliquoted into 0.5 mL volumes, and then stored frozen at −80° C. until shipment to the testing site. 300 μl serum samples are thawed and processed for RNA extraction. Reverse transcription and cDNA synthesis are conducted for the extracted RNA, followed by quantitative PCR analysis of expression levels of 5 marker miRNAs (miR-17-5p, miR-22-3p, miR-20a-5p, miR-34c-3p, and miR-155-5p). QuantStudio™ 7 Flex Real-Time PCR System by ThermoFisher are used for qPCR and expression profile analysis. Quality controls within the qPCR include no template controls for contamination. Internal molecular standards including known endometriosis-positive and endometriosis-negative serum controls, no template controls, and miRNA normalization controls are in each qPCR run. Data is collected and analyzed using software developed by Heranova.

The biomarker identification process is based on the machine learning process of sample data collection of subjects in 3 cohorts. The process involves miRNA profiling, data pre-processing, data splitting (Training and Testing Sets), feature engineering, model selection, and model evaluation.

Data from medical records is used to complete an e-Case Report Form (eCRF). The eCRF includes serum CA-125 and progesterone levels measured using the same serum samples from all 3 cohorts using existing FDA-cleared tests. Each miRNA marker and CA125 protein level are analyzed using a bioinformatic algorithm in 3 cohorts to demonstrate accuracy in distinguishing between patients and both control groups. The analysis results are compared to histopathology results generated following biopsy in a blinded manner to generate AUC, accuracy, sensitivity, specificity, and positive and negative predictive values. Blinded manner means blood serum is coded, and the clinical testing site is blinded to disease status as part of patient de-identification. Clinical site performing the test is blinded to pathology results until concordance data analysis has been completed.

Analysis is performed according to a separate Statistical Analysis Plan. Discordant results are excluded from the analysis.

| TEST | CLINICAL CONDITION | | |
|---|---|---|---|
| OUTCOME | POSITIVE | NEGATIVE | |
| POSITIVE | True | False | Positive Predictive |
| | Positive (TP) | Positive (FP) | Value |
| | | | TP / (TP + FP) |
| NEGATIVE | False | True | Negative Predictive |
| | Negative (FN) | Negative (TN) | Value |
| | | | TN / (TN + FN) |
| | Sensitivity | Specificity | |
| | TP / (TP + FN) | TN / (FP + TN) | |

True Positive (TP): The number of subjects/specimens with true positive test results. False Positive (FP): A positive test result for a subject in whom the condition of interest is absent. True Negative (TN): The number of subjects/specimens with true negative test results. False Negative (FN): A negative test result for a subject in whom the condition of interest is present. Positive Predictive Value (PPV): PPV=TP/(TP+FP)×100%. The proportion of test positive patients who have the target condition. Negative Predictive Value (NPV): NPV=TN/(TN+FN)×100%. The proportion of test negative patients who do not have the target condition.

Positive Percent Agreement (PPA): The proportion of non-reference standard positive subjects in whom the new test is positive. Negative Percent Agreement (NPA): The proportion of non-reference standard negative subjects in whom the new test is negative.

### 7.9.9 Example 9: Predictive Performance of miRNAs and CA-125 Combination in Secretory and Proliferative Phases

151 blood samples were drawn from 50 patients and 42 patient controls in proliferative phase, and 34 patients and 25 patient controls in secretory phase. The phases were distinguished by progesterone level. Expression levels of a series of miRNAs and protein CA-125 (M9) were tested and analyzed, and at the same time, machine learning model generated.

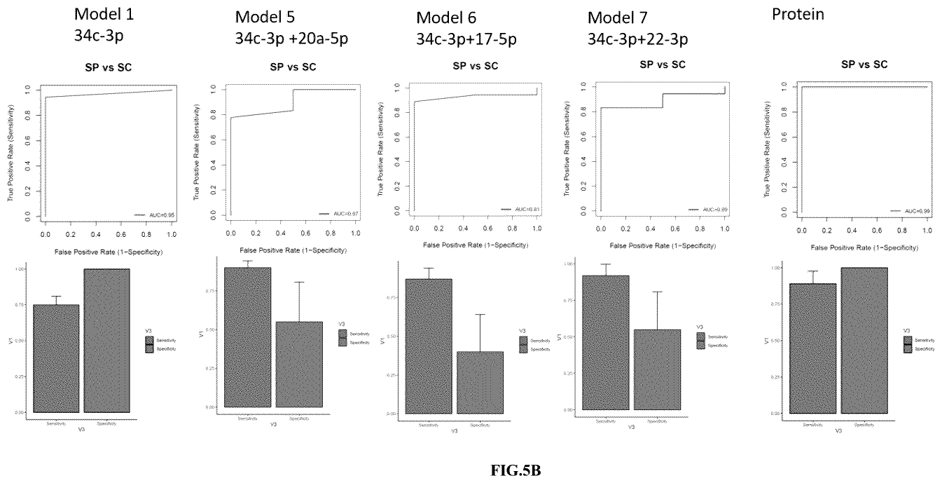

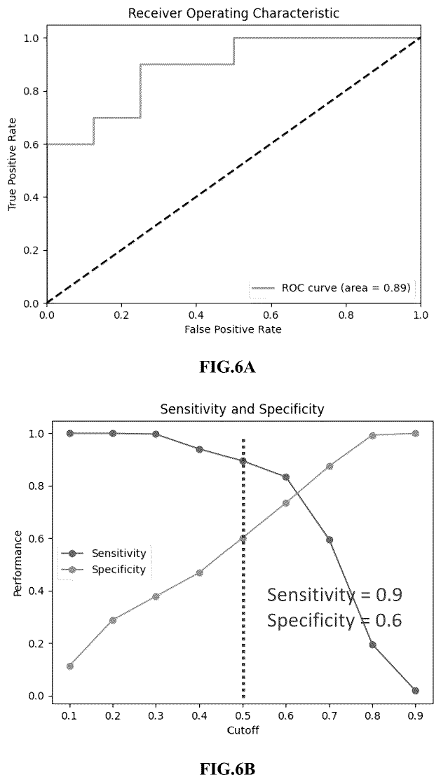

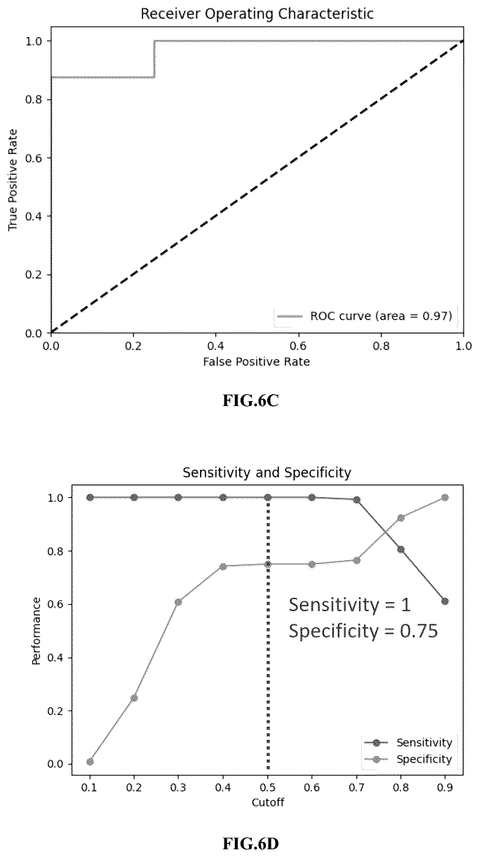

As shown in FIG. 6A and FIG. 6B, when blood samples were collected during the proliferative phase, the combination of miR-17-5p, miR-23a-3p, miR-34c-3p, and the protein CA-125 in a random forest prediction model yielded an AUC of 0.89 (FIG. 6A), with sensitivity and specificity reaching 0.9 and 0.6, respectively (FIG. 6B). As shown in FIG. 6C and FIG. 6D, for blood samples collected during the secretory phase, the combination of miR-20a-5p, miR-22-3p, miR-34c-3p, and the protein CA-125 in a random forest prediction model resulted in an AUC of 0.97 (FIG. 6C), with sensitivity and specificity of 1 and 0.75, respectively (FIG. 6D).

### 7.9.10 Example 10: Detecting miRNA Biomarkers in Serum/Plasma Samples by ddPCR

The following protocol was followed for detecting the expression of miRNA in serum/plasma samples by ddPCR:

### Sample Preparation

Peripheral blood samples were processed within 2 hours after they were obtained from patients to separate serum and plasma. The processed serum/plasma samples were then frozen and stored/shipped for analysis.

### RNA Extraction

The RNA Spike-In Kit (Qiagen), miRNeasy Serum/Plasma Advanced Kit (Qiagen) and RNase-Free DNase Set (Qiagen) were used for RNA extraction.

1. Serum/plasma samples were thawed and centrifuged for 5 min at 3000×g and 4° C.; 200 μl supernatant was transferred into empty tubes.

2 (optional). 2 μl DNase I (Qiagen) was added to the 200 μl plasma/serum sample, incubated at room temperature for 15 minutes, then centrifuged briefly.

3. 2 μl spike-in mix (cel-miR-39-5p) was combined with 60 μl lysis Buffer RPL, and added to the 200 μl plasma/serum sample. The mixture was vortexed for >5 s and incubated at room temperature for 3 min. 20 μl Buffer RPP was added. The mixture was vortexed for >20 s, incubated at room temperature for 3 min, and centrifuged at 12000×g for 3 min at room temperature to pellet the precipitate.

4. The supernatant (˜230 l) was transferred to a new tube, added with 1 volume isopropanol, and mixed by upside down 10-20 times. The entire sample was transferred to RNeasy UCP MinElute column (Qiagen), and centrifuged for 15 s at 8000×g. The column was then added with 700 μl Buffer RWT, centrifuged for 15 s at 8000×g, and added with 500 μl Buffer RPE, centrifuged for 15 s at 8000×g, added with 500 μl of 80% ethanol, and then centrifuged for 2 min at 8000×g.

5. The RNeasy UCP MinElute column was placed in a new collection tube, centrifuged at full speed for 5 min to dry the membrane. Then the column was placed in a new collection tube, added with 20 μl RNase-free water, incubated for 2 min, and centrifuged for 1 min at 15,000×g to elute the RNA.

Measurement of miRNA
cDNA Reverse Transcription

The FastKing RT Kit II (Tiangen) were used for cDNA reverse transcription.

Reverse transcription reaction (RT reaction) mixtures were prepared, with each 10 μl RT reaction mixture including: 4.5 μl RNase-free water; 1 μl 10×King RT Buffer II; 0.5 μl FastKing RT Enzyme MixII; 2 μl Specific primers (2.5 μM); and 2 μl Template RNA. The PCR tubes were then placed into the thermocycler with program: 42° C. 15 min, following 85° C. 1 min to synthesis the cDNA.

ddPCR

The Magic 2xdPCR EvaGreen Master Mix (Rox) (Magic Bio) and target miRNA marker primers were used for qPCR. Specific Primers used included hsa-miR-17-5p; hsa-miR-19b-3p, hsa-miR-21-5p, hsa-miR-15b-5p, hsa-miR-19a-5p, hsa-miR-664a-3p, hsa-miR-381-3p, hsa-miR-23a-3p, hsa-miR-654a-3p, hsa-miR-24-3p, has-miR-92a-3p, has-miR-652-3p, has-miR-92b-3p, has-miR-532-5p, has-miR-338-5p, has-miR-374a-5p, has-miR-421.

The qPCR reaction mixtures were prepared, which included 11 μl 2× ddPCR Mix, 1 μl specific forward primer, 1 μl specific reverse primer, 0.5 μl Evagreen, 0.5 μl ROX, 2 μl cDNA, and 6 μl RNase-free water. The tubes with ddPCR reaction mixtures were then placed into digital droplet PCR system with program: 60° C. 5 min; 95° C. 5 min; 45 cycles of 95° C. 20 s and 56° C. 30 s.

### 7.9.11 Example 11: Identification and Verification of Markers Useful in Endometriosis Detection Using qPCR and ddPCR—Part I

A custom miRNA-seq pipeline was designed to identify both novel housekeeping miRNA(s) and diagnostic biomarkers capable of distinguishing individuals with endometriosis from those without the condition. The pipeline integrated advanced computational approaches to ensure that the selected housekeeping miRNA(s) exhibited consistent expression across samples, regardless of disease status or experimental conditions. This step was critical for establishing a robust normalization framework suitable for downstream qPCR and ddPCR workflows.

The identified biomarkers must meet stringent criteria, including the ability to retain the biological signal observed in the differential expression analysis of NGS data when normalized using the novel housekeeping miRNA(s). This ensured that the diagnostic markers were both reproducible and clinically meaningful, addressing the common issue where skewed normalization undermines the validity of qPCR/ddPCR results. By bridging the gap between NGS-based findings and validation in qPCR/ddPCR assays, this approach enhanced the potential for robust biomarker translation into clinical diagnostics for endometriosis.

To identify robust biomarkers and housekeeping miRNAs for endometriosis diagnostics, miRNA-sequencing (miRNA-seq) was conducted on 40 subjects, including 20 individuals diagnosed with endometriosis and 20 without. This balanced cohort ensured adequate representation of biological variance across the two groups, enhancing the robustness of downstream analyses. The raw sequencing data were processed using the miRge3 pipeline with default parameters to generate high-quality, preprocessed miRNA counts. Differential expression analysis was then performed using DESeq2 to identify miRNAs with statistically significant differences between the disease and control groups. Only miRNAs with a p-value<0.05 were retained for further analysis ensuring a stringent threshold for biomarker identification.

Candidate housekeeping miRNAs were identified as novel endogenous controls for qPCR/ddPCR workflows through analysis. Selection criteria included a p-value>0.8, log 2-fold-change within ±0.02 to ensure minimal variation between disease and control groups, and an average read count exceeding 500 to confirm sufficient detectability in qPCR/ddPCR assays. 15 potential housekeeping miRNAs with stable expression across all samples were identified.

The utility of these housekeeping miRNAs was verified by simulating a qPCR/ddPCR normalization workflow. All miRNAs were normalized against each of the 15 candidate housekeeping miRNAs, and the statistical significance of differences between disease and control groups was re-evaluated using Wilcoxon rank-sum tests. Only miRNAs with a p-value<0.05 following normalization were retained as candidate biomarkers.

The biomarkers identified through the simulated qPCR/ddPCR workflow were compared to those identified through the initial NGS-based differential expression analysis. miRNAs common to both methods were retained as high-priority candidates for further study. To confirm the reproducibility and robustness of the identified biomarkers and housekeeping miRNAs, a second round of miRNA-seq was conducted on an independent validation cohort. This validation batch focused specifically on the biomarkers and housekeeping miRNAs identified in early analysis. Consistency in the direction of effect, data distribution, and statistical significance across the two batches was used as the criterion for final selection. Biomarkers and housekeeping miRNAs that demonstrated consistent trends were designated as “high-confidence” candidates. High-confidence biomarkers and housekeeping miRNAs identified through this comprehensive pipeline were then validated using qPCR/ddPCR assays.

All samples were obtained at secretory phase.

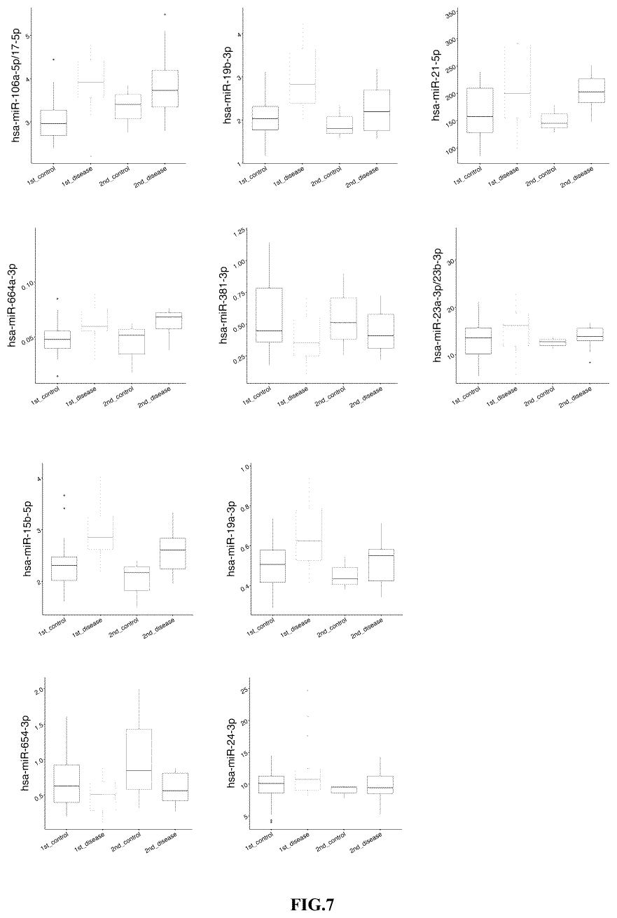

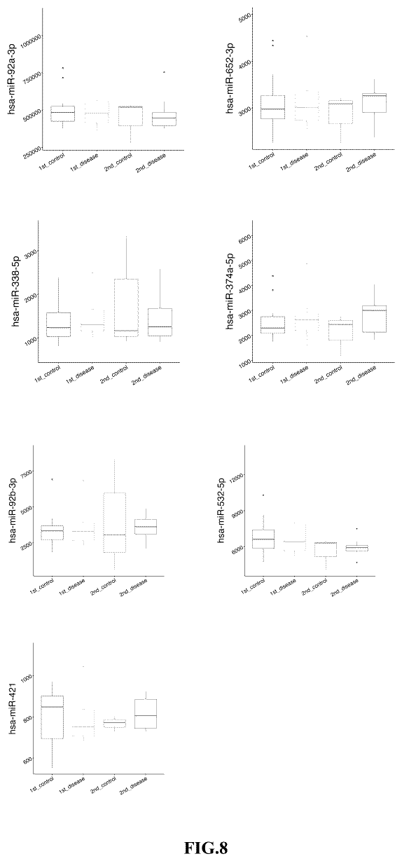

A total of 10 miRNA biomarkers and 7 miRNA endogenous controls were identified through the implementation of the bioinformatics pipeline described above. The miRNA biomarkers were miR-17-5p, miR-19b-3p, miR-21-5p, miR-15b-5p, miR-19a-3p, miR-664a-3p, miR-381-3p, miR-23b-3p, miR-654-3p and miR-24-3p (FIG. 7), and the endogenous controls were miR-92a-3p, miR-652-3p, miR-92b-3p, miR-532-5p, miR-338-5p, miR-374a-5p, and miR-421 (FIG. 8). Each of these markers had at least an average count of 500 based on NGS data to ensure detectability via qPCR/ddPCR platforms. As shown in FIG. 7 and the table below, among these 10 biomarkers, eight are upregulated in subjects with endometriosis (miR-17-5p, miR-19b-3p, miR-21-5p, miR-15b-5p, miR-19a-3p, miR-664a-3p, miR-23b-3p, miR-24-3p), while two are downregulated (miR-381-3p, miR-654-3p).

| Biomarker | p-Value | Count Range | Negative Binomial |
|---|---|---|---|
| miR-17-5p | 0.0016 | 8000-13000 | <0.01 |
| miR-19b-3p | 0.0035 | 6000-14000 | <0.01 |
| miR-21-5p | 0.0061 | 400000-900000 | <0.01 |
| miR-15b-5p | 0.0087 | 7000-12000 | <0.01 |
| miR-19a-3p | 0.03 | 1500-3000 | <0.01 |
| miR-664a-3p | 0.00093 | 100-220 | <0.01 |
| miR-381-3p | 0.046 | 800-2500 | <0.05 |
| miR-23a-3p | 0.06 | 30000-50000 | <0.05 |
| miR-654-3p | 0.086 | 1000-3000 | <0.05 |
| miR-24-3p | 0.15 | 30000-50000 | <0.05 |

### 7.9.12 Example 12: Identification and Verification of Markers Useful in Endometriosis Detection Using qPCR and ddPCR—Part II

The same study described in Example 11 was carried out in parallel with samples taken at proliferative phase of the menstrual cycle, and miRNAs were identified as high-confidence candidates as biomarkers for endometriosis and endogenous controls, respectively.

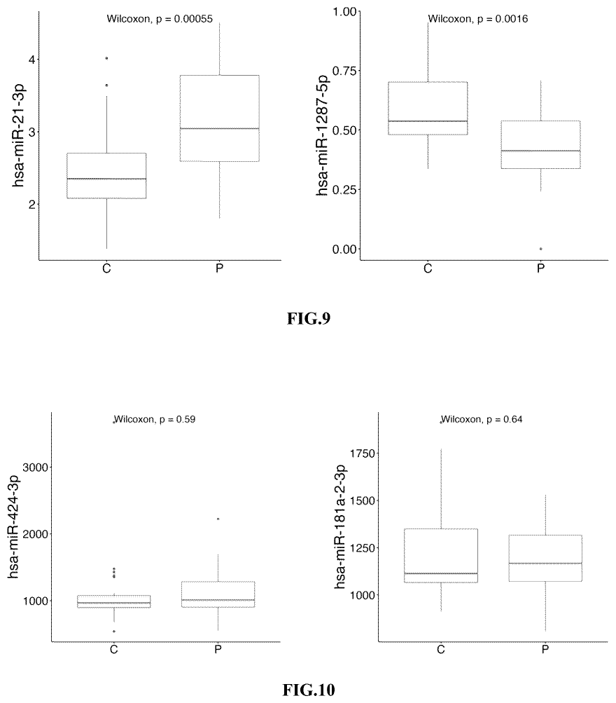

The miRNA biomarkers for endometriosis included miR-21-3p, miR-22-5p, miR-1287-5p, miR-625-5p, miR-1294-5p, miR-7704, miR-221-5p, miR-340-5p, miR-450b-5p, miR-548e-3p, and miR-502-3p (representative results in FIG. 9), and the endogenous controls included miR-181a-2-3p, miR-181d-5p, Let-7g-5p, miR-181a-3p, miR-24-2-5p, miR-130b-3p, miR-628-3p, miR-19a-3p, miR-181a-5p and miR-424-3p (representative in FIG. 10). Each of these markers had at least an average count of 500 based on NGS data to ensure detectability via qPCR/ddPCR platforms. Among these 11 biomarkers, eight are upregulated in subjects with endometriosis (miR-21-3p, miR-22-5p, miR-1287-5p, miR-221-5p, miR-340-5p, miR-450b-5p, miR-548e-3p, and miR-502-3p), while three are downregulated (miR-625-5p, miR-1294-5p, miR-7704).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Unless the context indicates otherwise, it is specifically intended that the various features described herein can be used in any combination.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

1-209. (canceled)

210. A method of detecting a gynecological disease in a subject, comprising
(a) measuring a level of at least one miRNA selected from: miR-17, miR-19b, miR-21, miR-15b, miR-19a, miR-664a, miR-381, miR-23a, miR-654, miR-24, Let-7b, miR-20a, miR-22, miR-34c, miR-1287, miR-625, miR-1294, miR-7704, miR-221, miR-340, miR-450b, miR-548e, miR-502, miR-574, miR-9, miR-299, miR-6789, miR-593, miR-346, miR-34a, miR-449a, miR-7109, miR-3907, miR-557, miR-6801, miR-4420, miR-570, miR-155, miR-199, miR-520d, miR-424, and miR-200, in a sample of the subject, and
(b) comparing the level to a reference level; wherein an altered level indicates that the subject has a gynecological disease;
wherein optionally: miR-17 comprises miR-17-5p, miR-17-3p, or both; miR-19b comprises miR-19b-5p, miR-19b-3p, or both; miR-21 comprises miR-21-5p, miR-21-3p, or both; miR-15b comprises miR-15b-5p, miR-15b-3p, or both; miR-19a comprises miR-19a-5p, miR-19a-3p, or both; miR-664a comprises miR-664a-5p, miR-664a-3p, or both; miR-381 comprises miR-381-5p, miR-381-3p, or both; miR-23a comprises miR-23a-5p, miR-23a-3p, or both; miR-654 comprises miR-654-5p, miR-654-3p, or both; miR-24 comprises miR-24-5p, miR-24-3p, or both; Let-7b comprises Let-7b-5p, Let-7b-3p, or both; miR-20a comprises miR-20a-5p, miR-20a-3p, or both; miR-22 comprises miR-22-5p, miR-22-3p, or both; miR-34c comprises miR-34c-5p, miR-34c-3p, or both; miR-1287 comprises miR-1287-5p, miR-1287-3p, or both; miR-625 comprises miR-625-5p, miR-625-3p, or both; miR-1294 comprises miR-1294-5p, miR-1294-3p, or both; miR-7704 comprises miR-7704; miR-221 comprises miR-221-5p, miR-221-3p, or both; miR-340 comprises miR-340-5p, miR-340-3p, or both; miR-450b comprises miR-450b-5p, miR-450b-3p, or both; miR-548e comprises miR-548e-5p, miR-548e-3p, or both; miR-502 comprises miR-502-5p, miR-502-3p, or both; miR-574 comprises miR-574-5p, miR-574-3p, or both; miR-9 comprises miR-9-5p, miR-9-3p, or both; miR-299 comprises miR-299-5p, miR-299-3p, or both; miR-6789 comprises miR-6789-5p, miR-6789-3p, or both; miR-593 comprises miR-593-5p, miR-593-3p, or both; miR-346 comprises miR-346-5p, miR-346-3p, or both; miR-34a comprises miR-34a-5p, miR-34a-3p, or both; miR-449a comprises miR-449a-5p, miR-449a-3p, or both; miR-7109 comprises miR-7109-5p, miR-7109-3p, or both; miR-3907 comprises miR-3907; miR-557 comprises miR-557; miR-6801 comprises miR-6801-5p, miR-6801-3p or both; miR-4420 comprises miR-4420; miR-570 comprises miR-570-5p, miR-570-3p, or both; miR-155 comprises miR-155-5p, miR-155-3p, or both; miR-199 comprises miR-199-5p, miR-199-3p, or both; miR-520d comprises miR-520d-5p, miR-520d-3p, or both; miR-424 comprises miR-424-5p, miR-424-3p, or both; miR-200 comprises miR-200a-5p, miR-200a-3p, miR-200b-5p, miR-200b-3p, miR-200c-5p, or miR-200c-3p, or any combination thereof;
wherein optionally: miR-17 comprises miR-17-5p; miR-19b comprises miR-19b-3p; miR-21 comprises miR-21-5p, miR-21-3p, or both; miR-15b comprises miR-15b-5p; miR-19a comprises miR-19a-3p; miR-664a comprises miR-664a-3p; miR-381 comprises miR-381-3p; miR-23a comprises miR-23a-3p; miR-654 comprises miR-654-3p; miR-24 comprises miR-24-3p; Let-7b comprises Let-7b-5p; miR-20a comprises miR-20a-5p; miR-22 comprises miR-22-5p, miR-22-3p, or both; miR-34c comprises miR-34c-3p; miR-1287 comprises miR-1287-5p; miR-625 comprises miR-625-5p; miR-1294 comprises miR-1294-5p; miR-7704 comprises miR-7704; miR-221 comprises miR-221-5p; miR-340 comprises miR-340-5p; miR-450b comprises miR-450b-5p; miR-548e comprises miR-548e-3p; miR-502 comprises miR-502-3p; miR-574 comprises miR-574-3p; miR-9 comprises miR-9-5p; miR-299 comprises miR-299-5p, miR-299-3p, or both; miR-6789 comprises miR-6789-5p; miR-593 comprises miR-593-5p; miR-346 comprises miR-346-5p, miR-346-3p, or both; miR-34a comprises miR-34a-5p; miR-449a comprises miR-449a-5p, miR-449a-3p, or both; miR-7109 comprises miR-7109-5p; miR-3907 comprises miR-3907; miR-557 comprises miR-557; miR-6801 miR-6801-3p; miR-4420 comprises miR-4420; miR-570 comprises miR-570-3p; miR-155 comprises miR-155-5p; miR-199 comprises miR-199-5p; miR-520d comprises miR-520d-5p; miR-424 comprises miR-424-5p; miR-200 comprises miR-200a-5p, miR-200a-3p, miR-200b-5p, miR-200b-3p, miR-200c-5p, or miR-200c-3p, or any combination thereof.

211. The method of claim 210, comprising
(1) measuring the level of miR-17,
wherein optionally miR-17 comprises miR-17-5p;
(2) measuring the level of miR-19b,
wherein optionally miR-19b comprises miR-19b-3p;
(3) measuring the level of miR-21,
wherein optionally miR-21 comprises miR-21-5p or miR-21-3p;
(4) measuring the level of miR-15b,
wherein optionally miR-15b comprises miR-15b-5p;
(5) measuring the level of miR-19a,
wherein optionally miR-19a comprises miR-19a-3p;
(6) measuring the level of miR-664a,
wherein optionally miR-664a comprises miR-664a-3p;
(7) measuring the level of miR-381,
wherein optionally miR-381 comprises miR-381-3p;
(8) measuring the level of miR-23a,
wherein optionally miR-23a comprises miR-23a-3p;
(9) measuring the level of miR-654,
wherein optionally miR-654 comprises miR-654-3p;
(10) measuring the level of miR-24,
wherein optionally miR-24 comprises miR-24-3p;
(11) measuring the level of Let-7b,
wherein optionally Let-7b comprises Let-7b-5p;
(12) measuring the level of miR-20a,
wherein optionally miR-20a comprises miR-20a-5p;
(13) measuring the level of miR-22,
wherein optionally miR-22 comprises miR-22-5p or miR-22-3p;
(14) measuring the level of miR-34c,
wherein optionally miR-34c comprises miR-34c-3p;
(15) measuring the level of miR-155,
wherein optionally miR-155 comprises miR-155-5p;
(16) measuring the level of miR-1287,
wherein optionally miR-1287 comprises miR-1287-5p;
(17) measuring the level of miR-625,
wherein optionally miR-625 comprises miR-625-5p;
(18) measuring the level of miR-1294,
wherein optionally miR-1294 comprises miR-1294-5p;
(19) measuring the level of miR-7704;
(20) measuring the level of miR-221,
wherein optionally miR-221 comprises miR-221-5p;
(21) measuring the level of miR-340,
wherein optionally miR-340 comprises miR-340-5p;
(22) measuring the level of miR-450b,
wherein optionally miR-450b comprises miR-450b-5p;
(23) measuring the level of miR-548e,
wherein optionally miR-548e comprises miR-548e-3p; or
(24) measuring the level of miR-502,
wherein optionally miR-502 comprises miR-502-3p.

212. The method of claim 210, wherein
(1) the miRNA is selected from: miR-17, miR-19b, miR-21, miR-15b, miR-19a, miR-664a, miR-381, miR-23a, miR-654, and miR-24;
wherein optionally the miRNA is selected from: miR-17-5p, miR-19b-3p, miR-21-5p, miR-15b-5p, miR-19a-3p, miR-664a-3p, miR-381-3p, miR-23a-3p, miR-654-3p, and miR-24-3p;
(2) the miRNA is selected from: miR-21, miR-23a, miR-1287, miR-625, miR-1294, miR-7704, miR-221, miR-340, miR-450b, miR-548e, and miR-502;
wherein optionally the miRNA is selected from: miR-21-3p, miR-22-5p, miR-1287-5p, miR-625-5p, miR-1294-5p, miR-7704, miR-221-5p, miR-340-5p, miR-450b-5p, miR-548e-3p, and miR-502-3p;
(3) the miRNA is selected from: miR-17, miR-23a, Let-7b, miR-20a, miR-22, miR-155, miR-34c, and miR-199;
wherein optionally the miRNA is selected from: miR-17-5p, miR-23a-3p, Let-7b-5p, miR-20a-5p, miR-22-3p, miR-155-5p, miR-34c-3p, and miR-199-5p;
(4) the miRNA is selected from: miR-17, miR-23a, Let-7b, miR-20a, miR-22, and miR-155;
wherein optionally the miRNA is selected from: miR-17-5p, miR-23a-3p, Let-7b-5p, miR-20a-5p, miR-22-3p, and miR-155-5p; or
(5) the miRNA is selected from: Let-7b, miR-20a, miR-22, miR-34c, miR-574, miR-9, miR-299, miR-6789, miR-593, miR-346, miR-34a, miR-449a, miR-7109, miR-3907, miR-557, miR-6801, miR-4420, and miR-570;
wherein optionally the miRNA is selected from: Let-7b-5p, miR-20a-5p, miR-22-3p, miR-34c-3p, miR-574-3p, miR-9-5p, miR-299-3p, miR-299-5p, miR-6789-5p, miR-593-5p, miR-346, miR-34a-5p, miR-449a, miR-7109-5p, miR-3907, miR-557, miR-6801-3p, miR-4420, and miR-570-3p.

213. The method of claim 210, comprising measuring levels of at least two, at least three, at least four, at least five, at least six, at least seven, at least eight, at least nine, or at least ten miRNAs, wherein optionally the method comprises
(1) measuring levels of miR-17, miR-19b, miR-21, miR-15b, miR-19a, miR-664a, miR-381, miR-23a, miR-654, and miR-24;
optionally measuring levels of: miR-17-5p, miR-19b-3p, miR-21-5p, miR-15b-5p, miR-19a-3p, miR-664a-3p, miR-381-3p, miR-23a-3p, miR-654-3p, and miR-24-3p;
(2) measuring levels of miRNAs that are selected from: miR-21, miR-23a, miR-1287, miR-625, miR-1294, miR-7704, miR-221, miR-340, miR-450b, miR-548e, and miR-502;
wherein optionally the miRNAs are selected from: miR-21-3p, miR-22-5p, miR-1287-5p, miR-625-5p, miR-1294-5p, miR-7704, miR-221-5p, miR-340-5p, miR-450b-5p, miR-548e-3p, and miR-502-3p;
(3) measuring levels of: miR-17, miR-23a, Let-7b, miR-20a, miR-22, miR-155, miR-34c, and miR-199;
optionally measuring levels of: miR-17-5p, miR-23a-3p, Let-7b-5p, miR-20a-5p, miR-22-3p, miR-155-5p, miR-34c-3p, and miR-199-5p;
(4) measuring levels of: miR-17, miR-23a, Let-7b, miR-20a, miR-22, and miR-155;
optionally measuring levels of: miR-17-5p, miR-23a-3p, Let-7b-5p, miR-20a-5p, miR-22-3p, and miR-155-5p;
optionally further measuring levels of miR-574 and miR-520d;
optionally further measuring levels of miR-574-3p and miR-520d-5p; or
(5) measuring levels of at least two miRNAs selected from: miR-34c, miR-20a, miR-17, and miR-22;
optionally measuring levels of at least two miRNAs selected from: miR-34c-3p, miR-20a-5p, miR-17-5p, and miR-22-3p;
optionally measuring levels of miR-20a-5p and miR-34c-3p, levels of miR-17-5p and miR-34c-3p, levels of miR-22-3p and miR-34c-3p, or levels of miR-17-5p, miR-20a-5p, miR-22-3p and miR-34c-3p.

214. The method of claim 210, wherein the miRNA level is normalized using an endogenous control;
wherein optionally the endogenous control is (1) selected from: miR-16-5p, miR-92a-3p, miR-652-3p, miR-92b-3p, miR-532-5p, miR-338-5p, miR-374a-5p, and miR-421; or
(2) selected from: miR-181a-2-3p, miR-181d-5p, Let-7g-5p, miR-181a-3p, miR-24-2-5p, miR-130b-3p, miR-628-3p, miR-19a-3p, miR-181a-5p and miR-424-3p.

215. The method of claim 210, wherein the gynecological disease is endometriosis;
optionally further comprising determining the difference between the measured level and the reference level, and comparing the difference with a cutoff value, wherein a higher difference than the cutoff value indicates that the subject also has adenomyosis.

216. The method of claim 210, wherein the miRNA comprises miR-155, miR-22, Let-7b, miR-23a, miR-17, or miR-20a, or any combination thereof, and wherein the method comprises determining the difference between the measured level and the reference level and comparing the difference with a cutoff value, wherein a higher difference than the cutoff value indicates the subject has endometriosis, and a lower difference than the cutoff value indicates the subject has a different gynecological disease;
wherein optionally the miRNA comprises miR-155-5p, miR-22-3p, Let-7b-5p, miR-23a-3p, miR-17-5p, miR-20a-5p, or any combination thereof.

217. The method of claim 210, wherein the level of the miRNA is measured using in situ RNA hybridization (e.g., fluorescence in situ hybridization, or FISH), quantitative polymerase chain reaction (qPCR), real-time polymerase chain reaction (RT-PCR), droplet digital PCR (ddPCR), microarray analysis, next generation sequencing (NGS), northern blotting, or a luminex-based assay;
wherein optionally the miRNA level is measured using NGS, qPCR, or ddPCR;
wherein optionally the sample is treated with DNase before the miRNA level is measured.

218. The method of claim 210, wherein the sample is a peripheral blood sample, a menstrual blood sample, a pap smear sample, a uterus biopsy sample, an endometrial biopsy sample, a plasma sample or a serum sample;
optionally further comprising obtaining the sample from the subject;
wherein optionally the sample is obtained during the secretory phase or proliferative phase of a menstrual cycle;
wherein optionally the sample is a serum sample;
wherein optionally the serum sample is obtained within 2 hours of blood draw.

219. The method of claim 210, further comprising measuring the expression level of CA-125 (Cancer Antigen 125) in a sample of the subject, comparing the expression level to a reference level; wherein an increased expression level indicates that the subject is likely to have endometriosis;
wherein optionally the expression level is increased by at least 20%, at least 50%, at least 80%, or at least 90% compared to the reference level;
wherein optionally measuring the protein level of CA-125;
wherein optionally the protein level is measured by immunohistochemistry (IHC), immunocytochemistry (ICC), an enzyme-linked immunosorbent assay (ELISA), immunoblotting assay (e.g., Western blot), flow cytometry (FACS), a fluorescent immunosorbent assay (FIA), a chemiluminescence immunoassay (CIA), an electrochemiluminescence immunoassay (ECLIA), a radioimmunoassay (RIA), a solid phase radioimmunoassay (SPROA), or a dot/line-immunoblot assay;
wherein optionally the sample for measuring CA-125 is a blood sample or a peritoneal fluid sample;
wherein optionally the sample for measuring CA-125 is a serum sample;
wherein optionally the serum sample is obtained within 2 hours of blood draw;
wherein optionally the reference level is 15 U/mL, 20 U/mL, 30 U/mL, or 35 U/mL.

220. A method of assessing whether a subject has endometriosis, comprising
(a) measuring the expression level of CA-125 (Cancer Antigen 125) in a sample of the subject, wherein the sample is obtained during the secretory phase of a menstrual cycle; and
(b) comparing the expression level to a reference level; wherein an increased expression level indicates that the subject is likely to have endometriosis;
wherein optionally the expression level is increased by at least 20%, at least 50%, at least 80%, or at least 90% compared to the reference level;
wherein optionally measuring the protein level of CA-125;
wherein optionally the protein level is measured by immunohistochemistry (IHC), immunocytochemistry (ICC), an enzyme-linked immunosorbent assay (ELISA), immunoblotting assay (e.g., Western blot), flow cytometry (FACS), a fluorescent immunosorbent assay (FIA), a chemiluminescence immunoassay (CIA), an electrochemiluminescence immunoassay (ECLIA), a radioimmunoassay (RIA), a solid phase radioimmunoassay (SPROA), or a dot/line-immunoblot assay;
wherein optionally the sample for measuring CA-125 is a blood sample or a peritoneal fluid sample;
wherein optionally the sample for measuring CA-125 is a serum sample;
wherein optionally the serum sample is obtained within 2 hours of blood draw;
wherein optionally the reference level is 15 U/mL, 20 U/mL, 30 U/mL, or 35 U/mL.

221. The method of claim 210, wherein the subject is a human female;
wherein optionally the subject has a clinical indicator for endometriosis, wherein the indicator is dysmenorrhea, lower abdominal pain, chronic pelvic pain, deep dyspareunia, dysuria, dyschezia, fatigue, abnormal menstrual bleeding, or infertility, or any combination thereof.

222. The method of claim 210, further comprising administering a treatment for endometriosis to the subject;
wherein optionally the treatment for endometriosis is (1) pain medication, a hormone therapy, or a surgical procedure, optionally a surgical procedure selected from: laparoscopy, laparotomy, presacral neurectomy, laparoscopic uterine nerve ablation (LUNA), and hysterectomy; or
(2) a hormonal treatment, an oral contraceptive, a progestin, a GnRH agonist, an androgen, an aromatase inhibitor, a non-steroidal anti-inflammatory drug, or any combination thereof.

223. A kit for assessing whether a subject has a gynecological disease comprising a means for measuring a level of a miRNA selected from: miR-17, miR-19b, miR-21, miR-15b, miR-19a, miR-664a, miR-381, miR-23a, miR-654, miR-24, Let-7b, miR-20a, miR-22, miR-34c, miR-1287, miR-625, miR-1294, miR-7704, miR-221, miR-340, miR-450b, miR-548e, miR-502, miR-574, miR-9, miR-299, miR-6789, miR-593, miR-346, miR-34a, miR-449a, miR-7109, miR-3907, miR-557, miR-6801, miR-4420, miR-570, miR-155, miR-199, miR-520d, miR-424, and miR-200 in a sample of the subject, and an ancillary reagent;
wherein optionally: miR-17 comprises miR-17-5p, miR-17-3p, or both; miR-19b comprises miR-19b-5p, miR-19b-3p, or both; miR-21 comprises miR-21-5p, miR-21-3p, or both; miR-15b comprises miR-15b-5p, miR-15b-3p, or both; miR-19a comprises miR-19a-5p, miR-19a-3p, or both; miR-664a comprises miR-664a-5p, miR-664a-3p, or both; miR-381 comprises miR-381-5p, miR-381-3p, or both; miR-23a comprises miR-23a-5p, miR-23a-3p, or both; miR-654 comprises miR-654-5p, miR-654-3p, or both; miR-24 comprises miR-24-5p, miR-24-3p, or both; Let-7b comprises Let-7b-5p, Let-7b-3p, or both; miR-20a comprises miR-20a-5p, miR-20a-3p, or both; miR-22 comprises miR-22-5p, miR-22-3p, or both; miR-34c comprises miR-34c-5p, miR-34c-3p, or both; miR-1287 comprises miR-1287-5p, miR-1287-3p, or both; miR-625 comprises miR-625-5p, miR-625-3p, or both; miR-1294 comprises miR-1294-5p, miR-1294-3p, or both; miR-7704 comprises miR-7704; miR-221 comprises miR-221-5p, miR-221-3p, or both; miR-340 comprises miR-340-5p, miR-340-3p, or both; miR-450b comprises miR-450b-5p, miR-450b-3p, or both; miR-548e comprises miR-548e-5p, miR-548e-3p, or both; miR-502 comprises miR-502-5p, miR-502-3p, or both; miR-574 comprises miR-574-5p, miR-574-3p, or both; miR-9 comprises miR-9-5p, miR-9-3p, or both; miR-299 comprises miR-299-5p, miR-299-3p, or both; miR-6789 comprises miR-6789-5p, miR-6789-3p, or both; miR-593 comprises miR-593-5p, miR-593-3p, or both; miR-346 comprises miR-346-5p, miR-346-3p, or both; miR-34a comprises miR-34a-5p, miR-34a-3p, or both; miR-449a comprises miR-449a-5p, miR-449a-3p, or both; miR-7109 comprises miR-7109-5p, miR-7109-3p, or both; miR-3907 comprises miR-3907; miR-557 comprises miR-557; miR-6801 comprises miR-6801-5p, miR-6801-3p or both; miR-4420 comprises miR-4420; miR-570 comprises miR-570-5p, miR-570-3p, or both; miR-155 comprises miR-155-5p, miR-155-3p, or both; miR-199 comprises miR-199-5p, miR-199-3p, or both; miR-520d comprises miR-520d-5p, miR-520d-3p, or both; miR-424 comprises miR-424-5p, miR-424-3p, or both; miR-200 comprises miR-200a-5p, miR-200a-3p, miR-200b-5p, miR-200b-3p, miR-200c-5p, or miR-200c-3p, or any combination thereof,
wherein optionally: miR-17 comprises miR-17-5p; miR-19b comprises miR-19b-3p; miR-21 comprises miR-21-5p, miR-21-3p, or both; miR-15b comprises miR-15b-5p; miR-19a comprises miR-19a-3p; miR-664a comprises miR-664a-3p; miR-381 comprises miR-381-3p; miR-23a comprises miR-23a-3p; miR-654 comprises miR-654-3p; miR-24 comprises miR-24-3p; Let-7b comprises Let-7b-5p; miR-20a comprises miR-20a-5p; miR-22 comprises miR-22-5p, miR-22-3p, or both; miR-34c comprises miR-34c-3p; miR-1287 comprises miR-1287-5p; miR-625 comprises miR-625-5p; miR-1294 comprises miR-1294-5p; miR-7704 comprises miR-7704; miR-221 comprises miR-221-5p; miR-340 comprises miR-340-5p; miR-450b comprises miR-450b-5p; miR-548e comprises miR-548e-3p; miR-502 comprises miR-502-3p; miR-574 comprises miR-574-3p; miR-9 comprises miR-9-5p; miR-299 comprises miR-299-5p, miR-299-3p, or both; miR-6789 comprises miR-6789-5p; miR-593 comprises miR-593-5p; miR-346 comprises miR-346-5p, miR-346-3p, or both; miR-34a comprises miR-34a-5p; miR-449a comprises miR-449a-5p, miR-449a-3p, or both; miR-7109 comprises miR-7109-5p; miR-3907 comprises miR-3907; miR-557 comprises miR-557; miR-6801 miR-6801-3p; miR-4420 comprises miR-4420; miR-570 comprises miR-570-3p; miR-155 comprises miR-155-5p; miR-199 comprises miR-199-5p; miR-520d comprises miR-520d-5p; miR-424 comprises miR-424-5p; miR-200 comprises miR-200a-5p, miR-200a-3p, miR-200b-5p, miR-200b-3p, miR-200c-5p, or miR-200c-3p, or any combination thereof.

224. The kit of claim 223, comprising
(1) a means for measuring the level of miR-17,
wherein optionally miR-17 comprises miR-17-5p;
(2) a means for measuring the level of miR-19b,
wherein optionally miR-19b comprises miR-19b-3p;
(3) a means for measuring the level of miR-21,
wherein optionally miR-21 comprises miR-21-5p or miR-21-3p;
(4) a means for measuring the level of miR-15b,
wherein optionally miR-15b comprises miR-15b-5p;
(5) a means for measuring the level of miR-19a,
wherein optionally miR-19a comprises miR-19a-3p;
(6) a means for measuring the level of miR-664a,
wherein optionally miR-664a comprises miR-664a-3p;
(7) a means for measuring the level of miR-381,
wherein optionally miR-381 comprises miR-381-3p;
(8) a means for measuring the level of miR-23a,
wherein optionally miR-23a comprises miR-23a-3p;
(9) a means for measuring the level of miR-654,
wherein optionally miR-654 comprises miR-654-3p;
(10) a means for measuring the level of miR-24,
wherein optionally miR-24 comprises miR-24-3p;
(11) a means for measuring the level of Let-7b,
wherein optionally Let-7b comprises Let-7b-5p;
(12) a means for measuring the level of miR-20a,
wherein optionally miR-20a comprises miR-20a-5p;
(13) a means for measuring the level of miR-22,
wherein optionally miR-22 comprises miR-22-5p or miR-22-3p;
(14) a means for measuring the level of miR-34c,
wherein optionally miR-34c comprises miR-34c-3p;
(15) a means for measuring the level of miR-155,
wherein optionally miR-155 comprises miR155-5p;
(16) a means for measuring the level of miR-1287,
wherein optionally miR-1287 comprises miR-1287-5p;
(17) a means for measuring the level of miR-625,
wherein optionally miR-625 comprises miR-625-5p;
(18) a means for measuring the level of miR-1294,
wherein optionally miR-1294 comprises miR-1294-5p;
(19) a means for measuring the level of miR-7704;
(20) a means for measuring the level of miR-221,
wherein optionally miR-221 comprises miR-221-5p;
(21) a means for measuring the level of miR-340,
wherein optionally miR-340 comprises miR-340-5p;
(22) a means for measuring the level of miR-450b,
wherein optionally miR-450b comprises miR-450b-5p;
(23) a means for measuring the level of miR-548e,
wherein optionally miR-548e comprises miR-548e-3p; or
(24) a means for measuring the level of miR-502,
wherein optionally miR-502 comprises miR-502-3p.

225. The kit of claim 223, wherein
(1) the miRNA is selected from: miR-17, miR-19b, miR-21, miR-15b, miR-19a, miR-664a, miR-381, miR-23a, miR-654, and miR-24;
wherein optionally the miRNA is selected from: miR-17-5p, miR-19b-3p, miR-21-5p, miR-15b-5p, miR-19a-3p, miR-664a-3p, miR-381-3p, miR-23a-3p, miR-654-3p, and miR-24-3p;
(2) the miRNA is selected from: miR-21, miR-23a, miR-1287, miR-625, miR-1294, miR-7704, miR-221, miR-340, miR-450b, miR-548e, and miR-502;
wherein optionally the miRNA is selected from: miR-21-3p, miR-22-5p, miR-1287-5p, miR-625-5p, miR-1294-5p, miR-7704, miR-221-5p, miR-340-5p, miR-450b-5p, miR-548e-3p, and miR-502-3p;
(3) the miRNA is selected from: miR-17, miR-23a, Let-7b, miR-20a, miR-22, miR-155, miR-34c, and miR-199;
wherein optionally the miRNA is selected from: miR-17-5p, miR-23a-3p, Let-7b-5p, miR-20a-5p, miR-22-3p, miR-155-5p, miR-34c-3p, and miR-199-5p;
(4) the miRNA is selected from: miR-17, miR-23a, Let-7b, miR-20a, miR-22, and miR-155;
wherein optionally the miRNA is selected from: miR-17-5p, miR-23a-3p, Let-7b-5p, miR-20a-5p, miR-22-3p, and miR-155-5p; or
(5) the miRNA is selected from: Let-7b, miR-20a, miR-22, miR-34c, miR-574, miR-9, miR-299, miR-6789, miR-593, miR-346, miR-34a, miR-449a, miR-7109, miR-3907, miR-557, miR-6801, miR-4420, and miR-570;
wherein optionally the miRNA is selected from: Let-7b-5p, miR-20a-5p, miR-22-3p, miR-34c-3p, miR-574-3p, miR-9-5p, miR-299-3p, miR-299-5p, miR-6789-5p, miR-593-5p, miR-346, miR-34a-5p, miR-449a, miR-7109-5p, miR-3907, miR-557, miR-6801-3p, miR-4420, and miR-570-3p.

226. The kit of claim 223, comprising means for measuring levels of at least two, at least three, at least four, at least five, at least six, at least seven, or at least eight miRNAs; wherein optionally the kit comprises
(1) means for measuring levels of miR-17, miR-19b, miR-21, miR-15b, miR-19a, miR-664a, miR-381, miR-23a, miR-654, and miR-24;
optionally means for measuring levels of: miR-17-5p, miR-19b-3p, miR-21-5p, miR-15b-5p, miR-19a-3p, miR-664a-3p, miR-381-3p, miR-23a-3p, miR-654-3p, and miR-24-3p;
(2) means for measuring levels of: miR-21, miR-23a, miR-1287, miR-625, miR-1294, miR-7704, miR-221, miR-340, miR-450b, miR-548e, and miR-502;
optionally means for measuring levels of: miR-21-3p, miR-22-5p, miR-1287-5p, miR-625-5p, miR-1294-5p, miR-7704, miR-221-5p, miR-340-5p, miR-450b-5p, miR-548e-3p, and miR-502-3p;
(3) means for measuring levels of: miR-17, miR-23a, Let-7b, miR-20a, miR-22, miR-155, miR-34c, and miR-199;
optionally means for measuring levels of: miR-17-5p, miR-23a-3p, Let-7b-5p, miR-20a-5p, miR-22-3p, miR-155-5p, miR-34c-3p, and miR-199-5p;
(4) means for measuring levels of: miR-17, miR-23a, Let-7b, miR-20a, miR-22, and miR-155;
optionally means for measuring levels of: miR-17-5p, miR-23a-3p, Let-7b-5p, miR-20a-5p, miR-22-3p, and miR-155-5p;
optionally further comprising means for measuring levels of miR-574 and miR-520d;
optionally further comprising means for measuring levels of miR-574-3p and miR-520d-5p; or
(5) means for measuring levels of at least two miRNAs selected from: miR-34c, miR-20a, miR-17, and miR-22;
optionally means for measuring levels of at least two miRNAs selected from: miR-34c-3p, miR-20a-5p, miR-17-5p, and miR-22-3p;
optionally means for measuring levels of miR-20a-5p and miR-34c-3p, levels of miR-17-5p and miR-34c-3p, levels of miR-22-3p and miR-34c-3p, or levels of miR-17-5p, miR-22-3p and miR-34c-3p.

227. The kit of claim 223, further comprising means for measuring an endogenous control;
wherein optionally the endogenous control is (1) selected from: miR-16-5p, miR-92a-3p, miR-652-3p, miR-92b-3p, miR-532-5p, miR-338-5p, miR-374a-5p, and miR-421; or
(2) selected from: miR-181a-2-3p, miR-181d-5p, Let-7g-5p, miR-181a-3p, miR-24-2-5p, miR-130b-3p, miR-628-3p, miR-19a-3p, miR-181a-5p and miR-424-3p.

228. The kit of claim 223, wherein the gynecological disease is endometriosis;
wherein optionally the gynecological disease is endometriosis in combination with adenomyosis.

229. The kit of claim 223, wherein (1) the means comprises one or more nucleic acid probes for detecting the miRNA; or
(2) the means comprises one or more primer pairs for amplifying the miRNA;
wherein optionally the primer pair comprises a forward primer and a reverse primer having nucleotide sequences of (1) SEQ ID NOs:3 and 1, respectively; (2) SEQ ID NOs:4 and 1, respectively; (3) SEQ ID NOs:5 and 1, respectively; (4) SEQ ID NOs:6 and 1, respectively; (5) SEQ ID NOs:7 and 1, respectively; or (6) SEQ ID NOs:8 and 1, respectively;
optionally further comprising a primer pair for measuring an endogenous control, comprising a forward primer and a reverse primer having the nucleotide sequences of (1) SEQ ID NOs:9 and 1, respectively; (2) SEQ ID NOs:10 and 2, respectively; or (3) SEQ ID NOs:11 and 1, respectively;
optionally further comprising a reverse transcriptase, a DNA polymerase, or a DNA polymerase with reverse transcriptase activity;
optionally further comprising a DNase.

230. The kit of claim 223, wherein
(1) the ancillary reagent comprises a reaction buffer, a dilution buffer, or a wash buffer, or any combination thereof;
(2) said kit further comprises a solid support;
(3) said kit further comprises a container for sample collection; or
(4) said kit further comprises instructions; or any combination of (1)-(4).
